SEQUENCE LISTING (1040 sequences)

SEQ ID NO: 1 - DNA, 23 bp, synthetic construct
  source 1..23: mol_type = other DNA
gtggagactggaagcattctcaa

SEQ ID NO: 2 - DNA, 22 bp, synthetic construct
  source 1..22: mol_type = other DNA
tgcagagaagctctggaacttg

SEQ ID NO: 3 - DNA, 22 bp, synthetic construct
  source 1..22: mol_type = other DNA
aagcattgcccaggagaatagg

SEQ ID NO: 4 - DNA, 22 bp, synthetic construct
  source 1..22: mol_type = other DNA
ctccctggtcttcatctcacca

SEQ ID NO: 5 - DNA, 20 bp, synthetic construct
  source 1..20: mol_type = other DNA
agcgctcagtgaccttttgg

SEQ ID NO: 6 - DNA, 23 bp, synthetic construct
  source 1..23: mol_type = other DNA
tgcatcgctccctttagagacag

SEQ ID NO: 7 - DNA, 20 bp, synthetic construct
  source 1..20: mol_type = other DNA
cactgggagcaggagagtct

SEQ ID NO: 8 - DNA, 18 bp, synthetic construct
  source 1..18: mol_type = other DNA
ccggcacctctctcccat

SEQ ID NO: 9 - DNA, 24 bp, synthetic construct
  source 1..24: mol_type = other DNA
ttctcgtagttgggcaggttgtct

SEQ ID NO: 10 - DNA, 23 bp, synthetic construct
  source 1..23: mol_type = other DNA
tcagtcagctccctactcaccat

SEQ ID NO: 11 - DNA, 23 bp, synthetic construct
  source 1..23: mol_type = other DNA
aagatgacgcgggcattgttcct

SEQ ID NO: 12 - DNA, 23 bp, synthetic construct
  source 1..23: mol_type = other DNA
gacccaagcagttccaagtaagg

SEQ ID NO: 13 - DNA, 23 bp, synthetic construct
  source 1..23: mol_type = other DNA
tggtctacgcgcatagcaaggtg

SEQ ID NO: 14 - DNA, 22 bp, synthetic construct
  source 1..22: mol_type = other DNA
ggcgaggtcagaggggctgtct

SEQ ID NO: 15 - DNA, 27 bp, synthetic construct
  source 1..27: mol_type = other DNA
gctccaagaaattcttggctatatgga

SEQ ID NO: 16 - DNA, 24 bp, synthetic construct
  source 1..24: mol_type = other DNA
tcttctataggacaaggtgcaacg

SEQ ID NO: 17 - DNA, 23 bp, synthetic construct
  source 1..23: mol_type = other DNA
ttgtgggaagggcgttcatccat

SEQ ID NO: 18 - DNA, 22 bp, synthetic construct
  source 1..22: mol_type = other DNA
aggccagcaagtgtgacagtca

SEQ ID NO: 19 - DNA, 23 bp, synthetic construct
  source 1..23: mol_type = other DNA
ccattcttctccccagaatgcat

SEQ ID NO: 20 - DNA, 27 bp, synthetic construct
  source 1..27: mol_type = other DNA
ttgtgggaccgtatgtaaagaagatcc

SEQ ID NO: 21 - DNA, 22 bp, synthetic construct
  source 1..22: mol_type = other DNA
tggaagtaggcagtcagacgta

SEQ ID NO: 22 - DNA, 22 bp, synthetic construct
  source 1..22: mol_type = other DNA
tgtcttcctcaggtcatgttgg

SEQ ID NO: 23 - DNA, 27 bp, synthetic construct
  source 1..27: mol_type = other DNA
tcctgttatcactcatgagagtttctc

SEQ ID NO: 24 - DNA, 26 bp, synthetic construct
  source 1..26: mol_type = other DNA
ccaaggtcgcttaaaaacccaacaag

SEQ ID NO: 25 - DNA, 25 bp, synthetic construct
  source 1..25: mol_type = other DNA
agatcagcgtatgatgctcttccaa

SEQ ID NO: 26 - DNA, 25 bp, synthetic construct
  source 1..25: mol_type = other DNA
aaacctacatggccaacaactttgg

SEQ ID NO: 27 - DNA, 23 bp, synthetic construct
  source 1..23: mol_type = other DNA
aatcccagccacaccaaggtagt

SEQ ID NO: 28 - DNA, 25 bp, synthetic construct
  source 1..25: mol_type = other DNA
aaaatttccctgccttcttcccttc

SEQ ID NO: 29 - DNA, 24 bp, synthetic construct
  source 1..24: mol_type = other DNA
tgaggttgtgacagcaactgaggg

SEQ ID NO: 30 - DNA, 24 bp, synthetic construct
  source 1..24: mol_type = other DNA
caacctgtttcaggatctccagga

SEQ ID NO: 31 - DNA, 22 bp, synthetic construct
  source 1..22: mol_type = other DNA
tatttgaggagaggcagctcca

SEQ ID NO: 32 - DNA, 22 bp, synthetic construct
  source 1..22: mol_type = other DNA
ctgcaaactcgccatcaaactc

SEQ ID NO: 33 - DNA, 22 bp, synthetic construct
  source 1..22: mol_type = other DNA
tgagactgcagaggtatgatgc

SEQ ID NO: 34 - DNA, 25 bp, synthetic construct
  source 1..25: mol_type = other DNA
tccaggatctccaggtcatagccat

SEQ ID NO: 35 - DNA, 30 bp, synthetic construct
  source 1..30: mol_type = other DNA
acgtttaaatgacttctattcttttcccat

SEQ ID NO: 36 - DNA, 28 bp, synthetic construct
  source 1..28: mol_type = other DNA
agattaccttttcccgaacttcatcaga

SEQ ID NO: 37 - DNA, 23 bp, synthetic construct
  source 1..23: mol_type = other DNA
gcatctattcacaaggtcgcttc

SEQ ID NO: 38 - DNA, 26 bp, synthetic construct
  source 1..26: mol_type = other DNA
acagcagggactataaagaaccaggt

SEQ ID NO: 39 - DNA, 23 bp, synthetic construct
  source 1..23: mol_type = other DNA
tatggaatcccccagtctccatc

SEQ ID NO: 40 - DNA, 22 bp, synthetic construct
  source 1..22: mol_type = other DNA
aatttgggcatcattgcgcatg

SEQ ID NO: 41 - DNA, 24 bp, synthetic construct
  source 1..24: mol_type = other DNA
agaatggcttttaccggcacagag

SEQ ID NO: 42 - DNA, 22 bp, synthetic construct
  source 1..22: mol_type = other DNA
acaagagggaaggaaggaagga

SEQ ID NO: 43 - DNA, 24 bp, synthetic construct
  source 1..24: mol_type = other DNA
ccacttgtctctcatttggtcgaa

SEQ ID NO: 44 - DNA, 23 bp, synthetic construct
  source 1..23: mol_type = other DNA
cccttatgtgaaaatgggggtga

SEQ ID NO: 45 - DNA, 21 bp, synthetic construct
  source 1..21: mol_type = other DNA
agtttggttgaggcagcacaa

SEQ ID NO: 46 - DNA, 24 bp, synthetic construct
  source 1..24: mol_type = other DNA
accaggtgcctctcaaattcttgc

SEQ ID NO: 47 - DNA, 25 bp, synthetic construct
  source 1..25: mol_type = other DNA
ggacaggcttattgactgctgtggc

SEQ ID NO: 48 - DNA, 25 bp, synthetic construct
  source 1..25: mol_type = other DNA
ttaccagacattccacatgcttcct

SEQ ID NO: 49 - DNA, 22 bp, synthetic construct
  source 1..22: mol_type = other DNA
aagggttatagggcaccttcca

SEQ ID NO: 50 - DNA, 25 bp, synthetic construct
  source 1..25: mol_type = other DNA
tgaagccacattctctactcggtga

SEQ ID NO: 51 - DNA, 24 bp, synthetic construct
  source 1..24: mol_type = other DNA
tcctgtacacagctctgtgtctct

SEQ ID NO: 52 - DNA, 24 bp, synthetic construct
  source 1..24: mol_type = other DNA
ttttcttcaggtccttgcacttgg

SEQ ID NO: 53 - DNA, 28 bp, synthetic construct
  source 1..28: mol_type = other DNA
ccataactgaacccctttcttagttgaa

SEQ ID NO: 54 - DNA, 28 bp, synthetic construct
  source 1..28: mol_type = other DNA
gagcaatgtgaattcataccttggtcag

SEQ ID NO: 55 - DNA, 27 bp, synthetic construct
  source 1..27: mol_type = other DNA
gtttattcacgcaggattaaaagggaa

SEQ ID NO: 56 - DNA, 33 bp, synthetic construct
  source 1..33: mol_type = other DNA
ctttgctatttgctgaagtttgatatctatgtg

SEQ ID NO: 57 - DNA, 26 bp, synthetic construct
  source 1..26: mol_type = other DNA
cctataggtggtttcttcagctctat

SEQ ID NO: 58 - DNA, 27 bp, synthetic construct
  source 1..27: mol_type = other DNA
aatacaggatgacattggattcctacc

SEQ ID NO: 59 - DNA, 23 bp, synthetic construct
  source 1..23: mol_type = other DNA
agaaagacaagtgccctgaagac

SEQ ID NO: 60 - DNA, 23 bp, synthetic construct
  source 1..23: mol_type = other DNA
tttggatgtactgggagtggaga

SEQ ID NO: 61 - DNA, 21 bp, synthetic construct
  source 1..21: mol_type = other DNA
tctcgccgccatttatccttc

SEQ ID NO: 62 - DNA, 22 bp, synthetic construct
  source 1..22: mol_type = other DNA
gattttggcactcagagcacca

SEQ ID NO: 63 - DNA, 22 bp, synthetic construct
  source 1..22: mol_type = other DNA
tcatgatggctctgtggaggta

SEQ ID NO: 64 - DNA, 22 bp, synthetic construct
  source 1..22: mol_type = other DNA
aggatactgaggaagccaggtt

SEQ ID NO: 65 - DNA, 20 bp, synthetic construct
  source 1..20: mol_type = other DNA
aattgagcttggctggcagt

SEQ ID NO: 66 - DNA, 21 bp, synthetic construct
  source 1..21: mol_type = other DNA
ttctgccttttcccgctatgg

SEQ ID NO: 67 - DNA, 24 bp, synthetic construct
  source 1..24: mol_type = other DNA
tgttaggtcgtgacagagtggtgg

SEQ ID NO: 68 - DNA, 23 bp, synthetic construct
  source 1..23: mol_type = other DNA
aatgaacactatgggcgctcttg

SEQ ID NO: 69 - DNA, 25 bp, synthetic construct
  source 1..25: mol_type = other DNA
ccctctcgtctgaaacaaaggttac

SEQ ID NO: 70 - DNA, 22 bp, synthetic construct
  source 1..22: mol_type = other DNA
ttttcctccttctcctcctcct

SEQ ID NO: 71 - DNA, 24 bp, synthetic construct
  source 1..24: mol_type = other DNA
ggggattgacgatgtgattgagac

SEQ ID NO: 72 - DNA, 24 bp, synthetic construct
  source 1..24: mol_type = other DNA
ggccaactgttgccgttttctctg

SEQ ID NO: 73 - DNA, 23 bp, synthetic construct
  source 1..23: mol_type = other DNA
aagtgccagagtcagcattctgg

SEQ ID NO: 74 - DNA, 24 bp, synthetic construct
  source 1..24: mol_type = other DNA
aaatgtttgctgacctctccctga

SEQ ID NO: 75 - DNA, 25 bp, synthetic construct
  source 1..25: mol_type = other DNA
cagcagagctttagcaaggaagcaa

SEQ ID NO: 76 - DNA, 24 bp, synthetic construct
  source 1..24: mol_type = other DNA
gggaaagattagagcctgggcact

SEQ ID NO: 77 - DNA, 20 bp, synthetic construct
  source 1..20: mol_type = other DNA
tagagaggcagctgcggaac

SEQ ID NO: 78 - DNA, 20 bp, synthetic construct
  source 1..20: mol_type = other DNA
gcccggcatccccggagaga

SEQ ID NO: 79 - DNA, 27 bp, synthetic construct
  source 1..27: mol_type = other DNA
ctggtcggacatgaaataacttgctat

SEQ ID NO: 80 - DNA, 28 bp, synthetic construct
  source 1..28: mol_type = other DNA
ctactttgacccatgttaggaactcaga

SEQ ID NO: 81 - DNA, 23 bp, synthetic construct
  source 1..23: mol_type = other DNA
aggtaagcccagaagacaaagca

SEQ ID NO: 82 - DNA, 24 bp, synthetic construct
  source 1..24: mol_type = other DNA
tgaagataggccctagtgctgagt

SEQ ID NO: 83 - DNA, 26 bp, synthetic construct
  source 1..26: mol_type = other DNA
gccatctgactgtttctcagagtcaa

SEQ ID NO: 84 - DNA, 22 bp, synthetic construct
  source 1..22: mol_type = other DNA
ctacctggatttgccgaagcaa

SEQ ID NO: 85 - DNA, 23 bp, synthetic construct
  source 1..23: mol_type = other DNA
ttccatgtggattccacctgtgt

SEQ ID NO: 86 - DNA, 24 bp, synthetic construct
  source 1..24: mol_type = other DNA
taagtaatcggggatgttccctga

SEQ ID NO: 87 - DNA, 24 bp, synthetic construct
  source 1..24: mol_type = other DNA
cacatcctggatggaaaacatgcc

SEQ ID NO: 88 - DNA, 23 bp, synthetic construct
  source 1..23: mol_type = other DNA
gcctttgctgtccagttatctct

SEQ ID NO: 89 - DNA, 27 bp, synthetic construct
  source 1..27: mol_type = other DNA
tctcccccagaaggtaagaagtactgc

SEQ ID NO: 90 - DNA, 23 bp, synthetic construct
  source 1..23: mol_type = other DNA
caacctccctgtataggcacagt

SEQ ID NO: 91 - DNA, 23 bp, synthetic construct
  source 1..23: mol_type = other DNA
gggcaagtctacgcttatgagac

SEQ ID NO: 92 - DNA, 27 bp, synthetic construct
  source 1..27: mol_type = other DNA
aaagtcagccagtgacttaataggaag

SEQ ID NO: 93 - DNA, 27 bp, synthetic construct
  source 1..27: mol_type = other DNA
tgaaagacatggcagcatatattacct

SEQ ID NO: 94 - DNA, 23 bp, synthetic construct
  source 1..23: mol_type = other DNA
gagccttttgtgtagggagacag

SEQ ID NO: 95 - DNA, 26 bp, synthetic construct
  source 1..26: mol_type = other DNA
gtgagagggagcacttcttacagtta

SEQ ID NO: 96 - DNA, 22 bp, synthetic construct
  source 1..22: mol_type = other DNA
aatcaggcaactgggactgaag

SEQ ID NO: 97 - DNA, 25 bp, synthetic construct
  source 1..25: mol_type = other DNA
gcatgagctctggttgtaagaagta

SEQ ID NO: 98 - DNA, 23 bp, synthetic construct
  source 1..23: mol_type = other DNA
gagtacaggcacttccaaaatgc

SEQ ID NO: 99 - DNA, 19 bp, synthetic construct
  source 1..19: mol_type = other DNA
caggctgggcaccacagca

SEQ ID NO: 100 - DNA, 18 bp, synthetic construct
  source 1..18: mol_type = other DNA
gggacggcagctcctcct

SEQ ID NO: 101 - DNA, 25 bp, synthetic construct
  source 1..25: mol_type = other DNA
tggtgcttctgaatgctcagacttc

SEQ ID NO: 102 - DNA, 23 bp, synthetic construct
  source 1..23: mol_type = other DNA
ttgggggagatgtagcaaacatg

SEQ ID NO: 103 - DNA, 30 bp, synthetic construct
  source 1..30: mol_type = other DNA
ccccttatatctttgtgcattttctcaaac

SEQ ID NO: 104 - DNA, 28 bp, synthetic construct
  source 1..28: mol_type = other DNA
ctgtaattctgtggcctcttttaggtga

SEQ ID NO: 105 - RNA, 25 nt, synthetic construct
  source 1..25: mol_type = other RNA
tcccgatacagtctctacgttggag

SEQ ID NO: 106 - DNA, 24 bp, synthetic construct
  source 1..24: mol_type = other DNA
gaagaggggaggaaatatcgagga

SEQ ID NO: 107 - DNA, 23 bp, synthetic construct
  source 1..23: mol_type = other DNA
acgtcattgcttatggtcactca

SEQ ID NO: 108 - DNA, 23 bp, synthetic construct
  source 1..23: mol_type = other DNA
acgtcattgcttatggtcactca

SEQ ID NO: 109 - DNA, 28 bp, synthetic construct
  source 1..28: mol_type = other DNA
ccagctcttaactctcttcatctcattt

SEQ ID NO: 110 - DNA, 24 bp, synthetic construct
  source 1..24: mol_type = other DNA
cttttctctccaagacgcaacagt

SEQ ID NO: 111 - DNA, 33 bp, synthetic construct
  source 1..33: mol_type = other DNA
ggctgttatcttcagtttctataaaaagagaga

SEQ ID NO: 112 - DNA, 28 bp, synthetic construct
  source 1..28: mol_type = other DNA
cagagctatttccacatagtagtgcatc

SEQ ID NO: 113 - DNA, 28 bp, synthetic construct
  source 1..28: mol_type = other DNA
gccatttgattttaagctctgggatgtg

SEQ ID NO: 114 - DNA, 27 bp, synthetic construct
  source 1..27: mol_type = other DNA
caagtcctcccatcactttctaacatg

SEQ ID NO: 115 - DNA, 23 bp, synthetic construct
  source 1..23: mol_type = other DNA
tactggtagtgaacggagaccac

SEQ ID NO: 116 - DNA, 22 bp, synthetic construct
  source 1..22: mol_type = other DNA
tcgtaaggtcacagctgtacac

SEQ ID NO: 117 - DNA, 23 bp, synthetic construct
  source 1..23: mol_type = other DNA
gtgctccacttttcactcccttt

SEQ ID NO: 118 - DNA, 27 bp, synthetic construct
  source 1..27: mol_type = other DNA
tgtagctgtattacgggtcacatcaac

SEQ ID NO: 119 - DNA, 23 bp, synthetic construct
  source 1..23: mol_type = other DNA
ctctgcatcacgtttttgtacca

SEQ ID NO: 120 - DNA, 23 bp, synthetic construct
  source 1..23: mol_type = other DNA
ctctgcatcacgtttttgtacca

SEQ ID NO: 121 - DNA, 25 bp, synthetic construct
  source 1..25: mol_type = other DNA
acctttaggtggtggttcaaccctt

SEQ ID NO: 122 - DNA, 22 bp, synthetic construct
  source 1..22: mol_type = other DNA
gatatgccatgttgtgggtggt

SEQ ID NO: 123 - DNA, 24 bp, synthetic construct
  source 1..24: mol_type = other DNA
tcactcggtgctacattgatgatc

SEQ ID NO: 124 - DNA, 27 bp, synthetic construct
  source 1..27: mol_type = other DNA
ccagctactgtacactccaaggtacag

SEQ ID NO: 125 - DNA, 29 bp, synthetic construct
  source 1..29: mol_type = other DNA
gatcctaagtctgatcaattgtttgtcag

SEQ ID NO: 126 - DNA, 23 bp, synthetic construct
  source 1..23: mol_type = other DNA
ttcttgcacatcatcagagctgt

SEQ ID NO: 127 - DNA, 30 bp, synthetic construct
  source 1..30: mol_type = other DNA
ctcccattggaagattcttcaatagacatg

SEQ ID NO: 128 - DNA, 25 bp, synthetic construct
  source 1..25: mol_type = other DNA
tacaagagcagcaacaaagtttcca

SEQ ID NO: 129 - DNA, 24 bp, synthetic construct
  source 1..24: mol_type = other DNA
tgtgggatatggatagggggaaag

SEQ ID NO: 130 - DNA, 24 bp, synthetic construct
  source 1..24: mol_type = other DNA
tgtcttctttgaacaggagcctct

SEQ ID NO: 131 - DNA, 19 bp, synthetic construct
  source 1..19: mol_type = other DNA
atccagcagccgctgtcca

SEQ ID NO: 132 - DNA, 18 bp, synthetic construct
  source 1..18: mol_type = other DNA
aaggcaaggcgggacaag

SEQ ID NO: 133 - DNA, 30 bp, synthetic construct
  source 1..30: mol_type = other DNA
agttgctttcttaggaaatgtagttgcttt

SEQ ID NO: 134 - DNA, 25 bp, synthetic construct
  source 1..25: mol_type = other DNA
taaaagatgggtgtttgaaggagca

SEQ ID NO: 135 - DNA, 25 bp, synthetic construct
  source 1..25: mol_type = other DNA
accttcttattcagccccgactgga

SEQ ID NO: 136 - DNA, 23 bp, synthetic construct
  source 1..23: mol_type = other DNA
ttccttttccagtgggtctgcta

SEQ ID NO: 137 - DNA, 27 bp, synthetic construct
  source 1..27: mol_type = other DNA
ggtatgtcgccctagaaaaatgaacaa

SEQ ID NO: 138 - DNA, 29 bp, synthetic construct
  source 1..29: mol_type = other DNA
atcgttttctcttaaggctactgttcatg

SEQ ID NO: 139 - DNA, 19 bp, synthetic construct
  source 1..19: mol_type = other DNA
gtcgtttccactgccagct

SEQ ID NO: 140 - DNA, 21 bp, synthetic construct
  source 1..21: mol_type = other DNA
agtgcccttcttttccccttg

SEQ ID NO: 141 - DNA, 20 bp, synthetic construct
  source 1..20: mol_type = other DNA
cgcctggaagcggacttcac

SEQ ID NO: 142 - DNA, 21 bp, synthetic construct
  source 1..21: mol_type = other DNA
tgaggagcgttggggacgaag

SEQ ID NO: 143 - DNA, 25 bp, synthetic construct
  source 1..25: mol_type = other DNA
cgaataggacatggccaagacaaaa

SEQ ID NO: 144 - DNA, 23 bp, synthetic construct
  source 1..23: mol_type = other DNA
ccaacagtcctgtggggttgact

SEQ ID NO: 145 - DNA, 30 bp, synthetic construct
  source 1..30: mol_type = other DNA
cgaagtgcattttctctattaacaagttca

SEQ ID NO: 146 - DNA, 25 bp, synthetic construct
  source 1..25: mol_type = other DNA
gttttgatgcagtgtttctcgatcc

SEQ ID NO: 147 - DNA, 24 bp, synthetic construct
  source 1..24: mol_type = other DNA
acaatcactttgctgagcgatcat

SEQ ID NO: 148 - DNA, 26 bp, synthetic construct
  source 1..26: mol_type = other DNA
caccctgaacttctttaaggagagca

SEQ ID NO: 149 - DNA, 21 bp, synthetic construct
  source 1..21: mol_type = other DNA
ggctgctgcagaagtgacacc

SEQ ID NO: 150 - DNA, 21 bp, synthetic construct
  source 1..21: mol_type = other DNA
acctccctgaggaaggacaac

SEQ ID NO: 151 - DNA, 20 bp, synthetic construct
  source 1..20: mol_type = other DNA
gatcttgggaggggctgttg

SEQ ID NO: 152 - DNA, 23 bp, synthetic construct
  source 1..23: mol_type = other DNA
tggtaacctgcacctcaatgtga

SEQ ID NO: 153 - DNA, 23 bp, synthetic construct
  source 1..23: mol_type = other DNA
actgtaagccatgatgcccttcc

SEQ ID NO: 154 - DNA, 23 bp, synthetic construct
  source 1..23: mol_type = other DNA
ccacagaggtacgcactgacaca

SEQ ID NO: 155 - DNA, 21 bp, synthetic construct
  source 1..21: mol_type = other DNA
aagagagtccacctcctgcat

SEQ ID NO: 156 - DNA, 21 bp, synthetic construct
  source 1..21: mol_type = other DNA
tcagataccaggaagccccat

SEQ ID NO: 157 - DNA, 24 bp, synthetic construct
  source 1..24: mol_type = other DNA
tgaggatgcaaaggctcagagaga

SEQ ID NO: 158 - DNA, 26 bp, synthetic construct
  source 1..26: mol_type = other DNA
aaggaaaagtccttccccatcattcc

SEQ ID NO: 159 - DNA, 23 bp, synthetic construct
  source 1..23: mol_type = other DNA
tgtctctgattccagagccagta

SEQ ID NO: 160 - DNA, 22 bp, synthetic construct
  source 1..22: mol_type = other DNA
atcagccctgtaggacttctga

SEQ ID NO: 161 - DNA, 25 bp, synthetic construct
  source 1..25: mol_type = other DNA
ctgtggtttgcttctctctccttcc

SEQ ID NO: 162 - DNA, 25 bp, synthetic construct
  source 1..25: mol_type = other DNA
tgctgtctgggcatttctgttctct

SEQ ID NO: 163 - DNA, 24 bp, synthetic construct
  source 1..24: mol_type = other DNA
tgtacccagaatctgtgctcaagt

SEQ ID NO: 164 - DNA, 26 bp, synthetic construct
  source 1..26: mol_type = other DNA
catcacatggaacgaactgtgtcatg

SEQ ID NO: 165 - DNA, 24 bp, synthetic construct
  source 1..24: mol_type = other DNA
accctgagtcagatccattgccac

SEQ ID NO: 166 - DNA, 24 bp, synthetic construct
  source 1..24: mol_type = other DNA
tggttctgaaccctccaacccaag

SEQ ID NO: 167 - DNA, 26 bp, synthetic construct
  source 1..26: mol_type = other DNA
caggttgtttccgtttacagagaaca

SEQ ID NO: 168 - DNA, 28 bp, synthetic construct
  source 1..28: mol_type = other DNA
ccaggatccccctctacatttaaagttg

SEQ ID NO: 169 - DNA, 22 bp, synthetic construct
  source 1..22: mol_type = other DNA
ttgctaggtccactctgggatc

SEQ ID NO: 170 - DNA, 23 bp, synthetic construct
  source 1..23: mol_type = other DNA
ggaagcatcaaactcgacctcct

SEQ ID NO: 171 - DNA, 25 bp, synthetic construct
  source 1..25: mol_type = other DNA
ctctcccatcttcctcctctcttct

SEQ ID NO: 172 - DNA, 21 bp, synthetic construct
  source 1..21: mol_type = other DNA
gagcccgatgtccaaaaggta

SEQ ID NO: 173 - DNA, 21 bp, synthetic construct
  source 1..21: mol_type = other DNA
ccaaaacatgatgcccctcca

SEQ ID NO: 174 - DNA, 22 bp, synthetic construct
  source 1..22: mol_type = other DNA
gtttaaggagcgcatcggctac

SEQ ID NO: 175 - DNA, 25 bp, synthetic construct
  source 1..25: mol_type = other DNA
tgtatcatctctctgttggggctac

SEQ ID NO: 176 - DNA, 23 bp, synthetic construct
  source 1..23: mol_type = other DNA
gtcaccgggacagcaataaagct

SEQ ID NO: 177 - DNA, 35 bp, synthetic construct
  source 1..35: mol_type = other DNA
gggaagctaatacaatatgtaaagttttctcataa

SEQ ID NO: 178 - DNA, 36 bp, synthetic construct
  source 1..36: mol_type = other DNA
tagaggcacatatttaaaataatttgaaatacggaa

SEQ ID NO: 179 - DNA, 24 bp, synthetic construct
  source 1..24: mol_type = other DNA
gctctggtattcctgcaggttcag

SEQ ID NO: 180 - DNA, 24 bp, synthetic construct
  source 1..24: mol_type = other DNA
acatggccagtcttagaggtactg

SEQ ID NO: 181 - DNA, 24 bp, synthetic construct
  source 1..24: mol_type = other DNA
ggacactacatgaagtcctgtgta

SEQ ID NO: 182 - DNA, 21 bp, synthetic construct
  source 1..21: mol_type = other DNA
ctcacactctcctctggcctt

SEQ ID NO: 183 - DNA, 22 bp, synthetic construct
  source 1..22: mol_type = other DNA
gaagctgtgcttctccatgaga

SEQ ID NO: 184 - DNA, 23 bp, synthetic construct
  source 1..23: mol_type = other DNA
aatcagtttctgccaaagggact

SEQ ID NO: 185 - DNA, 24 bp, synthetic construct
  source 1..24: mol_type = other DNA
ccaccctgaccctttttaagcaga

SEQ ID NO: 186 - DNA, 23 bp, synthetic construct
  source 1..23: mol_type = other DNA
ccctcttacgggaagcccttctg

SEQ ID NO: 187 - DNA, 21 bp, synthetic construct
  source 1..21: mol_type = other DNA
gcacacgtggcactaccatgg

SEQ ID NO: 188 - DNA, 21 bp, synthetic construct
  source 1..21: mol_type = other DNA
ccggatgtacatcgccctgac

SEQ ID NO: 189 - DNA, 25 bp, synthetic construct
  source 1..25: mol_type = other DNA
tgaaagaagagaaccgggtatgctt

SEQ ID NO: 190 - DNA, 24 bp, synthetic construct
  source 1..24: mol_type = other DNA
aggctgtgtgggtccattgagcat

SEQ ID NO: 191 - DNA, 23 bp, synthetic construct
  source 1..23: mol_type = other DNA
acaaacaatcagggggccaatta

SEQ ID NO: 192 - DNA, 22 bp, synthetic construct
  source 1..22: mol_type = other DNA
agggcctcaggaattttggaga

SEQ ID NO: 193 - DNA, 25 bp, synthetic construct
  source 1..25: mol_type = other DNA
atgcagcgtttatgcagatacccat

SEQ ID NO: 194 - DNA, 23 bp, synthetic construct
  source 1..23: mol_type = other DNA
gatggagactttgggtaagtgct

SEQ ID NO: 195 - DNA, 31 bp, synthetic construct
  source 1..31: mol_type = other DNA
gtagatactcctgacaatttcctacagttct

SEQ ID NO: 196 - DNA, 28 bp, synthetic construct
  source 1..28: mol_type = other DNA
taccacttctttcactacatctagtgac

SEQ ID NO: 197 - DNA, 25 bp, synthetic construct
  source 1..25: mol_type = other DNA
agggtctactatttcagcagcttgg

SEQ ID NO: 198 - DNA, 25 bp, synthetic construct
  source 1..25: mol_type = other DNA
tagaccaacttcatttccacatcca

SEQ ID NO: 199 - DNA, 21 bp, synthetic construct
  source 1..21: mol_type = other DNA
tgaacgctttcaggcagtcac

SEQ ID NO: 200 - DNA, 27 bp, synthetic construct
  source 1..27: mol_type = other DNA
tcaggtactagtgaatcaggcagaacc

SEQ ID NO: 201 - DNA, 25 bp, synthetic construct
  source 1..25: mol_type = other DNA
tgttcatcgaagagatccctgtcat

SEQ ID NO: 202 - DNA, 25 bp, synthetic construct
  source 1..25: mol_type = other DNA
cacgatcagtggtgaagtgtttctg

SEQ ID NO: 203 - DNA, 25 bp, synthetic construct
  source 1..25: mol_type = other DNA
ataggttgcttgggagaccctacaa

SEQ ID NO: 204 - DNA, 22 bp, synthetic construct
  source 1..22: mol_type = other DNA
agtggaatgactttgcggagga

SEQ ID NO: 205 - DNA, 28 bp, synthetic construct
  source 1..28: mol_type = other DNA
aggagagagaatactctgaacagctaat

SEQ ID NO: 206 - DNA, 25 bp, synthetic construct
  source 1..25: mol_type = other DNA
gcctaatacttgaattcccctatcg

SEQ ID NO: 207 - DNA, 26 bp, synthetic construct
  source 1..26: mol_type = other DNA
gctgttaattaacatgccctggtggt

SEQ ID NO: 208 - DNA, 26 bp, synthetic construct
  source 1..26: mol_type = other DNA
tgattgagtttctgaagctgcaagtc

SEQ ID NO: 209 - DNA, 23 bp, synthetic construct
  source 1..23: mol_type = other DNA
accaatccaggttggtgagtagg

SEQ ID NO: 210 - DNA, 23 bp, synthetic construct
  source 1..23: mol_type = other DNA
ctgtccctgtccttcctttcttc

SEQ ID NO: 211 - DNA, 36 bp, synthetic construct
  source 1..36: mol_type = other DNA
gtgagaccctatctcaataaataaataattgtcttc

SEQ ID NO: 212 - DNA, 28 bp, synthetic construct
  source 1..28: mol_type = other DNA
tcttcaaggatgttcagtgaaaagcatg

SEQ ID NO: 213 - DNA, 25 bp, synthetic construct
  source 1..25: mol_type = other DNA
ggcctggtctataaggattgcttaa

SEQ ID NO: 214 - DNA, 23 bp, synthetic construct
  source 1..23: mol_type = other DNA
acctgattttcaccccaggccat

SEQ ID NO: 215 - DNA, 32 bp, synthetic construct
  source 1..32: mol_type = other DNA
agagtatcttaaactttctggcttttgtacac

SEQ ID NO: 216 - DNA, 32 bp, synthetic construct; identical to SEQ ID NO: 215
  source 1..32: mol_type = other DNA

SEQ ID NO: 217 - DNA, 25 bp, synthetic construct
  source 1..25: mol_type = other DNA
gtttgctctcaaagcctcagtggaa

SEQ ID NO: 218 - DNA, 24 bp, synthetic construct
  source 1..24: mol_type = other DNA
taatagctcgaggctactgacact

SEQ ID NO: 219 - DNA, 26 bp, synthetic construct
  source 1..26: mol_type = other DNA
tccccaacatgaagattatggactgt

SEQ ID NO: 220 - DNA, 26 bp, synthetic construct
  source 1..26: mol_type = other DNA
gcacttcgtgaggcagatgtagactg

SEQ ID NO: 221 - DNA, 25 bp, synthetic construct
  source 1..25: mol_type = other DNA
gcaggccactttcatgaaactccat

SEQ ID NO: 222 - DNA, 27 bp, synthetic construct
  source 1..27: mol_type = other DNA
aggccgcatacatttaatgtaattgga

SEQ ID NO: 223 - DNA, 22 bp, synthetic construct
  source 1..22: mol_type = other DNA
aggacttgaggcatgtgcaaac

SEQ ID NO: 224 - DNA, 25 bp, synthetic construct
  source 1..25: mol_type = other DNA
gctgttatagatgcttgggggcatg

SEQ ID NO: 225 - DNA, 24 bp, synthetic construct
  source 1..24: mol_type = other DNA
tgtcacattcaccatcgtgcagcc

SEQ ID NO: 226 - DNA, 22 bp, synthetic construct
  source 1..22: mol_type = other DNA
catgacggcagtaactccctca

SEQ ID NO: 227 - DNA, 24 bp, synthetic construct
  source 1..24: mol_type = other DNA
cggttcaccttcactgtttctgat

SEQ ID NO: 228 - DNA, 22 bp, synthetic construct
  source 1..22: mol_type = other DNA
gacactggaggaggtagcctag

SEQ ID NO: 229 - DNA, 24 bp, synthetic construct
  source 1..24: mol_type = other DNA
aggtgttttcctgggagtctgaat

SEQ ID NO: 230 - DNA, 25 bp, synthetic construct
  source 1..25: mol_type = other DNA
ggcccagatgccattgcgattctga

SEQ ID NO: 231 - DNA, 23 bp, synthetic construct
  source 1..23: mol_type = other DNA
agtctggtttccatgtccatgag

SEQ ID NO: 232 - DNA, 34 bp, synthetic construct
  source 1..34: mol_type = other DNA
aagcattacatcaatttctcaaatagtcaagtac

SEQ ID NO: 233 - DNA, 22 bp, synthetic construct
  source 1..22: mol_type = other DNA
gtagtgttggctggaaaggcta

SEQ ID NO: 234 - DNA, 24 bp, synthetic construct
  source 1..24: mol_type = other DNA
tgtggactcctgttctgagaacca

SEQ ID NO: 235 - DNA, 16 bp, synthetic construct
  source 1..16: mol_type = other DNA
tgctcgtcgcctgcca

SEQ ID NO: 236 - DNA, 20 bp, synthetic construct
  source 1..20: mol_type = other DNA
gcgtgagtaaccgccgcggt

SEQ ID NO: 237 - DNA, 23 bp, synthetic construct
  source 1..23: mol_type = other DNA
ggacaggagtaaaaccgaggatc

SEQ ID NO: 238 - DNA, 22 bp, synthetic construct
  source 1..22: mol_type = other DNA
cccaaggagaatgcagagccat

SEQ ID NO: 239 - DNA, 25 bp, synthetic construct
  source 1..25: mol_type = other DNA
ctgagacgaagaaaaaccgaggaaa

SEQ ID NO: 240 - DNA, 25 bp, synthetic construct
  source 1..25: mol_type = other DNA
atgggaaaaggggagaagattgtgt

SEQ ID NO: 241 - DNA, 25 bp, synthetic construct
  source 1..25: mol_type = other DNA
ctgtcttcattactctgcaaagcag

SEQ ID NO: 242 - DNA, 25 bp, synthetic construct
  source 1..25: mol_type = other DNA
atatgaaaccatttctgcaccccat

SEQ ID NO: 243 - DNA, 31 bp, synthetic construct
  source 1..31: mol_type = other DNA
gtggtaacatgttatttacccattcagtatt

SEQ ID NO: 244 - DNA, 27 bp, synthetic construct
  source 1..27: mol_type = other DNA
agtgtctgtctgtggagtgtatccttc

SEQ ID NO: 245 - DNA, 25 bp, synthetic construct
  source 1..25: mol_type = other DNA
actcttgtccaagaagctatccaag

SEQ ID NO: 246 - DNA, 26 bp, synthetic construct
  source 1..26: mol_type = other DNA
tgctctaccagttctgcaagaggaga

SEQ ID NO: 247 - DNA, 31 bp, synthetic construct
  source 1..31: mol_type = other DNA
gtctaacaaaatattcatgagttgtcctgtt

SEQ ID NO: 248 - DNA, 22 bp, synthetic construct
  source 1..22: mol_type = other DNA
gaagaagctcttacaggctgca

SEQ ID NO: 249 - DNA, 24 bp, synthetic construct
  source 1..24: mol_type = other DNA
agcaatctaaagcatagccaccat

SEQ ID NO: 250 - DNA, 25 bp, synthetic construct
  source 1..25: mol_type = other DNA
ttaatgagagctggttacaggacac

SEQ ID NO: 251 - DNA, 23 bp, synthetic construct
  source 1..23: mol_type = other DNA
gatcgcattgtgaccattcactc

SEQ ID NO: 252 - DNA, 31 bp, synthetic construct
  source 1..31: mol_type = other DNA
ataatgaccattgtgtatatctgtttccaag

SEQ ID NO: 253 - DNA, 25 bp, synthetic construct
  source 1..25: mol_type = other DNA
gaatgcatggttgaaaggctaccag

SEQ ID NO: 254 - DNA, 26 bp, synthetic construct
  source 1..26: mol_type = other DNA
agatataactctgaggcagggtcaag

SEQ ID NO: 255 - DNA, 25 bp, synthetic construct
  source 1..25: mol_type = other DNA
ttcctgttgcaccaggttcttcgaa

SEQ ID NO: 256 - DNA, 22 bp, synthetic construct
  source 1..22: mol_type = other DNA
gtcgttatgttctgggcagaag

SEQ ID NO: 257 - DNA, 28 bp, synthetic construct
  source 1..28: mol_type = other DNA
gaaacagagaaaaatgcttccaaagcaa

SEQ ID NO: 258 - DNA, 23 bp, synthetic construct
  source 1..23: mol_type = other DNA
gcaaattggcatgtgtacatggt

SEQ ID NO: 259 - DNA, 26 bp, synthetic construct
  source 1..26: mol_type = other DNA
cacaggttttcctttggaggaagatt

SEQ ID NO: 260 - DNA, 23 bp, synthetic construct
  source 1..23: mol_type = other DNA
atttagggacacccagacttcac

SEQ ID NO: 261 - DNA, 27 bp, synthetic construct
  source 1..27: mol_type = other DNA
gcctctaagtaactggctttaatctct

SEQ ID NO: 262 - DNA, 26 bp, synthetic construct
  source 1..26: mol_type = other DNA
aacatagactctgatcctgatggtga

SEQ ID NO: 263 - DNA, 24 bp, synthetic construct
  source 1..24: mol_type = other DNA
ctgccactgtttgcttacttcatc

SEQ ID NO: 264 - DNA, 25 bp, synthetic construct
  source 1..25: mol_type = other DNA
cccactggcaggatgatttctgtct

SEQ ID NO: 265 - DNA, 25 bp, synthetic construct
  source 1..25: mol_type = other DNA
ctaagagtacagcttcctttgaggt

SEQ ID NO: 266 - DNA, 22 bp, synthetic construct
  source 1..22: mol_type = other DNA
catgttcagggaacacgtgcat

SEQ ID NO: 267 - DNA, 25 bp, synthetic construct
  source 1..25: mol_type = other DNA
tcctaaccttcttcatggagcctat

SEQ ID NO: 268 - DNA, 19 bp, synthetic construct
  source 1..19: mol_type = other DNA
caacaggttgctgccgact

SEQ ID NO: 269 - DNA, 20 bp, synthetic construct
  source 1..20: mol_type = other DNA
cccgtcttcccctatggtct

SEQ ID NO: 270 - DNA, 21 bp, synthetic construct
  source 1..21: mol_type = other DNA
gccagcttcacgggcagcttc

SEQ ID NO: 271 - DNA, 21 bp, synthetic construct
  source 1..21: mol_type = other DNA
tttccctttgcccccatgtag

SEQ ID NO: 272 - DNA, 23 bp, synthetic construct
  source 1..23: mol_type = other DNA
ttctgcgagaacatccatgatgg

SEQ ID NO: 273 - DNA, 29 bp, synthetic construct
  source 1..29: mol_type = other DNA
ctccatgtatatatgcagtatgcttccta

SEQ ID NO: 274 - DNA, 29 bp, synthetic construct
  source 1..29: mol_type = other DNA
acaactttacctggatgtttagttggatc

SEQ ID NO: 275 - DNA, 26 bp, synthetic construct
  source 1..26: mol_type = other DNA
gtgggatcacatgttcactgttgact

SEQ ID NO: 276 - DNA, 26 bp, synthetic construct
  source 1..26: mol_type = other DNA
ttgaaggaaacagctggaagtctgag

SEQ ID NO: 277 - DNA, 20 bp, synthetic construct
  source 1..20: mol_type = other DNA
gtggcccctctcctttcttg

SEQ ID NO: 278 - DNA, 26 bp, synthetic construct
  source 1..26: mol_type = other DNA
tacaggtctggttcagtgagaagatg

SEQ ID NO: 279 - DNA, 24 bp, synthetic construct
  source 1..24: mol_type = other DNA
caaagccctagggaaccacaggaa

SEQ ID NO: 280 - DNA, 21 bp, synthetic construct
  source 1..21: mol_type = other DNA
agcagaggccctctgttgatg

SEQ ID NO: 281 - DNA, 25 bp, synthetic construct
  source 1..25: mol_type = other DNA
cctctttccccaaaagctacgaact

SEQ ID NO: 282 - DNA, 20 bp, synthetic construct
  source 1..20: mol_type = other DNA
gcccgcattgacaaaaagca

SEQ ID NO: 283 - DNA, 29 bp, synthetic construct
  source 1..29: mol_type = other DNA
gaagcttttgttccttttctaagggattt

SEQ ID NO: 284 - DNA, 28 bp, synthetic construct
  source 1..28: mol_type = other DNA
atgcaatgtagatactgtgaagtagact

SEQ ID NO: 285 - DNA, 24 bp, synthetic construct
  source 1..24: mol_type = other DNA
tgacaacctcccaggatgttgttc

SEQ ID NO: 286 - DNA, 23 bp, synthetic construct
  source 1..23: mol_type = other DNA
gaaagtgtcctcctagaggtgga

SEQ ID NO: 287 - DNA, 27 bp, synthetic construct
  source 1..27: mol_type = other DNA
gtgccaactgcttacaatttattgcca

SEQ ID NO: 288 - DNA, 28 bp, synthetic construct
  source 1..28: mol_type = other DNA
acacacttacctatcaatgtactcagca

SEQ ID NO: 289 - DNA, 28 bp, synthetic construct
  source 1..28: mol_type = other DNA
aactactgtttgcctctaagattttctc

SEQ ID NO: 290 - DNA, 25 bp, synthetic construct
  source 1..25: mol_type = other DNA
ttcaccatttaatactgctccacct

SEQ ID NO: 291 - DNA, 25 bp, synthetic construct
  source 1..25: mol_type = other DNA
tccagctcctttttagactgttctg

SEQ ID NO: 292 - DNA, 30 bp, synthetic construct
  source 1..30: mol_type = other DNA
tcaccattctcttttaagaagttctctgtg

SEQ ID NO: 293 - DNA, 25 bp, synthetic construct
  source 1..25: mol_type = other DNA
tggcagctgttagttcatctacctg

SEQ ID NO: 294 - DNA, 24 bp, synthetic construct
  source 1..24: mol_type = other DNA
ctgcatgatgtagcctgtgtgatc

SEQ ID NO: 295 - DNA, 21 bp, synthetic construct
  source 1..21: mol_type = other DNA
ctgatgcaggtgccattgttg

SEQ ID NO: 296 - DNA, 24 bp, synthetic construct
  source 1..24: mol_type = other DNA
aggtctagaatgcagcccctgaac

SEQ ID NO: 297 - DNA, 31 bp, synthetic construct
  source 1..31: mol_type = other DNA
acattcttcaaaaaccaacatggattttttg

SEQ ID NO: 298 - DNA, 34 bp, synthetic construct
  source 1..34: mol_type = other DNA
ataacataaacagctaatttttaaaaccacagtg

SEQ ID NO: 299 - DNA, 22 bp, synthetic construct
  source 1..22: mol_type = other DNA
tttaaggcgcgtttatcccttg

SEQ ID NO: 300 - DNA, 26 bp, synthetic construct
  source 1..26: mol_type = other DNA
gatttagcagatcaggcaacaaaacc

SEQ ID NO: 301 - DNA, 24 bp, synthetic construct
  source 1..24: mol_type = other DNA
cttaccggtttgcctgaatttccc

SEQ ID NO: 302 - DNA, 23 bp, synthetic construct
  source 1..23: mol_type = other DNA
atcaatctgccttacccagactg

SEQ ID NO: 303 - DNA, 23 bp, synthetic construct
  source 1..23: mol_type = other DNA
tcagctgcttttactgctggttt

SEQ ID NO: 304 - DNA, 24 bp, synthetic construct
  source 1..24: mol_type = other DNA
catgttcttgatctctgctgcctt

SEQ ID NO: 305 - DNA, 33 bp, synthetic construct
  source 1..33: mol_type = other DNA
ctttctttgtaccttgattttagtcaagttgta

SEQ ID NO: 306 - DNA, 29 bp, synthetic construct
  source 1..29: mol_type = other DNA
aatgaatcttttccagtctgtaacaagtg

SEQ ID NO: 307 - DNA, 24 bp, synthetic construct
  source 1..24: mol_type = other DNA
catgcagatcagttcctgagactt

SEQ ID NO: 308 - DNA, 25 bp, synthetic construct
  source 1..25: mol_type = other DNA
atttcaggttcatgtgatccgagag

SEQ ID NO: 309 - DNA, 22 bp, synthetic construct
  source 1..22: mol_type = other DNA
ttttggtagcagccgtcgaagg

SEQ ID NO: 310 - DNA, 23 bp, synthetic construct
  source 1..23: mol_type = other DNA
ggtaggaagagcccccaaaaggt

SEQ ID NO: 311 - DNA, 26 bp, synthetic construct
  source 1..26: mol_type = other DNA
ccttgcagtgtaatcgacataaaccc

SEQ ID NO: 312 - DNA, 26 bp, synthetic construct
  source 1..26: mol_type = other DNA
gaacatctgccttgtctcttttgttg

SEQ ID NO: 313 - DNA, 26 bp, synthetic construct
  source 1..26: mol_type = other DNA
gagttgaatgtcagccaatttccata

SEQ ID NO: 314 - DNA, 28 bp, synthetic construct
  source 1..28: mol_type = other DNA
ggggtatttgggatctgtagtgagcctt

SEQ ID NO: 315 - DNA, 24 bp, synthetic construct
  source 1..24: mol_type = other DNA
ccatagccattgtacccatccaag

SEQ ID NO: 316 - DNA, 24 bp, synthetic construct
  source 1..24: mol_type = other DNA
gcagtgcagcagataaggtcacag

SEQ ID NO: 317 - DNA, 25 bp, synthetic construct
  source 1..25: mol_type = other DNA
aggaaaagcggattcaaaattgcag

SEQ ID NO: 318 - DNA, 25 bp, synthetic construct
  source 1..25: mol_type = other DNA
ccttactttttcccttgggttgttc

SEQ ID NO: 319 - DNA, 28 bp, synthetic construct
  source 1..28: mol_type = other DNA
cccaataccattggagagaaatgttcta

SEQ ID NO: 320 - DNA, 24 bp, synthetic construct
  source 1..24: mol_type = other DNA
ttgtagagctctgtaattgggcat

SEQ ID NO: 321 - DNA, 25 bp, synthetic construct
  source 1..25: mol_type = other DNA
gccacaacctgtgattcagagacta

SEQ ID NO: 322 - DNA, 25 bp, synthetic construct
  source 1..25: mol_type = other DNA
tagggaaaacctccctcaattctgt

SEQ ID NO: 323 - DNA, 33 bp, synthetic construct
  source 1..33: mol_type = other DNA
agagagtgaaaatcaaagtacatatgtaatacg

SEQ ID NO: 324 - DNA, 25 bp, synthetic construct
  source 1..25: mol_type = other DNA
aggaggggatcgctcctttatctca

SEQ ID NO: 325 - DNA, 36 bp, synthetic construct
  source 1..36: mol_type = other DNA
catgattttacacatttgtattaatcacagtaccaa

SEQ ID NO: 326 - DNA, 30 bp, synthetic construct
  source 1..30: mol_type = other DNA
ctgtatacagtacctgtatgctggttgttc

SEQ ID NO: 327 - DNA, 23 bp, synthetic construct
  source 1..23: mol_type = other DNA
ttcagcacctacttggagatggt

SEQ ID NO: 328 - DNA, 24 bp, synthetic construct
  source 1..24: mol_type = other DNA
taggcctgacagaattgatcactg

SEQ ID NO: 329 - DNA, 25 bp, synthetic construct
  source 1..25: mol_type = other DNA
gacccaaacccatcatcacctaatg

SEQ ID NO: 330 - DNA, 25 bp, synthetic construct
  source 1..25: mol_type = other DNA
ttttgcagaattttccacacccatc

SEQ ID NO: 331 - DNA, 25 bp, synthetic construct
  source 1..25: mol_type = other DNA
agatcccttttctgctgctgtgtaa

SEQ ID NO: 332 - DNA, 22 bp, synthetic construct
  source 1..22: mol_type = other DNA
ttgatctggagctcgatggtct

SEQ ID NO: 333 - DNA, 24 bp, synthetic construct
  source 1..24: mol_type = other DNA
caggggaaactgttctcttccatt

SEQ ID NO: 334 - DNA, 23 bp, synthetic construct
  source 1..23: mol_type = other DNA
ctcctgtatgttactggtggcaa

SEQ ID NO: 335 - DNA, 21 bp, synthetic construct
  source 1..21: mol_type = other DNA
aggtggacagctgtactctct

SEQ ID NO: 336 - DNA, 28 bp, synthetic construct
  source 1..28: mol_type = other DNA
tttctttttggagactgagttttgctct

SEQ ID NO: 337 - DNA, 24 bp, synthetic construct
  source 1..24: mol_type = other DNA
cttggttgccctgttagactgacg

SEQ ID NO: 338 - DNA, 25 bp, synthetic construct
  source 1..25: mol_type = other DNA
ttagcctcaatttgttcaccacgtt

SEQ ID NO: 339 - DNA, 22 bp, synthetic construct
  source 1..22: mol_type = other DNA
taactcaagggtgctgagcatg

SEQ ID NO: 340 - DNA, 25 bp, synthetic construct
  source 1..25: mol_type = other DNA
gcagatcatagaggcgattgctgct

SEQ ID NO: 341 - DNA, 23 bp, synthetic construct
  source 1..23: mol_type = other DNA
catcaagcgactgttggtttcca

SEQ ID NO: 342 - DNA, 32 bp, synthetic construct
  source 1..32: mol_type = other DNA
cattactgaaaggatcattttgttagagggta

SEQ ID NO: 343 - DNA, 23 bp, synthetic construct
  source 1..23: mol_type = other DNA
tgggtgttacccagaaaaccttc

SEQ ID NO: 344 - DNA, 24 bp, synthetic construct
  source 1..24: mol_type = other DNA
tttcttaggatctgtctggctcca

SEQ ID NO: 345 - DNA, 27 bp, synthetic construct
  source 1..27: mol_type = other DNA
cctggcctgattaattttccatagcta

SEQ ID NO: 346 - DNA, 20 bp, synthetic construct
  source 1..20: mol_type = other DNA
cagcaaatggattccgcagt

SEQ ID NO: 347 - DNA, 21 bp, synthetic construct
  source 1..21: mol_type = other DNA
gtgctggttgtgctccgaagc

SEQ ID NO: 348 - DNA, 21 bp, synthetic construct
  source 1..21: mol_type = other DNA
acctgcgtatgtggagtaggt

SEQ ID NO: 349 - DNA, 25 bp, synthetic construct
  source 1..25: mol_type = other DNA
ggcttaggggctcctaaatgtcacc

SEQ ID NO: 350 - DNA, 19 bp, synthetic construct
  source 1..19: mol_type = other DNA
ttgaccgagtgcacgaagc

SEQ ID NO: 351 - DNA, 26 bp, synthetic construct
  source 1..26: mol_type = other DNA
gggtgttgaaggtctcaaacatgatc

SEQ ID NO: 352 - DNA, 21 bp, synthetic construct
  source 1..21: mol_type = other DNA
aagaacacggctaagtgtgct

SEQ ID NO: 353 - DNA, 28 bp, synthetic construct
  source 1..28: mol_type = other DNA
caaatgaaccacactacagtgcttgtcc

SEQ ID NO: 354 - DNA, 27 bp, synthetic construct
  source 1..27: mol_type = other DNA
gacaccttcctcaacaaatggtctcac

SEQ ID NO: 355 - DNA, 20 bp, synthetic construct
  source 1..20: mol_type = other DNA
tctgcagggaggcggaagcc

SEQ ID NO: 356 - DNA, 20 bp, synthetic construct
  source 1..20: mol_type = other DNA
cgctagcctcgggccgatgc

SEQ ID NO: 357 - DNA, 28 bp, synthetic construct
  source 1..28: mol_type = other DNA
cttgggtgtaaggaaagaagaattgggc

SEQ ID NO: 358 - DNA, 23 bp, synthetic construct
  source 1..23: mol_type = other DNA
ggttgtccagcttgaggaactgt

SEQ ID NO: 359 - DNA, 22 bp, synthetic construct
  source 1..22: mol_type = other DNA
ggctctgtgtaaggtttcctga

SEQ ID NO: 360 - DNA, 24 bp, synthetic construct
  source 1..24: mol_type = other DNA
cagcatagtccaagacctagccaa

SEQ ID NO: 361 - DNA, 34 bp, synthetic construct
  source 1..34: mol_type = other DNA
gaaaatttatccaaaagtaacaggaaaagacttt

SEQ ID NO: 362 - DNA, 35 bp, synthetic construct
  source 1..35: mol_type = other DNA
ggtaaatgttcattatagaaactttgaaaaatgca

SEQ ID NO: 363 - DNA, 26 bp, synthetic construct
  source 1..26: mol_type = other DNA
ccaggagtagctatgactgatcttgc

SEQ ID NO: 364 - DNA, 27 bp, synthetic construct
  source 1..27: mol_type = other DNA
tgattgtaacctactgtcatcccaggt

SEQ ID NO: 365 - DNA, 24 bp, synthetic construct
  source 1..24: mol_type = other DNA
cagccgatggatcactttggtccg

SEQ ID NO: 366 - DNA, 24 bp, synthetic construct
  source 1..24: mol_type = other DNA
aacatagcgaaggcattggtacct

SEQ ID NO: 367 - DNA, 21 bp, synthetic construct
  source 1..21: mol_type = other DNA
ccattctcccgcagaaatcct

SEQ ID NO: 368 - DNA, 22 bp, synthetic construct
  source 1..22: mol_type = other DNA
atgtcgatggacttccagaacc

SEQ ID NO: 369 - DNA, 17 bp, synthetic construct
  source 1..17: mol_type = other DNA
aggagctgctgcgcaag

SEQ ID NO: 370 - DNA, 22 bp, synthetic construct
  source 1..22: mol_type = other DNA
gccccaatcagccccatctcac

SEQ ID NO: 371 - DNA, 27 bp, synthetic construct
  source 1..27: mol_type = other DNA
ccagcagcaacttaaaattacaacaga

SEQ ID NO: 372 - DNA, 29 bp, synthetic construct
  source 1..29: mol_type = other DNA
ttaaagggcaaaatggttataatgttgct

SEQ ID NO: 373 - DNA, 24 bp, synthetic construct
  source 1..24: mol_type = other DNA
cacttacacagacgtgcacagaag

SEQ ID NO: 374 - DNA, 22 bp, synthetic construct
  source 1..22: mol_type = other DNA
taaacaaagcatcaggggcaca

SEQ ID NO: 375 - DNA, 23 bp, synthetic construct
  source 1..23: mol_type = other DNA
gaccatgctatttctttggcaca

SEQ ID NO: 376 - DNA, 25 bp, synthetic construct
  source 1..25: mol_type = other DNA
ctatggatatgtgtctggaggttcc

SEQ ID NO: 377 - DNA, 23 bp, synthetic construct
  source 1..23: mol_type = other DNA
ccaggaaggacatgaagtccaac

SEQ ID NO: 378 - DNA, 23 bp, synthetic construct
  source 1..23: mol_type = other DNA
tttcctagaggggaaggaacagt

SEQ ID NO: 379 - DNA, 24 bp, synthetic construct
  source 1..24: mol_type = other DNA
ttgcagttggtggtaggagtcaaa

SEQ ID NO: 380 - DNA, 23 bp, synthetic construct
  source 1..23: mol_type = other DNA
aaacacaagtgccaacatgctgt

SEQ ID NO: 381 - DNA, 31 bp, synthetic construct
  source 1..31: mol_type = other DNA
agtcacactggtaagtatgtacttcttcatg

SEQ ID NO: 382 - DNA, 27 bp, synthetic construct
  source 1..27: mol_type = other DNA
ctgtaagtgtagttctctgtccaactg

SEQ ID NO: 383 - DNA, 22 bp, synthetic construct
  source 1..22: mol_type = other DNA
gcaagtgaatcctgagcgtgat

SEQ ID NO: 384 - DNA, 23 bp, synthetic construct
  source 1..23: mol_type = other DNA
agaactggagccttcagagggta

SEQ ID NO: 385 - DNA, 25 bp, synthetic construct
  source 1..25: mol_type = other DNA
gcttgacatctggggttttagttgc

SEQ ID NO: 386 - DNA, 26 bp, synthetic construct
  source 1..26: mol_type = other DNA
aaatcgattttggtgtaagcgtgaag

SEQ ID NO: 387 - DNA, 24 bp, synthetic construct
  source 1..24: mol_type = other DNA
tgacgtagaccacaggaacatggc

SEQ ID NO: 388 - DNA, 23 bp, synthetic construct
  source 1..23: mol_type = other DNA
tcgcattatagtccctgtcccta

SEQ ID NO: 389 - DNA, 21 bp, synthetic construct
  source 1..21: mol_type = other DNA
gcggctgcatgacattgagag

SEQ ID NO: 390 - DNA, 24 bp, synthetic construct
  source 1..24: mol_type = other DNA
agagagggcaggaaacaaagtcca

SEQ ID NO: 391 - DNA, 23 bp, synthetic construct
  source 1..23: mol_type = other DNA
accaccgtgatttgcccatcccc

SEQ ID NO: 392 - DNA, 24 bp, synthetic construct
  source 1..24: mol_type = other DNA
tgaagttctaggcgtgcagagaca

SEQ ID NO: 393 - DNA, 31 bp, synthetic construct
  source 1..31: mol_type = other DNA
gcctcggtttactcttaaatgtaaatagaac

SEQ ID NO: 394 - DNA, 26 bp, synthetic construct
  source 1..26: mol_type = other DNA
ggcctatgttgaagtgtggtcatcca

SEQ ID NO: 395 - DNA, 28 bp, synthetic construct
  source 1..28: mol_type = other DNA
cttacagtttgatgtggacatcatagtc

SEQ ID NO: 396 - DNA, 24 bp, synthetic construct
  source 1..24: mol_type = other DNA
tgaattattcaccgtggcagtcac

SEQ ID NO: 397 - DNA, 24 bp, synthetic construct
  source 1..24: mol_type = other DNA
agcaagcccaaatttcagaagttc

SEQ ID NO: 398 - DNA, 30 bp, synthetic construct
  source 1..30: mol_type = other DNA
tgtcttctgctactattgaagtacaaatgg

SEQ ID NO: 399 - DNA, 23 bp, synthetic construct
  source 1..23: mol_type = other DNA
gccaggaaagttgccaagtaggc

SEQ ID NO: 400 - DNA, 29 bp, synthetic construct
  source 1..29: mol_type = other DNA
agcatgctttgaaatctagttcttggaca

SEQ ID NO: 401 - DNA, 22 bp, synthetic construct
  source 1..22: mol_type = other DNA
gaggcgcaggtaacctctggag

SEQ ID NO: 402 - DNA, 21 bp, synthetic construct
  source 1..21: mol_type = other DNA
aatgtcacggggaaagagctt

SEQ ID NO: 403 - DNA, 21 bp, synthetic construct
  source 1..21: mol_type = other DNA
tgcgcccttacgaaaaaggta

SEQ ID NO: 404 - DNA, 25 bp, synthetic construct
  source 1..25: mol_type = other DNA
acagaatctcagagcctcatccttc

SEQ ID NO: 405 - DNA, 25 bp, synthetic construct
  source 1..25: mol_type = other DNA
aagtcccaaaggaagccagtacccc

SEQ ID NO: 406 - DNA, 20 bp, synthetic construct
  source 1..20: mol_type = other DNA
ggccgaggcgcttacctggt

SEQ ID NO: 407 - DNA, 22 bp, synthetic construct
  source 1..22: mol_type = other DNA
ctgctcccggtgatgcaaaccc

SEQ ID NO: 408 - DNA, 20 bp, synthetic construct
  source 1..20: mol_type = other DNA
gtgcgtcatgggcaacatcc

SEQ ID NO: 409 - DNA, 23 bp, synthetic construct
  source 1..23: mol_type = other DNA
gtggttcagtgtcacaatgcatt

SEQ ID NO: 410 - DNA, 24 bp, synthetic construct
  source 1..24: mol_type = other DNA
aggcaaaagcagcaggtcaccaga

SEQ ID NO: 411 - DNA, 20 bp, synthetic construct
  source 1..20: mol_type = other DNA
ttgccctgggcacattgttc

SEQ ID NO: 412 - DNA, 24 bp, synthetic construct
  source 1..24: mol_type = other DNA
catcctcaacatggagagcatggt

SEQ ID NO: 413 - DNA, 28 bp, synthetic construct
  source 1..28: mol_type = other DNA
gaaaataactctacaggcccattgcctc

SEQ ID NO: 414 - DNA, 25 bp, synthetic construct
  source 1..25: mol_type = other DNA
ggcaagatgtgcagtgctgactacg

SEQ ID NO: 415 - DNA, 29 bp, synthetic construct
  source 1..29: mol_type = other DNA
ccaggacaactgattattgttgcatccaa

SEQ ID NO: 416 - DNA, 27 bp, synthetic construct
  source 1..27: mol_type = other DNA
gagcactgaagaggtctttatgggtag

SEQ ID NO: 417 - DNA, 24 bp, synthetic construct
  source 1..24: mol_type = other DNA
cttgcctggacatcaaactcttgc

SEQ ID NO: 418 - DNA, 26 bp, synthetic construct
  source 1..26: mol_type = other DNA
gggacatatgggcctttctatgacct

SEQ ID NO: 419 - DNA, 22 bp, synthetic construct
  source 1..22: mol_type = other DNA
ggtgaggacatggccacaccct

SEQ ID NO: 420 - DNA, 19 bp, synthetic construct
  source 1..19: mol_type = other DNA
gggtggcggggcttcagct

SEQ ID NO: 421 - DNA, 22 bp, synthetic construct
  source 1..22: mol_type = other DNA
ttctgactccggctctgcatcc

SEQ ID NO: 422 - DNA, 24 bp, synthetic construct
  source 1..24: mol_type = other DNA
aaaagaactggctgtgacctttgc

SEQ ID NO: 423 - DNA, 20 bp, synthetic construct
  source 1..20: mol_type = other DNA
cttggggctctcgagcaggg

SEQ ID NO: 424 - DNA, 17 bp, synthetic construct
  source 1..17: mol_type = other DNA
cgggcgacgtacctggc

SEQ ID NO: 425 - DNA, 22 bp, synthetic construct
  source 1..22: mol_type = other DNA
gcctgtcagagctgatcactgc

SEQ ID NO: 426 - DNA, 21 bp, synthetic construct
  source 1..21: mol_type = other DNA
cacacggccatagaggaagtc

SEQ ID NO: 427 - DNA, 17 bp, synthetic construct
  source 1..17: mol_type = other DNA
ccactgcagctggcaga

SEQ ID NO: 428 - DNA, 19 bp, synthetic construct
  source 1..19: mol_type = other DNA
ccaagcacacagccaaagg

SEQ ID NO: 429 - DNA, 23 bp, synthetic construct
  source 1..23: mol_type = other DNA
ccagccaacttctagcaaattcg

SEQ ID NO: 430 - DNA, 23 bp, synthetic construct
  source 1..23: mol_type = other DNA
gttatgatttgggctgtgcgttg

SEQ ID NO: 431 - DNA, 22 bp, synthetic construct
  source 1..22: mol_type = other DNA
gagccatcattgactggtcgta

SEQ ID NO: 432 - DNA, 23 bp, synthetic construct
  source 1..23: mol_type = other DNA
tacgtatatggcaacgttcaggt

SEQ ID NO: 433 - DNA, 22 bp, synthetic construct
  source 1..22: mol_type = other DNA
ttatagaaggcagggcagacct

SEQ ID NO: 434 - DNA, 22 bp, synthetic construct
  source 1..22: mol_type = other DNA
actggctgcattggactttgca

SEQ ID NO: 435 - DNA, 22 bp, synthetic construct
  source 1..22: mol_type = other DNA
agccagctgacgtaattcagag

SEQ ID NO: 436 - DNA, 23 bp, synthetic construct
  source 1..23: mol_type = other DNA
ctgcctaagtggttctggaacag

SEQ ID NO: 437 - DNA, 25 bp, synthetic construct
  source 1..25: mol_type = other DNA
ctctgaatgcaatgccctcagaaag

SEQ ID NO: 438 - DNA, 22 bp, synthetic construct
  source 1..22: mol_type = other DNA
ttcagatgtcctcttggctcga

SEQ ID NO: 439 - DNA, 28 bp, synthetic construct
  source 1..28: mol_type = other DNA
ctatgtgggagacatactcattgctctt

SEQ ID NO: 440 - DNA, 25 bp, synthetic construct
  source 1..25: mol_type = other DNA
gggtgcattctgttacttcatacac

SEQ ID NO: 441 - DNA, 23 bp, synthetic construct
  source 1..23: mol_type = other DNA
gaaaccaccaatgctgtggagag

SEQ ID NO: 442 - DNA, 25 bp, synthetic construct
  source 1..25: mol_type = other DNA
agtaagccccaaacagaagtctgtc

SEQ ID NO: 443 - DNA, 23 bp, synthetic construct
  source 1..23: mol_type = other DNA
tgtggtctacccagtgtctgtct

SEQ ID NO: 444 - DNA, 22 bp, synthetic construct
  source 1..22: mol_type = other DNA
tcgcgctttcatttgtccttcg

SEQ ID NO: 445 - DNA, 23 bp, synthetic construct
  source 1..23: mol_type = other DNA
atctccagaatgcgtcctccttc

SEQ ID NO: 446 - DNA, 27 bp, synthetic construct
  source 1..27: mol_type = other DNA
attaatgatgctagcagagaaagagca

SEQ ID NO: 447 - DNA, 26 bp, synthetic construct
  source 1..26: mol_type = other DNA
gtctggtaccactgtgtcattaggaa

SEQ ID NO: 448 - DNA, 25 bp, synthetic construct
  source 1..25: mol_type = other DNA
caaccagaagtcatctctagccctt

SEQ ID NO: 449 - DNA, 26 bp, synthetic construct
  source 1..26: mol_type = other DNA
ggccaacaagtcctgtttacaagatg

SEQ ID NO: 450 - DNA, 26 bp, synthetic construct
  source 1..26: mol_type = other DNA
atacagtattcacatggcaggtacct

SEQ ID NO: 451 - DNA, 23 bp, synthetic construct
  source 1..23: mol_type = other DNA
cctcacagctctgcaaaagacta

SEQ ID NO: 452 - DNA, 26 bp, synthetic construct
  source 1..26: mol_type = other DNA
ctttctttgcgaacttcctcaagtga

SEQ ID NO: 453 - DNA, 24 bp, synthetic construct
  source 1..24: mol_type = other DNA
tcttgtagcttttctggggcaaag

SEQ ID NO: 454 - DNA, 26 bp, synthetic construct
  source 1..26: mol_type = other DNA
ggctggatcagagctctacaaaagca

SEQ ID NO: 455 - DNA, 19 bp, synthetic construct
  source 1..19: mol_type = other DNA
cccaacccacagcacttcc

SEQ ID NO: 456 - DNA, 21 bp, synthetic construct
  source 1..21: mol_type = other DNA
gtaggctgcggatgatgtctg

SEQ ID NO: 457 - DNA, 23 bp, synthetic construct
  source 1..23: mol_type = other DNA
tggtgataactgcactggtgaag

SEQ ID NO: 458 - DNA, 24 bp, synthetic construct
  source 1..24: mol_type = other DNA
actgtcttcagcagcagcatctac

SEQ ID NO: 459 - DNA, 24 bp, synthetic construct
  source 1..24: mol_type = other DNA
ctgctttcttctccttgccctttc

SEQ ID NO: 460 - DNA, 22 bp, synthetic construct
  source 1..22: mol_type = other DNA
acgtgacccactcttttccaca

SEQ ID NO: 461 - DNA, 25 bp, synthetic construct
  source 1..25: mol_type = other DNA
aaggaaggatccatcacctccacca

SEQ ID NO: 462 - DNA, 23 bp, synthetic construct
  source 1..23: mol_type = other DNA
cgagcatagtggggcacaaaagg

SEQ ID NO: 463 - DNA, 23 bp, synthetic construct
  source 1..23: mol_type = other DNA
atttctcatccttcccgccatcg

SEQ ID NO: 464 - DNA, 24 bp, synthetic construct
  source 1..24: mol_type = other DNA
acgattacgaacctgagctgctgt

SEQ ID NO: 465 - DNA, 24 bp, synthetic construct
  source 1..24: mol_type = other DNA
agcaaccatttttgtgcccatgtt

SEQ ID NO: 466 - DNA, 25 bp, synthetic construct
  source 1..25: mol_type = other DNA
tggtaccgagtgatccgtcatacga

SEQ ID NO: 467 - DNA, 28 bp, synthetic construct
  source 1..28: mol_type = other DNA
accaccttttattactccagctatagtg

SEQ ID NO: 468 - DNA, 30 bp, synthetic construct
  source 1..30: mol_type = other DNA
gattgctgcatatttcagatatttctttcc

SEQ ID NO: 469 - DNA, 23 bp, synthetic construct
  source 1..23: mol_type = other DNA
ctattttggagaggcaccacctg

SEQ ID NO: 470 - DNA, 22 bp, synthetic construct
  source 1..22: mol_type = other DNA
aaagggtggccttgagttacag

SEQ ID NO: 471 - DNA, 25 bp, synthetic construct
  source 1..25: mol_type = other DNA
accaaacaatcccatccccttcccc

SEQ ID NO: 472 - DNA, 23 bp, synthetic construct
  source 1..23: mol_type = other DNA
gcctggaagttggttgcattgac

SEQ ID NO: 473 - DNA, 23 bp, synthetic construct
  source 1..23: mol_type = other DNA
gttgtggtagtttgtggggagat

SEQ ID NO: 474 - DNA, 25 bp, synthetic construct
  source 1..25: mol_type = other DNA
tcggggtctttcggaagtttgcaac

SEQ ID NO: 475 - DNA, 22 bp, synthetic construct
  source 1..22: mol_type = other DNA
ccttccacaaaaatggccagtg

SEQ ID NO: 476 - DNA, 21 bp, synthetic construct
  source 1..21: mol_type = other DNA
taccgggaagtcctgatgtga

SEQ ID NO: 477 - DNA, 29 bp, synthetic construct
  source 1..29: mol_type = other DNA
gctacaaacaatttctgtgactttcagtc

SEQ ID NO: 478 - DNA, 25 bp, synthetic construct
  source 1..25: mol_type = other DNA
ctgtttcccagcctgaaaacaaacc

SEQ ID NO: 479 - DNA, 25 bp, synthetic construct
  source 1..25: mol_type = other DNA
gcatgtctcacaagacaaccaagga

SEQ ID NO: 480 - DNA, 23 bp, synthetic construct
  source 1..23: mol_type = other DNA
ccaactgcttgaacgttggtctc

SEQ ID NO: 481 - DNA, 22 bp, synthetic construct
  source 1..22: mol_type = other DNA
ttcccagacgtgatctcaggag

SEQ ID NO: 482 - DNA, 24 bp, synthetic construct
  source 1..24: mol_type = other DNA
ttcttgttgttcctgtgggggtag

SEQ ID NO: 483 - DNA, 22 bp, synthetic construct
  source 1..22: mol_type = other DNA
caacaccagcatccagttcaac

SEQ ID NO: 484 - DNA, 22 bp, synthetic construct
  source 1..22: mol_type = other DNA
accaagggagaacactgatcct

SEQ ID NO: 485 - DNA, 24 bp, synthetic construct
  source 1..24: mol_type = other DNA
catggagattccagtggtaagcat

SEQ ID NO: 486 - DNA, 24 bp, synthetic construct
  source 1..24: mol_type = other DNA
agtaggatggaatggtttcccgaa

SEQ ID NO: 487 - DNA, 23 bp, synthetic construct
  source 1..23: mol_type = other DNA
gaccgggcaaactttttctgaca

SEQ ID NO: 488 - DNA, 22 bp, synthetic construct
  source 1..22: mol_type = other DNA
aactatcaaggcccgagtgaag

SEQ ID NO: 489 - DNA, 25 bp, synthetic construct
  source 1..25: mol_type = other DNA
ggaggtttgggatgttgtttctgct

SEQ ID NO: 490 - DNA, 22 bp, synthetic construct
  source 1..22: mol_type = other DNA
gagcatctgtggttgtaccaga

SEQ ID NO: 491 - DNA, 23 bp, synthetic construct
  source 1..23: mol_type = other DNA
gcagtgtggtgtcacaagcatga

SEQ ID NO: 492 - DNA, 23 bp, synthetic construct
  source 1..23: mol_type = other DNA
attcaagtgtcctgagtgcacag

SEQ ID NO: 493 - DNA, 23 bp, synthetic construct
  source 1..23: mol_type = other DNA
atacaccccaaggtggagagaaa

SEQ ID NO: 494 - DNA, 21 bp, synthetic construct
  source 1..21: mol_type = other DNA
gcgcctaaagttccctacctt

SEQ ID NO: 495 - DNA, 23 bp, synthetic construct
  source 1..23: mol_type = other DNA
ttaacctctctgagcctcagcat

SEQ ID NO: 496 - DNA, 22 bp, synthetic construct
  source 1..22: mol_type = other DNA
ttcatcctcacctgtgtgtcca

SEQ ID NO: 497 - DNA, 22 bp, synthetic construct
  source 1..22: mol_type = other DNA
tgtctgatgacggagggtgagg

SEQ ID NO: 498 - DNA, 21 bp, synthetic construct
  source 1..21: mol_type = other DNA
cttcctggactcacccacaca

SEQ ID NO: 499 - DNA, 23 bp, synthetic construct
  source 1..23: mol_type = other DNA
tcagtgctcagggctttacaacc

SEQ ID NO: 500 - DNA, 22 bp, synthetic construct
  source 1..22: mol_type = other DNA
aagaagtagcccttgaccatgg

SEQ ID NO: 501 - DNA, 20 bp, synthetic construct
  source 1..20: mol_type = other DNA
acgacggtgagttctggtga

SEQ ID NO: 502 - DNA, 24 bp, synthetic construct
  source 1..24: mol_type = other DNA
atgccccatcatcaatttgctgtg

SEQ ID NO: 503 - DNA, 24 bp, synthetic construct
  source 1..24: mol_type = other DNA
gaaggtagaagggctgagaggagt

SEQ ID NO: 504 - DNA, 25 bp, synthetic construct
  source 1..25: mol_type = other DNA
gcccttccttcattggacatcagct

SEQ ID NO: 505 - DNA, 23 bp, synthetic construct
  source 1..23: mol_type = other DNA
atggcagagctggcttttccctc

SEQ ID NO: 506 - DNA, 24 bp, synthetic construct
  source 1..24: mol_type = other DNA
cccccaggatatcctcaccactca

SEQ ID NO: 507 - DNA, 25 bp, synthetic construct
  source 1..25: mol_type = other DNA
ggctatacctgtaaatggtggtcta

SEQ ID NO: 508 - DNA, 26 bp, synthetic construct
  source 1..26: mol_type = other DNA
agaccaaaggcaatgttaccctcttg

SEQ ID NO: 509 - DNA, 34 bp, synthetic construct
  source 1..34: mol_type = other DNA
cagtttttaataaagattccttcacaaatcctat

SEQ ID NO: 510 - DNA, 28 bp, synthetic construct
  source 1..28: mol_type = other DNA
ataatgtcttttggattagctacccgat

SEQ ID NO: 511 - DNA, 25 bp, synthetic construct
  source 1..25: mol_type = other DNA
aaaggatgagctggagtcacagagg

SEQ ID NO: 512 - DNA, 24 bp, synthetic construct
  source 1..24: mol_type = other DNA
aacgggaatttggagttcacacac

SEQ ID NO: 513 - DNA, 25 bp, synthetic construct
  source 1..25: mol_type = other DNA
agggagtggccaatttcatgagcag

SEQ ID NO: 514 - DNA, 24 bp, synthetic construct
  source 1..24: mol_type = other DNA
caggtctgctgtgcattgtattgg

SEQ ID NO: 515 - DNA, 24 bp, synthetic construct
  source 1..24: mol_type = other DNA
ctctcagcaaagtcatcagaggaa

SEQ ID NO: 516 - DNA, 26 bp, synthetic construct
  source 1..26: mol_type = other DNA
tgttataagtgactcctccatcacac

SEQ ID NO: 517 - DNA, 25 bp, synthetic construct
  source 1..25: mol_type = other DNA
agatggttctgacccacttcacccc

SEQ ID NO: 518 - DNA, 24 bp, synthetic construct
  source 1..24: mol_type = other DNA
agcagcacctgttgagagaagtga

SEQ ID NO: 519 - DNA, 25 bp, synthetic construct
  source 1..25: mol_type = other DNA
tagaagccaccagtctcaggttcac

SEQ ID NO: 520 - DNA, 27 bp, synthetic construct
  source 1..27: mol_type = other DNA
caggaggcagggttagaactcttcaag

SEQ ID NO: 521 - DNA, 22 bp, synthetic construct
  source 1..22: mol_type = other DNA
aaaggaccgaagtagggtggtg

SEQ ID NO: 522 - DNA, 23 bp, synthetic construct
  source 1..23: mol_type = other DNA
gcggaattgggtgaaggcacgat

SEQ ID NO: 523 - DNA, 22 bp, synthetic construct
  source 1..22: mol_type = other DNA
gctcctcgtcttcatgtttcca

SEQ ID NO: 524 - DNA, 21 bp, synthetic construct
  source 1..21: mol_type = other DNA
gcaagctaccctcattcgtgt

SEQ ID NO: 525 - DNA, 23 bp, synthetic construct
  source 1..23: mol_type = other DNA
ttctctcctgctctcctttccat

SEQ ID NO: 526 - DNA, 22 bp, synthetic construct
  source 1..22: mol_type = other DNA
aatcttgcttttggcagccttg

SEQ ID NO: 527 - DNA, 25 bp, synthetic construct
  source 1..25: mol_type = other DNA
gtccagagctaaagatgacctgacc

SEQ ID NO: 528 - DNA, 24 bp, synthetic construct
  source 1..24: mol_type = other DNA
tcaggaaagcaaacccctctaggt

SEQ ID NO: 529 - DNA, 27 bp, synthetic construct
  source 1..27: mol_type = other DNA
tcgcctttgaaatacaagtcatgtctt

SEQ ID NO: 530 - DNA, 26 bp, synthetic construct
  source 1..26: mol_type = other DNA
agtagtgagagatgcatttccggaag

SEQ ID NO: 531 - DNA, 22 bp, synthetic construct
  source 1..22: mol_type = other DNA
gacactcttttgcactcctcct

SEQ ID NO: 532 - DNA, 23 bp, synthetic construct
  source 1..23: mol_type = other DNA
tgctatagggtgccagggagctt

SEQ ID NO: 533 - DNA, 23 bp, synthetic construct
  source 1..23: mol_type = other DNA
ctcacctctttttcaggtgtcga

SEQ ID NO: 534 - DNA, 24 bp, synthetic construct
  source 1..24: mol_type = other DNA
gggacttgtggtgacatgcctcag

SEQ ID NO: 535 - DNA, 23 bp, synthetic construct
  source 1..23: mol_type = other DNA
caggtggctctaaaagcactcac

SEQ ID NO: 536 - DNA, 25 bp, synthetic construct
  source 1..25: mol_type = other DNA
tcttcactacgtagcacaggtaggt

SEQ ID NO: 537 - DNA, 23 bp, synthetic construct
  source 1..23: mol_type = other DNA
caggaggctttactttctgagga

SEQ ID NO: 538 - DNA, 28 bp, synthetic construct
  source 1..28: mol_type = other DNA
ccttttctacattttcatttcaggtgac

SEQ ID NO: 539 - DNA, 23 bp, synthetic construct
  source 1..23: mol_type = other DNA
ccgaagccctctgtaatttctga

SEQ ID NO: 540 - DNA, 24 bp, synthetic construct
  source 1..24: mol_type = other DNA
taggaaatcaggcccaatactcac

SEQ ID NO: 541 - DNA, 25 bp, synthetic construct
  source 1..25: mol_type = other DNA
aagaacaagcacgtgcaggtaggac

SEQ ID NO: 542 - DNA, 26 bp, synthetic construct
  source 1..26: mol_type = other DNA
gcatgctgggcactaaataatctcct

SEQ ID NO: 543 - DNA, 25 bp, synthetic construct
  source 1..25: mol_type = other DNA
accctgagggaagagaaaaccagcc

SEQ ID NO: 544 - DNA, 25 bp, synthetic construct
  source 1..25: mol_type = other DNA
ctgcccaagaggaatttgctgtggt

SEQ ID NO: 545 - DNA, 23 bp, synthetic construct
  source 1..23: mol_type = other DNA
cttgaaatccttgaggcccctaa

SEQ ID NO: 546 - DNA, 24 bp, synthetic construct
  source 1..24: mol_type = other DNA
cagatacttacaggagcacctgca

SEQ ID NO: 547 - DNA, 21 bp, synthetic construct
  source 1..21: mol_type = other DNA
tgagggacgcatggaaagcag

SEQ ID NO: 548 - DNA, 21 bp, synthetic construct
  source 1..21: mol_type = other DNA
cccgaggcgaatcatggctca

SEQ ID NO: 549 - DNA, 22 bp, synthetic construct
  source 1..22: mol_type = other DNA
aaggggcttggtgttaaggtac

SEQ ID NO: 550 - DNA, 21 bp, synthetic construct
  source 1..21: mol_type = other DNA
agaatggccattgttgggaca

SEQ ID NO: 551 - DNA, 24 bp, synthetic construct
  source 1..24: mol_type = other DNA
ccgagttcgtcaaccactactgct

SEQ ID NO: 552 - DNA, 22 bp, synthetic construct
  source 1..22: mol_type = other DNA
cttccctcctttcctctcatgc

SEQ ID NO: 553 - DNA, 23 bp, synthetic construct
  source 1..23: mol_type = other DNA
ggacatgaagaaggtggtgtgtg

SEQ ID NO: 554 - DNA, 24 bp, synthetic construct
  source 1..24: mol_type = other DNA
gggatttctgggcccaggaacttg

SEQ ID NO: 555 - DNA, 22 bp, synthetic construct
  source 1..22: mol_type = other DNA
ctgcactgggaagacttttcca

SEQ ID NO: 556 - DNA, 21 bp, synthetic construct
  source 1..21: mol_type = other DNA
cattctccacctcggctgtca

SEQ ID NO: 557 - DNA, 22 bp, synthetic construct
  source 1..22: mol_type = other DNA
ctttgggtgtggaaggtgtcat

SEQ ID NO: 558 - DNA, 24 bp, synthetic construct
  source 1..24: mol_type = other DNA
atatctgaaatgacgagggggtct

SEQ ID NO: 559 - DNA, 23 bp, synthetic construct
  source 1..23: mol_type = other DNA
aggtggaacgtgaacatatgtgt

SEQ ID NO: 560 - DNA, 28 bp, synthetic construct
  source 1..28: mol_type = other DNA
gagacattatgcaattaaatgcagggat

SEQ ID NO: 561 - DNA, 24 bp, synthetic construct
  source 1..24: mol_type = other DNA
tggtgattatggggaagtgattcc

SEQ ID NO: 562 - DNA, 26 bp, synthetic construct
  source 1..26: mol_type = other DNA
gagcatgccaaggttgattagcagtg

SEQ ID NO: 563 - DNA, 21 bp, synthetic construct
  source 1..21: mol_type = other DNA
acctgtgcagagccatgtgac

SEQ ID NO: 564 - DNA, 24 bp, synthetic construct
  source 1..24: mol_type = other DNA
tggctgtcaatggatgcaggggaa

SEQ ID NO: 565 - DNA, 20 bp, synthetic construct
  source 1..20: mol_type = other DNA
gatggccatggcacttacca

SEQ ID NO: 566 - DNA, 18 bp, synthetic construct
  source 1..18: mol_type = other DNA
tggcctacctgggggatg

SEQ ID NO: 567 - DNA, 17 bp, synthetic construct
  source 1..17: mol_type = other DNA
tcaggcgcggcgagtgc

SEQ ID NO: 568 - DNA, 20 bp, synthetic construct
  source 1..20: mol_type = other DNA
cacctgctggcctcgccatc

SEQ ID NO: 569 - DNA, 25 bp, synthetic construct
  source 1..25: mol_type = other DNA
aaggtgcatggaccatacttccgtt

SEQ ID NO: 570 - DNA, 25 bp, synthetic construct
  source 1..25: mol_type = other DNA
gagggtgtggttttcctcagtcagt

SEQ ID NO: 571 - DNA, 31 bp, synthetic construct
  source 1..31: mol_type = other DNA
gtctttgataagtgtgttagtaactgacaat

SEQ ID NO: 572 - DNA, 31 bp, synthetic construct
  source 1..31: mol_type = other DNA
tttagtacgatgtgattaacgtagaatagct

SEQ ID NO: 573 - DNA, 25 bp, synthetic construct
  source 1..25: mol_type = other DNA
tgactgttatgtgctagacagtgtt

SEQ ID NO: 574 - DNA, 28 bp, synthetic construct
  source 1..28: mol_type = other DNA
cctcatagagcttacattatgaggttgg

SEQ ID NO: 575 - DNA, 20 bp, synthetic construct
  source 1..20: mol_type = other DNA
gccagcagagactacaccaa

SEQ ID NO: 576 - DNA, 19 bp, synthetic construct
  source 1..19: mol_type = other DNA
cctcccgctccatgttcgt

SEQ ID NO: 577 - DNA, 24 bp, synthetic construct
  source 1..24: mol_type = other DNA
cgattttcagagtcgactgtccag

SEQ ID NO: 578 - DNA, 23 bp, synthetic construct
  source 1..23: mol_type = other DNA
tagggagttcgtagttgagctca

SEQ ID NO: 579 - DNA, 29 bp, synthetic construct
  source 1..29: mol_type = other DNA
gcctgtgcattttatatcaaagggaaatt

SEQ ID NO: 580 - DNA, 23 bp, synthetic construct
  source 1..23: mol_type = other DNA
tgggagctctcagtaaaattgca

SEQ ID NO: 581 - DNA, 27 bp, synthetic construct
  source 1..27: mol_type = other DNA
ggattccaggaccctaaagaactctgc

SEQ ID NO: 582 - DNA, 23 bp, synthetic construct
  source 1..23: mol_type = other DNA
gtcctcaattgctgagcgtttgg

SEQ ID NO: 583 - DNA, 23 bp, synthetic construct
  source 1..23: mol_type = other DNA
acccttagagcctgtgattcctg

SEQ ID NO: 584 - DNA, 22 bp, synthetic construct
  source 1..22: mol_type = other DNA
cccctcaatgttctggaaagca

SEQ ID NO: 585 - DNA, 24 bp, synthetic construct
  source 1..24: mol_type = other DNA
cctgaaaccgaagagagaagcagt

SEQ ID NO: 586 - DNA, 24 bp, synthetic construct
  source 1..24: mol_type = other DNA
acacttacgggactccctttttcc

SEQ ID NO: 587 - DNA, 24 bp, synthetic construct
  source 1..24: mol_type = other DNA
tgggaaaggaaacagggaagtcga

SEQ ID NO: 588 - DNA, 24 bp, synthetic construct
  source 1..24: mol_type = other DNA
gaatacctggaacgaagggtactc

SEQ ID NO: 589 - DNA, 24 bp, synthetic construct
  source 1..24: mol_type = other DNA
ctccaggactgcatggattcccag

SEQ ID NO: 590 - DNA, 21 bp, synthetic construct
  source 1..21: mol_type = other DNA
taaacgagtggggtcctcact

SEQ ID NO: 591 - DNA, 25 bp, synthetic construct
  source 1..25: mol_type = other DNA
ccagctcatgcacattctttcctcc

SEQ ID NO: 592 - DNA, 22 bp, synthetic construct
  source 1..22: mol_type = other DNA
caccaactcatctctggcctct

SEQ ID NO: 593 - DNA, 22 bp, synthetic construct
  source 1..22: mol_type = other DNA
ttctccagcatctctctctcct

SEQ ID NO: 594 - DNA, 23 bp, synthetic construct
  source 1..23: mol_type = other DNA
attcccactgggatttctcctca

SEQ ID NO: 595 - DNA, 21 bp, synthetic construct
  source 1..21: mol_type = other DNA
ctctcacgaagaggacgagga

SEQ ID NO: 596 - DNA, 19 bp, synthetic construct
  source 1..19: mol_type = other DNA
cttagcaacacggccagct

SEQ ID NO: 597 - DNA, 22 bp, synthetic construct
  source 1..22: mol_type = other DNA
ccaaaccatgcctttgctgaag

SEQ ID NO: 598 - DNA, 25 bp, synthetic construct
  source 1..25: mol_type = other DNA
cgcaggtccttgtagagatcggtga

SEQ ID NO: 599 - DNA, 29 bp, synthetic construct
  source 1..29: mol_type = other DNA
caaaatgtcttttggtcagcaatagctta

SEQ ID NO: 600 - DNA, 24 bp, synthetic construct
  source 1..24: mol_type = other DNA
gaagaccgcagtgggataaatctc

SEQ ID NO: 601 - DNA, 26 bp, synthetic construct
  source 1..26: mol_type = other DNA
ctctctgttggttgtaggaagctgta

SEQ ID NO: 602 - DNA, 23 bp, synthetic construct
  source 1..23: mol_type = other DNA
tgtgtaagtcaagggtagccatg

SEQ ID NO: 603 - DNA, 28 bp, synthetic construct
  source 1..28: mol_type = other DNA
gtcaggggtctctatgtatacatcacac

SEQ ID NO: 604 - DNA, 25 bp, synthetic construct
  source 1..25: mol_type = other DNA
tgtcatttcacagcacatgcttcca

SEQ ID NO: 605 - DNA, 24 bp, synthetic construct
  source 1..24: mol_type = other DNA
ccacctccacacatttctgtcttt

SEQ ID NO: 606 - DNA, 21 bp, synthetic construct
  source 1..21: mol_type = other DNA
ctcacagtggttgtccagctt

SEQ ID NO: 607 - DNA, 22 bp, synthetic construct
  source 1..22: mol_type = other DNA
gtacaagaagggtgtgtggctt

SEQ ID NO: 608 - DNA, 24 bp, synthetic construct
  source 1..24: mol_type = other DNA
ccggttgctttcgctctttgaacg

SEQ ID NO: 609 - DNA, 21 bp, synthetic construct
  source 1..21: mol_type = other DNA
agtcagcagccccttacctcc

SEQ ID NO: 610 - DNA, 19 bp, synthetic construct
  source 1..19: mol_type = other DNA
ttgatgccacgtcccatgg

SEQ ID NO: 611 - DNA, 24 bp, synthetic construct
  source 1..24: mol_type = other DNA
tctttgagctgacatcccactgag

SEQ ID NO: 612 - DNA, 25 bp, synthetic construct
  source 1..25: mol_type = other DNA
gcacgatctggaaagaagcacagga

SEQ ID NO: 613 - DNA, 22 bp, synthetic construct
  source 1..22: mol_type = other DNA
gcttgcagttcgttcatccatc

SEQ ID NO: 614 - DNA, 26 bp, synthetic construct
  source 1..26: mol_type = other DNA
aacaacgtagtccaccagattaatcc

SEQ ID NO: 615 - DNA, 27 bp, synthetic construct
  source 1..27: mol_type = other DNA
tcggtgtcgagagtatttgtcatctgg

SEQ ID NO: 616 - DNA, 28 bp, synthetic construct
  source 1..28: mol_type = other DNA
gtatgtcagtacaggagtgatgaaatgg

SEQ ID NO: 617 - DNA, 28 bp, synthetic construct
  source 1..28: mol_type = other DNA
gtctttcagtcctcttgatcataccacc

SEQ ID NO: 618 - DNA, 26 bp, synthetic construct
  source 1..26: mol_type = other DNA
ctctctccagatactgcatagaacgt

SEQ ID NO: 619 - DNA, 22 bp, synthetic construct
  source 1..22: mol_type = other DNA
gtgcttcccagtttcactccat

SEQ ID NO: 620 - DNA, 24 bp, synthetic construct
  source 1..24: mol_type = other DNA
ggcacctattctacttcccaaagg

SEQ ID NO: 621 - DNA, 27 bp, synthetic construct
  source 1..27: mol_type = other DNA
ggcaaatatcaggatctagtgcacatt

SEQ ID NO: 622 - DNA, 28 bp, synthetic construct
  source 1..28: mol_type = other DNA
aaatgtaatgaccttaggagctacagga

SEQ ID NO: 623 - DNA, 24 bp, synthetic construct
  source 1..24: mol_type = other DNA
agaggggacattaagtcagacgat

SEQ ID NO: 624 - DNA, 21 bp, synthetic construct
  source 1..21: mol_type = other DNA
accagcttctgcttctgttgc

SEQ ID NO: 625 - DNA, 25 bp, synthetic construct
  source 1..25: mol_type = other DNA
ggctgagagtcaaaccatccttttc

SEQ ID NO: 626 - DNA, 24 bp, synthetic construct
  source 1..24: mol_type = other DNA
atcccaatcagatgtcacgtctgt

SEQ ID NO: 627 - DNA, 22 bp, synthetic construct
  source 1..22: mol_type = other DNA
tccagcaggaactgagtagtct

SEQ ID NO: 628 - DNA, 24 bp, synthetic construct
  source 1..24: mol_type = other DNA
ggcatctaactgatgatcggttgc

SEQ ID NO: 629 - DNA, 24 bp, synthetic construct
  source 1..24: mol_type = other DNA
gcgttcatgttgggaatcttgagg

SEQ ID NO: 630 - DNA, 27 bp, synthetic construct
  source 1..27: mol_type = other DNA
ggagcttagtctttctggtatccagct

SEQ ID NO: 631 - DNA, 24 bp, synthetic construct
  source 1..24: mol_type = other DNA
ttcagtccgctctgcatacagggc

SEQ ID NO: 632 - DNA, 22 bp, synthetic construct
  source 1..22: mol_type = other DNA
ctagccaagaccgaggataggt

SEQ ID NO: 633 - DNA, 23 bp, synthetic construct
  source 1..23: mol_type = other DNA
tttaggcagcctgtcatcctctc

SEQ ID NO: 634 - DNA, 25 bp, synthetic construct
  source 1..25: mol_type = other DNA
tgcgtatcattcatgtgcatcgatg

SEQ ID NO: 635 - DNA, 22 bp, synthetic construct
  source 1..22: mol_type = other DNA
ggcaaacatgcacctgaaagag

SEQ ID NO: 636 - DNA, 23 bp, synthetic construct
  source 1..23: mol_type = other DNA
aacaccccatctgttcacttagc

SEQ ID NO: 637 - DNA, 23 bp, synthetic construct
  source 1..23: mol_type = other DNA
tcttctcggacagctccttctcc

SEQ ID NO: 638 - DNA, 25 bp, synthetic construct
  source 1..25: mol_type = other DNA
ggacagaaagggccggatgttaagg

SEQ ID NO: 639 - DNA, 22 bp, synthetic construct
  source 1..22: mol_type = other DNA
tttccttctgttccggtgatgg

SEQ ID NO: 640 - DNA, 22 bp, synthetic construct
  source 1..22: mol_type = other DNA
tatttgagaaccctggggcagt

SEQ ID NO: 641 - DNA, 23 bp, synthetic construct
  source 1..23: mol_type = other DNA
gcctctggggaagatttttgagg

SEQ ID NO: 642 - DNA, 23 bp, synthetic construct
  source 1..23: mol_type = other DNA
cacagagtatgatgggcaggatg

SEQ ID NO: 643 - DNA, 25 bp, synthetic construct
  source 1..25: mol_type = other DNA
gtcttcctcacttgaggagatgagg

SEQ ID NO: 644 - DNA, 22 bp, synthetic construct
  source 1..22: mol_type = other DNA
ttcatgacggtcaggatgaacc

SEQ ID NO: 645 - DNA, 22 bp, synthetic construct
  source 1..22: mol_type = other DNA
aggaggagtctggaatcacaga

SEQ ID NO: 646 - DNA, 24 bp, synthetic construct
  source 1..24: mol_type = other DNA
ctgtacctgtgcaagatgtctcca

SEQ ID NO: 647 - DNA, 26 bp, synthetic construct
  source 1..26: mol_type = other DNA
agctattttggcaagagtgtggactt

SEQ ID NO: 648 - DNA, 24 bp, synthetic construct
  source 1..24: mol_type = other DNA
aattcaggtaagtgaggaggaggt

SEQ ID NO: 649 - DNA, 22 bp, synthetic construct
  source 1..22: mol_type = other DNA
tcttgtccagggctttgttcac

SEQ ID NO: 650 - DNA, 23 bp, synthetic construct
  source 1..23: mol_type = other DNA
ccggttattctcaatgggctcca

SEQ ID NO: 651 - DNA, 22 bp, synthetic construct
  source 1..22: mol_type = other DNA
gctcttcccttcacataccagt

SEQ ID NO: 652 - DNA, 23 bp, synthetic construct
  source 1..23: mol_type = other DNA
ccatggtcaatccactgcctcag

SEQ ID NO: 653 - DNA, 24 bp, synthetic construct
  source 1..24: mol_type = other DNA
ctgatagggcctcacagtgttggg

SEQ ID NO: 654 - DNA, 25 bp, synthetic construct
  source 1..25: mol_type = other DNA
aaggcctgaggagcttttgaggtag

SEQ ID NO: 655 - DNA, 22 bp, synthetic construct
  source 1..22: mol_type = other DNA
aaaggcctcctccacaaggacc

SEQ ID NO: 656 - DNA, 22 bp, synthetic construct
  source 1..22: mol_type = other DNA
ccaacccacccaaaccgtctct

SEQ ID NO: 657 - DNA, 31 bp, synthetic construct
  source 1..31: mol_type = other DNA
ccaatccaaatctgtttttctgaatctgttt

SEQ ID NO: 658 - DNA, 28 bp, synthetic construct
  source 1..28: mol_type = other DNA
acactggaagagaatattcttctgacct

SEQ ID NO: 659 - DNA, 28 bp, synthetic construct
  source 1..28: mol_type = other DNA
ctatcttcaggaggaggagaagtctcct

SEQ ID NO: 660 - DNA, 28 bp, synthetic construct
  source 1..28: mol_type = other DNA
aaattggcagcaaatgtaacagttactc

SEQ ID NO: 661 - DNA, 21 bp, synthetic construct
  source 1..21: mol_type = other DNA
tcctttagagaggccctgctg

SEQ ID NO: 662 - DNA, 23 bp, synthetic construct
  source 1..23: mol_type = other DNA
aagctcatcgcagatgcctggaa

SEQ ID NO: 663 - DNA, 21 bp, synthetic construct
  source 1..21: mol_type = other DNA
tggcctagaaagaggctcgac

SEQ ID NO: 664 - DNA, 25 bp, synthetic construct
  source 1..25: mol_type = other DNA
tggggctatcatctccgtggtcatc

SEQ ID NO: 665 - DNA, 22 bp, synthetic construct
  source 1..22: mol_type = other DNA
tggccgattttgtccagacaag

SEQ ID NO: 666 - DNA, 26 bp, synthetic construct
  source 1..26: mol_type = other DNA
gggactgtcactcttcctgtctaggt

SEQ ID NO: 667 - DNA, 22 bp, synthetic construct
  source 1..22: mol_type = other DNA
taaatgtgtggcccactcttgg

SEQ ID NO: 668 - DNA, 23 bp, synthetic construct
  source 1..23: mol_type = other DNA
tacctgttccaccaactcttgct

SEQ ID NO: 669 - DNA, 25 bp, synthetic construct
  source 1..25: mol_type = other DNA
cccagcagtcctacctttaaacagc

SEQ ID NO: 670 - DNA, 23 bp, synthetic construct
  source 1..23: mol_type = other DNA
ccatgggggtaaaacggatgact

SEQ ID NO: 671 - DNA, 22 bp, synthetic construct
  source 1..22: mol_type = other DNA
cacgaagaacagccataggaca

SEQ ID NO: 672 - DNA, 25 bp, synthetic construct
  source 1..25: mol_type = other DNA
gctcttcaccagttggatcaggtag

SEQ ID NO: 673 - DNA, 30 bp, synthetic construct
  source 1..30: mol_type = other DNA
cggtttgctttagagtgcattaacttcttt

SEQ ID NO: 674 - DNA, 29 bp, synthetic construct
  source 1..29: mol_type = other DNA
aatagcttctgtgtgtttcctatttttgg

SEQ ID NO: 675 - DNA, 23 bp, synthetic construct
  source 1..23: mol_type = other DNA
ttccagtgcctggaggagacccc

SEQ ID NO: 676 - DNA, 22 bp, synthetic construct
  source 1..22: mol_type = other DNA
acacaccacttctccctcagca

SEQ ID NO: 677 - DNA, 23 bp, synthetic construct
  source 1..23: mol_type = other DNA
gcgacgtggtggaagtctacctg

SEQ ID NO: 678 - DNA, 21 bp, synthetic construct
  source 1..21: mol_type = other DNA
ctgtgggctcctgtgaatgag

SEQ ID NO: 679 - DNA, 29 bp, synthetic construct
  source 1..29: mol_type = other DNA
cagtacatccagaatgtcacaacagatta

SEQ ID NO: 680 - DNA, 26 bp, synthetic construct
  source 1..26: mol_type = other DNA
tatctctggttttgaggtagagagct

SEQ ID NO: 681 - DNA, 22 bp, synthetic construct
  source 1..22: mol_type = other DNA
gcccagttgattgagctccaaa

SEQ ID NO: 682 - DNA, 23 bp, synthetic construct
  source 1..23: mol_type = other DNA
cctgctctgctgcaaaaggcaag

SEQ ID NO: 683 - DNA, 24 bp, synthetic construct
  source 1..24: mol_type = other DNA
ctccgggataggatggggtagacc

SEQ ID NO: 684 - DNA, 22 bp, synthetic construct
  source 1..22: mol_type = other DNA
cctgctccctcactatgccact

SEQ ID NO: 685 - DNA, 20 bp, synthetic construct
  source 1..20: mol_type = other DNA
agtctcagcggacttgtcca

SEQ ID NO: 686 - DNA, 21 bp, synthetic construct
  source 1..21: mol_type = other DNA
tcttaaggcagtacgggctct

SEQ ID NO: 687 - DNA, 23 bp, synthetic construct
  source 1..23: mol_type = other DNA
ctttgttcccagcctgacactgc

SEQ ID NO: 688 - DNA, 23 bp, synthetic construct
  source 1..23: mol_type = other DNA
acacagaagccttcatgggcatc

SEQ ID NO: 689 - DNA, 23 bp, synthetic construct
  source 1..23: mol_type = other DNA
gttgggggagagggaaagttctt

SEQ ID NO: 690 - DNA, 23 bp, synthetic construct
  source 1..23: mol_type = other DNA
acgaagaaggaggccgtcttgag

SEQ ID NO: 691 - DNA, 24 bp, synthetic construct
  source 1..24: mol_type = other DNA
gaattgtccctcagggattgcact

SEQ ID NO: 692 - DNA, 25 bp, synthetic construct
  source 1..25: mol_type = other DNA
acatgtagttcaggcctctcttcct

SEQ ID NO: 693 - DNA, 26 bp, synthetic construct
  source 1..26: mol_type = other DNA
ctccagtcacaactcctgataagtcc

SEQ ID NO: 694 - DNA, 24 bp, synthetic construct
  source 1..24: mol_type = other DNA
tgcagacaaactgattcaggtcgt

SEQ ID NO: 695 - DNA, 25 bp, synthetic construct
  source 1..25: mol_type = other DNA
gctgctcatctccgaagaagatggg

SEQ ID NO: 696 - DNA, 25 bp, synthetic construct
  source 1..25: mol_type = other DNA
agacttttcagagtcagctggcaca

SEQ ID NO: 697 - DNA, 23 bp, synthetic construct
  source 1..23: mol_type = other DNA
gcctctcttcccaaaacccatct

SEQ ID NO: 698 - DNA, 24 bp, synthetic construct
  source 1..24: mol_type = other DNA
gggaacactgaggcatggaatcct

SEQ ID NO: 699 - DNA, 23 bp, synthetic construct
  source 1..23: mol_type = other DNA
tggaaaaatgtcttgctggggta

SEQ ID NO: 700 - DNA, 24 bp, synthetic construct
  source 1..24: mol_type = other DNA
aagtacagatgtggaccaagagga

SEQ ID NO: 701 - DNA, 26 bp, synthetic construct
  source 1..26: mol_type = other DNA
agaggacctgaaggtattccacaatg

SEQ ID NO: 702 - DNA, 28 bp, synthetic construct
  source 1..28: mol_type = other DNA
taactagactatatcctggcctccctag

SEQ ID NO: 703 - DNA, 22 bp, synthetic construct
  source 1..22: mol_type = other DNA
gtggaaaacagggtccttgttg

SEQ ID NO: 704 - DNA, 24 bp, synthetic construct
  source 1..24: mol_type = other DNA
gtcatgggccattagtttggcttc

SEQ ID NO: 705 - DNA, 24 bp, synthetic construct
  source 1..24: mol_type = other DNA
gatgttctccaccgacgagtacag

SEQ ID NO: 706 - DNA, 24 bp, synthetic construct
  source 1..24: mol_type = other DNA
acctccaccctgtagcaattccat

SEQ ID NO: 707 - DNA, 25 bp, synthetic construct
  source 1..25: mol_type = other DNA
agagcctggttctatgatgaagatg

SEQ ID NO: 708 - DNA, 36 bp, synthetic construct
  source 1..36: mol_type = other DNA
ttacattatcaaatggtcatatatatggtaaagctt

SEQ ID NO: 709 - DNA, 24 bp, synthetic construct
  source 1..24: mol_type = other DNA
ctcaccaacatcgcgattcctatc

SEQ ID NO: 710 - DNA, 24 bp, synthetic construct
  source 1..24: mol_type = other DNA
agtcttcaccctccaagatcacca

SEQ ID NO: 711 - DNA, 24 bp, synthetic construct
  source 1..24: mol_type = other DNA
agggctaaaacatgagcaggtaca

SEQ ID NO: 712 - DNA, 26 bp, synthetic construct
  source 1..26: mol_type = other DNA
aaagggtcacatttctgatgagctct

SEQ ID NO: 713 - DNA, 21 bp, synthetic construct
  source 1..21: mol_type = other DNA
ttagccccaaacaccccactc

SEQ ID NO: 714 - DNA, 23 bp, synthetic construct
  source 1..23: mol_type = other DNA
ctgtcacttcccgaactggcagt

SEQ ID NO: 715 - DNA, 23 bp, synthetic construct
  source 1..23: mol_type = other DNA
ccagagcaaaatgggacacagta

SEQ ID NO: 716 - DNA, 24 bp, synthetic construct
  source 1..24: mol_type = other DNA
gtcatgttcagccagtaccatcgt

SEQ ID NO: 717 - DNA, 22 bp, synthetic construct
  source 1..22: mol_type = other DNA
aattcgagggcaggagattacc

SEQ ID NO: 718 - DNA, 22 bp, synthetic construct
  source 1..22: mol_type = other DNA
gccaaggtttgctaatgctgct

SEQ ID NO: 719 - DNA, 25 bp, synthetic construct
  source 1..25: mol_type = other DNA
caacaccagcaaacacaccttccaa

SEQ ID NO: 720 - DNA, 20 bp, synthetic construct
  source 1..20: mol_type = other DNA
cagtcctgtgcgtccaaagc

SEQ ID NO: 721 - DNA, 22 bp, synthetic construct
  source 1..22: mol_type = other DNA
ccctggagtaatcacctgctct

SEQ ID NO: 722 - DNA, 25 bp, synthetic construct
  source 1..25: mol_type = other DNA
gctgtatgtccacccggattctcct

SEQ ID NO: 723 - DNA, 23 bp, synthetic construct
  source 1..23: mol_type = other DNA
agacctgctgcctatacagtcac

SEQ ID NO: 724 - DNA, 23 bp, synthetic construct
  source 1..23: mol_type = other DNA
caaacccctaccagatctgacga

SEQ ID NO: 725 - DNA, 19 bp, synthetic construct
  source 1..19: mol_type = other DNA
tggcacaaggccctcaacc

SEQ ID NO: 726 - DNA, 19 bp, synthetic construct
  source 1..19: mol_type = other DNA
agttccacgaggcactgtg

SEQ ID NO: 727 - DNA, 22 bp, synthetic construct
  source 1..22: mol_type = other DNA
aggactccatccctggggattg

SEQ ID NO: 728 - DNA, 24 bp, synthetic construct
  source 1..24: mol_type = other DNA
cccacctctctgaggagctcatgc

SEQ ID NO: 729 - DNA, 27 bp, synthetic construct
  source 1..27: mol_type = other DNA
ctccctcatctctatgaggtctgtgtt

SEQ ID NO: 730 - DNA, 23 bp, synthetic construct
  source 1..23: mol_type = other DNA
ggccttgagaagtggttctctgt

SEQ ID NO: 731 - DNA, 22 bp, synthetic construct
  source 1..22: mol_type = other DNA
atggtcccacatccatgtgtgg

SEQ ID NO: 732 - DNA, 21 bp, synthetic construct
  source 1..21: mol_type = other DNA
agatccagctaacaggcgcta

SEQ ID NO: 733 - DNA, 22 bp, synthetic construct
  source 1..22: mol_type = other DNA
tccagtctttgctcgagacttg

SEQ ID NO: 734 - DNA, 33 bp, synthetic construct
  source 1..33: mol_type = other DNA
tttacttttgtacagatgttaaatttgtgacac

SEQ ID NO: 735 - DNA, 22 bp, synthetic construct
  source 1..22: mol_type = other DNA
ccatcccaaggtctttgaaggt

SEQ ID NO: 736 - DNA, 22 bp, synthetic construct
  source 1..22: mol_type = other DNA
ttcatctggacacctcctctca

SEQ ID NO: 737 - DNA, 23 bp, synthetic construct
  source 1..23: mol_type = other DNA
ccataagtgactgtgacgttgca

SEQ ID NO: 738 - DNA, 24 bp, synthetic construct
  source 1..24: mol_type = other DNA
aagcagacaacccgaagtaaacac

SEQ ID NO: 739 - DNA, 21 bp, synthetic construct
  source 1..21: mol_type = other DNA
agggacagaacacagtgaacc

SEQ ID NO: 740 - DNA, 21 bp, synthetic construct
  source 1..21: mol_type = other DNA
caacaggcacccgattttctc

SEQ ID NO: 741 - DNA, 26 bp, synthetic construct
  source 1..26: mol_type = other DNA
gcctcaggatagaattccctttcagt

SEQ ID NO: 742 - DNA, 23 bp, synthetic construct
  source 1..23: mol_type = other DNA
gaccatggccaaatacatcctga

SEQ ID NO: 743 - DNA, 26 bp, synthetic construct
  source 1..26: mol_type = other DNA
gaagaaatggcagcatctgaacccgt

SEQ ID NO: 744 - DNA, 23 bp, synthetic construct
  source 1..23: mol_type = other DNA
cccaatgttgtagtaaccgaagc

SEQ ID NO: 745 - DNA, 25 bp, synthetic construct
  source 1..25: mol_type = other DNA
catcctgcctttagtcctgagttct

SEQ ID NO: 746 - DNA, 22 bp, synthetic construct
  source 1..22: mol_type = other DNA
gcaatgggaggaatgttcatgg

SEQ ID NO: 747 - DNA, 26 bp, synthetic construct
  source 1..26: mol_type = other DNA
tcagctaggagaggggaataaggcac

SEQ ID NO: 748 - DNA, 23 bp, synthetic construct
  source 1..23: mol_type = other DNA
caagaagagctggcacctagatc

SEQ ID NO: 749 - DNA, 22 bp, synthetic construct
  source 1..22: mol_type = other DNA
gcatggaaagacagaggacgag

SEQ ID NO: 750 - DNA, 22 bp, synthetic construct
  source 1..22: mol_type = other DNA
ttcatgtggatgcaggctacag

SEQ ID NO: 751 - DNA, 29 bp, synthetic construct
  source 1..29: mol_type = other DNA
gcagtagagtatgagttcttcctttccta

SEQ ID NO: 752 - DNA, 27 bp, synthetic construct
  source 1..27: mol_type = other DNA
tccctctttactatgtgcaggtgctct

SEQ ID NO: 753 - DNA, 22 bp, synthetic construct
  source 1..22: mol_type = other DNA
ttctgttggtgatcaccctcct

SEQ ID NO: 754 - DNA, 27 bp, synthetic construct
  source 1..27: mol_type = other DNA
caccagtgatatacttgcccaggtcag

SEQ ID NO: 755 - DNA, 22 bp, synthetic construct
  source 1..22: mol_type = other DNA
ggcaatctgggagagttttgca

SEQ ID NO: 756 - DNA, 23 bp, synthetic construct
  source 1..23: mol_type = other DNA
ggacagagaaatccgatgcggtt

SEQ ID NO: 757 - DNA, 20 bp, synthetic construct
  source 1..20: mol_type = other DNA
cctggaggacgcgtgagtgc

SEQ ID NO: 758 - DNA, 20 bp, synthetic construct
  source 1..20: mol_type = other DNA
cgacgggaaggtgcaggttc

SEQ ID NO: 759 - DNA, 19 bp, synthetic construct
  source 1..19: mol_type = other DNA
agggccgtcaagatcctca

SEQ ID NO: 760 - DNA, 21 bp, synthetic construct
  source 1..21: mol_type = other DNA
cacggtgctgatggttctgtc

SEQ ID NO: 761 - DNA, 25 bp, synthetic construct
  source 1..25: mol_type = other DNA
atggtagaaccgggtacagctttcc

SEQ ID NO: 762 - DNA, 24 bp, synthetic construct
  source 1..24: mol_type = other DNA
agatgctaggtcttcaacccctca

SEQ ID NO: 763 - DNA, 26 bp, synthetic construct
  source 1..26: mol_type = other DNA
caccagtccatggaaaaaccatcttc

SEQ ID NO: 764 - DNA, 22 bp, synthetic construct
  source 1..22: mol_type = other DNA
cattggtcttcagggcaatgtc

SEQ ID NO: 765 - DNA, 21 bp, synthetic construct
  source 1..21: mol_type = other DNA
tatgagcgtggccggatctcc

SEQ ID NO: 766 - DNA, 21 bp, synthetic construct
  source 1..21: mol_type = other DNA
gagcatcaggaggtcacaagc

SEQ ID NO: 767 - DNA, 19 bp, synthetic construct
  source 1..19: mol_type = other DNA
tgtggccaggtggtggtgg

SEQ ID NO: 768 - DNA, 24 bp, synthetic construct
  source 1..24: mol_type = other DNA
ccttaagcagctagtcctgctcca

SEQ ID NO: 769 - DNA, 20 bp, synthetic construct
  source 1..20: mol_type = other DNA
tggtgggaccagagaaccag

SEQ ID NO: 770 - DNA, 23 bp, synthetic construct
  source 1..23: mol_type = other DNA
tcaagaaggatggggagtggaag

SEQ ID NO: 771 - DNA, 22 bp, synthetic construct
  source 1..22: mol_type = other DNA
gacagaggatgtgggccatgct

SEQ ID NO: 772 - DNA, 22 bp, synthetic construct
  source 1..22: mol_type = other DNA
gaagatgggctgctcatcactg

SEQ ID NO: 773 - DNA, 20 bp, synthetic construct
  source 1..20: mol_type = other DNA
ggaactttcggagccgcaag

SEQ ID NO: 774 - DNA, 21 bp, synthetic construct
  source 1..21: mol_type = other DNA
accaggcagggaacgtgtcca

SEQ ID NO: 775 - DNA, 21 bp, synthetic construct
  source 1..21: mol_type = other DNA
aaagcatttctccccgcagtc

SEQ ID NO: 776 - DNA, 24 bp, synthetic construct
  source 1..24: mol_type = other DNA
tgccgagtttttgcactgcaagtc

SEQ ID NO: 777 - DNA, 24 bp, synthetic construct
  source 1..24: mol_type = other DNA
catgttccagcacatcgaggtgag

SEQ ID NO: 778 - DNA, 26 bp, synthetic construct
  source 1..26: mol_type = other DNA
ctcgacagctctttaaaaacagactc

SEQ ID NO: 779 - DNA, 24 bp, synthetic construct
  source 1..24: mol_type = other DNA
ctctccaggtgcttttctgctagg

SEQ ID NO: 780 - DNA, 25 bp, synthetic construct
  source 1..25: mol_type = other DNA
acctctccttatccacttgtgtgtc

SEQ ID NO: 781 - DNA, 20 bp, synthetic construct
  source 1..20: mol_type = other DNA
tgagaagtaggtggcctcca

SEQ ID NO: 782 - DNA, 20 bp, synthetic construct
  source 1..20: mol_type = other DNA
tgagaagtaggtggcctcca

SEQ ID NO: 783 - DNA, 23 bp, synthetic construct
  source 1..23: mol_type = other DNA
tctgaggtttgcttccagtgtgt

SEQ ID NO: 784 - DNA, 20 bp, synthetic construct
  source 1..20: mol_type = other DNA
aaaacacagggcacacgtga

SEQ ID NO: 785 - DNA, 24 bp, synthetic construct
  source 1..24: mol_type = other DNA
ggacaatgggcagatacacagtag

SEQ ID NO: 786 - DNA, 23 bp, synthetic construct
  source 1..23: mol_type = other DNA
cttctgtggacaatgacccagtc

SEQ ID NO: 787 - DNA, 21 bp, synthetic construct
  source 1..21: mol_type = other DNA
actcactggggtcacagtcat

SEQ ID NO: 788 - DNA, 24 bp, synthetic construct
  source 1..24: mol_type = other DNA
ctgccaggttggtgcagataccat

SEQ ID NO: 789 - DNA, 21 bp, synthetic construct
  source 1..21: mol_type = other DNA
tatgatgctgggaccaggtga

SEQ ID NO: 790 - DNA, 21 bp, synthetic construct
  source 1..21: mol_type = other DNA
cgaggggctgcagcttttgtc

SEQ ID NO: 791 - DNA, 21 bp, synthetic construct
  source 1..21: mol_type = other DNA
tgacgtagttcaccagcatgg

SEQ ID NO: 792 - DNA, 21 bp, synthetic construct
  source 1..21: mol_type = other DNA
cggcttcgttaaagagcctct

SEQ ID NO: 793 - DNA, 18 bp, synthetic construct
  source 1..18: mol_type = other DNA
acaggacaggctggctct

SEQ ID NO: 794 - DNA, 21 bp, synthetic construct
  source 1..21: mol_type = other DNA
agcatgcctgtcccccacaga

SEQ ID NO: 795 - DNA, 22 bp, synthetic construct
  source 1..22: mol_type = other DNA
atcctcaacgagttcgccaaga

SEQ ID NO: 796 - DNA, 23 bp, synthetic construct
  source 1..23: mol_type = other DNA
gcccagcccaataaacctgcact

SEQ ID NO: 797 - DNA, 22 bp, synthetic construct
  source 1..22: mol_type = other DNA
cagtgctcactgtctttgtgtg

SEQ ID NO: 798 - DNA, 21 bp, synthetic construct
  source 1..21: mol_type = other DNA
agtcctggtcatcctctcctt

SEQ ID NO: 799 - DNA, 23 bp, synthetic construct
  source 1..23: mol_type = other DNA
tccctatctatgccagcctcagt

SEQ ID NO: 800 - DNA, 22 bp, synthetic construct
  source 1..22: mol_type = other DNA
ggcacctgctagccccgttctg

SEQ ID NO: 801 - DNA, 25 bp, synthetic construct
  source 1..25: mol_type = other DNA
tcagtgtgatctcccttgtggtgcc

SEQ ID NO: 802 - DNA, 18 bp, synthetic construct
  source 1..18: mol_type = other DNA
ttccgcaggtcctggctt

SEQ ID NO: 803 - DNA, 24 bp, synthetic construct
  source 1..24: mol_type = other DNA
ccctcctgcttctcatagaagttg

SEQ ID NO: 804 - DNA, 23 bp, synthetic construct
  source 1..23: mol_type = other DNA
cagtctcttccccagcttcccaa

SEQ ID NO: 805 - DNA, 23 bp, synthetic construct
  source 1..23: mol_type = other DNA
cccaataaaccccgcactgtgcc

SEQ ID NO: 806 - DNA, 24 bp, synthetic construct
  source 1..24: mol_type = other DNA
cggctgatgatgtccgggtacttc

SEQ ID NO: 807 - DNA, 25 bp, synthetic construct
  source 1..25: mol_type = other DNA
accagaaactcactggaggtcttta

SEQ ID NO: 808 - DNA, 26 bp, synthetic construct
  source 1..26: mol_type = other DNA
ctgacgatagacagaacggtcagatg

SEQ ID NO: 809 - DNA, 26 bp, synthetic construct
  source 1..26: mol_type = other DNA
gaggacttggagaaggaaagactcag

SEQ ID NO: 810 - DNA, 25 bp, synthetic construct
  source 1..25: mol_type = other DNA
cagctgctcctggaacagaaatcca

SEQ ID NO: 811 - DNA, 19 bp, synthetic construct
  source 1..19: mol_type = other DNA
ccacaccccattccccaac

SEQ ID NO: 812 - DNA, 20 bp, synthetic construct
  source 1..20: mol_type = other DNA
cagtagccggcacagatggt

SEQ ID NO: 813 - DNA, 20 bp, synthetic construct
  source 1..20: mol_type = other DNA
caggatggcattgatggggt

SEQ ID NO: 814 - DNA, 19 bp, synthetic construct
  source 1..19: mol_type = other DNA
accgcagccccgagtcctg

SEQ ID NO: 815 - DNA, 21 bp, synthetic construct
  source 1..21: mol_type = other DNA
gctgctgtattgctggggatc

SEQ ID NO: 816 - DNA, 21 bp, synthetic construct
  source 1..21: mol_type = other DNA
ggtggatgttgttgtggaggt

SEQ ID NO: 817 - DNA, 23 bp, synthetic construct
  source 1..23: mol_type = other DNA
gttcctgctgtgtgtcactctga

SEQ ID NO: 818 - DNA, 22 bp, synthetic construct
  source 1..22: mol_type = other DNA
gatcctgacacccaatgccatc

SEQ ID NO: 819 - DNA, 22 bp, synthetic construct
  source 1..22: mol_type = other DNA
tgacgaaacagaagacccgaac

SEQ ID NO: 820 - DNA, 24 bp, synthetic construct
  source 1..24: mol_type = other DNA
ggaaggcaatgacagctgacagag

SEQ ID NO: 821 - DNA, 24 bp, synthetic construct
  source 1..24: mol_type = other DNA
gcctaccgtaggtgttttaggtta

SEQ ID NO: 822 - DNA, 28 bp, synthetic construct
  source 1..28: mol_type = other DNA
cgcctctgagagatatctacagccaagc

SEQ ID NO: 823 - DNA, 23 bp, synthetic construct
  source 1..23: mol_type = other DNA
gcataagcgcggtagttggagga

SEQ ID NO: 824 - DNA, 21 bp, synthetic construct
  source 1..21: mol_type = other DNA
ccttaccgtaccgcaccctct

SEQ ID NO: 825 - DNA, 25 bp, synthetic construct
  source 1..25: mol_type = other DNA
cagaatggcaagaggactgacagtc

SEQ ID NO: 826 - DNA, 21 bp, synthetic construct
  source 1..21: mol_type = other DNA
gtttgacatcaccccctccaa

SEQ ID NO: 827 - DNA, 23 bp, synthetic construct
  source 1..23: mol_type = other DNA
tcaccttgaacacctccttgacg

SEQ ID NO: 828 - DNA, 29 bp, synthetic construct
  source 1..29: mol_type = other DNA
gggggaagaggggagaagtaaagatagag

SEQ ID NO: 829 - DNA, 20 bp, synthetic construct
  source 1..20: mol_type = other DNA
gcgtctccttcacgttcaca

SEQ ID NO: 830 - DNA, 23 bp, synthetic construct
  source 1..23: mol_type = other DNA
ccctatgtcccatgtggccagag

SEQ ID NO: 831 - DNA, 22 bp, synthetic construct
  source 1..22: mol_type = other DNA
gtggtgatgtatttgcccacag

SEQ ID NO: 832 - DNA, 23 bp, synthetic construct
  source 1..23: mol_type = other DNA
acgtaccatcagtgaagtccaag

SEQ ID NO: 833 - DNA, 21 bp, synthetic construct
  source 1..21: mol_type = other DNA
gcaatgcttctgttcccacca

SEQ ID NO: 834 - DNA, 21 bp, synthetic construct
  source 1..21: mol_type = other DNA
aaatgagagagggcccagctt

SEQ ID NO: 835 - DNA, 24 bp, synthetic construct
  source 1..24: mol_type = other DNA
tgcagaagaggaacctgtcatcac

SEQ ID NO: 836 - DNA, 25 bp, synthetic construct
  source 1..25: mol_type = other DNA
cctcctaaggtagaaagtcccagtg

SEQ ID NO: 837 - DNA, 24 bp, synthetic construct
  source 1..24: mol_type = other DNA
tctatacagtgctgggaggactct

SEQ ID NO: 838 - DNA, 21 bp, synthetic construct
  source 1..21: mol_type = other DNA
attcagcaagcctgaggagtg

SEQ ID NO: 839 - DNA, 23 bp, synthetic construct
  source 1..23: mol_type = other DNA
atcatgcccgagtgagaagcctt

SEQ ID NO: 840 - DNA, 24 bp, synthetic construct
  source 1..24: mol_type = other DNA
cttcttccctgaccattttggctt

SEQ ID NO: 841 - DNA, 23 bp, synthetic construct
  source 1..23: mol_type = other DNA
ctacccagagacaacctgttacc

SEQ ID NO: 842 - DNA, 23 bp, synthetic construct
  source 1..23: mol_type = other DNA
atgtgtgcatgtgagtcgtttgg

SEQ ID NO: 843 - DNA, 23 bp, synthetic construct
  source 1..23: mol_type = other DNA
cgggtgtgtgtttgtatccagtc

SEQ ID NO: 844 - DNA, 22 bp, synthetic construct
  source 1..22: mol_type = other DNA
cctttctcctgcacaactgact

SEQ ID NO: 845 - DNA, 22 bp, synthetic construct
  source 1..22: mol_type = other DNA
acccagagctaccaatgacttc

SEQ ID NO: 846 - DNA, 22 bp, synthetic construct
  source 1..22: mol_type = other DNA
gcactctaggaaaagtcccaca

SEQ ID NO: 847 - DNA, 23 bp, synthetic construct
  source 1..23: mol_type = other DNA
agctgccacaatgggacatgtgc

SEQ ID NO: 848 - DNA, 21 bp, synthetic construct
  source 1..21: mol_type = other DNA
atcggcaggtttctcagggat

SEQ ID NO: 849 - DNA, 24 bp, synthetic construct
  source 1..24: mol_type = other DNA
gaaacaaaaggccagtcgttggca

SEQ ID NO: 850 - DNA, 22 bp, synthetic construct
  source 1..22: mol_type = other DNA
cccaagcacacagcttcaggat

SEQ ID NO: 851 - DNA, 23 bp, synthetic construct
  source 1..23: mol_type = other DNA
ccgaatatgttgctgcagcatca

SEQ ID NO: 852 - DNA, 22 bp, synthetic construct
  source 1..22: mol_type = other DNA
ccccaaggtgtgtcttcaggtt

SEQ ID NO: 853 - DNA, 23 bp, synthetic construct
  source 1..23: mol_type = other DNA
gtttgctccaactgacacttcag

SEQ ID NO: 854 - DNA, 24 bp, synthetic construct
  source 1..24: mol_type = other DNA
agatatggaattgtgggtgcatgc

SEQ ID NO: 855 - DNA, 19 bp, synthetic construct
  source 1..19: mol_type = other DNA
cctgacatccgcggcatgg

SEQ ID NO: 856 - DNA, 21 bp, synthetic construct
  source 1..21: mol_type = other DNA
tagggtgggagagcacaaagc

SEQ ID NO: 857 - DNA, 19 bp, synthetic construct
  source 1..19: mol_type = other DNA
ctcacagggagaggctggt

SEQ ID NO: 858 - DNA, 19 bp, synthetic construct
  source 1..19: mol_type = other DNA
ctgggtgggttggtcactg

SEQ ID NO: 859 - DNA, 20 bp, synthetic construct
  source 1..20: mol_type = other DNA
ttgcccagggctgaacatgg

SEQ ID NO: 860 - DNA, 19 bp, synthetic construct
  source 1..19: mol_type = other DNA
gaggaggtgggtgacacct

SEQ ID NO: 861 - DNA, 23 bp, synthetic construct
  source 1..23: mol_type = other DNA
ttcctgcggtgtctgaaacatca

SEQ ID NO: 862 - DNA, 20 bp, synthetic construct
  source 1..20: mol_type = other DNA
aggagggacagggtttgcat

SEQ ID NO: 863 - DNA, 26 bp, synthetic construct
  source 1..26: mol_type = other DNA
gagctgggaggtagttctaagtgcac

SEQ ID NO: 864 - DNA, 30 bp, synthetic construct
  source 1..30: mol_type = other DNA
aaatttagagacaggatctcattatgttgc

SEQ ID NO: 865 - DNA, 25 bp, synthetic construct
  source 1..25: mol_type = other DNA
agtcaccttgtcatctggcaagagg

SEQ ID NO: 866 - DNA, 25 bp, synthetic construct
  source 1..25: mol_type = other DNA
cagacccgacgtggtaactttacac

SEQ ID NO: 867 - DNA, 22 bp, synthetic construct
  source 1..22: mol_type = other DNA
tccccgtggtaccaaagctgaa

SEQ ID NO: 868 - DNA, 21 bp, synthetic construct
  source 1..21: mol_type = other DNA
gtatgacctgctgcccttcct

SEQ ID NO: 869 - DNA, 23 bp, synthetic construct
  source 1..23: mol_type = other DNA
caaagtcttctcggtggccatca

SEQ ID NO: 870 - DNA, 20 bp, synthetic construct
  source 1..20: mol_type = other DNA
tgggaccgtcttttggtcct

SEQ ID NO: 871 - DNA, 22 bp, synthetic construct
  source 1..22: mol_type = other DNA
catgtgcctgggacatgtgtcc

SEQ ID NO: 872 - DNA, 21 bp, synthetic construct
  source 1..21: mol_type = other DNA
ttcccactgactcgtctccat

SEQ ID NO: 873 - DNA, 22 bp, synthetic construct
  source 1..22: mol_type = other DNA
ttggtctgagaccctcagctta

SEQ ID NO: 874 - DNA, 21 bp, synthetic construct
  source 1..21: mol_type = other DNA
tggccctatcggacctaaagg

SEQ ID NO: 875 - DNA, 26 bp, synthetic construct
  source 1..26: mol_type = other DNA
tagctcctgagagaatggatcccggg

SEQ ID NO: 876 - DNA, 19 bp, synthetic construct
  source 1..19: mol_type = other DNA
ctccaagccagcagggttc

SEQ ID NO: 877 - DNA, 23 bp, synthetic construct
  source 1..23: mol_type = other DNA
gctggaggatgccttcctgaaga

SEQ ID NO: 878 - DNA, 19 bp, synthetic construct
  source 1..19: mol_type = other DNA
gcgtaggtgcacgcggggc

SEQ ID NO: 879 - DNA, 24 bp, synthetic construct
  source 1..24: mol_type = other DNA
gtaagtggccttgtcagggtacgg

SEQ ID NO: 880 - DNA, 23 bp, synthetic construct
  source 1..23: mol_type = other DNA
agagggcctttctggaaacactg

SEQ ID NO: 881 - DNA, 18 bp, synthetic construct
  source 1..18: mol_type = other DNA
aggcagggtgcgaatgga

SEQ ID NO: 882 - DNA, 21 bp, synthetic construct
  source 1..21: mol_type = other DNA
cagagctgtccgtggcacagt

SEQ ID NO: 883 - DNA, 22 bp, synthetic construct
  source 1..22: mol_type = other DNA
ttgccattactgacctggagag

SEQ ID NO: 884 - DNA, 23 bp, synthetic construct
  source 1..23: mol_type = other DNA
ctgttatccccacagggcacagt

SEQ ID NO: 885 - DNA, 24 bp, synthetic construct
  source 1..24: mol_type = other DNA
ggcatttcctgtgggaaactgact

SEQ ID NO: 886 - DNA, 24 bp, synthetic construct
  source 1..24: mol_type = other DNA
atcaccgagaaattcagtgcggaa

SEQ ID NO: 887 - DNA, 19 bp, synthetic construct
  source 1..19: mol_type = other DNA
tctggcccacgtggtcaga

SEQ ID NO: 888 - DNA, 21 bp, synthetic construct
  source 1..21: mol_type = other DNA
gcagagccagctggaggagga

SEQ ID NO: 889 - DNA, 20 bp, synthetic construct
  source 1..20: mol_type = other DNA
aggatggtgggggtctctgg

SEQ ID NO: 890 - DNA, 21 bp, synthetic construct
  source 1..21: mol_type = other DNA
ctgacctatgaggagcgggtt

SEQ ID NO: 891 - DNA, 22 bp, synthetic construct
  source 1..22: mol_type = other DNA
gctaacaagcccctcttctctc

SEQ ID NO: 892 - DNA, 21 bp, synthetic construct
  source 1..21: mol_type = other DNA
ctctcacactgtgggcatctg

SEQ ID NO: 893 - DNA, 22 bp, synthetic construct
  source 1..22: mol_type = other DNA
cactccagccggagtttgagca

SEQ ID NO: 894 - DNA, 22 bp, synthetic construct
  source 1..22: mol_type = other DNA
cgtagctgtagcggtaacaacc

SEQ ID NO: 895 - DNA, 21 bp, synthetic construct
  source 1..21: mol_type = other DNA
agaggagggaagtgcatctca

SEQ ID NO: 896 - DNA, 24 bp, synthetic construct
  source 1..24: mol_type = other DNA
cccactggcttccctatggtcaga

SEQ ID NO: 897 - DNA, 24 bp, synthetic construct
  source 1..24: mol_type = other DNA
ccaggttcaagtatgtcctggtca

SEQ ID NO: 898 - DNA, 20 bp, synthetic construct
  source 1..20: mol_type = other DNA
gcagtggtggtggtgatgct

SEQ ID NO: 899 - DNA, 24 bp, synthetic construct
  source 1..24: mol_type = other DNA
ggttaccctgggtgaagaagtgag

SEQ ID NO: 900 - DNA, 24 bp, synthetic construct
  source 1..24: mol_type = other DNA
ggacgggtagaagaagagagactg

SEQ ID NO: 901 - DNA, 24 bp, synthetic construct
  source 1..24: mol_type = other DNA
ctacaaacctgtccagaaagaggt

SEQ ID NO: 902 - DNA, 27 bp, synthetic construct
  source 1..27: mol_type = other DNA
agactggtataagctgaaaaaggacag

SEQ ID NO: 903 - DNA, 25 bp, synthetic construct
  source 1..25: mol_type = other DNA
cagtagcaaatagaagccatcaagg

SEQ ID NO: 904 - DNA, 26 bp, synthetic construct
  source 1..26: mol_type = other DNA
tcagtgatagcagtctccaaaacaag

SEQ ID NO: 905 - DNA, 22 bp, synthetic construct
  source 1..22: mol_type = other DNA
cttccctgtggaagtgatgctg

SEQ ID NO: 906 - DNA, 22 bp, synthetic construct
  source 1..22: mol_type = other DNA
cactgtggcaagagggaatgtc

SEQ ID NO: 907 - DNA, 22 bp, synthetic construct
  source 1..22: mol_type = other DNA
agttctcatgagccttgctcac

SEQ ID NO: 908 - DNA, 25 bp, synthetic construct
  source 1..25: mol_type = other DNA
cctggatcacaacaggctttctggt

SEQ ID NO: 909 - DNA, 26 bp, synthetic construct
  source 1..26: mol_type = other DNA
gggcattcattttcaaaggaggtgat

SEQ ID NO: 910 - DNA, 26 bp, synthetic construct
  source 1..26: mol_type = other DNA
cctctaaagctcagccttacttgaca

SEQ ID NO: 911 - DNA, 29 bp, synthetic construct
  source 1..29: mol_type = other DNA
ccggtaaccaatctttcttgattctccct

SEQ ID NO: 912 - DNA, 26 bp, synthetic construct
  source 1..26: mol_type = other DNA
gcaatatttgtcttcccatgcctgac

SEQ ID NO: 913 - DNA, 28 bp, synthetic construct
  source 1..28: mol_type = other DNA
agttcctggtttcatttgtcacaacatt

SEQ ID NO: 914 - DNA, 24 bp, synthetic construct
  source 1..24: mol_type = other DNA
ggggatttctggccagtcatgagt

SEQ ID NO: 915 - DNA, 27 bp, synthetic construct
  source 1..27: mol_type = other DNA
gactctaaaacagggctgaatcaagca

SEQ ID NO: 916 - DNA, 24 bp, synthetic construct
  source 1..24: mol_type = other DNA
cttatgacccattacttcccagca

SEQ ID NO: 917 - DNA, 26 bp, synthetic construct
  source 1..26: mol_type = other DNA
ctggccagtttttgtagaacatccct

SEQ ID NO: 918 - DNA, 23 bp, synthetic construct
  source 1..23: mol_type = other DNA
ggatagcacagtggtgatatgct

SEQ ID NO: 919 - DNA, 22 bp, synthetic construct
  source 1..22: mol_type = other DNA
gtaagacagacgggcagatgtc

SEQ ID NO: 920 - DNA, 22 bp, synthetic construct
  source 1..22: mol_type = other DNA
cactctggagagaactgggaga

SEQ ID NO: 921 - DNA, 24 bp, synthetic construct
  source 1..24: mol_type = other DNA
gggacggaaaagttgcattcagtg

SEQ ID NO: 922 - DNA, 24 bp, synthetic construct
  source 1..24: mol_type = other DNA
gctggtggatggatagttccctga

SEQ ID NO: 923 - DNA, 30 bp, synthetic construct
  source 1..30: mol_type = other DNA
cctcagtcacattagccaaatttcaagggt

SEQ ID NO: 924 - DNA, 26 bp, synthetic construct
  source 1..26: mol_type = other DNA
tgaacctcataagacagggtctgttc

SEQ ID NO: 925 - DNA, 20 bp, synthetic construct
  source 1..20: mol_type = other DNA
tgtcttgtccaatgccgaga

SEQ ID NO: 926 - DNA, 20 bp, synthetic construct
  source 1..20: mol_type = other DNA
ggcaaattgactggaagcca

SEQ ID NO: 927 - DNA, 23 bp, synthetic construct
  source 1..23: mol_type = other DNA
gcaaatatgttggcaagacgtat

SEQ ID NO: 928 - DNA, 20 bp, synthetic construct
  source 1..20: mol_type = other DNA
cccttccccaccttggatag

SEQ ID NO: 929 - DNA, 21 bp, synthetic construct
  source 1..21: mol_type = other DNA
catcgcccaaggagtgatata

SEQ ID NO: 930 - DNA, 21 bp, synthetic construct
  source 1..21: mol_type = other DNA
ctctgggtatattcagaagcc

SEQ ID NO: 931 - DNA, 22 bp, synthetic construct
  source 1..22: mol_type = other DNA
tctttggctcccctaaattgct

SEQ ID NO: 932 - DNA, 24 bp, synthetic construct
  source 1..24: mol_type = other DNA
atgaaccgagttttgatttggaat

SEQ ID NO: 933 - DNA, 22 bp, synthetic construct
  source 1..22: mol_type = other DNA
tgggaagaccaaaaacaagcaa

SEQ ID NO: 934 - DNA, 21 bp, synthetic construct
  source 1..21: mol_type = other DNA
tgccccacagagattttagtg

SEQ ID NO: 935 - DNA, 21 bp, synthetic construct
  source 1..21: mol_type = other DNA
gtacgagatgactgaccacca

SEQ ID NO: 936 - DNA, 21 bp, synthetic construct
  source 1..21: mol_type = other DNA
ctgactaagtggagacaaccg

SEQ ID NO: 937 - DNA, 22 bp, synthetic construct
  source 1..22: mol_type = other DNA
gtctctcatatccagtgtaggt

SEQ ID NO: 938 - DNA, 23 bp, synthetic construct
  source 1..23: mol_type = other DNA
tgcctcaaactctttcttctcat

SEQ ID NO: 939 - DNA, 20 bp, synthetic construct
  source 1..20: mol_type = other DNA
tgtcccacacctacagtgcg

SEQ ID NO: 940 - DNA, 22 bp, synthetic construct
  source 1..22: mol_type = other DNA
acctgtgcatgcatttgtgtac

SEQ ID NO: 941 - DNA, 24 bp, synthetic construct
  source 1..24: mol_type = other DNA
tgtccttcatcagaaaaaattctc

SEQ ID NO: 942 - DNA, 22 bp, synthetic construct
  source 1..22: mol_type = other DNA
caacacggctctatatcactga

SEQ ID NO: 943 - DNA, 22 bp, synthetic construct
  source 1..22: mol_type = other DNA
gaagatttactcaaatagagtg

SEQ ID NO: 944 - DNA, 22 bp, synthetic construct
  source 1..22: mol_type = other DNA
tccattgttcatttcaaggaat

SEQ ID NO: 945 - DNA, 23 bp, synthetic construct
  source 1..23: mol_type = other DNA
ttttagagggattcagacacaaa

SEQ ID NO: 946 - DNA, 20 bp, synthetic construct
  source 1..20: mol_type = other DNA
cgctttgcaattggttgtat

SEQ ID NO: 947 - DNA, 30 bp, synthetic construct
  source 1..30: mol_type = other DNA
acgttggatgtgaagaattcttgggtctgc

SEQ ID NO: 948 - DNA, 30 bp, synthetic construct
  source 1..30: mol_type = other DNA
acgttggatgaggaaataagagtacgctcc

SEQ ID NO: 949 - DNA, 30 bp, synthetic construct
  source 1..30: mol_type = other DNA
acgttggatggttgaaatggtagtgttcag

SEQ ID NO: 950 - DNA, 30 bp, synthetic construct
  source 1..30: mol_type = other DNA
acgttggatgtttcagaccatgcatctagg

SEQ ID NO: 951 - DNA, 30 bp, synthetic construct
  source 1..30: mol_type = other DNA
acgttggatgtattctctcttaggcactgg

SEQ ID NO: 952 - DNA, 30 bp, synthetic construct
  source 1..30: mol_type = other DNA
acgttggatgcttagtcacatcaaaggagc

SEQ ID NO: 953 - DNA, 30 bp, synthetic construct
  source 1..30: mol_type = other DNA
acgttggatgatcaccccaagaagaaaccc

SEQ ID NO: 954 - DNA, 30 bp, synthetic construct
  source 1..30: mol_type = other DNA
acgttggatgggaggggttggaaaaatatg

SEQ ID NO: 955 - DNA, 31 bp, synthetic construct
  source 1..31: mol_type = other DNA
acgttggatggagttaaaaattctcccatcc

SEQ ID NO: 956 - DNA, 30 bp, synthetic construct
  source 1..30: mol_type = other DNA
acgttggatgctgtgacagttggtaaggtg

SEQ ID NO: 957 - DNA, 22 bp, synthetic construct
  source 1..22: mol_type = other DNA
cagcaggaatttatctgagcac

SEQ ID NO: 958 - DNA, 21 bp, synthetic construct
  source 1..21: mol_type = other DNA
ggcactaaggcatacacaaca

SEQ ID NO: 959 - DNA, 25 bp, synthetic construct
  source 1..25: mol_type = other DNA
gaatttacctttactggagaacgct

SEQ ID NO: 960 - DNA, 22 bp, synthetic construct
  source 1..22: mol_type = other DNA
aggattctgggtagtacctcaa

SEQ ID NO: 961 - DNA, 23 bp, synthetic construct
  source 1..23: mol_type = other DNA
tacaggtagaagaaggctccaag

SEQ ID NO: 962 - DNA, 22 bp, synthetic construct
  source 1..22: mol_type = other DNA
atcaaatcctcaggctaatgct

SEQ ID NO: 963 - DNA, 22 bp, synthetic construct
  source 1..22: mol_type = other DNA
ttgatttcagagtgcccagagt

SEQ ID NO: 964 - DNA, 22 bp, synthetic construct
  source 1..22: mol_type = other DNA
cagtgtggctttgtgtgtagga

SEQ ID NO: 965 - DNA, 24 bp, synthetic construct
  source 1..24: mol_type = other DNA
ggtgggatgttgtttttctctcct

SEQ ID NO: 966 - DNA, 24 bp, synthetic construct
  source 1..24: mol_type = other DNA
gcattcgacttactgttgctgaag

SEQ ID NO: 967 - DNA, 25 bp, synthetic construct
  source 1..25: mol_type = other DNA
agctagtgtgaggaacatagcaaga

SEQ ID NO: 968 - DNA, 27 bp, synthetic construct
  source 1..27: mol_type = other DNA
tatccctggaaatcaatttgttccctt

SEQ ID NO: 969 - DNA, 24 bp, synthetic construct
  source 1..24: mol_type = other DNA
ctgtgcaagttgtgtgatcttggt

SEQ ID NO: 970 - DNA, 26 bp, synthetic construct
  source 1..26: mol_type = other DNA
gaggaactaccttgggatttggggtt

SEQ ID NO: 971 - DNA, 22 bp, synthetic construct
  source 1..22: mol_type = other DNA
gcttgcagggagccaaaattgt

SEQ ID NO: 972 - DNA, 28 bp, synthetic construct
  source 1..28: mol_type = other DNA
acagaaatatcctttgtccccacaatct

SEQ ID NO: 973 - DNA, 21 bp, synthetic construct
  source 1..21: mol_type = other DNA
agccaacatactcgccaatcc

SEQ ID NO: 974 - DNA, 22 bp, synthetic construct
  source 1..22: mol_type = other DNA
gcctactagcagggctctggaa

SEQ ID NO: 975 - DNA, 27 bp, synthetic construct
  source 1..27: mol_type = other DNA
tcagcttccagatattaccagcatgca

SEQ ID NO: 976 - DNA, 28 bp, synthetic construct
  source 1..28: mol_type = other DNA
taaagaggatgaatgggaggaaaaagag

SEQ ID NO: 977 - DNA, 28 bp, synthetic construct
  source 1..28: mol_type = other DNA
caaggggtcgattgattaaaataacgaa

SEQ ID NO: 978 - DNA, 25 bp, synthetic construct
  source 1..25: mol_type = other DNA
tactttaggttcaagggcacatgtg

SEQ ID NO: 979 - DNA, 26 bp, synthetic construct
  source 1..26: mol_type = other DNA
gctttctacggcatagaaagtttgtg

SEQ ID NO: 980 - DNA, 25 bp, synthetic construct
  source 1..25: mol_type = other DNA
gccaccgcttcagagttgttctagg

SEQ ID NO: 981 - DNA, 28 bp, synthetic construct
  source 1..28: mol_type = other DNA
tctcgcaaatagtagcagaaaccttatt

SEQ ID NO: 982 - DNA, 27 bp, synthetic construct
  source 1..27: mol_type = other DNA
gctcctaccacatgtcaaatgttagca

SEQ ID NO: 983 - DNA, 31 bp, synthetic construct
  source 1..31: mol_type = other DNA
ggaaaatgtgggctcgttttaattatattca

SEQ ID NO: 984 - DNA, 32 bp, synthetic construct
  source 1..32: mol_type = other DNA
gatcgtcaaagaattcatacctgtaatttctg

SEQ ID NO: 985 - DNA, 24 bp, synthetic construct
  source 1..24: mol_type = other DNA
tcctgcaaatcttaggggaaccca

SEQ ID NO: 986 - DNA, 22 bp, synthetic construct
  source 1..22: mol_type = other DNA
gcttacaatcccagcactttgg

SEQ ID NO: 987 - DNA, 26 bp, synthetic construct
  source 1..26: mol_type = other DNA
catggtctctttgggtgaaagactac

SEQ ID NO: 988 - DNA, 29 bp, synthetic construct
  source 1..29: mol_type = other DNA
cctggaaaatgtagactggtgttacatgc

SEQ ID NO: 989 - DNA, 27 bp, synthetic construct
  source 1..27: mol_type = other DNA
accccattctttctcaagttaattgca

SEQ ID NO: 990 - DNA, 22 bp, synthetic construct
  source 1..22: mol_type = other DNA
gcacagcattctcacggacatg

SEQ ID NO: 991 - DNA, 22 bp, synthetic construct
  source 1..22: mol_type = other DNA
tggttcacactcccttctgtgt

SEQ ID NO: 992 - DNA, 25 bp, synthetic construct
  source 1..25: mol_type = other DNA
attcaacagtcaaggccagaaagac

SEQ ID NO: 993 - DNA, 29 bp, synthetic construct
  source 1..29: mol_type = other DNA
tgagaaaagattgctctaatcagcctatt

SEQ ID NO: 994 - DNA, 28 bp, synthetic construct
  source 1..28: mol_type = other DNA
gctcttcggaagtgtaaagtcatttagc

SEQ ID NO: 995 - DNA, 26 bp, synthetic construct
  source 1..26: mol_type = other DNA
gggaaaacattgctttcacctctgag

SEQ ID NO: 996 - DNA, 27 bp, synthetic construct
  source 1..27: mol_type = other DNA
gaaaggttattacccttctgctgtcct

SEQ ID NO: 997 - DNA, 27 bp, synthetic construct
  source 1..27: mol_type = other DNA
gacaaacccaaccaagaagacatccct

SEQ ID NO: 998 - DNA, 25 bp, synthetic construct
  source 1..25: mol_type = other DNA
gctgaaattctaacccacagtactg

SEQ ID NO: 999 - DNA, 25 bp, synthetic construct
  source 1..25: mol_type = other DNA
agtgtgggatacagtgaaatcagtg

SEQ ID NO: 1000 - DNA, 24 bp, synthetic construct
  source 1..24: mol_type = other DNA
gggtcaagggcagaaactgacaac

SEQ ID NO: 1001 - DNA, 27 bp, synthetic construct
  source 1..27: mol_type = other DNA
ggcaaaaataaaatttcccccaaccat

SEQ ID NO: 1002 - DNA, 22 bp, synthetic construct
  source 1..22: mol_type = other DNA
catttctcaccatgcccactca

SEQ ID NO: 1003 - DNA, 24 bp, synthetic construct
  source 1..24: mol_type = other DNA
tcctggattcagctctcttcctaa

SEQ ID NO: 1004 - DNA, 22 bp, synthetic construct
  source 1..22: mol_type = other DNA
agcctctcctgatactctgtgt

SEQ ID NO: 1005 - DNA, 24 bp, synthetic construct
  source 1..24: mol_type = other DNA
tgccagaagagcaaatacggtaat

SEQ ID NO: 1006 - DNA, 25 bp, synthetic construct
  source 1..25: mol_type = other DNA
tgaaaacacaaagctactggcaagc

SEQ ID NO: 1007 - DNA, 33 bp, synthetic construct
  source 1..33: mol_type = other DNA
agaaaggaaacgagataaaatattaaacgactt

SEQ ID NO: 1008 - DNA, 27 bp, synthetic construct
  source 1..27: mol_type = other DNA
tggtatcacaatagaaggggaatgatc

SEQ ID NO: 1009 - DNA, 24 bp, synthetic construct
  source 1..24: mol_type = other DNA
cttgggcaagtactgtagaccatc

SEQ ID NO: 1010 - DNA, 23 bp, synthetic construct
  source 1..23: mol_type = other DNA
ttggtgcaggctgaattttcagt

SEQ ID NO: 1011 - DNA, 27 bp, synthetic construct
  source 1..27: mol_type = other DNA
ctacatggaaatggttggctattgagt

SEQ ID NO: 1012 - DNA, 28 bp, synthetic construct
  source 1..28: mol_type = other DNA
aaaaatacagcacaatacagcacattcg

SEQ ID NO: 1013 - DNA, 22 bp, synthetic construct
  source 1..22: mol_type = other DNA
ttacttagaggcagggtgttgc

SEQ ID NO: 1014 - DNA, 24 bp, synthetic construct
  source 1..24: mol_type = other DNA
taagacattgcactcagcctcaag

SEQ ID NO: 1015 - DNA, 23 bp, synthetic construct
  source 1..23: mol_type = other DNA
tagtttcggagctagcccgtttt

SEQ ID NO: 1016 - DNA, 22 bp, synthetic construct
  source 1..22: mol_type = other DNA
ttggccactccaaagagtgaac

SEQ ID NO: 1017 - DNA, 30 bp, synthetic construct
  source 1..30: mol_type = other DNA
acgttggatgtatagcggcatacttgcctc

SEQ ID NO: 1018 - DNA, 30 bp, synthetic construct
  source 1..30: mol_type = other DNA
acgttggatgggttcttgattaggctaagg

SEQ ID NO: 1019 - DNA, 22 bp, synthetic construct
  source 1..22: mol_type = other DNA
agatcatcagctctgtgactgg

SEQ ID NO: 1020 - DNA, 20 bp, synthetic construct
  source 1..20: mol_type = other DNA
ccatcgatttccagcatttc

SEQ ID NO: 1021 - DNA, 21 bp, synthetic construct
  source 1..21: mol_type = other DNA
aattgccaggtggtgttacag

SEQ ID NO: 1022 - DNA, 20 bp, synthetic construct
  source 1..20: mol_type = other DNA
tcatggctgttttgtctcca

SEQ ID NO: 1023 - DNA, 24 bp, synthetic construct
  source 1..24: mol_type = other DNA
ttttctatgtaggggagttgtgag

SEQ ID NO: 1024 - DNA, 21 bp, synthetic construct
  source 1..21: mol_type = other DNA
aagctgcaaaattgagtgagc

SEQ ID NO: 1025 - DNA, 20 bp, synthetic construct
  source 1..20: mol_type = other DNA
gagaggaaggaaccctccag

SEQ ID NO: 1026 - DNA, 20 bp, synthetic construct
  source 1..20: mol_type = other DNA
attgcaggaaaagggtggaa

SEQ ID NO: 1027 - DNA, 22 bp, synthetic construct
  source 1..22: mol_type = other DNA
tctcactatgatccaggcaatg

SEQ ID NO: 1028 - DNA, 24 bp, synthetic construct
  source 1..24: mol_type = other DNA
cataaattacccagattcgggaag

SEQ ID NO: 1029 - DNA, 23 bp, synthetic construct
  source 1..23: mol_type = other DNA
acaatgctcaggcttccacgaga

SEQ ID NO: 1030 - DNA, 24 bp, synthetic construct
  source 1..24: mol_type = other DNA
tgaggggccagacttgacttgtgt

SEQ ID NO: 1031 - DNA, 20 bp, synthetic construct
  source 1..20: mol_type = other DNA
atcaactggccatgtgagtg

SEQ ID NO: 1032 - DNA, 25 bp, synthetic construct
  source 1..25: mol_type = other DNA
tgctgactaacatattaccccaaac

SEQ ID NO: 1033 - DNA, 30 bp, synthetic construct
  source 1..30: mol_type = other DNA
acgttggatgggccccaaatccaactaatg

SEQ ID NO: 1034 - DNA, 30 bp, synthetic construct
  source 1..30: mol_type = other DNA
acgttggatggtgagaccacgaagagactg

SEQ ID NO: 1035 - DNA, 30 bp, synthetic construct
  source 1..30: mol_type = other DNA
acgttggatgacactgaccaacttgagagg

SEQ ID NO: 1036 - DNA, 30 bp, synthetic construct
  source 1..30: mol_type = other DNA
acgttggatgagacttttctgggacctgag

SEQ ID NO: 1037 - DNA, 30 bp, synthetic construct
  source 1..30: mol_type = other DNA
acgttggatggaattcaaagagggagaaac

SEQ ID NO: 1038 - DNA, 30 bp, synthetic construct
  source 1..30: mol_type = other DNA
acgttggatggagtttcttcctccactgac

SEQ ID NO: 1039 - DNA, 30 bp, synthetic construct
  source 1..30: mol_type = other DNA
acgttggatggggatcaaatggagttcctg

SEQ ID NO: 1040 - DNA, 30 bp, synthetic construct
  source 1..30: mol_type = other DNA
acgttggatgtctcctaagcctacaggttg

(19) United States
(12) Patent Application Publication
(10) Pub. No.: US 2025/0166727 A1
(43) Pub. Date: May 22, 2025

(54) SNP LOCUS COMBINATION FOR PATERNITY TESTING, DETECTION PRIMER PAIRS AND APPLICATION THEREOF

(71) Applicant: Fudan University, Shanghai (CN)

(72) Inventors: SuHua Zhang, Shanghai (CN); ChengTao Li, Shanghai (CN); AnQi Chen, Shanghai (CN)

(21) Appl. No.: 18/939,430

(22) Filed: Nov. 6, 2024

(30) Foreign Application Priority Data
Nov. 22, 2023 (CN) 2023115675333

(51) Int. Cl. G16B 20/20; C12N 15/10; C12Q 1/6806; C12Q 1/686; C12Q 1/6869

(52) U.S. Cl. CPC G16B 20/20; C12N 15/1093; C12Q 1/6806; C12Q 1/686; C12Q 1/6869

(57) ABSTRACT

A combination of SNP loci for paternity testing, a detection primer pair and an application thereof are provided, belonging to the application research of forensic molecular genetics. The combination of SNP loci includes 520 SNP loci. Based on the combination of SNP loci, all the selected loci in the population conform to Hardy-Weinberg equilibrium (corrected by Bonferroni), and each locus is not linked and is in linkage equilibrium. When selecting loci, references are made to various databases, relevant literature, and genetic markers widely used in forensic science, which allows for obtaining more genetic information and higher forensic efficacy. It is conducive to forensic applications such as individual identification of difficult biological samples and prenatal paternity test of maternal peripheral blood.

### CROSS-REFERENCE TO RELATED APPLICATION

This application claims priority to Chinese Patent Application No. 202311567533.3, filed Nov. 22, 2023, which is herein incorporated by reference in its entirety.

### TECHNICAL FIELD

The disclosure relates to the field of studies on application of molecular genetics in judicial expertise, and more particularly to a single nucleotide polymorphism (SNP) locus combination for paternity testing, detection primer pairs and an application thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 24060THXT-USP1-SL.xml. The XML file is 922,258 bytes; is created on Oct. 12, 2024; and is being submitted electronically via patent center.

### BACKGROUND

At present, short tandem repeat (STR) is usually applied as a genetic marker in the field of forensic science, and polymerase chain reaction (PCR) and capillary electrophoresis (CE) technologies are combined for detection and analysis. Although STR is widely used, it is prone to stutter peaks in detection, and has disadvantages such as large amplification fragments and high mutation rate. In addition, when using PCR-CE technique is less likely to yield satisfactory results in the detection of STR in difficult forensic samples such as degraded, trace and mixed samples.

Single nucleotide polymorphisms (SNP) are variations such as deletion, insertion or conversion and transversion of a single nucleotide in the genome. Compared with STR, SNP does not produce stutter peaks and has shorter amplification fragments, so it is more suitable for the detection and analysis of difficult biological samples. In addition, SNP has a low mutation rate and high genetic stability. Its biallelic nature is conducive to genotyping and easy to realize batch and automated analysis. The rapid development of second-generation sequencing (SGS) technology provides technical support for the parallel detection and precise genotyping of a large number of SNP genetic markers.

In recent years, with the improvement of women's legal literacy and self-protection awareness, the demand for prenatal paternity testing involving pregnant women in judicial cases has been increasing continuously. At present, commonly used invasive detection methods such as amniocentesis and chorionic villus sampling have certain risks, and the time for implementation of detection is also very limited, which is not conducive to the physical and mental health of pregnant women. In 1997, Lo, Y. M. et al. (Lo, Y. M. et al., “Presence of fetal DNA in maternal plasma and serum”, The Lancet, 1997, pages 485-487, Vol. 350, No. 9076.) confirmed the existence of cell-free fetal DNA (cffDNA) in peripheral blood of pregnant women, providing a new research idea for non-invasive prenatal diagnosis. The extraction of cell-free DNA (cfDNA) from plasma of pregnant women is a typical mixed sample, which mainly contains a large amount of genome DNA (gDNA) derived from pregnant women themselves and a small amount of cfDNA from fetuses. It is generally believed that cffDNA mainly comes from apoptotic placental trophoblast cells. Therefore, cffDNA also has distinct characteristics of degraded forensic materials, which makes non-invasive prenatal paternity testing extremely difficult. In 2016, Haojun Jiang et al. (Haojun Jiang et al., “Noninvasive Prenatal Paternity Testing (NIPAT) through Maternal Plasma DNA Sequencing: A Pilot Study”, PLOS One, 2016, e0159385, Vol. 11, No. 9.) developed a method for library construction and high-throughput sequencing of cfDNA in maternal plasma based on SGS technology. In 2019, Liao Chang et al. (Liao Chang et al., “Development and comprehensive evaluation of a noninvasive prenatal paternity testing method through a scaled trial”, Forensic Science International: Genetics, 2019, 102158, Vol. 43.) developed a new system containing 5,457 SNP loci and evaluated its effectiveness, reliability and sensitivity on the basis of a large-scale test. In 2020, Jacqueline Chor Wing Tam et al. (Jacqueline Chor Wing Tam et al., “Noninvasive prenatal paternity testing by means of SNP-based targeted sequencing”, Prenat Diagn, 2020, pages 497-506, Vol. 40, No. 4.) developed a method for detecting cfDNA in maternal plasma by targeting sequencing, based on the selection of SNPs with high heterozygosity to enhance discrimination. A set of 356 individual identification SNP loci was established according to population genetic data. After enrichment of cfDNA in maternal plasma using the QIAseq Targeted DNA Panel with unique molecular identifiers (UMI), targeted sequencing and data analysis were performed.

These studies have initially constructed the basic method of non-invasive prenatal paternity testing based on cffDNA, but there are still problems such as low detection efficiency, high cost, complex methods and poor repeatability. Moreover, since relevant industry standards have not been established at present, most methods are costly and difficult to achieve automation and standardization, and cannot be popularized, which seriously hinders its application in forensic practice.

### SUMMARY

For the above problems, one of the purposes of the disclosure is to provide a single nucleotide polymorphism (SNP) locus combination for paternity testing. 520 SNP loci with good polymorphism are selected in the SNP loci combination according to the genetic characteristics of the Chinese population. A second-generation sequencing (SGS) platform can be used to simultaneously perform deep parallel sequencing of 520 SNP loci of multiple samples.

To achieve the above purpose, the disclosure can adopt the following technical solutions.

In a first aspect, the disclosure provides a SNP locus combination for paternity testing, including 520 SNP loci:
rs868163, rs3007429, rs3007430, rs2076605, rs7531163, rs607254, rs2361660, rs7516939, rs494198, rs6588052, rs11208265, rs472908, rs3736296, rs2274570, rs3811003, rs9428083, rs9427094, rs703116, rs1920149, rs7536561, rs647347, rs2274064, rs12042179, rs4844573, rs2076355, rs12031285, rs630120, rs12042900, rs7527925, rs11578981, rs7552783, rs2045955, rs2618702, rs2805396, rs1332777, rs10802626, rs2256242, rs10927011, rs2280132, rs2271621, rs7561070, rs4072407, rs455060, rs3738984, rs3771274, rs2020911, rs2276625, rs2244182, rs2271244, rs72481904, rs1061305, rs16830136, rs2288201, rs12620077, rs1867864, rs1542484, rs830973, rs12613075, rs2366751, rs2303838, rs2042995, rs2562839, rs12997453, rs3024866, rs1208081, rs2242074, rs17552879, rs3213784, rs6704780, rs12991025, rs34490902, rs2304774, rs17868324, rs1105879, rs4234097, rs6756901, rs456168, rs2733532, rs3729587, rs3816526, rs2507940, rs13097628, rs7641844, rs1799987, rs936175, rs13066214, rs749512, rs6445873, rs4681982, rs2290174, rs533852, rs1463138, rs2713604, rs1683787, rs2681092, rs16861582, rs2303909, rs9290259, rs1515442, rs2593813, rs2028572, rs9851685, rs2286342, rs899691, rs2306400, rs13121363, rs13144326, rs7655165, rs12650452, rs2125314, rs225170, rs6856362, rs7660664, rs34840208, rs7677541, rs227365, rs17009761, rs62333013, rs2166337, rs4429853, rs13170062, rs162850, rs7710284, rs6451206, rs11957963, rs476569, rs1801033, rs706714, rs182190, rs309557, rs4916683, rs274557, rs2303068, rs4072924, rs438464, rs2236277, rs2842949, rs2071303, rs3737095, rs3818554, rs167772, rs3920621, rs10948667, rs9474140, rs9367689, rs4712138, rs2230862, rs4710457, rs1936439, rs2305837, rs519068, rs1875845, rs9448893, rs3828753, rs3734353, rs6922753, rs2273752, rs7754205, rs3813328, rs902373, rs3798663, rs6914831, rs3762000, rs22908, rs6904364, rs3818110, rs9371581, rs9383985, rs9397102, rs1873176, rs214968, rs3734440, rs9356399, rs4716346, rs2527509, rs852423, rs6461252, rs7782939, rs1023542, rs2240401, rs10261071, rs12669315, rs6950777, rs2072454, rs2286822, rs733957, rs6957975, rs7779997, rs362726, rs607755, rs2520272, rs213950, rs3815221, rs3779032, rs7830, rs3802100, rs1550697, rs3020221, rs2073574, rs12675283, rs2272730, rs2737335, rs7842714, rs3098659, rs6511, rs10093032, rs2261147, rs2069568, rs2272653, rs11780911, rs3817681, rs17855765, rs4244610, rs4251691, rs12683181, rs11243704, rs12358961, rs10828545, rs10491056, rs3817419, rs3740232, rs2435352, rs7089209, rs3812658, rs2105702, rs3750863, rs3758626, rs10999338, rs780668, rs7917781, rs4747203, rs2131956, rs7074064, rs1903858, rs1409332, rs10786229, rs3740486, rs813944, rs196294, rs2278202, rs2272599, rs7942159, rs3758680, rs1799937, rs11037882, rs3740878, rs3729802, rs589691, rs630394, rs736894, rs11604536, rs1109977, rs883223, rs2196168, rs641936, rs1784424, rs591058, rs10895417, rs693147, rs2276424, rs1954787, rs2241501, rs880054, rs10848683, rs2267549, rs216868, rs12426675, rs2518144, rs3759236, rs10772008, rs7955902, rs2276456, rs1034762, rs3803184, rs4075913, rs2071218, rs706815, rs2270267, rs7397862, rs2302629, rs6606734, rs2464195, rs3741591, rs2075784, rs9564692, rs3752451, rs9534342, rs2296533, rs6313, rs9518061, rs661585, rs589985, rs482757, rs3803236, rs2281974, rs178642, rs2073349, rs1152590, rs229581, rs3759753, rs2075179, rs12432149, rs3742656, rs3751582, rs1800419, rs1800404, rs173224, rs422388, rs2279580, rs1801376, rs140605, rs2306187, rs58156069, rs1147129, rs762551, rs1370275, rs9806604, rs2307438, rs9672296, rs3815003, rs2272037, rs7496614, rs224204, rs1641010, rs2280764, rs3115446, rs215901, rs2072061, rs249954, rs7203525, rs243834, rs2304483, rs1532625, rs11076207, rs483053, rs3991715, rs2548861, rs12918876, rs3803679, rs3752827, rs238238, rs12453262, rs17671352, rs1565817, rs2304908, rs876660, rs3744586, rs1736219, rs548973, rs2952976, rs9894648, rs3110641, rs8067890, rs8176161, rs850730, rs2289677, rs2258689, rs3809863, rs2256835, rs3744093, rs758596, rs4342, rs4362, rs3744024, rs11077379, rs3816256, rs3213926, rs7232329, rs3748422, rs8097342, rs2510344, rs12960081, rs1058396, rs1450425, rs1788027, rs2298712, rs1893788, rs6567271, rs1629038, rs3764640, rs2277984, rs2860178, rs620744, rs539887, rs8104339, rs2114724, rs280520, rs3826803, rs2075021, rs2738447, rs6413504, rs2304154, rs8113582, rs1043996, rs2302603, rs10424582, rs55742440, rs1469699, rs3745859, rs10880, rs2246095, rs17 997 83, rs392891, rs20580, rs3730849, rs2387588, rs4002462, rs1062798, rs12981413, rs3745405, rs269950, rs3729836, rs2076653, rs2073193, rs2281575, rs3803953, rs2295435, rs2295179, rs363050, rs2273060, rs2273061, rs143383, rs2072792, rs4608591, rs2425906, rs6021437, rs2234487, rs3746657, rs6062700, rs41309367, rs2073421, rs2073425, rs8130564, rs872331, rs754507, rs62215498, rs2839077, rs7283989, rs2236486, rs7279347, rs7279622, rs9982233, rs2839226, rs2839257, rs112544, rs2072708, rs2235324, rs5756516, rs1135360, rs743616, rs6639398, rs5980274, rs1458178, rs5986751, rs9781645, rs2209420, rs5984589, rs2808742, rs5917032, rs6529455, rs5931302, rs6643690, rs1781104, rs1781116, rs318173, rs4825889, rs933315, rs1991961, rs1937193, rs652, rs5964206, rs149910, rs1299087, rs5920670, rs6631828, rs973212, rs5933388, rs6649211, rs5916197, rs4898214, rs2071182, rs4826645, rs35284970, rs13447361, rs2267801, rs9786139, rs2075181, rs17316007, rs17269396, rs8179021, rs16980601, rs9341278, rs917759, rs17269816, rs17316543, rs17316592, rs17269928, rs17276358, rs16980641, rs11096432, rs9786502, rs2032652, rs16980426, rs13447443, rs17323322, rs13447354, rs2033003, rs16980610, rs3848982, rs2075640, rs16980711, rs9786707, rs17276345, rs9786479, rs9306845, rs11096433, rs52812045, rs4589047, rs17250121, and rs2032650.

In a second aspect, the disclosure provides a detection primer pair for detecting the above SNP locus combination for paternity testing.

In a third aspect, the disclosure provides a detection reagent, which includes the above detection primer pair.

In a fourth aspect, the disclosure provides a detection kit, which includes the above detection primer pair or the above detection reagent.

In a fifth aspect, the disclosure provides an application of the above detection primer pair or the above detection reagent in the preparation of products for prenatal paternity testing.

In a sixth aspect, the disclosure provides a method for prenatal paternity testing, which includes: step (1) DNA extraction: cell-free DNA of a fetus in maternal plasma, genomic DNA of a pregnant woman, and genomic DNA of a suspected father; step (2) multiplex polymerase chain reaction (PCR) amplification: performing the PCR amplification on a to-be-tested sample using the detection primer pair, the detection reagent or the detection kit to obtain a multiplex PCR amplification product; step (3) library construction: performing library construction on the multiplex PCR amplification product sequentially through two rounds of multiplex PCR reactions and two rounds of magnetic bead purification processes; step (4) library quality control: measuring and recording a concentration and a fragment length of the library after the library is constructed to prepare for sequencing on a machine; step (5) sequencing on the machine: performing sequencing on the machine by using a sequencing platform to obtain sequencing results; and step (6) data analysis: comparing the sequencing results with a human reference genome for bioinformatics analysis and determination of kinship by using a software, analyzing genotyping results of each SNP locus of the fetus, the pregnant woman and the suspected father, and performing comparison to identify whether there is a kinship between the suspected father and the fetus.

The beneficial effects of the disclosure at least are as follows.

(1) In the SNP locus combination for paternity testing provided by the disclosure, the selected loci all conform to Hardy-Weinberg equilibrium in the population (after Bonferroni correction), the loci are not linked and are in a state of linkage equilibrium. When selecting loci, different databases and relevant literature are referenced and combined with genetic markers widely used in forensic medicine to obtain more genetic information and higher forensic efficacy. The application of SGS technology makes it easier to detect degraded, trace, and mixed samples, and allows for the parallel detection of multiple loci in multiple samples, saving time, labor, and economic costs, which is conducive to the identification of individuals with difficult biological samples and prenatal paternity testing in maternal peripheral blood and other forensic science applications.

(2) The disclosure uses TruSeq® Custom Amplicon technology for primer design and library construction, and uses a two-step PCR amplification method to complete target region amplification and library construction. The experimental process is simple, the experimental method is reliable, and the experimental results are highly accurate.

(3) The disclosure is applicable to various biological samples, such as genomic DNA of common human biological samples, and cell-free DNA extracted from maternal plasma.

### BRIEF DESCRIPTION OF DRAWINGS

FIG. 1 illustrates a specific flowchart of non-invasive prenatal paternity testing based on detection of SNP loci by second-generation sequencing (SGS) technology according to an embodiment of the disclosure.

FIG. 2 illustrates a specific flowchart of library construction according to the embodiment of the disclosure.

### DETAILED DESCRIPTION OF EMBODIMENTS

Embodiments provided are intended to illustrate the disclosure more clearly, but the content of the disclosure is not limited only to the embodiments provided. Therefore, non-essential improvements and adjustments made by those skilled in the art to the implementation scheme according to the above disclosure content still fall within the protection scope of the disclosure.

The terms used in the disclosure are only for describing specific embodiments and are not intended to limit this disclosure. Unless there is a clearly different meaning in the context, expressions in the singular form include expressions in the plural form. As used herein, it should be understood that terms such as “including”, “having”, and “containing” are intended to indicate the existence of features, numbers, operations, components, parts, elements, materials or combinations thereof. The terms of the disclosure are disclosed in the specification and are not intended to exclude the possibility that one or more other features, numbers, operations, components, parts, elements, materials or combinations thereof may exist or can be added. As used herein, depending on the situation, “/” can be interpreted as “and” or “or”.

An embodiment of the disclosure provides a SNP locus combination for paternity testing, including 520 SNP loci:
rs868163, rs3007429, rs3007430, rs2076605, rs7531163, rs607254, rs2361660, rs7516939, rs494198, rs6588052, rs11208265, rs472908, rs3736296, rs2274570, rs3811003, rs9428083, rs9427094, rs703116, rs1920149, rs7536561, rs647347, rs2274064, rs12042179, rs4844573, rs2076355, rs12031285, rs630120, rs12042900, rs7527925, rs11578981, rs7552783, rs2045955, rs2618702, rs2805396, rs1332777, rs10802626, rs2256242, rs10927011, rs2280132, rs2271621, rs7561070, rs4072407, rs455060, rs3738984, rs3771274, rs2020911, rs2276625, rs2244182, rs2271244, rs72481904, rs1061305, rs16830136, rs2288201, rs12620077, rs1867864, rs1542484, rs830973, rs12613075, rs2366751, rs2303838, rs2042995, rs2562839, rs12997453, rs3024866, rs1208081, rs2242074, rs17552879, rs3213784, rs6704780, rs12991025, rs34490902, rs2304774, rs17868324, rs1105879, rs4234097, rs6756901, rs456168, rs2733532, rs3729587, rs3816526, rs2507940, rs13097628, rs7641844, rs1799987, rs936175, rs13066214, rs749512, rs6445873, rs4681982, rs2290174, rs533852, rs1463138, rs2713604, rs1683787, rs2681092, rs16861582, rs2303909, rs9290259, rs1515442, rs2593813, rs2028572, rs9851685, rs2286342, rs899691, rs2306400, rs13121363, rs13144326, rs7655165, rs12650452, rs2125314, rs225170, rs6856362, rs7660664, rs34840208, rs7677541, rs227365, rs17009761, rs62333013, rs2166337, rs4429853, rs13170062, rs162850, rs7710284, rs6451206, rs11957963, rs476569, rs1801033, rs706714, rs182190, rs309557, rs4916683, rs274557, rs2303068, rs4072924, rs438464, rs2236277, rs2842949, rs2071303, rs3737095, rs3818554, rs167772, rs3920621, rs10948667, rs9474140, rs9367689, rs4712138, rs2230862, rs4710457, rs1936439, rs2305837, rs519068, rs1875845, rs9448893, rs3828753, rs3734353, rs6922753, rs2273752, rs7754205, rs3813328, rs902373, rs3798663, rs6914831, rs3762000, rs22908, rs6904364, rs3818110, rs9371581, rs9383985, rs9397102, rs1873176, rs214968, rs3734440, rs9356399, rs4716346, rs2527509, rs852423, rs6461252, rs7782939, rs1023542, rs2240401, rs10261071, rs12669315, rs6950777, rs2072454, rs2286822, rs733957, rs6957975, rs7779997, rs362726, rs607755, rs2520272, rs213950, rs3815221, rs3779032, rs7830, rs3802100, rs1550697, rs3020221, rs2073574, rs12675283, rs2272730, rs2737335, rs7842714, rs3098659, rs6511, rs10093032, rs2261147, rs2069568, rs2272653, rs11780911, rs3817681, rs17855765, rs4244610, rs4251691, rs12683181, rs11243704, rs12358961, rs10828545, rs10491056, rs3817419, rs3740232, rs2435352, rs7089209, rs3812658, rs2105702, rs3750863, rs3758626, rs10999338, rs780668, rs7917781, rs4747203, rs2131956, rs7074064, rs1903858, rs1409332, rs10786229, rs3740486, rs813944, rs196294, rs2278202, rs2272599, rs7942159, rs3758680, rs1799937, rs11037882, rs3740878, rs3729802, rs589691, rs630394, rs736894, rs11604536, rs1109977, rs883223, rs2196168, rs641936, rs1784424, rs591058, rs10895417, rs693147, rs2276424, rs1954787, rs2241501, rs880054, rs10848683, rs2267549, rs216868, rs12426675, rs2518144, rs3759236, rs10772008, rs7955902, rs2276456, rs1034762, rs3803184, rs4075913, rs2071218, rs706815, rs2270267, rs7397862, rs2302629, rs6606734, rs2464195, rs3741591, rs2075784, rs9564692, rs3752451, rs9534342, rs2296533, rs6313, rs9518061, rs661585, rs589985, rs482757, rs3803236, rs2281974, rs178642, rs2073349, rs1152590, rs229581, rs3759753, rs2075179, rs12432149, rs3742656, rs3751582, rs1800419, rs1800404, rs173224, rs422388, rs2279580, rs1801376, rs140605, rs2306187, rs58156069, rs1147129, rs762551, rs1370275, rs9806604, rs2307438, rs9672296, rs3815003, rs2272037, rs7496614, rs224204, rs1641010, rs2280764, rs3115446, rs215901, rs2072061, rs249954, rs7203525, rs243834, rs2304483, rs1532625, rs11076207, rs483053, rs3991715, rs2548861, rs12918876, rs3803679, rs3752827, rs238238, rs12453262, rs17671352, rs1565817, rs2304908, rs876660, rs3744586, rs1736219, rs548973, rs2952976, rs9894648, rs3110641, rs8067890, rs8176161, rs850730, rs2289677, rs2258689, rs3809863, rs2256835, rs3744093, rs758596, rs4342, rs4362, rs3744024, rs11077379, rs3816256, rs3213926, rs7232329, rs3748422, rs8097342, rs2510344, rs12960081, rs1058396, rs1450425, rs1788027, rs2298712, rs1893788, rs6567271, rs1629038, rs3764640, rs2277984, rs2860178, rs620744, rs539887, rs8104339, rs2114724, rs280520, rs3826803, rs2075021, rs2738447, rs6413504, rs2304154, rs8113582, rs1043996, rs2302603, rs10424582, rs55742440, rs1469699, rs3745859, rs10880, rs2246095, rs17 997 83, rs392891, rs20580, rs3730849, rs2387588, rs4002462, rs1062798, rs12981413, rs3745405, rs269950, rs3729836, rs2076653, rs2073193, rs2281575, rs3803953, rs2295435, rs2295179, rs363050, rs2273060, rs2273061, rs143383, rs2072792, rs4608591, rs2425906, rs6021437, rs2234487, rs3746657, rs6062700, rs41309367, rs2073421, rs2073425, rs8130564, rs872331, rs754507, rs62215498, rs2839077, rs7283989, rs2236486, rs7279347, rs7279622, rs9982233, rs2839226, rs2839257, rs112544, rs2072708, rs2235324, rs5756516, rs1135360, rs743616, rs6639398, rs5980274, rs1458178, rs5986751, rs9781645, rs2209420, rs5984589, rs2808742, rs5917032, rs6529455, rs5931302, rs6643690, rs1781104, rs1781116, rs318173, rs4825889, rs933315, rs1991961, rs1937193, rs652, rs5964206, rs149910, rs1299087, rs5920670, rs6631828, rs973212, rs5933388, rs6649211, rs5916197, rs4898214, rs2071182, rs4826645, rs35284970, rs13447361, rs2267801, rs9786139, rs2075181, rs17316007, rs17269396, rs8179021, rs16980601, rs9341278, rs917759, rs17269816, rs17316543, rs17316592, rs17269928, rs17276358, rs16980641, rs11096432, rs9786502, rs2032652, rs16980426, rs13447443, rs17323322, rs13447354, rs2033003, rs16980610, rs3848982, rs2075640, rs16980711, rs9786707, rs17276345, rs9786479, rs9306845, rs11096433, rs52812045, rs4589047, rs17250121, and rs2032650.

Specifically, the disclosure has constructed a composite detection system containing 520 SNP loci based on second-generation sequencing (SGS) technology. The selected loci all conform to Hardy-Weinberg equilibrium in the population (after Bonferroni correction), the loci are not linked and are in a state of linkage equilibrium. When selecting loci, different databases and relevant literature are referenced and combined with genetic markers widely used in forensic medicine to obtain more genetic information and higher forensic efficacy. The application of SGS technology makes it easier to detect degraded, trace, and mixed samples, and allows for the parallel detection of multiple loci in multiple samples, saving time, labor, and economic costs, which is conducive to the identification of individuals with difficult biological samples and prenatal paternity testing in maternal peripheral blood and other forensic science applications.

Another embodiment of the disclosure provides a detection primer pair for detecting the above SNP locus combination for paternity testing. In some specific embodiments, the nucleotide sequences can be as shown in Table 1 below.

TABLE 1
Detection primer pair for SNP locus combination

| SNP locus | chromosome | the nucleotide sequences of the detection primer pair |
|---|---|---|
| rs868163 | chr1 | GTGGAGACTGGAAGCATTCTCAA (SEQ ID NO: 1) |
| | | TGCAGAGAAGCTCTGGAACTTG (SEQ ID NO: 2) |
| rs3007429 | chr1 | AAGCATTGCCCAGGAGAATAGG (SEQ ID NO: 3) |
| | | CTCCCTGGTCTTCATCTCACCA (SEQ ID NO: 4) |
| rs3007430 | chr1 | AGCGCTCAGTGACCTTTTGG (SEQ ID NO: 5) |
| | | TGCATCGCTCCCTTTAGAGACAG (SEQ ID NO: 6) |
| rs2076605 | chr1 | CACTGGGAGCAGGAGAGTCT (SEQ ID NO: 7) |
| | | CCGGCACCTCTCTCCCAT (SEQ ID NO: 8) |
| rs7531163 | chr1 | TTCTCGTAGTTGGGCAGGTTGTCT (SEQ ID NO: 9) |
| | | TCAGTCAGCTCCCTACTCACCAT (SEQ ID NO: 10) |
| rs607254 | chr1 | AAGATGACGCGGGCATTGTTCCT (SEQ ID NO: 11) |
| | | GACCCAAGCAGTTCCAAGTAAGG (SEQ ID NO: 12) |
| rs2361660 | chr1 | TGGTCTACGCGCATAGCAAGGTG (SEQ ID NO: 13) |
| | | GGCGAGGTCAGAGGGGCTGTCT (SEQ ID NO: 14) |
| rs7516939 | chr1 | GCTCCAAGAAATTCTTGGCTATATGGA (SEQ ID NO: 15) |
| | | TCTTCTATAGGACAAGGTGCAACG (SEQ ID NO: 16) |
| rs494198 | chr1 | TTGTGGGAAGGGCGTTCATCCAT (SEQ ID NO: 17) |
| | | AGGCCAGCAAGTGTGACAGTCA (SEQ ID NO: 18) |
| rs6588052 | chr1 | CCATTCTTCTCCCCAGAATGCAT (SEQ ID NO: 19) |
| | | TTGTGGGACCGTATGTAAAGAAGATCC (SEQ ID NO: 20) |
| rs11208265 | chr1 | TGGAAGTAGGCAGTCAGACGTA (SEQ ID NO: 21) |
| | | TGTCTTCCTCAGGTCATGTTGG (SEQ ID NO: 22) |
| rs472908 | chr1 | TCCTGTTATCACTCATGAGAGTTTCTC (SEQ ID NO: 23) |
| | | CCAAGGTCGCTTAAAAACCCAACAAG (SEQ ID NO: 24) |
| rs3736296 | chr1 | AGATCAGCGTATGATGCTCTTCCAA (SEQ ID NO: 25) |
| | | AAACCTACATGGCCAACAACTTTGG (SEQ ID NO: 26) |
| rs2274570 | chr1 | AATCCCAGCCACACCAAGGTAGT (SEQ ID NO: 27) |
| | | AAAATTTCCCTGCCTTCTTCCCTTC (SEQ ID NO: 28) |
| rs3811003 | chr1 | TGAGGTTGTGACAGCAACTGAGGG (SEQ ID NO: 29) |
| | | CAACCTGTTTCAGGATCTCCAGGA (SEQ ID NO: 30) |
| rs9428083 | chr1 | TATTTGAGGAGAGGCAGCTCCA (SEQ ID NO: 31) |
| | | CTGCAAACTCGCCATCAAACTC (SEQ ID NO: 32) |
| rs9427094 | chr1 | TGAGACTGCAGAGGTATGATGC (SEQ ID NO: 33) |
| | | TCCAGGATCTCCAGGTCATAGCCAT (SEQ ID NO: 34) |
| rs703116 | chr1 | ACGTTTAAATGACTTCTATTCTTTTCCCAT (SEQ ID NO: 35) |
| | | AGATTACCTTTTCCCGAACTTCATCAGA (SEQ ID NO: 36) |
| rs1920149 | chr1 | GCATCTATTCACAAGGTCGCTTC (SEQ ID NO: 37) |
| | | ACAGCAGGGACTATAAAGAACCAGGT (SEQ ID NO: 38) |
| rs7536561 | chr1 | TATGGAATCCCCCAGTCTCCATC (SEQ ID NO: 39) |
| | | AATTTGGGCATCATTGCGCATG (SEQ ID NO: 40) |
| rs647347 | chr1 | AGAATGGCTTTTACCGGCACAGAG (SEQ ID NO: 41) |
| | | ACAAGAGGGAAGGAAGGAAGGA (SEQ ID NO: 42) |
| rs2274064 | chr1 | CCACTTGTCTCTCATTTGGTCGAA (SEQ ID NO: 43) |
| | | CCCTTATGTGAAAATGGGGGTGA (SEQ ID NO: 44) |
| rs12042179 | chr1 | AGTTTGGTTGAGGCAGCACAA (SEQ ID NO: 45) |
| | | ACCAGGTGCCTCTCAAATTCTTGC (SEQ ID NO: 46) |
| rs4844573 | chr1 | GGACAGGCTTATTGACTGCTGTGGC (SEQ ID NO: 47) |
| | | TTACCAGACATTCCACATGCTTCCT (SEQ ID NO: 48) |
| rs2076355 | chr1 | AAGGGTTATAGGGCACCTTCCA (SEQ ID NO: 49) |
| | | TGAAGCCACATTCTCTACTCGGTGA (SEQ ID NO: 50) |
| rs12031285 | chr1 | TCCTGTACACAGCTCTGTGTCTCT (SEQ ID NO: 51) |
| | | TTTTCTTCAGGTCCTTGCACTTGG (SEQ ID NO: 52) |
| rs630120 | chr1 | CCATAACTGAACCCCTTTCTTAGTTGAA (SEQ ID NO: 53) |
| | | GAGCAATGTGAATTCATACCTTGGTCAG (SEQ ID NO: 54) |
| rs12042900 | chr1 | GTTTATTCACGCAGGATTAAAAGGGAA (SEQ ID NO: 55) |
| | | CTTTGCTATTTGCTGAAGTTTGATATCTATGTG (SEQ ID NO: 56) |
| rs7527925 | chr1 | CCTATAGGTGGTTTCTTCAGCTCTAT (SEQ ID NO: 57) |
| | | AATACAGGATGACATTGGATTCCTACC (SEQ ID NO: 58) |
| rs11578981 | chr1 | AGAAAGACAAGTGCCCTGAAGAC (SEQ ID NO: 59) |
| | | TTTGGATGTACTGGGAGTGGAGA (SEQ ID NO: 60) |
| rs7552783 | chr1 | TCTCGCCGCCATTTATCCTTC (SEQ ID NO: 61) |
| | | GATTTTGGCACTCAGAGCACCA (SEQ ID NO: 62) |
| rs2045955 | chr1 | TCATGATGGCTCTGTGGAGGTA (SEQ ID NO: 63) |
| | | AGGATACTGAGGAAGCCAGGTT (SEQ ID NO: 64) |
| rs2618702 | chr1 | AATTGAGCTTGGCTGGCAGT (SEQ ID NO: 65) |
| | | TTCTGCCTTTTCCCGCTATGG (SEQ ID NO: 66) |
| rs2805396 | chr1 | TGTTAGGTCGTGACAGAGTGGTGG (SEQ ID NO: 67) |
| | | AATGAACACTATGGGCGCTCTTG (SEQ ID NO: 68) |
| rs1332777 | chr1 | CCCTCTCGTCTGAAACAAAGGTTAC (SEQ ID NO: 69) |
| | | TTTTCCTCCTTCTCCTCCTCCT (SEQ ID NO: 70) |
| rs10802626 | chr1 | GGGGATTGACGATGTGATTGAGAC (SEQ ID NO: 71) |
| | | GGCCAACTGTTGCCGTTTTCTCTG (SEQ ID NO: 72) |
| rs2256242 | chr1 | AAGTGCCAGAGTCAGCATTCTGG (SEQ ID NO: 73) |
| | | AAATGTTTGCTGACCTCTCCCTGA (SEQ ID NO: 74) |
| rs10927011 | chr1 | CAGCAGAGCTTTAGCAAGGAAGCAA (SEQ ID NO: 75) |
| | | GGGAAAGATTAGAGCCTGGGCACT (SEQ ID NO: 76) |
| rs2280132 | chr2 | TAGAGAGGCAGCTGCGGAAC (SEQ ID NO: 77) |
| | | GCCCGGCATCCCCGGAGAGA (SEQ ID NO: 78) |
| rs2271621 | chr2 | CTGGTCGGACATGAAATAACTTGCTAT (SEQ ID NO: 79) |
| | | CTACTTTGACCCATGTTAGGAACTCAGA (SEQ ID NO: 80) |
| rs7561070 | chr2 | AGGTAAGCCCAGAAGACAAAGCA (SEQ ID NO: 81) |
| | | TGAAGATAGGCCCTAGTGCTGAGT (SEQ ID NO: 82) |
| rs4072407 | chr2 | GCCATCTGACTGTTTCTCAGAGTCAA (SEQ ID NO: 83) |
| | | CTACCTGGATTTGCCGAAGCAA (SEQ ID NO: 84) |
| rs455060 | chr2 | TTCCATGTGGATTCCACCTGTGT (SEQ ID NO: 85) |
| | | TAAGTAATCGGGGATGTTCCCTGA (SEQ ID NO: 86) |
| rs3738984 | chr2 | CACATCCTGGATGGAAAACATGCC (SEQ ID NO: 87) |
| | | GCCTTTGCTGTCCAGTTATCTCT (SEQ ID NO: 88) |
| rs3771274 | chr2 | TCTCCCCCAGAAGGTAAGAAGTACTGC (SEQ ID NO: 89) |
| | | CAACCTCCCTGTATAGGCACAGT (SEQ ID NO: 90) |
| rs2020911 | chr2 | GGGCAAGTCTACGCTTATGAGAC (SEQ ID NO: 91) |
| | | AAAGTCAGCCAGTGACTTAATAGGAAG (SEQ ID NO: 92) |
| rs2276625 | chr2 | TGAAAGACATGGCAGCATATATTACCT (SEQ ID NO: 93) |
| | | GAGCCTTTTGTGTAGGGAGACAG (SEQ ID NO: 94) |
| rs2244182 | chr2 | GTGAGAGGGAGCACTTCTTACAGTTA (SEQ ID NO: 95) |
| | | AATCAGGCAACTGGGACTGAAG (SEQ ID NO: 96) |
| rs2271244 | chr2 | GCATGAGCTCTGGTTGTAAGAAGTA (SEQ ID NO: 97) |
| | | GAGTACAGGCACTTCCAAAATGC (SEQ ID NO: 98) |
| rs72481904 | chr2 | CAGGCTGGGCACCACAGCA (SEQ ID NO: 99) |
| | | GGGACGGCAGCTCCTCCT (SEQ ID NO: 100) |
| rs1061305 | chr2 | TGGTGCTTCTGAATGCTCAGACTTC (SEQ ID NO: 101) |
| | | TTGGGGGAGATGTAGCAAACATG (SEQ ID NO: 102) |
| rs16830136 | chr2 | CCCCTTATATCTTTGTGCATTTTCTCAAAC (SEQ ID NO: 103) |
| | | CTGTAATTCTGTGGCCTCTTTTAGGTGA (SEQ ID NO: 104) |
| rs2288201 | chr2 | TCCCGATACAGTCTCTACGTTGGAG (SEQ ID NO: 105) |
| | | GAAGAGGGGAGGAAATATCGAGGA (SEQ ID NO: 106) |
| rs12620077 | chr2 | ACGTCATTGCTTATGGTCACTCA (SEQ ID NO: 107) |
| | | TTGGCCAATTCAAAGCTCATGGCAT (SEQ ID NO: 108) |
| rs1867864 | chr2 | CCAGCTCTTAACTCTCTTCATCTCATTT (SEQ ID NO: 109) |
| | | CTTTTCTCTCCAAGACGCAACAGT (SEQ ID NO: 110) |
| rs1542484 | chr2 | GGCTGTTATCTTCAGTTTCTATAAAAAGAGAGA (SEQ ID NO: 111) |
| | | CAGAGCTATTTCCACATAGTAGTGCATC (SEQ ID NO: 112) |
| rs830973 | chr2 | GCCATTTGATTTTAAGCTCTGGGATGTG (SEQ ID NO: 113) |
| | | CAAGTCCTCCCATCACTTTCTAACATG (SEQ ID NO: 114) |
| rs12613075 | chr2 | TACTGGTAGTGAACGGAGACCAC (SEQ ID NO: 115) |
| | | TCGTAAGGTCACAGCTGTACAC (SEQ ID NO: 116) |
| rs2366751 | chr2 | GTGCTCCACTTTTCACTCCCTTT (SEQ ID NO: 117) |
| | | TGTAGCTGTATTACGGGTCACATCAAC (SEQ ID NO: 118) |
| rs2303838 | chr2 | CTCTGCATCACGTTTTTGTACCA (SEQ ID NO: 119) |
| | | TGGCCCAGGAGTATCTGGATAAATAGTAGG (SEQ ID NO: 120) |
| rs2042995 | chr2 | ACCTTTAGGTGGTGGTTCAACCCTT (SEQ ID NO: 121) |
| | | GATATGCCATGTTGTGGGTGGT (SEQ ID NO: 122) |
| rs2562839 | chr2 | TCACTCGGTGCTACATTGATGATC (SEQ ID NO: 123) |
| | | CCAGCTACTGTACACTCCAAGGTACAG (SEQ ID NO: 124) |
| rs12997453 | chr2 | GATCCTAAGTCTGATCAATTGTTTGTCAG (SEQ ID NO: 125) |
| | | TTCTTGCACATCATCAGAGCTGT (SEQ ID NO: 126) |
| rs3024866 | chr2 | CTCCCATTGGAAGATTCTTCAATAGACATG (SEQ ID NO: 127) |
| | | TACAAGAGCAGCAACAAAGTTTCCA (SEQ ID NO: 128) |
| rs1208081 | chr2 | TGTGGGATATGGATAGGGGGAAAG (SEQ ID NO: 129) |
| | | TGTCTTCTTTGAACAGGAGCCTCT (SEQ ID NO: 130) |
| rs2242074 | chr2 | ATCCAGCAGCCGCTGTCCA (SEQ ID NO: 131) |
| | | AAGGCAAGGCGGGACAAG (SEQ ID NO: 132) |
| rs17552879 | chr2 | AGTTGCTTTCTTAGGAAATGTAGTTGCTTT (SEQ ID NO: 133) |
| | | TAAAAGATGGGTGTTTGAAGGAGCA (SEQ ID NO: 134) |
| rs3213784 | chr2 | ACCTTCTTATTCAGCCCCGACTGGA (SEQ ID NO: 135) |
| | | TTCCTTTTCCAGTGGGTCTGCTA (SEQ ID NO: 136) |
| rs6704780 | chr2 | GGTATGTCGCCCTAGAAAAATGAACAA (SEQ ID NO: 137) |
| | | ATCGTTTTCTCTTAAGGCTACTGTTCATG (SEQ ID NO: 138) |
| rs12991025 | chr2 | GTCGTTTCCACTGCCAGCT (SEQ ID NO: 139) |
| | | AGTGCCCTTCTTTTCCCCTTG (SEQ ID NO: 140) |
| rs34490902 | chr2 | CGCCTGGAAGCGGACTTCAC (SEQ ID NO: 141) |
| | | TGAGGAGCGTTGGGGACGAAG (SEQ ID NO: 142) |
| rs2304774 | chr2 | CGAATAGGACATGGCCAAGACAAAA (SEQ ID NO: 143) |
| | | CCAACAGTCCTGTGGGGTTGACT (SEQ ID NO: 144) |
| rs17868324 | chr2 | CGAAGTGCATTTTCTCTATTAACAAGTTCA (SEQ ID NO: 145) |
| | | GTTTTGATGCAGTGTTTCTCGATCC (SEQ ID NO: 146) |
| rs1105879 | chr2 | ACAATCACTTTGCTGAGCGATCAT (SEQ ID NO: 147) |
| | | CACCCTGAACTTCTTTAAGGAGAGCA (SEQ ID NO: 148) |
| rs4234097 | chr2 | GGCTGCTGCAGAAGTGACACC (SEQ ID NO: 149) |
| | | ACCTCCCTGAGGAAGGACAAC (SEQ ID NO: 150) |
| rs6756901 | chr2 | GATCTTGGGAGGGGCTGTTG (SEQ ID NO: 151) |
| | | TGGTAACCTGCACCTCAATGTGA (SEQ ID NO: 152) |
| rs456168 | chr3 | ACTGTAAGCCATGATGCCCTTCC (SEQ ID NO: 153) |
| | | CCACAGAGGTACGCACTGACACA (SEQ ID NO: 154) |
| rs2733532 | chr3 | AAGAGAGTCCACCTCCTGCAT (SEQ ID NO: 155) |
| | | TCAGATACCAGGAAGCCCCAT (SEQ ID NO: 156) |
| rs3729587 | chr3 | TGAGGATGCAAAGGCTCAGAGAGA (SEQ ID NO: 157) |
| | | AAGGAAAAGTCCTTCCCCATCATTCC (SEQ ID NO: 158) |
| rs3816526 | chr3 | TGTCTCTGATTCCAGAGCCAGTA (SEQ ID NO: 159) |
| | | ATCAGCCCTGTAGGACTTCTGA (SEQ ID NO: 160) |
| rs2507940 | chr3 | CTGTGGTTTGCTTCTCTCTCCTTCC (SEQ ID NO: 161) |
| | | TGCTGTCTGGGCATTTCTGTTCTCT (SEQ ID NO: 162) |
| rs13097628 | chr3 | TGTACCCAGAATCTGTGCTCAAGT (SEQ ID NO: 163) |
| | | CATCACATGGAACGAACTGTGTCATG (SEQ ID NO: 164) |
| rs7641844 | chr3 | ACCCTGAGTCAGATCCATTGCCAC (SEQ ID NO: 165) |
| | | TGGTTCTGAACCCTCCAACCCAAG (SEQ ID NO: 166) |
| rs1799987 | chr3 | CAGGTTGTTTCCGTTTACAGAGAACA (SEQ ID NO: 167) |
| | | CCAGGATCCCCCTCTACATTTAAAGTTG (SEQ ID NO: 168) |
| rs936175 | chr3 | TTGCTAGGTCCACTCTGGGATC (SEQ ID NO: 169) |
| | | GGAAGCATCAAACTCGACCTCCT (SEQ ID NO: 170) |
| rs13066214 | chr3 | CTCTCCCATCTTCCTCCTCTCTTCT (SEQ ID NO: 171) |
| | | GAGCCCGATGTCCAAAAGGTA (SEQ ID NO: 172) |
| rs749512 | chr3 | CCAAAACATGATGCCCCTCCA (SEQ ID NO: 173) |
| | | GTTTAAGGAGCGCATCGGCTAC (SEQ ID NO: 174) |
| rs6445873 | chr3 | TGTATCATCTCTCTGTTGGGGCTAC (SEQ ID NO: 175) |
| | | GTCACCGGGACAGCAATAAAGCT (SEQ ID NO: 176) |
| rs4681982 | chr3 | GGGAAGCTAATACAATATGTAAAGTTTTCTCATAA (SEQ ID NO: 177) |
| | | TAGAGGCACATATTTAAAATAATTTGAAATACGGAA (SEQ ID NO: 178) |
| rs2290174 | chr3 | GCTCTGGTATTCCTGCAGGTTCAG (SEQ ID NO: 179) |
| | | ACATGGCCAGTCTTAGAGGTACTG (SEQ ID NO: 180) |
| rs533852 | chr3 | GGACACTACATGAAGTCCTGTGTA (SEQ ID NO: 181) |
| | | CTCACACTCTCCTCTGGCCTT (SEQ ID NO: 182) |
| rs1463138 | chr3 | GAAGCTGTGCTTCTCCATGAGA (SEQ ID NO: 183) |
| | | AATCAGTTTCTGCCAAAGGGACT (SEQ ID NO: 184) |
| rs2713604 | chr3 | CCACCCTGACCCTTTTTAAGCAGA (SEQ ID NO: 185) |
| | | CCCTCTTACGGGAAGCCCTTCTG (SEQ ID NO: 186) |
| rs1683787 | chr3 | GCACACGTGGCACTACCATGG (SEQ ID NO: 187) |
| | | CCGGATGTACATCGCCCTGAC (SEQ ID NO: 188) |
| rs2681092 | chr3 | TGAAAGAAGAGAACCGGGTATGCTT (SEQ ID NO: 189) |
| | | AGGCTGTGTGGGTCCATTGAGCAT (SEQ ID NO: 190) |
| rs16861582 | chr3 | ACAAACAATCAGGGGGCCAATTA (SEQ ID NO: 191) |
| | | AGGGCCTCAGGAATTTTGGAGA (SEQ ID NO: 192) |
| rs2303909 | chr3 | ATGCAGCGTTTATGCAGATACCCAT (SEQ ID NO: 193) |
| | | GATGGAGACTTTGGGTAAGTGCT (SEQ ID NO: 194) |
| rs9290259 | chr3 | GTAGATACTCCTGACAATTTCCTACAGTTCT (SEQ ID NO: 195) |
| | | TACCACTTCTTTCACTACATCTAGTGAC (SEQ ID NO: 196) |
| rs1515442 | chr3 | AGGGTCTACTATTTCAGCAGCTTGG (SEQ ID NO: 197) |
| | | TAGACCAACTTCATTTCCACATCCA (SEQ ID NO: 198) |
| rs2593813 | chr3 | TGAACGCTTTCAGGCAGTCAC (SEQ ID NO: 199) |
| | | TCAGGTACTAGTGAATCAGGCAGAACC (SEQ ID NO: 200) |
| rs2028572 | chr3 | TGTTCATCGAAGAGATCCCTGTCAT (SEQ ID NO: 201) |
| | | CACGATCAGTGGTGAAGTGTTTCTG (SEQ ID NO: 202) |
| rs9851685 | chr3 | ATAGGTTGCTTGGGAGACCCTACAA (SEQ ID NO: 203) |
| | | AGTGGAATGACTTTGCGGAGGA (SEQ ID NO: 204) |
| rs2286342 | chr4 | AGGAGAGAGAATACTCTGAACAGCTAAT (SEQ ID NO: 205) |
| | | GCCTAATACTTGAATTCCCCTATCG (SEQ ID NO: 206) |
| rs899691 | chr4 | GCTGTTAATTAACATGCCCTGGTGGT (SEQ ID NO: 207) |
| | | TGATTGAGTTTCTGAAGCTGCAAGTC (SEQ ID NO: 208) |
| rs2306400 | chr4 | ACCAATCCAGGTTGGTGAGTAGG (SEQ ID NO: 209) |
| | | CTGTCCCTGTCCTTCCTTTCTTC (SEQ ID NO: 210) |
| rs13121363 | chr4 | GTGAGACCCTATCTCAATAAATAAATAATTGTCTTC (SEQ ID NO: 211) |
| | | TCTTCAAGGATGTTCAGTGAAAAGCATG (SEQ ID NO: 212) |
| rs13144326 | chr4 | GGCCTGGTCTATAAGGATTGCTTAA (SEQ ID NO: 213) |
| | | ACCTGATTTTCACCCCAGGCCAT (SEQ ID NO: 214) |
| rs7655165 | chr4 | AGAGTATCTTAAACTTTCTGGCTTTTGTACAC (SEQ ID NO: 215) |
| | | TTGTTTTGTGGAAAAAGATTCAGATTTTTGC (SEQ ID NO: 216) |
| rs12650452 | chr4 | GTTTGCTCTCAAAGCCTCAGTGGAA (SEQ ID NO: 217) |
| | | TAATAGCTCGAGGCTACTGACACT (SEQ ID NO: 218) |
| rs2125314 | chr4 | TCCCCAACATGAAGATTATGGACTGT (SEQ ID NO: 219) |
| | | GCACTTCGTGAGGCAGATGTAGACTG (SEQ ID NO: 220) |
| rs225170 | chr4 | GCAGGCCACTTTCATGAAACTCCAT (SEQ ID NO: 221) |
| | | AGGCCGCATACATTTAATGTAATTGGA (SEQ ID NO: 222) |
| rs6856362 | chr4 | AGGACTTGAGGCATGTGCAAAC (SEQ ID NO: 223) |
| | | GCTGTTATAGATGCTTGGGGGCATG (SEQ ID NO: 224) |
| rs7660664 | chr4 | TGTCACATTCACCATCGTGCAGCC (SEQ ID NO: 225) |
| | | CATGACGGCAGTAACTCCCTCA (SEQ ID NO: 226) |
| rs34840208 | chr4 | CGGTTCACCTTCACTGTTTCTGAT (SEQ ID NO: 227) |
| | | GACACTGGAGGAGGTAGCCTAG (SEQ ID NO: 228) |
| rs7677541 | chr4 | AGGTGTTTTCCTGGGAGTCTGAAT (SEQ ID NO: 229) |
| | | GGCCCAGATGCCATTGCGATTCTGA (SEQ ID NO: 230) |
| rs227365 | chr4 | AGTCTGGTTTCCATGTCCATGAG (SEQ ID NO: 231) |
| | | AAGCATTACATCAATTTCTCAAATAGTCAAGTAC (SEQ ID NO: 232) |
| rs17009761 | chr4 | GTAGTGTTGGCTGGAAAGGCTA (SEQ ID NO: 233) |
| | | TGTGGACTCCTGTTCTGAGAACCA (SEQ ID NO: 234) |
| rs62333013 | chr4 | TGCTCGTCGCCTGCCA (SEQ ID NO: 235) |
| | | GCGTGAGTAACCGCCGCGGT (SEQ ID NO: 236) |
| rs2166337 | chr5 | GGACAGGAGTAAAACCGAGGATC (SEQ ID NO: 237) |
| | | CCCAAGGAGAATGCAGAGCCAT (SEQ ID NO: 238) |
| rs4429853 | chr5 | CTGAGACGAAGAAAAACCGAGGAAA (SEQ ID NO: 239) |
| | | ATGGGAAAAGGGGAGAAGATTGTGT (SEQ ID NO: 240) |
| rs13170062 | chr5 | CTGTCTTCATTACTCTGCAAAGCAG (SEQ ID NO: 241) |
| | | ATATGAAACCATTTCTGCACCCCAT (SEQ ID NO: 242) |
| rs162850 | chr5 | GTGGTAACATGTTATTTACCCATTCAGTATT (SEQ ID NO: 243) |
| | | AGTGTCTGTCTGTGGAGTGTATCCTTC (SEQ ID NO: 244) |
| rs7710284 | chr5 | ACTCTTGTCCAAGAAGCTATCCAAG (SEQ ID NO: 245) |
| | | TGCTCTACCAGTTCTGCAAGAGGAGA (SEQ ID NO: 246) |
| rs6451206 | chr5 | GTCTAACAAAATATTCATGAGTTGTCCTGTT (SEQ ID NO: 247) |
| | | GAAGAAGCTCTTACAGGCTGCA (SEQ ID NO: 248) |
| rs11957963 | chr5 | AGCAATCTAAAGCATAGCCACCAT (SEQ ID NO: 249) |
| | | TTAATGAGAGCTGGTTACAGGACAC (SEQ ID NO: 250) |
| rs476569 | chr5 | GATCGCATTGTGACCATTCACTC (SEQ ID NO: 251) |
| | | ATAATGACCATTGTGTATATCTGTTTCCAAG (SEQ ID NO: 252) |
| rs1801033 | chr5 | GAATGCATGGTTGAAAGGCTACCAG (SEQ ID NO: 253) |
| | | AGATATAACTCTGAGGCAGGGTCAAG (SEQ ID NO: 254) |
| rs706714 | chr5 | TTCCTGTTGCACCAGGTTCTTCGAA (SEQ ID NO: 255) |
| | | GTCGTTATGTTCTGGGCAGAAG (SEQ ID NO: 256) |
| rs182190 | chr5 | GAAACAGAGAAAAATGCTTCCAAAGCAA (SEQ ID NO: 257) |
| | | GCAAATTGGCATGTGTACATGGT (SEQ ID NO: 258) |
| rs309557 | chr5 | CACAGGTTTTCCTTTGGAGGAAGATT (SEQ ID NO: 259) |
| | | ATTTAGGGACACCCAGACTTCAC (SEQ ID NO: 260) |
| rs4916683 | chr5 | GCCTCTAAGTAACTGGCTTTAATCTCT (SEQ ID NO: 261) |
| | | AACATAGACTCTGATCCTGATGGTGA (SEQ ID NO: 262) |
| rs274557 | chr5 | CTGCCACTGTTTGCTTACTTCATC (SEQ ID NO: 263) |
| | | CCCACTGGCAGGATGATTTCTGTCT (SEQ ID NO: 264) |
| rs2303068 | chr5 | CTAAGAGTACAGCTTCCTTTGAGGT (SEQ ID NO: 265) |
| | | CATGTTCAGGGAACACGTGCAT (SEQ ID NO: 266) |
| rs4072924 | chr5 | TCCTAACCTTCTTCATGGAGCCTAT (SEQ ID NO: 267) |
| | | CAACAGGTTGCTGCCGACT (SEQ ID NO: 268) |
| rs438464 | chr5 | CCCGTCTTCCCCTATGGTCT (SEQ ID NO: 269) |
| | | GCCAGCTTCACGGGCAGCTTC (SEQ ID NO: 270) |
| rs2236277 | chr6 | TTTCCCTTTGCCCCCATGTAG (SEQ ID NO: 271) |
| | | TTCTGCGAGAACATCCATGATGG (SEQ ID NO: 272) |
| rs2842949 | chr6 | CTCCATGTATATATGCAGTATGCTTCCTA (SEQ ID NO: 273) |
| | | ACAACTTTACCTGGATGTTTAGTTGGATC (SEQ ID NO: 274) |
| rs2071303 | chr6 | GTGGGATCACATGTTCACTGTTGACT (SEQ ID NO: 275) |
| | | TTGAAGGAAACAGCTGGAAGTCTGAG (SEQ ID NO: 276) |
| rs3737095 | chr6 | GTGGCCCCTCTCCTTTCTTG (SEQ ID NO: 277) |
| | | TACAGGTCTGGTTCAGTGAGAAGATG (SEQ ID NO: 278) |
| rs3818554 | chr6 | CAAAGCCCTAGGGAACCACAGGAA (SEQ ID NO: 279) |
| | | AGCAGAGGCCCTCTGTTGATG (SEQ ID NO: 280) |
| rs167772 | chr6 | CCTCTTTCCCCAAAAGCTACGAACT (SEQ ID NO: 281) |
| | | GCCCGCATTGACAAAAAGCA (SEQ ID NO: 282) |
| rs3920621 | chr6 | GAAGCTTTTGTTCCTTTTCTAAGGGATTT (SEQ ID NO: 283) |
| | | ATGCAATGTAGATACTGTGAAGTAGACT (SEQ ID NO: 284) |
| rs10948667 | chr6 | TGACAACCTCCCAGGATGTTGTTC (SEQ ID NO: 285) |
| | | GAAAGTGTCCTCCTAGAGGTGGA (SEQ ID NO: 286) |
| rs9474140 | chr6 | GTGCCAACTGCTTACAATTTATTGCCA (SEQ ID NO: 287) |
| | | ACACACTTACCTATCAATGTACTCAGCA (SEQ ID NO: 288) |
| rs9367689 | chr6 | AACTACTGTTTGCCTCTAAGATTTTCTC (SEQ ID NO: 289) |
| | | TTCACCATTTAATACTGCTCCACCT (SEQ ID NO: 290) |
| rs4712138 | chr6 | TCCAGCTCCTTTTTAGACTGTTCTG (SEQ ID NO: 291) |
| | | TCACCATTCTCTTTTAAGAAGTTCTCTGTG (SEQ ID NO: 292) |
| rs2230862 | chr6 | TGGCAGCTGTTAGTTCATCTACCTG (SEQ ID NO: 293) |
| | | CTGCATGATGTAGCCTGTGTGATC (SEQ ID NO: 294) |
| rs4710457 | chr6 | CTGATGCAGGTGCCATTGTTG (SEQ ID NO: 295) |
| | | AGGTCTAGAATGCAGCCCCTGAAC (SEQ ID NO: 296) |
| rs1936439 | chr6 | ACATTCTTCAAAAACCAACATGGATTTTTTG (SEQ ID NO: 297) |
| | | ATAACATAAACAGCTAATTTTTAAAACCACAGTG (SEQ ID NO: 298) |
| rs2305837 | chr6 | TTTAAGGCGCGTTTATCCCTTG (SEQ ID NO: 299) |
| | | GATTTAGCAGATCAGGCAACAAAACC (SEQ ID NO: 300) |
| rs519068 | chr6 | CTTACCGGTTTGCCTGAATTTCCC (SEQ ID NO: 301) |
| | | ATCAATCTGCCTTACCCAGACTG (SEQ ID NO: 302) |
| rs1875845 | chr6 | TCAGCTGCTTTTACTGCTGGTTT (SEQ ID NO: 303) |
| | | CATGTTCTTGATCTCTGCTGCCTT (SEQ ID NO: 304) |
| rs9448893 | chr6 | CTTTCTTTGTACCTTGATTTTAGTCAAGTTGTA (SEQ ID NO: 305) |
| | | AATGAATCTTTTCCAGTCTGTAACAAGTG (SEQ ID NO: 306) |
| rs3828753 | chr6 | CATGCAGATCAGTTCCTGAGACTT (SEQ ID NO: 307) |
| | | ATTTCAGGTTCATGTGATCCGAGAG (SEQ ID NO: 308) |
| rs3734353 | chr6 | TTTTGGTAGCAGCCGTCGAAGG (SEQ ID NO: 309) |
| | | GGTAGGAAGAGCCCCCAAAAGGT (SEQ ID NO: 310) |
| rs6922753 | chr6 | CCTTGCAGTGTAATCGACATAAACCC (SEQ ID NO: 311) |
| | | GAACATCTGCCTTGTCTCTTTTGTTG (SEQ ID NO: 312) |
| rs2273752 | chr6 | GAGTTGAATGTCAGCCAATTTCCATA (SEQ ID NO: 313) |
| | | GGGGTATTTGGGATCTGTAGTGAGCCTT (SEQ ID NO: 314) |
| rs7754205 | chr6 | CCATAGCCATTGTACCCATCCAAG (SEQ ID NO: 315) |
| | | GCAGTGCAGCAGATAAGGTCACAG (SEQ ID NO: 316) |
| rs3813328 | chr6 | AGGAAAAGCGGATTCAAAATTGCAG (SEQ ID NO: 317) |
| | | CCTTACTTTTTCCCTTGGGTTGTTC (SEQ ID NO: 318) |
| rs902373 | chr6 | CCCAATACCATTGGAGAGAAATGTTCTA (SEQ ID NO: 319) |
| | | TTGTAGAGCTCTGTAATTGGGCAT (SEQ ID NO: 320) |
| rs3798663 | chr6 | GCCACAACCTGTGATTCAGAGACTA (SEQ ID NO: 321) |
| | | TAGGGAAAACCTCCCTCAATTCTGT (SEQ ID NO: 322) |
| rs6914831 | chr6 | AGAGAGTGAAAATCAAAGTACATATGTAATACG (SEQ ID NO: 323) |
| | | AGGAGGGGATCGCTCCTTTATCTCA (SEQ ID NO: 324) |
| rs3762000 | chr6 | CATGATTTTACACATTTGTATTAATCACAGTACCAA (SEQ ID NO: 325) |
| | | CTGTATACAGTACCTGTATGCTGGTTGTTC (SEQ ID NO: 326) |
| rs22908 | chr6 | TTCAGCACCTACTTGGAGATGGT (SEQ ID NO: 327) |
| | | TAGGCCTGACAGAATTGATCACTG (SEQ ID NO: 328) |
| rs6904364 | chr6 | GACCCAAACCCATCATCACCTAATG (SEQ ID NO: 329) |
| | | TTTTGCAGAATTTTCCACACCCATC (SEQ ID NO: 330) |
| rs3818110 | chr6 | AGATCCCTTTTCTGCTGCTGTGTAA (SEQ ID NO: 331) |
| | | TTGATCTGGAGCTCGATGGTCT (SEQ ID NO: 332) |
| rs9371581 | chr6 | CAGGGGAAACTGTTCTCTTCCATT (SEQ ID NO: 333) |
| | | CTCCTGTATGTTACTGGTGGCAA (SEQ ID NO: 334) |
| rs9383985 | chr6 | AGGTGGACAGCTGTACTCTCT (SEQ ID NO: 335) |
| | | TTTCTTTTTGGAGACTGAGTTTTGCTCT (SEQ ID NO: 336) |
| rs9397102 | chr6 | CTTGGTTGCCCTGTTAGACTGACG (SEQ ID NO: 337) |
| | | TTAGCCTCAATTTGTTCACCACGTT (SEQ ID NO: 338) |
| rs1873176 | chr6 | TAACTCAAGGGTGCTGAGCATG (SEQ ID NO: 339) |
| | | GCAGATCATAGAGGCGATTGCTGCT (SEQ ID NO: 340) |
| rs214968 | chr6 | CATCAAGCGACTGTTGGTTTCCA (SEQ ID NO: 341) |
| | | CATTACTGAAAGGATCATTTTGTTAGAGGGTA (SEQ ID NO: 342) |
| rs3734440 | chr6 | TGGGTGTTACCCAGAAAACCTTC (SEQ ID NO: 343) |
| | | TTTCTTAGGATCTGTCTGGCTCCA (SEQ ID NO: 344) |
| rs9356399 | chr6 | CCTGGCCTGATTAATTTTCCATAGCTA (SEQ ID NO: 345) |
| | | CAGCAAATGGATTCCGCAGT (SEQ ID NO: 346) |
| rs4716346 | chr6 | GTGCTGGTTGTGCTCCGAAGC (SEQ ID NO: 347) |
| | | ACCTGCGTATGTGGAGTAGGT (SEQ ID NO: 348) |
| rs2527509 | chr7 | GGCTTAGGGGCTCCTAAATGTCACC (SEQ ID NO: 349) |
| | | TTGACCGAGTGCACGAAGC (SEQ ID NO: 350) |
| rs852423 | chr7 | GGGTGTTGAAGGTCTCAAACATGATC (SEQ ID NO: 351) |
| | | AAGAACACGGCTAAGTGTGCT (SEQ ID NO: 352) |
| rs6461252 | chr7 | CAAATGAACCACACTACAGTGCTTGTCC (SEQ ID NO: 353) |
| | | GACACCTTCCTCAACAAATGGTCTCAC (SEQ ID NO: 354) |
| rs7782939 | chr7 | TCTGCAGGGAGGCGGAAGCC (SEQ ID NO: 355) |
| | | CGCTAGCCTCGGGCCGATGC (SEQ ID NO: 356) |
| rs1023542 | chr7 | CTTGGGTGTAAGGAAAGAAGAATTGGGC (SEQ ID NO: 357) |
| | | GGTTGTCCAGCTTGAGGAACTGT (SEQ ID NO: 358) |
| rs2240401 | chr7 | GGCTCTGTGTAAGGTTTCCTGA (SEQ ID NO: 359) |
| | | CAGCATAGTCCAAGACCTAGCCAA (SEQ ID NO: 360) |
| rs10261071 | chr7 | GAAAATTTATCCAAAAGTAACAGGAAAAGACTTT (SEQ ID NO: 361) |
| | | GGTAAATGTTCATTATAGAAACTTTGAAAAATGCA (SEQ ID NO: 362) |
| rs12669315 | chr7 | CCAGGAGTAGCTATGACTGATCTTGC (SEQ ID NO: 363) |
| | | TGATTGTAACCTACTGTCATCCCAGGT (SEQ ID NO: 364) |
| rs6950777 | chr7 | CAGCCGATGGATCACTTTGGTCCG (SEQ ID NO: 365) |
| | | AACATAGCGAAGGCATTGGTACCT (SEQ ID NO: 366) |
| rs2072454 | chr7 | CCATTCTCCCGCAGAAATCCT (SEQ ID NO: 367) |
| | | ATGTCGATGGACTTCCAGAACC (SEQ ID NO: 368) |
| rs2286822 | chr7 | AGGAGCTGCTGCGCAAG (SEQ ID NO: 369) |
| | | GCCCCAATCAGCCCCATCTCAC (SEQ ID NO: 370) |
| rs733957 | chr7 | CCAGCAGCAACTTAAAATTACAACAGA (SEQ ID NO: 371) |
| | | TTAAAGGGCAAAATGGTTATAATGTTGCT (SEQ ID NO: 372) |
| rs6957975 | chr7 | CACTTACACAGACGTGCACAGAAG (SEQ ID NO: 373) |
| | | TAAACAAAGCATCAGGGGCACA (SEQ ID NO: 374) |
| rs7779997 | chr7 | GACCATGCTATTTCTTTGGCACA (SEQ ID NO: 375) |
| | | CTATGGATATGTGTCTGGAGGTTCC (SEQ ID NO: 376) |
| rs362726 | chr7 | CCAGGAAGGACATGAAGTCCAAC (SEQ ID NO: 377) |
| | | TTTCCTAGAGGGGAAGGAACAGT (SEQ ID NO: 378) |
| rs607755 | chr7 | TTGCAGTTGGTGGTAGGAGTCAAA (SEQ ID NO: 379) |
| | | AAACACAAGTGCCAACATGTGT (SEQ ID NO: 380) |
| rs2520272 | chr7 | AGTCACACTGGTAAGTATGTACTTCTTCATG (SEQ ID NO: 381) |
| | | CTGTAAGTGTAGTTCTCTGTCCAACTG (SEQ ID NO: 382) |
| rs213950 | chr7 | GCAAGTGAATCCTGAGCGTGAT (SEQ ID NO: 383) |
| | | AGAACTGGAGCCTTCAGAGGGTA (SEQ ID NO: 384) |
| rs3815221 | chr7 | GCTTGACATCTGGGGTTTTAGTTGC (SEQ ID NO: 385) |
| | | AAATCGATTTTGGTGTAAGCGTGAAG (SEQ ID NO: 386) |
| rs3779032 | chr7 | TGACGTAGACCACAGGAACATGGC (SEQ ID NO: 387) |
| | | TCGCATTATAGTCCCTGTCCCTA (SEQ ID NO: 388) |
| rs7830 | chr7 | GCGGCTGCATGACATTGAGAG (SEQ ID NO: 389) |
| | | AGAGAGGGCAGGAAACAAAGTCCA (SEQ ID NO: 390) |
| rs3802100 | chr7 | ACCACCGTGATTTGCCCATCCCC (SEQ ID NO: 391) |
| | | TGAAGTTCTAGGCGTGCAGAGACA (SEQ ID NO: 392) |
| rs1550697 | chr8 | GCCTCGGTTTACTCTTAAATGTAAATAGAAC (SEQ ID NO: 393) |
| | | GGCCTATGTTGAAGTGTGGTCATCCA (SEQ ID NO: 394) |
| rs3020221 | chr8 | CTTACAGTTTGATGTGGACATCATAGTC (SEQ ID NO: 395) |
| | | TGAATTATTCACCGTGGCAGTCAC (SEQ ID NO: 396) |
| rs2073574 | chr8 | AGCAAGCCCAAATTTCAGAAGTTC (SEQ ID NO: 397) |
| | | TGTCTTCTGCTACTATTGAAGTACAAATGG (SEQ ID NO: 398) |
| rs12675283 | chr8 | GCCAGGAAAGTTGCCAAGTAGGC (SEQ ID NO: 399) |
| | | AGCATGCTTTGAAATCTAGTTCTTGGACA (SEQ ID NO: 400) |
| rs2272730 | chr8 | GAGGCGCAGGTAACCTCTGGAG (SEQ ID NO: 401) |
| | | AATGTCACGGGGAAAGAGCTT (SEQ ID NO: 402) |
| rs2737335 | chr8 | TGCGCCCTTACGAAAAAGGTA (SEQ ID NO: 403) |
| | | ACAGAATCTCAGAGCCTCATCCTTC (SEQ ID NO: 404) |
| rs7842714 | chr8 | AAGTCCCAAAGGAAGCCAGTACCCC (SEQ ID NO: 405) |
| | | GGCCGAGGCGCTTACCTGGT (SEQ ID NO: 406) |
| rs3098659 | chr8 | CTGCTCCCGGTGATGCAAACCC (SEQ ID NO: 407) |
| | | GTGCGTCATGGGCAACATCC (SEQ ID NO: 408) |
| rs6511 | chr8 | GTGGTTCAGTGTCACAATGCATT (SEQ ID NO: 409) |
| | | AGGCAAAAGCAGCAGGTCACCAGA (SEQ ID NO: 410) |
| rs10093032 | chr8 | TTGCCCTGGGCACATTGTTC (SEQ ID NO: 411) |
| | | CATCCTCAACATGGAGAGCATGGT (SEQ ID NO: 412) |
| rs2261147 | chr8 | GAAAATAACTCTACAGGCCCATTGCCTC (SEQ ID NO: 413) |
| | | GGCAAGATGTGCAGTGCTGACTACG (SEQ ID NO: 414) |
| rs2069568 | chr8 | CCAGGACAACTGATTATTGTTGCATCCAA (SEQ ID NO: 415) |
| | | GAGCACTGAAGAGGTCTTTATGGGTAG (SEQ ID NO: 416) |
| rs2272653 | chr8 | CTTGCCTGGACATCAAACTCTTGC (SEQ ID NO: 417) |
| | | GGGACATATGGGCCTTTCTATGACCT (SEQ ID NO: 418) |
| rs11780911 | chr8 | GGTGAGGACATGGCCACACCCT (SEQ ID NO: 419) |
| | | GGGTGGCGGGGCTTCAGCT (SEQ ID NO: 420) |
| rs3817681 | chr8 | TTCTGACTCCGGCTCTGCATCC (SEQ ID NO: 421) |
| | | AAAAGAACTGGCTGTGACCTTTGC (SEQ ID NO: 422) |
| rs17855765 | chr8 | CTTGGGGCTCTCGAGCAGGG (SEQ ID NO: 423) |
| | | CGGGCGACGTACCTGGC (SEQ ID NO: 424) |
| rs4244610 | chr8 | GCCTGTCAGAGCTGATCACTGC (SEQ ID NO: 425) |
| | | CACACGGCCATAGAGGAAGTC (SEQ ID NO: 426) |
| rs4251691 | chr8 | CCACTGCAGCTGGCAGA (SEQ ID NO: 427) |
| | | CCAAGCACACAGCCAAAGG (SEQ ID NO: 428) |
| rs12683181 | chr9 | CCAGCCAACTTCTAGCAAATTCG (SEQ ID NO: 429) |
| | | GTTATGATTTGGGCTGTGCGTTG (SEQ ID NO: 430) |
| rs11243704 | chr9 | GAGCCATCATTGACTGGTCGTA (SEQ ID NO: 431) |
| | | TACGTATATGGCAACGTTCAGGT (SEQ ID NO: 432) |
| rs12358961 | chr10 | TTATAGAAGGCAGGGCAGACCT (SEQ ID NO: 433) |
| | | ACTGGCTGCATTGGACTTTGCA (SEQ ID NO: 434) |
| rs10828545 | chr10 | AGCCAGCTGACGTAATTCAGAG (SEQ ID NO: 435) |
| | | CTGCCTAAGTGGTTCTGGAACAG (SEQ ID NO: 436) |
| rs10491056 | chr10 | CTCTGAATGCAATGCCCTCAGAAAG (SEQ ID NO: 437) |
| | | TTCAGATGTCCTCTTGGCTCGA (SEQ ID NO: 438) |
| rs3817419 | chr10 | CTATGTGGGAGACATACTCATTGCTCTT (SEQ ID NO: 439) |
| | | GGGTGCATTCTGTTACTTCATACAC (SEQ ID NO: 440) |
| rs3740232 | chr10 | GAAACCACCAATGCTGTGGAGAG (SEQ ID NO: 441) |
| | | AGTAAGCCCCAAACAGAAGTCTGTC (SEQ ID NO: 442) |
| rs2435352 | chr10 | TGTGGTCTACCCAGTGTCTGTCT (SEQ ID NO: 443) |
| | | TCGCGCTTTCATTTGTCCTTCG (SEQ ID NO: 444) |
| rs7089209 | chr10 | ATCTCCAGAATGCGTCCTCCTTC (SEQ ID NO: 445) |
| | | ATTAATGATGCTAGCAGAGAAAGAGCA (SEQ ID NO: 446) |
| rs3812658 | chr10 | GTCTGGTACCACTGTGTCATTAGGAA (SEQ ID NO: 447) |
| | | CAACCAGAAGTCATCTCTAGCCCTT (SEQ ID NO: 448) |
| rs2105702 | chr10 | GGCCAACAAGTCCTGTTTACAAGATG (SEQ ID NO: 449) |
| | | ATACAGTATTCACATGGCAGGTACCT (SEQ ID NO: 450) |
| rs3750863 | chr10 | CCTCACAGCTCTGCAAAAGACTA (SEQ ID NO: 451) |
| | | CTTTCTTTGCGAACTTCCTCAAGTGA (SEQ ID NO: 452) |
| rs3758626 | chr10 | TCTTGTAGCTTTTCTGGGGCAAAG (SEQ ID NO: 453) |
| | | GGCTGGATCAGAGCTCTACAAAAGCA (SEQ ID NO: 454) |
| rs10999338 | chr10 | CCCAACCCACAGCACTTCC (SEQ ID NO: 455) |
| | | GTAGGCTGCGGATGATGTCTG (SEQ ID NO: 456) |
| rs780668 | chr10 | TGGTGATAACTGCACTGGTGAAG (SEQ ID NO: 457) |
| | | ACTGTCTTCAGCAGCAGCATCTAC (SEQ ID NO: 458) |
| rs7917781 | chr10 | CTGCTTTCTTCTCCTTGCCCTTTC (SEQ ID NO: 459) |
| | | ACGTGACCCACTCTTTTCCACA (SEQ ID NO: 460) |
| rs4747203 | chr10 | AAGGAAGGATCCATCACCTCCACCA (SEQ ID NO: 461) |
| | | CGAGCATAGTGGGGCACAAAAGG (SEQ ID NO: 462) |
| rs2131956 | chr10 | ATTTCTCATCCTTCCCGCCATCG (SEQ ID NO: 463) |
| | | ACGATTACGAACCTGAGCTGCTGT (SEQ ID NO: 464) |
| rs7074064 | chr10 | AGCAACCATTTTTGTGCCCATGTT (SEQ ID NO: 465) |
| | | TGGTACCGAGTGATCCGTCATACGA (SEQ ID NO: 466) |
| rs1903858 | chr10 | ACCACCTTTTATTACTCCAGCTATAGTG (SEQ ID NO: 467) |
| | | GATTGCTGCATATTTCAGATATTTCTTTCC (SEQ ID NO: 468) |
| rs1409332 | chr10 | CTATTTTGGAGAGGCACCACCTG (SEQ ID NO: 469) |
| | | AAAGGGTGGCCTTGAGTTACAG (SEQ ID NO: 470) |
| rs10786229 | chr10 | ACCAAACAATCCCATCCCCTTCCCC (SEQ ID NO: 471) |
| | | GCCTGGAAGTTGGTTGCATTGAC (SEQ ID NO: 472) |
| rs3740486 | chr10 | GTTGTGGTAGTTTGTGGGGAGAT (SEQ ID NO: 473) |
| | | TCGGGGTCTTTCGGAAGTTTGCAAC (SEQ ID NO: 474) |
| rs813944 | chr10 | CCTTCCACAAAAATGGCCAGTG (SEQ ID NO: 475) |
| | | TACCGGGAAGTCCTGATGTGA (SEQ ID NO: 476) |
| rs196294 | chr10 | GCTACAAACAATTTCTGTGACTTTCAGTC (SEQ ID NO: 477) |
| | | CTGTTTCCCAGCCTGAAAACAAACC (SEQ ID NO: 478) |
| rs2278202 | chr10 | GCATGTCTCACAAGACAACCAAGGA (SEQ ID NO: 479) |
| | | CCAACTGCTTGAACGTTGGTCTC (SEQ ID NO: 480) |
| rs2272599 | chr10 | TTCCCAGACGTGATCTCAGGAG (SEQ ID NO: 481) |
| | | TTCTTGTTGTTCCTGTGGGGGTAG (SEQ ID NO: 482) |
| rs7942159 | chr11 | CAACACCAGCATCCAGTTCAAC (SEQ ID NO: 483) |
| | | ACCAAGGGAGAACACTGATCCT (SEQ ID NO: 484) |
| rs3758680 | chr11 | CATGGAGATTCCAGTGGTAAGCAT (SEQ ID NO: 485) |
| | | AGTAGGATGGAATGGTTTCCCGAA (SEQ ID NO: 486) |
| rs1799937 | chr11 | GACCGGGCAAACTTTTTCTGACA (SEQ ID NO: 487) |
| | | AACTATCAAGGCCCGAGTGAAG (SEQ ID NO: 488) |
| rs11037882 | chr11 | GGAGGTTTGGGATGTTGTTTCTGCT (SEQ ID NO: 489) |
| | | GAGCATCTGTGGTTGTACCAGA (SEQ ID NO: 490) |
| rs3740878 | chr11 | GCAGTGTGGTGTCACAAGCATGA (SEQ ID NO: 491) |
| | | ATTCAAGTGTCCTGAGTGCACAG (SEQ ID NO: 492) |
| rs3729802 | chr11 | ATACACCCCAAGGTGGAGAGAAA (SEQ ID NO: 493) |
| | | GCGCCTAAAGTTCCCTACCTT (SEQ ID NO: 494) |
| rs589691 | chr11 | TTAACCTCTCTGAGCCTCAGCAT (SEQ ID NO: 495) |
| | | TTCATCCTCACCTGTGTGTCCA (SEQ ID NO: 496) |
| rs630394 | chr11 | TGTCTGATGACGGAGGGTGAGG (SEQ ID NO: 497) |
| | | CTTCCTGGACTCACCCACACA (SEQ ID NO: 498) |
| rs736894 | chr11 | TCAGTGCTCAGGGCTTTACAACC (SEQ ID NO: 499) |
| | | AAGAAGTAGCCCTTGACCATGG (SEQ ID NO: 500) |
| rs11604536 | chr11 | ACGACGGTGAGTTCTGGTGA (SEQ ID NO: 501) |
| | | ATGCCCCATCATCAATTTGCTGTG (SEQ ID NO: 502) |
| rs1109977 | chr11 | GAAGGTAGAAGGGCTGAGAGGAGT (SEQ ID NO: 503) |
| | | GCCCTTCCTTCATTGGACATCAGCT (SEQ ID NO: 504) |
| rs883223 | chr11 | ATGGCAGAGCTGGCTTTTCCCTC (SEQ ID NO: 505) |
| | | CCCCCAGGATATCCTCACCACTCA (SEQ ID NO: 506) |
| rs2196168 | chr11 | GGCTATACCTGTAAATGGTGGTCTA (SEQ ID NO: 507) |
| | | AGACCAAAGGCAATGTTACCCTCTTG (SEQ ID NO: 508) |
| rs641936 | chr11 | CAGTTTTTAATAAAGATTCCTTCACAAATCCTAT (SEQ ID NO: 509) |
| | | ATAATGTCTTTTGGATTAGCTACCCGAT (SEQ ID NO: 510) |
| rs1784424 | chr11 | AAAGGATGAGCTGGAGTCACAGAGG (SEQ ID NO: 511) |
| | | AACGGGAATTTGGAGTTCACACAC (SEQ ID NO: 512) |
| rs591058 | chr11 | AGGGAGTGGCCAATTTCATGAGCAG (SEQ ID NO: 513) |
| | | CAGGTCTGCTGTGCATTGTATTGG (SEQ ID NO: 514) |
| rs10895417 | chr11 | CTCTCAGCAAAGTCATCAGAGGAA (SEQ ID NO: 515) |
| | | TGTTATAAGTGACTCCTCCATCACAC (SEQ ID NO: 516) |
| rs693147 | chr11 | AGATGGTTCTGACCCACTTCACCCC (SEQ ID NO: 517) |
| | | AGCAGCACCTGTTGAGAGAAGTGA (SEQ ID NO: 518) |
| rs2276424 | chr11 | TAGAAGCCACCAGTCTCAGGTTCAC (SEQ ID NO: 519) |
| | | CAGGAGGCAGGGTTAGAACTCTTCAAG (SEQ ID NO: 520) |
| rs1954787 | chr11 | AAAGGACCGAAGTAGGGTGGTG (SEQ ID NO: 521) |
| | | GCGGAATTGGGTGAAGGCACGAT (SEQ ID NO: 522) |
| rs2241501 | chr11 | GCTCCTCGTCTTCATGTTTCCA (SEQ ID NO: 523) |
| | | GCAAGCTACCCTCATTCGTGT (SEQ ID NO: 524) |
| rs880054 | chr12 | TTCTCTCCTGCTCTCCTTTCCAT (SEQ ID NO: 525) |
| | | AATCTTGCTTTTGGCAGCCTTG (SEQ ID NO: 526) |
| rs10848683 | chr12 | GTCCAGAGCTAAAGATGACCTGACC (SEQ ID NO: 527) |
| | | TCAGGAAAGCAAACCCCTCTAGGT (SEQ ID NO: 528) |
| rs2267549 | chr12 | TCGCCTTTGAAATACAAGTCATGTCTT (SEQ ID NO: 529) |
| | | AGTAGTGAGAGATGCATTTCCGGAAG (SEQ ID NO: 530) |
| rs216868 | chr12 | GACACTCTTTTGCACTCCTCCT (SEQ ID NO: 531) |
| | | TGCTATAGGGTGCCAGGGAGCTT (SEQ ID NO: 532) |
| rs12426675 | chr12 | CTCACCTCTTTTTCAGGTGTCGA (SEQ ID NO: 533) |
| | | GGGACTTGTGGTGACATGCCTCAG (SEQ ID NO: 534) |
| rs2518144 | chr12 | CAGGTGGCTCTAAAAGCACTCAC (SEQ ID NO: 535) |
| | | TCTTCACTACGTAGCACAGGTAGGT (SEQ ID NO: 536) |
| rs3759236 | chr12 | CAGGAGGCTTTACTTTCTGAGGA (SEQ ID NO: 537) |
| | | CCTTTTCTACATTTTCATTTCAGGTGAC (SEQ ID NO: 538) |
| rs10772008 | chr12 | CCGAAGCCCTCTGTAATTTCTGA (SEQ ID NO: 539) |
| | | TAGGAAATCAGGCCCAATACTCAC (SEQ ID NO: 540) |
| rs7955902 | chr12 | AAGAACAAGCACGTGCAGGTAGGAC (SEQ ID NO: 541) |
| | | GCATGCTGGGCACTAAATAATCTCCT (SEQ ID NO: 542) |
| rs2276456 | chr12 | ACCCTGAGGGAAGAGAAAACCAGCC (SEQ ID NO: 543) |
| | | CTGCCCAAGAGGAATTTGCTGTGGT (SEQ ID NO: 544) |
| rs1034762 | chr12 | CTTGAAATCCTTGAGGCCCCTAA (SEQ ID NO: 545) |
| | | CAGATACTTACAGGAGCACCTGCA (SEQ ID NO: 546) |
| rs3803184 | chr12 | TGAGGGACGCATGGAAAGCAG (SEQ ID NO: 547) |
| | | CCCGAGGCGAATCATGGCTCA (SEQ ID NO: 548) |
| rs4075913 | chr12 | AAGGGGCTTGGTGTTAAGGTAC (SEQ ID NO: 549) |
| | | AGAATGGCCATTGTTGGGACA (SEQ ID NO: 550) |
| rs2071218 | chr12 | CCGAGTTCGTCAACCACTACTGCT (SEQ ID NO: 551) |
| | | CTTCCCTCCTTTCCTCTCATGC (SEQ ID NO: 552) |
| rs706815 | chr12 | GGACATGAAGAAGGTGGTGTGTG (SEQ ID NO: 553) |
| | | GGGATTTCTGGGCCCAGGAACTTG (SEQ ID NO: 554) |
| rs2270267 | chr12 | CTGCACTGGGAAGACTTTTCCA (SEQ ID NO: 555) |
| | | CATTCTCCACCTCGGCTGTCA (SEQ ID NO: 556) |
| rs7397862 | chr12 | CTTTGGGTGTGGAAGGTGTCAT (SEQ ID NO: 557) |
| | | ATATCTGAAATGACGAGGGGGTCT (SEQ ID NO: 558) |
| rs2302629 | chr12 | AGGTGGAACGTGAACATATGTGT (SEQ ID NO: 559) |
| | | GAGACATTATGCAATTAAATGCAGGGAT (SEQ ID NO: 560) |
| rs6606734 | chr12 | TGGTGATTATGGGGAAGTGATTCC (SEQ ID NO: 561) |
| | | GAGCATGCCAAGGTTGATTAGCAGTG (SEQ ID NO: 562) |
| rs2464195 | chr12 | ACCTGTGCAGAGCCATGTGAC (SEQ ID NO: 563) |
| | | TGGCTGTCAATGGATGCAGGGGAA (SEQ ID NO: 564) |
| rs3741591 | chr12 | GATGGCCATGGCACTTACCA (SEQ ID NO: 565) |
| | | TGGCCTACCTGGGGGATG (SEQ ID NO: 566) |
| rs2075784 | chr12 | TCAGGCGCGGCGAGTGC (SEQ ID NO: 567) |
| | | CACCTGCTGGCCTCGCCATC (SEQ ID NO: 568) |
| rs9564692 | chr13 | AAGGTGCATGGACCATACTTCCGTT (SEQ ID NO: 569) |
| | | GAGGGTGTGGTTTTCCTCAGTCAGT (SEQ ID NO: 570) |
| rs3752451 | chr13 | GTCTTTGATAAGTGTGTTAGTAACTGACAAT (SEQ ID NO: 571) |
| | | TTTAGTACGATGTGATTAACGTAGAATAGCT (SEQ ID NO: 572) |
| rs9534342 | chr13 | TGACTGTTATGTGCTAGACAGTGTT (SEQ ID NO: 573) |
| | | CCTCATAGAGCTTACATTATGAGGTTGG (SEQ ID NO: 574) |
| rs2296533 | chr13 | GCCAGCAGAGACTACACCAA (SEQ ID NO: 575) |
| | | CCTCCCGCTCCATGTTCGT (SEQ ID NO: 576) |
| rs6313 | chr13 | CGATTTTCAGAGTCGACTGTCCAG (SEQ ID NO: 577) |
| | | TAGGGAGTTCGTAGTTGAGCTCA (SEQ ID NO: 578) |
| rs9518061 | chr13 | GCCTGTGCATTTTATATCAAAGGGAAATT (SEQ ID NO: 579) |
| | | TGGGAGCTCTCAGTAAAATTGCA (SEQ ID NO: 580) |
| rs661585 | chr13 | GGATTCCAGGACCCTAAAGAACTCTGC (SEQ ID NO: 581) |
| | | GTCCTCAATTGCTGAGCGTTTGG (SEQ ID NO: 582) |
| rs589985 | chr13 | ACCCTTAGAGCCTGTGATTCCTG (SEQ ID NO: 583) |
| | | CCCCTCAATGTTCTGGAAAGCA (SEQ ID NO: 584) |
| rs482757 | chr13 | CCTGAAACCGAAGAGAGAAGCAGT (SEQ ID NO: 585) |
| | | ACACTTACGGGACTCCCTTTTTCC (SEQ ID NO: 586) |
| rs3803236 | chr13 | TGGGAAAGGAAACAGGGAAGTCGA (SEQ ID NO: 587) |
| | | GAATACCTGGAACGAAGGGTACTC (SEQ ID NO: 588) |
| rs2281974 | chr13 | CTCCAGGACTGCATGGATTCCCAG (SEQ ID NO: 589) |
| | | TAAACGAGTGGGGTCCTCACT (SEQ ID NO: 590) |
| rs178642 | chr14 | CCAGCTCATGCACATTCTTTCCTCC (SEQ ID NO: 591) |
| | | CACCAACTCATCTCTGGCCTCT (SEQ ID NO: 592) |
| rs2073349 | chr14 | TTCTCCAGCATCTCTCTCTCCT (SEQ ID NO: 593) |
| | | ATTCCCACTGGGATTTCTCCTCA (SEQ ID NO: 594) |
| rs1152590 | chr14 | CTCTCACGAAGAGGACGAGGA (SEQ ID NO: 595) |
| | | CTTAGCAACACGGCCAGCT (SEQ ID NO: 596) |
| rs229581 | chr14 | CCAAACCATGCCTTTGCTGAAG (SEQ ID NO: 597) |
| | | CGCAGGTCCTTGTAGAGATCGGTGA (SEQ ID NO: 598) |
| rs3759753 | chr14 | CAAAATGTCTTTTGGTCAGCAATAGCTTA (SEQ ID NO: 599) |
| | | GAAGACCGCAGTGGGATAAATCTC (SEQ ID NO: 600) |
| rs2075179 | chr14 | CTCTCTGTTGGTTGTAGGAAGCTGTA (SEQ ID NO: 601) |
| | | TGTGTAAGTCAAGGGTAGCCATG (SEQ ID NO: 602) |
| rs12432149 | chr14 | GTCAGGGGTCTCTATGTATACATCACAC (SEQ ID NO: 603) |
| | | TGTCATTTCACAGCACATGCTTCCA (SEQ ID NO: 604) |
| rs3742656 | chr14 | CCACCTCCACACATTTCTGTCTTT (SEQ ID NO: 605) |
| | | CTCACAGTGGTTGTCCAGCTT (SEQ ID NO: 606) |
| rs3751582 | chr15 | GTACAAGAAGGGTGTGTGGCTT (SEQ ID NO: 607) |
| | | CCGGTTGCTTTCGCTCTTTGAACG (SEQ ID NO: 608) |
| rs1800419 | chr15 | AGTCAGCAGCCCCTTACCTCC (SEQ ID NO: 609) |
| | | TTGATGCCACGTCCCATGG (SEQ ID NO: 610) |
| rs1800404 | chr15 | TCTTTGAGCTGACATCCCACTGAG (SEQ ID NO: 611) |
| | | GCACGATCTGGAAAGAAGCACAGGA (SEQ ID NO: 612) |
| rs173224 | chr15 | GCTTGCAGTTCGTTCATCCATC (SEQ ID NO: 613) |
| | | AACAACGTAGTCCACCAGATTAATCC (SEQ ID NO: 614) |
| rs422388 | chr15 | TCGGTGTCGAGAGTATTTGTCATCTGG (SEQ ID NO: 615) |
| | | GTATGTCAGTACAGGAGTGATGAAATGG (SEQ ID NO: 616) |
| rs2279580 | chr15 | GTCTTTCAGTCCTCTTGATCATACCACC (SEQ ID NO: 617) |
| | | CTCTCTCCAGATACTGCATAGAACGT (SEQ ID NO: 618) |
| rs1801376 | chr15 | GTGCTTCCCAGTTTCACTCCAT (SEQ ID NO: 619) |
| | | GGCACCTATTCTACTTCCCAAAGG (SEQ ID NO: 620) |
| rs140605 | chr15 | GGCAAATATCAGGATCTAGTGCACATT (SEQ ID NO: 621) |
| | | AAATGTAATGACCTTAGGAGCTACAGGA (SEQ ID NO: 622) |
| rs2306187 | chr15 | AGAGGGGACATTAAGTCAGACGAT (SEQ ID NO: 623) |
| | | ACCAGCTTCTGCTTCTGTTGC (SEQ ID NO: 624) |
| rs58156069 | chr15 | GGCTGAGAGTCAAACCATCCTTTTC (SEQ ID NO: 625) |
| | | ATCCCAATCAGATGTCACGTCTGT (SEQ ID NO: 626) |
| rs1147129 | chr15 | TCCAGCAGGAACTGAGTAGTCT (SEQ ID NO: 627) |
| | | GGCATCTAACTGATGATCGGTTGC (SEQ ID NO: 628) |
| rs762551 | chr15 | GCGTTCATGTTGGGAATCTTGAGG (SEQ ID NO: 629) |
| | | GGAGCTTAGTCTTTCTGGTATCCAGCT (SEQ ID NO: 630) |
| rs1370275 | chr15 | TTCAGTCCGCTCTGCATACAGGGC (SEQ ID NO: 631) |
| | | CTAGCCAAGACCGAGGATAGGT (SEQ ID NO: 632) |
| rs9806604 | chr15 | TTTAGGCAGCCTGTCATCCTCTC (SEQ ID NO: 633) |
| | | TGCGTATCATTCATGTGCATCGATG (SEQ ID NO: 634) |
| rs2307438 | chr15 | GGCAAACATGCACCTGAAAGAG (SEQ ID NO: 635) |
| | | AACACCCCATCTGTTCACTTAGC (SEQ ID NO: 636) |
| rs9672296 | chr15 | TCTTCTCGGACAGCTCCTTCTCC (SEQ ID NO: 637) |
| | | GGACAGAAAGGGCCGGATGTTAAGG (SEQ ID NO: 638) |
| rs3815003 | chr15 | TTTCCTTCTGTTCCGGTGATGG (SEQ ID NO: 639) |
| | | TATTTGAGAACCCTGGGGCAGT (SEQ ID NO: 640) |
| rs2272037 | chr15 | GCCTCTGGGGAAGATTTTTGAGG (SEQ ID NO: 641) |
| | | CACAGAGTATGATGGGCAGGATG (SEQ ID NO: 642) |
| rs7496614 | chr15 | GTCTTCCTCACTTGAGGAGATGAGG (SEQ ID NO: 643) |
| | | TTCATGACGGTCAGGATGAACC (SEQ ID NO: 644) |
| rs224204 | chr16 | AGGAGGAGTCTGGAATCACAGA (SEQ ID NO: 645) |
| | | CTGTACCTGTGCAAGATGTCTCCA (SEQ ID NO: 646) |
| rs1641010 | chr16 | AGCTATTTTGGCAAGAGTGTGGACTT (SEQ ID NO: 647) |
| | | AATTCAGGTAAGTGAGGAGGAGGT (SEQ ID NO: 648) |
| rs2280764 | chr16 | TCTTGTCCAGGGCTTTGTTCAC (SEQ ID NO: 649) |
| | | CCGGTTATTCTCAATGGGCTCCA (SEQ ID NO: 650) |
| rs3115446 | chr16 | GCTCTTCCCTTCACATACCAGT (SEQ ID NO: 651) |
| | | CCATGGTCAATCCACTGCCTCAG (SEQ ID NO: 652) |
| rs215901 | chr16 | CTGATAGGGCCTCACAGTGTTGGG (SEQ ID NO: 653) |
| | | AAGGCCTGAGGAGCTTTTGAGGTAG (SEQ ID NO: 654) |
| rs2072061 | chr16 | AAAGGCCTCCTCCACAAGGACC (SEQ ID NO: 655) |
| | | CCAACCCACCCAAACCGTCTCT (SEQ ID NO: 656) |
| rs249954 | chr16 | CCAATCCAAATCTGTTTTTCTGAATCTGTTT (SEQ ID NO: 657) |
| | | ACACTGGAAGAGAATATTCTTCTGACCT (SEQ ID NO: 658) |
| rs7203525 | chr16 | CTATCTTCAGGAGGAGGAGAAGTCTCCT (SEQ ID NO: 659) |
| | | AAATTGGCAGCAAATGTAACAGTTACTC (SEQ ID NO: 660) |
| rs243834 | chr16 | TCCTTTAGAGAGGCCCTGCTG (SEQ ID NO: 661) |
| | | AAGCTCATCGCAGATGCCTGGAA (SEQ ID NO: 662) |
| rs2304483 | chr16 | TGGCCTAGAAAGAGGCTCGAC (SEQ ID NO: 663) |
| | | TGGGGCTATCATCTCCGTGGTCATC (SEQ ID NO: 664) |
| rs1532625 | chr16 | TGGCCGATTTTGTCCAGACAAG (SEQ ID NO: 665) |
| | | GGGACTGTCACTCTTCCTGTCTAGGT (SEQ ID NO: 666) |
| rs11076207 | chr16 | TAAATGTGTGGCCCACTCTTGG (SEQ ID NO: 667) |
| | | TACCTGTTCCACCAACTCTTGCT (SEQ ID NO: 668) |
| rs483053 | chr16 | CCCAGCAGTCCTACCTTTAAACAGC (SEQ ID NO: 669) |
| | | CCATGGGGGTAAAACGGATGACT (SEQ ID NO: 670) |
| rs3991715 | chr16 | CACGAAGAACAGCCATAGGACA (SEQ ID NO: 671) |
| | | GCTCTTCACCAGTTGGATCAGGTAG (SEQ ID NO: 672) |
| rs2548861 | chr16 | CGGTTTGCTTTAGAGTGCATTAACTTCTTT (SEQ ID NO: 673) |
| | | AATAGCTTCTGTGTGTTTCCTATTTTTGG (SEQ ID NO: 674) |
| rs12918876 | chr16 | TTCCAGTGCCTGGAGGAGACCCC (SEQ ID NO: 675) |
| | | ACACACCACTTCTCCCTCAGCA (SEQ ID NO: 676) |
| rs3803679 | chr16 | GCGACGTGGTGGAAGTCTACCTG (SEQ ID NO: 677) |
| | | CTGTGGGCTCCTGTGAATGAG (SEQ ID NO: 678) |
| rs3752827 | chr17 | CAGTACATCCAGAATGTCACAACAGATTA (SEQ ID NO: 679) |
| | | TATCTCTGGTTTTGAGGTAGAGAGCT (SEQ ID NO: 680) |
| rs238238 | chr17 | GCCCAGTTGATTGAGCTCCAAA (SEQ ID NO: 681) |
| | | CCTGCTCTGCTGCAAAAGGCAAG (SEQ ID NO: 682) |
| rs12453262 | chr17 | CTCCGGGATAGGATGGGGTAGACC (SEQ ID NO: 683) |
| | | CCTGCTCCCTCACTATGCCACT (SEQ ID NO: 684) |
| rs17671352 | chr17 | AGTCTCAGCGGACTTGTCCA (SEQ ID NO: 685) |
| | | TCTTAAGGCAGTACGGGCTCT (SEQ ID NO: 686) |
| rs1565817 | chr17 | CTTTGTTCCCAGCCTGACACTGC (SEQ ID NO: 687) |
| | | ACACAGAAGCCTTCATGGGCATC (SEQ ID NO: 688) |
| rs2304908 | chr17 | GTTGGGGGAGAGGGAAAGTTCTT (SEQ ID NO: 689) |
| | | ACGAAGAAGGAGGCCGTCTTGAG (SEQ ID NO: 690) |
| rs876660 | chr17 | GAATTGTCCCTCAGGGATTGCACT (SEQ ID NO: 691) |
| | | ACATGTAGTTCAGGCCTCTCTTCCT (SEQ ID NO: 692) |
| rs3744586 | chr17 | CTCCAGTCACAACTCCTGATAAGTCC (SEQ ID NO: 693) |
| | | TGCAGACAAACTGATTCAGGTCGT (SEQ ID NO: 694) |
| rs1736219 | chr17 | GCTGCTCATCTCCGAAGAAGATGGG (SEQ ID NO: 695) |
| | | AGACTTTTCAGAGTCAGCTGGCACA (SEQ ID NO: 696) |
| rs548973 | chr17 | GCCTCTCTTCCCAAAACCCATCT (SEQ ID NO: 697) |
| | | GGGAACACTGAGGCATGGAATCCT (SEQ ID NO: 698) |
| rs2952976 | chr17 | TGGAAAAATGTCTTGCTGGGGTA (SEQ ID NO: 699) |
| | | AAGTACAGATGTGGACCAAGAGGA (SEQ ID NO: 700) |
| rs9894648 | chr17 | AGAGGACCTGAAGGTATTCCACAATG (SEQ ID NO: 701) |
| | | TAACTAGACTATATCCTGGCCTCCCTAG (SEQ ID NO: 702) |
| rs3110641 | chr17 | GTGGAAAACAGGGTCCTTGTTG (SEQ ID NO: 703) |
| | | GTCATGGGCCATTAGTTTGGCTTC (SEQ ID NO: 704) |
| rs8067890 | chr17 | GATGTTCTCCACCGACGAGTACAG (SEQ ID NO: 705) |
| | | ACCTCCACCCTGTAGCAATTCCAT (SEQ ID NO: 706) |
| rs8176161 | chr17 | AGAGCCTGGTTCTATGATGAAGATG (SEQ ID NO: 707) |
| | | TTACATTATCAAATGGTCATATATATGGTAAAGCTT (SEQ ID NO: 708) |
| rs850730 | chr17 | CTCACCAACATCGCGATTCCTATC (SEQ ID NO: 709) |
| | | AGTCTTCACCCTCCAAGATCACCA (SEQ ID NO: 710) |
| rs2289677 | chr17 | AGGGCTAAAACATGAGCAGGTACA (SEQ ID NO: 711) |
| | | AAAGGGTCACATTTCTGATGAGCTCT (SEQ ID NO: 712) |
| rs2258689 | chr17 | TTAGCCCCAAACACCCCACTC (SEQ ID NO: 713) |
| | | CTGTCACTTCCCGAACTGGCAGT (SEQ ID NO: 714) |
| rs3809863 | chr17 | CCAGAGCAAAATGGGACACAGTA (SEQ ID NO: 715) |
| | | GTCATGTTCAGCCAGTACCATCGT (SEQ ID NO: 716) |
| rs2256835 | chr17 | AATTCGAGGGCAGGAGATTACC (SEQ ID NO: 717) |
| | | GCCAAGGTTTGCTAATGCTGCT (SEQ ID NO: 718) |
| rs3744093 | chr17 | CAACACCAGCAAACACACCTTCCAA (SEQ ID NO: 719) |
| | | CAGTCCTGTGCGTCCAAAGC (SEQ ID NO: 720) |
| rs758596 | chr17 | CCCTGGAGTAATCACCTGCTCT (SEQ ID NO: 721) |
| | | GCTGTATGTCCACCCGGATTCTCCT (SEQ ID NO: 722) |
| rs4342 | chr17 | AGACCTGCTGCCTATACAGTCAC (SEQ ID NO: 723) |
| | | CAAACCCCTACCAGATCTGACGA (SEQ ID NO: 724) |
| rs4362 | chr17 | TGGCACAAGGCCCTCAACC (SEQ ID NO: 725) |
| | | AGTTCCACGAGGCACTGTG (SEQ ID NO: 726) |
| rs3744024 | chr17 | AGGACTCCATCCCTGGGGATTG (SEQ ID NO: 727) |
| | | CCCACCTCTCTGAGGAGCTCATGC (SEQ ID NO: 728) |
| rs11077379 | chr17 | CTCCCTCATCTCTATGAGGTCTGTGTT (SEQ ID NO: 729) |
| | | GGCCTTGAGAAGTGGTTCTCTGT (SEQ ID NO: 730) |
| rs3816256 | chr17 | ATGGTCCCACATCCATGTGTGG (SEQ ID NO: 731) |
| | | AGATCCAGCTAACAGGCGCTA (SEQ ID NO: 732) |
| rs3213926 | chr18 | TCCAGTCTTTGCTCGAGACTTG (SEQ ID NO: 733) |
| | | TTTACTTTTGTACAGATGTTAAATTTGTGACAC (SEQ ID NO: 734) |
| rs7232329 | chr18 | CCATCCCAAGGTCTTTGAAGGT (SEQ ID NO: 735) |
| | | TTCATCTGGACACCTCCTCTCA (SEQ ID NO: 736) |
| rs3748422 | chr18 | CCATAAGTGACTGTGACGTTGCA (SEQ ID NO: 737) |
| | | AAGCAGACAACCCGAAGTAAACAC (SEQ ID NO: 738) |
| rs8097342 | chr18 | AGGGACAGAACACAGTGAACC (SEQ ID NO: 739) |
| | | CAACAGGCACCCGATTTTCTC (SEQ ID NO: 740) |
| rs2510344 | chr18 | GCCTCAGGATAGAATTCCCTTTCAGT (SEQ ID NO: 741) |
| | | GACCATGGCCAAATACATCCTGA (SEQ ID NO: 742) |
| rs12960081 | chr18 | GAAGAAATGGCAGCATCTGAACCCGT (SEQ ID NO: 743) |
| | | CCCAATGTTGTAGTAACCGAAGC (SEQ ID NO: 744) |
| rs1058396 | chr18 | CATCCTGCCTTTAGTCCTGAGTTCT (SEQ ID NO: 745) |
| | | GCAATGGGAGGAATGTTCATGG (SEQ ID NO: 746) |
| rs1450425 | chr18 | TCAGCTAGGAGAGGGGAATAAGGCAC (SEQ ID NO: 747) |
| | | CAAGAAGAGCTGGCACCTAGATC (SEQ ID NO: 748) |
| rs1788027 | chr18 | GCATGGAAAGACAGAGGACGAG (SEQ ID NO: 749) |
| | | TTCATGTGGATGCAGGCTACAG (SEQ ID NO: 750) |
| rs2298712 | chr18 | GCAGTAGAGTATGAGTTCTTCCTTTCCTA (SEQ ID NO: 751) |
| | | TCCCTCTTTACTATGTGCAGGTGCTCT (SEQ ID NO: 752) |
| rs1893788 | chr18 | TTCTGTTGGTGATCACCCTCCT (SEQ ID NO: 753) |
| | | CACCAGTGATATACTTGCCCAGGTCAG (SEQ ID NO: 754) |
| rs6567271 | chr18 | GGCAATCTGGGAGAGTTTTGCA (SEQ ID NO: 755) |
| | | GGACAGAGAAATCCGATGCGGTT (SEQ ID NO: 756) |
| rs1629038 | chr19 | CCTGGAGGACGCGTGAGTGC (SEQ ID NO: 757) |
| | | CGACGGGAAGGTGCAGGTTC (SEQ ID NO: 758) |
| rs3764640 | chr19 | AGGGCCGTCAAGATCCTCA (SEQ ID NO: 759) |
| | | CACGGTGCTGATGGTTCTGTC (SEQ ID NO: 760) |
| rs2277984 | chr19 | ATGGTAGAACCGGGTACAGCTTTCC (SEQ ID NO: 761) |
| | | AGATGCTAGGTCTTCAACCCCTCA (SEQ ID NO: 762) |
| rs2860178 | chr19 | CACCAGTCCATGGAAAAACCATCTTC (SEQ ID NO: 763) |
| | | CATTGGTCTTCAGGGCAATGTC (SEQ ID NO: 764) |
| rs620744 | chr19 | TATGAGCGTGGCCGGATCTCC (SEQ ID NO: 765) |
| | | GAGCATCAGGAGGTCACAAGC (SEQ ID NO: 766) |
| rs539887 | chr19 | TGTGGCCAGGTGGTGGTGG (SEQ ID NO: 767) |
| | | CCTTAAGCAGCTAGTCCTGCTCCA (SEQ ID NO: 768) |
| rs8104339 | chr19 | TGGTGGGACCAGAGAACCAG (SEQ ID NO: 769) |
| | | TCAAGAAGGATGGGGAGTGGAAG (SEQ ID NO: 770) |
| rs2114724 | chr19 | GACAGAGGATGTGGGCCATGCT (SEQ ID NO: 771) |
| | | GAAGATGGGCTGCTCATCACTG (SEQ ID NO: 772) |
| rs280520 | chr19 | GGAACTTTCGGAGCCGCAAG (SEQ ID NO: 773) |
| | | ACCAGGCAGGGAACGTGTCCA (SEQ ID NO: 774) |
| rs3826803 | chr19 | AAAGCATTTCTCCCCGCAGTC (SEQ ID NO: 775) |
| | | TGCCGAGTTTTTGCACTGCAAGTC (SEQ ID NO: 776) |
| rs2075021 | chr19 | CATGTTCCAGCACATCGAGGTGAG (SEQ ID NO: 777) |
| | | CTCGACAGCTCTTTAAAAACAGACTC (SEQ ID NO: 778) |
| rs2738447 | chr19 | CTCTCCAGGTGCTTTTCTGCTAGG (SEQ ID NO: 779) |
| | | ACCTCTCCTTATCCACTTGTGTGTC (SEQ ID NO: 780) |
| rs6413504 | chr19 | TGAGAAGTAGGTGGCCTCCA (SEQ ID NO: 781) |
| | | ACAGATGGTCAGTCTGGAGGATGAC (SEQ ID NO: 782) |
| rs2304154 | chr19 | TCTGAGGTTTGCTTCCAGTGTGT (SEQ ID NO: 783) |
| | | AAAACACAGGGCACACGTGA (SEQ ID NO: 784) |
| rs8113582 | chr19 | GGACAATGGGCAGATACACAGTAG (SEQ ID NO: 785) |
| | | CTTCTGTGGACAATGACCCAGTC (SEQ ID NO: 786) |
| rs1043996 | chr19 | ACTCACTGGGGTCACAGTCAT (SEQ ID NO: 787) |
| | | CTGCCAGGTTGGTGCAGATACCAT (SEQ ID NO: 788) |
| rs2302603 | chr19 | TATGATGCTGGGACCAGGTGA (SEQ ID NO: 789) |
| | | CGAGGGGCTGCAGCTTTTGTC (SEQ ID NO: 790) |
| rs10424582 | chr19 | TGACGTAGTTCACCAGCATGG (SEQ ID NO: 791) |
| | | CGGCTTCGTTAAAGAGCCTCT (SEQ ID NO: 792) |
| rs55742440 | chr19 | ACAGGACAGGCTGGCTCT (SEQ ID NO: 793) |
| | | AGCATGCCTGTCCCCCACAGA (SEQ ID NO: 794) |
| rs1469699 | chr19 | ATCCTCAACGAGTTCGCCAAGA (SEQ ID NO: 795) |
| | | GCCCAGCCCAATAAACCTGCACT (SEQ ID NO: 796) |
| rs3745859 | chr19 | CAGTGCTCACTGTCTTTGTGTG (SEQ ID NO: 797) |
| | | AGTCCTGGTCATCCTCTCCTT (SEQ ID NO: 798) |
| rs10880 | chr19 | TCCCTATCTATGCCAGCCTCAGT (SEQ ID NO: 799) |
| | | GGCACCTGCTAGCCCCGTTCTG (SEQ ID NO: 800) |
| rs2246095 | chr19 | TCAGTGTGATCTCCCTTGTGGTGCC (SEQ ID NO: 801) |
| | | TTCCGCAGGTCCTGGCTT (SEQ ID NO: 802) |
| rs1799783 | chr19 | CCCTCCTGCTTCTCATAGAAGTTG (SEQ ID NO: 803) |
| | | CAGTCTCTTCCCCAGCTTCCCAA (SEQ ID NO: 804) |
| rs392891 | chr19 | CCCAATAAACCCCGCACTGTGCC (SEQ ID NO: 805) |
| | | CGGCTGATGATGTCCGGGTACTTC (SEQ ID NO: 806) |
| rs20580 | chr19 | ACCAGAAACTCACTGGAGGTCTTTA (SEQ ID NO: 807) |
| | | CTGACGATAGACAGAACGGTCAGATG (SEQ ID NO: 808) |
| rs3730849 | chr19 | GAGGACTTGGAGAAGGAAAGACTCAG (SEQ ID NO: 809) |
| | | CAGCTGCTCCTGGAACAGAAATCCA (SEQ ID NO: 810) |
| rs2387588 | chr19 | CCACACCCCATTCCCCAAC (SEQ ID NO: 811) |
| | | CAGTAGCCGGCACAGATGGT (SEQ ID NO: 812) |
| rs4002462 | chr19 | CAGGATGGCATTGATGGGGT (SEQ ID NO: 813) |
| | | ACCGCAGCCCCGAGTCCTG (SEQ ID NO: 814) |
| rs1062798 | chr19 | GCTGCTGTATTGCTGGGGATC (SEQ ID NO: 815) |
| | | GGTGGATGTTGTTGTGGAGGT (SEQ ID NO: 816) |
| rs12981413 | chr19 | GTTCCTGCTGTGTGTCACTCTGA (SEQ ID NO: 817) |
| | | GATCCTGACACCCAATGCCATC (SEQ ID NO: 818) |
| rs3745405 | chr19 | TGACGAAACAGAAGACCCGAAC (SEQ ID NO: 819) |
| | | GGAAGGCAATGACAGCTGACAGAG (SEQ ID NO: 820) |
| rs269950 | chr19 | GCCTACCGTAGGTGTTTTAGGTTA (SEQ ID NO: 821) |
| | | CGCCTCTGAGAGATATCTACAGCCAAGC (SEQ ID NO: 822) |
| rs3729836 | chr19 | GCATAAGCGCGGTAGTTGGAGGA (SEQ ID NO: 823) |
| | | CCTTACCGTACCGCACCCTCT (SEQ ID NO: 824) |
| rs2076653 | chr20 | CAGAATGGCAAGAGGACTGACAGTC (SEQ ID NO: 825) |
| | | GTTTGACATCACCCCCTCCAA (SEQ ID NO: 826) |
| rs2073193 | chr20 | TCACCTTGAACACCTCCTTGACG (SEQ ID NO: 827) |
| | | GGGGGAAGAGGGGAGAAGTAAAGATAGAG (SEQ ID NO: 828) |
| rs2281575 | chr20 | GCGTCTCCTTCACGTTCACA (SEQ ID NO: 829) |
| | | CCCTATGTCCCATGTGGCCAGAG (SEQ ID NO: 830) |
| rs3803953 | chr20 | GTGGTGATGTATTTGCCCACAG (SEQ ID NO: 831) |
| | | ACGTACCATCAGTGAAGTCCAAG (SEQ ID NO: 832) |
| rs2295435 | chr20 | GCAATGCTTCTGTTCCCACCA (SEQ ID NO: 833) |
| | | AAATGAGAGAGGGCCCAGCTT (SEQ ID NO: 834) |
| rs2295179 | chr20 | TGCAGAAGAGGAACCTGTCATCAC (SEQ ID NO: 835) |
| | | CCTCCTAAGGTAGAAAGTCCCAGTG (SEQ ID NO: 836) |
| rs363050 | chr20 | TCTATACAGTGCTGGGAGGACTCT (SEQ ID NO: 837) |
| | | ATTCAGCAAGCCTGAGGAGTG (SEQ ID NO: 838) |
| rs2273060 | chr20 | ATCATGCCCGAGTGAGAAGCCTT (SEQ ID NO: 839) |
| | | CTTCTTCCCTGACCATTTTGGCTT (SEQ ID NO: 840) |
| rs2273061 | chr20 | CTACCCAGAGACAACCTGTTACC (SEQ ID NO: 841) |
| | | ATGTGTGCATGTGAGTCGTTTGG (SEQ ID NO: 842) |
| rs143383 | chr20 | CGGGTGTGTGTTTGTATCCAGTC (SEQ ID NO: 843) |
| | | CCTTTCTCCTGCACAACTGACT (SEQ ID NO: 844) |
| rs2072792 | chr20 | ACCCAGAGCTACCAATGACTTC (SEQ ID NO: 845) |
| | | GCACTCTAGGAAAAGTCCCACA (SEQ ID NO: 846) |
| rs4608591 | chr20 | AGCTGCCACAATGGGACATGTGC (SEQ ID NO: 847) |
| | | ATCGGCAGGTTTCTCAGGGAT (SEQ ID NO: 848) |
| rs2425906 | chr20 | GAAACAAAAGGCCAGTCGTTGGCA (SEQ ID NO: 849) |
| | | CCCAAGCACACAGCTTCAGGAT (SEQ ID NO: 850) |
| rs6021437 | chr20 | CCGAATATGTTGCTGCAGCATCA (SEQ ID NO: 851) |
| | | CCCCAAGGTGTGTCTTCAGGTT (SEQ ID NO: 852) |
| rs2234487 | chr20 | GTTTGCTCCAACTGACACTTCAG (SEQ ID NO: 853) |
| | | AGATATGGAATTGTGGGTGCATGC (SEQ ID NO: 854) |
| rs3746657 | chr20 | CCTGACATCCGCGGCATGG (SEQ ID NO: 855) |
| | | TAGGGTGGGAGAGCACAAAGC (SEQ ID NO: 856) |
| rs6062700 | chr20 | CTCACAGGGAGAGGCTGGT (SEQ ID NO: 857) |
| | | CTGGGTGGGTTGGTCACTG (SEQ ID NO: 858) |
| rs41309367 | chr20 | TTGCCCAGGGCTGAACATGG (SEQ ID NO: 859) |
| | | GAGGAGGTGGGTGACACCT (SEQ ID NO: 860) |
| rs2073421 | chr21 | TTCCTGCGGTGTCTGAAACATCA (SEQ ID NO: 861) |
| | | AGGAGGGACAGGGTTTGCAT (SEQ ID NO: 862) |
| rs2073425 | chr21 | GAGCTGGGAGGTAGTTCTAAGTGCAC (SEQ ID NO: 863) |
| | | AAATTTAGAGACAGGATCTCATTATGTTGC (SEQ ID NO: 864) |
| rs8130564 | chr21 | AGTCACCTTGTCATCTGGCAAGAGG (SEQ ID NO: 865) |
| | | CAGACCCGACGTGGTAACTTTACAC (SEQ ID NO: 866) |
| rs872331 | chr21 | TCCCCGTGGTACCAAAGCTGAA (SEQ ID NO: 867) |
| | | GTATGACCTGCTGCCCTTCCT (SEQ ID NO: 868) |
| rs754507 | chr21 | CAAAGTCTTCTCGGTGGCCATCA (SEQ ID NO: 869) |
| | | TGGGACCGTCTTTTGGTCCT (SEQ ID NO: 870) |
| rs62215498 | chr21 | CATGTGCCTGGGACATGTGTCC (SEQ ID NO: 871) |
| | | TTCCCACTGACTCGTCTCCAT (SEQ ID NO: 872) |
| rs2839077 | chr21 | TTGGTCTGAGACCCTCAGCTTA (SEQ ID NO: 873) |
| | | TGGCCCTATCGGACCTAAAGG (SEQ ID NO: 874) |
| rs7283989 | chr21 | TAGCTCCTGAGAGAATGGATCCCGGG (SEQ ID NO: 875) |
| | | CTCCAAGCCAGCAGGGTTC (SEQ ID NO: 876) |
| rs2236486 | chr21 | GCTGGAGGATGCCTTCCTGAAGA (SEQ ID NO: 877) |
| | | GCGTAGGTGCACGCGGGGC (SEQ ID NO: 878) |
| rs7279347 | chr21 | GTAAGTGGCCTTGTCAGGGTACGG (SEQ ID NO: 879) |
| | | AGAGGGCCTTTCTGGAAACACTG (SEQ ID NO: 880) |
| rs7279622 | chr21 | AGGCAGGGTGCGAATGGA (SEQ ID NO: 881) |
| | | CAGAGCTGTCCGTGGCACAGT (SEQ ID NO: 882) |
| rs9982233 | chr21 | TTGCCATTACTGACCTGGAGAG (SEQ ID NO: 883) |
| | | CTGTTATCCCCACAGGGCACAGT (SEQ ID NO: 884) |
| rs2839226 | chr21 | GGCATTTCCTGTGGGAAACTGACT (SEQ ID NO: 885) |
| | | ATCACCGAGAAATTCAGTGCGGAA (SEQ ID NO: 886) |
| rs2839257 | chr21 | TCTGGCCCACGTGGTCAGA (SEQ ID NO: 887) |
| | | GCAGAGCCAGCTGGAGGAGGA (SEQ ID NO: 888) |
| rs112544 | chr22 | AGGATGGTGGGGGTCTCTGG (SEQ ID NO: 889) |
| | | CTGACCTATGAGGAGCGGGTT (SEQ ID NO: 890) |
| rs2072708 | chr22 | GCTAACAAGCCCCTCTTCTCTC (SEQ ID NO: 891) |
| | | CTCTCACACTGTGGGCATCTG (SEQ ID NO: 892) |
| rs2235324 | chr22 | CACTCCAGCCGGAGTTTGAGCA (SEQ ID NO: 893) |
| | | CGTAGCTGTAGCGGTAACAACC (SEQ ID NO: 894) |
| rs5756516 | chr22 | AGAGGAGGGAAGTGCATCTCA (SEQ ID NO: 895) |
| | | CCCACTGGCTTCCCTATGGTCAGA (SEQ ID NO: 896) |
| rs1135360 | chr22 | CCAGGTTCAAGTATGTCCTGGTCA (SEQ ID NO: 897) |
| | | GCAGTGGTGGTGGTGATGCT (SEQ ID NO: 898) |
| rs743616 | chr22 | GGTTACCCTGGGTGAAGAAGTGAG (SEQ ID NO: 899) |
| | | GGACGGGTAGAAGAAGAGAGACTG (SEQ ID NO: 900) |
| rs6639398 | chrX | CTACAAACCTGTCCAGAAAGAGGT (SEQ ID NO: 901) |
| | | AGACTGGTATAAGCTGAAAAAGGACAG (SEQ ID NO: 902) |
| rs5980274 | chrX | CAGTAGCAAATAGAAGCCATCAAGG (SEQ ID NO: 903) |
| | | TCAGTGATAGCAGTCTCCAAAACAAG (SEQ ID NO: 904) |
| rs1458178 | chrX | CTTCCCTGTGGAAGTGATGCTG (SEQ ID NO: 905) |
| | | CACTGTGGCAAGAGGGAATGTC (SEQ ID NO: 906) |
| rs5986751 | chrX | AGTTCTCATGAGCCTTGCTCAC (SEQ ID NO: 907) |
| | | CCTGGATCACAACAGGCTTTCTGGT (SEQ ID NO: 908) |
| rs9781645 | chrX | GGGCATTCATTTTCAAAGGAGGTGAT (SEQ ID NO: 909) |
| | | CCTCTAAAGCTCAGCCTTACTTGACA (SEQ ID NO: 910) |
| rs2209420 | chrX | CCGGTAACCAATCTTTCTTGATTCTCCCT (SEQ ID NO: 911) |
| | | GCAATATTTGTCTTCCCATGCCTGAC (SEQ ID NO: 912) |
| rs5984589 | chrX | AGTTCCTGGTTTCATTTGTCACAACATT (SEQ ID NO: 913) |
| | | GGGGATTTCTGGCCAGTCATGAGT (SEQ ID NO: 914) |
| rs2808742 | chrX | GACTCTAAAACAGGGCTGAATCAAGCA (SEQ ID NO: 915) |
| | | CTTATGACCCATTACTTCCCAGCA (SEQ ID NO: 916) |
| rs5917032 | chrX | CTGGCCAGTTTTTGTAGAACATCCCT (SEQ ID NO: 917) |
| | | GGATAGCACAGTGGTGATATGCT (SEQ ID NO: 918) |
| rs6529455 | chrX | GTAAGACAGACGGGCAGATGTC (SEQ ID NO: 919) |
| | | CACTCTGGAGAGAACTGGGAGA (SEQ ID NO: 920) |
| rs5931302 | chrX | GGGACGGAAAAGTTGCATTCAGTG (SEQ ID NO: 921) |
| | | GCTGGTGGATGGATAGTTCCCTGA (SEQ ID NO: 922) |
| rs6643690 | chrX | CCTCAGTCACATTAGCCAAATTTCAAGGGT (SEQ ID NO: 923) |
| | | TGAACCTCATAAGACAGGGTCTGTTC (SEQ ID NO: 924) |
| rs1781104 | chrX | TGTCTTGTCCAATGCCGAGA (SEQ ID NO: 925) |
| | | GGCAAATTGACTGGAAGCCA (SEQ ID NO: 926) |
| rs1781116 | chrX | GCAAATATGTTGGCAAGACGTAT (SEQ ID NO: 927) |
| | | CCCTTCCCCACCTTGGATAG (SEQ ID NO: 928) |
| rs318173 | chrX | CATCGCCCAAGGAGTGATATA (SEQ ID NO: 929) |
| | | CTCTGGGTATATTCAGAAGCC (SEQ ID NO: 930) |
| rs4825889 | chrX | TCTTTGGCTCCCCTAAATTGCT (SEQ ID NO: 931) |
| | | ATGAACCGAGTTTTGATTTGGAAT (SEQ ID NO: 932) |
| rs933315 | chrX | TGGGAAGACCAAAAACAAGCAA (SEQ ID NO: 933) |
| | | TGCCCCACAGAGATTTTAGTG (SEQ ID NO: 934) |
| rs1991961 | chrX | GTACGAGATGACTGACCACCA (SEQ ID NO: 935) |
| | | CTGACTAAGTGGAGACAACCG (SEQ ID NO: 936) |
| rs1937193 | chrX | GTCTCTCATATCCAGTGTAGGT (SEQ ID NO: 937) |
| | | TGCCTCAAACTCTTTCTTCTCAT (SEQ ID NO: 938) |
| rs652 | chrX | TGTCCCACACCTACAGTGCG (SEQ ID NO: 939) |
| | | ACCTGTGCATGCATTTGTGTAC (SEQ ID NO: 940) |
| rs5964206 | chrX | TGTCCTTCATCAGAAAAAATTCTC (SEQ ID NO: 941) |
| | | CAACACGGCTCTATATCACTGA (SEQ ID NO: 942) |
| rs149910 | chrX | GAAGATTTACTCAAATAGAGTG (SEQ ID NO: 943) |
| | | TCCATTGTTCATTTCAAGGAAT (SEQ ID NO: 944) |
| rs1299087 | chrX | TTTTAGAGGGATTCAGACACAAA (SEQ ID NO: 945) |
| | | CGCTTTGCAATTGGTTGTAT (SEQ ID NO: 946) |
| rs5920670 | chrX | ACGTTGGATGTGAAGAATTCTTGGGTCTGC (SEQ ID NO: 947) |
| | | ACGTTGGATGAGGAAATAAGAGTACGCTCC (SEQ ID NO: 948) |
| rs6631828 | chrX | ACGTTGGATGGTTGAAATGGTAGTGTTCAG (SEQ ID NO: 949) |
| | | ACGTTGGATGTTTCAGACCATGCATCTAGG (SEQ ID NO: 950) |
| rs973212 | chrX | ACGTTGGATGTATTCTCTCTTAGGCACTGG (SEQ ID NO: 951) |
| | | ACGTTGGATGCTTAGTCACATCAAAGGAGC (SEQ ID NO: 952) |
| rs5933388 | chrX | ACGTTGGATGATCACCCCAAGAAGAAACCC (SEQ ID NO: 953) |
| | | ACGTTGGATGGGAGGGGTTGGAAAAATATG (SEQ ID NO: 954) |
| rs6649211 | chrX | ACGTTGGATGGAGTTAAAAATTCTCCCATCC (SEQ ID NO: 955) |
| | | ACGTTGGATGCTGTGACAGTTGGTAAGGTG (SEQ ID NO: 956) |
| rs5916197 | chrX | CAGCAGGAATTTATCTGAGCAC (SEQ ID NO: 957) |
| | | GGCACTAAGGCATACACAACA (SEQ ID NO: 958) |
| rs4898214 | chrX | GAATTTACCTTTACTGGAGAACGCT (SEQ ID NO: 959) |
| | | AGGATTCTGGGTAGTACCTCAA (SEQ ID NO: 960) |
| rs2071182 | chrX | TACAGGTAGAAGAAGGCTCCAAG (SEQ ID NO: 961) |
| | | ATCAAATCCTCAGGCTAATGCT (SEQ ID NO: 962) |
| rs4826645 | chrX | TTGATTTCAGAGTGCCCAGAGT (SEQ ID NO: 963) |
| | | CAGTGTGGCTTTGTGTGTAGGA (SEQ ID NO: 964) |
| rs35284970 | chrY | GGTGGGATGTTGTTTTTCTCTCCT (SEQ ID NO: 965) |
| | | GCATTCGACTTACTGTTGCTGAAG (SEQ ID NO: 966) |
| rs13447361 | chrY | AGCTAGTGTGAGGAACATAGCAAGA (SEQ ID NO: 967) |
| | | TATCCCTGGAAATCAATTTGTTCCCTT (SEQ ID NO: 968) |
| rs2267801 | chrY | CTGTGCAAGTTGTGTGATCTTGGT (SEQ ID NO: 969) |
| | | GAGGAACTACCTTGGGATTTGGGGTT (SEQ ID NO: 970) |
| rs9786139 | chrY | GCTTGCAGGGAGCCAAAATTGT (SEQ ID NO: 971) |
| | | ACAGAAATATCCTTTGTCCCCACAATCT (SEQ ID NO: 972) |
| rs2075181 | chrY | AGCCAACATACTCGCCAATCC (SEQ ID NO: 973) |
| | | GCCTACTAGCAGGGCTCTGGAA (SEQ ID NO: 974) |
| rs17316007 | chrY | TCAGCTTCCAGATATTACCAGCATGCA (SEQ ID NO: 975) |
| | | TAAAGAGGATGAATGGGAGGAAAAAGAG (SEQ ID NO: 976) |
| rs17269396 | chrY | CAAGGGGTCGATTGATTAAAATAACGAA (SEQ ID NO: 977) |
| | | TACTTTAGGTTCAAGGGCACATGTG (SEQ ID NO: 978) |
| rs8179021 | chrY | GCTTTCTACGGCATAGAAAGTTTGTG (SEQ ID NO: 979) |
| | | GCCACCGCTTCAGAGTTGTTCTAGG (SEQ ID NO: 980) |
| rs16980601 | chrY | TCTCGCAAATAGTAGCAGAAACCTTATT (SEQ ID NO: 981) |
| | | GCTCCTACCACATGTCAAATGTTAGCA (SEQ ID NO: 982) |
| rs9341278 | chrY | GGAAAATGTGGGCTCGTTTTAATTATATTCA (SEQ ID NO: 983) |
| | | GATCGTCAAAGAATTCATACCTGTAATTTCTG (SEQ ID NO: 984) |
| rs917759 | chrY | TCCTGCAAATCTTAGGGGAACCCA (SEQ ID NO: 985) |
| | | GCTTACAATCCCAGCACTTTGG (SEQ ID NO: 986) |
| rs17269816 | chrY | CATGGTCTCTTTGGGTGAAAGACTAC (SEQ ID NO: 987) |
| | | CCTGGAAAATGTAGACTGGTGTTACATGC (SEQ ID NO: 988) |
| rs17316543 | chrY | ACCCCATTCTTTCTCAAGTTAATTGCA (SEQ ID NO: 989) |
| | | GCACAGCATTCTCACGGACATG (SEQ ID NO: 990) |
| rs17316592 | chrY | TGGTTCACACTCCCTTCTGTGT (SEQ ID NO: 991) |
| | | ATTCAACAGTCAAGGCCAGAAAGAC (SEQ ID NO: 992) |
| rs17269928 | chrY | TGAGAAAAGATTGCTCTAATCAGCCTATT (SEQ ID NO: 993) |
| | | GCTCTTCGGAAGTGTAAAGTCATTTAGC (SEQ ID NO: 994) |
| rs17276358 | chrY | GGGAAAACATTGCTTTCACCTCTGAG (SEQ ID NO: 995) |
| | | GAAAGGTTATTACCCTTCTGCTGTCCT (SEQ ID NO: 996) |
| rs16980641 | chrY | GACAAACCCAACCAAGAAGACATCCCT (SEQ ID NO: 997) |
| | | GCTGAAATTCTAACCCACAGTACTG (SEQ ID NO: 998) |
| rs11096432 | chrY | AGTGTGGGATACAGTGAAATCAGTG (SEQ ID NO: 999) |
| | | GGGTCAAGGGCAGAAACTGACAAC (SEQ ID NO: 1000) |
| rs9786502 | chrY | GGCAAAAATAAAATTTCCCCCAACCAT (SEQ ID NO: 1001) |
| | | CATTTCTCACCATGCCCACTCA (SEQ ID NO: 1002) |
| rs2032652 | chrY | TCCTGGATTCAGCTCTCTTCCTAA (SEQ ID NO: 1003) |
| | | AGCCTCTCCTGATACTCTGTGT (SEQ ID NO: 1004) |
| rs16980426 | chrY | TGCCAGAAGAGCAAATACGGTAAT (SEQ ID NO: 1005) |
| | | TGAAAACACAAAGCTACTGGCAAGC (SEQ ID NO: 1006) |
| rs13447443 | chrY | AGAAAGGAAACGAGATAAAATATTAAACGACTT (SEQ ID NO: 1007) |
| | | TGGTATCACAATAGAAGGGGAATGATC (SEQ ID NO: 1008) |
| rs17323322 | chrY | CTTGGGCAAGTACTGTAGACCATC (SEQ ID NO: 1009) |
| | | TTGGTGCAGGCTGAATTTTCAGT (SEQ ID NO: 1010) |
| rs13447354 | chrY | CTACATGGAAATGGTTGGCTATTGAGT (SEQ ID NO: 1011) |
| | | AAAAATACAGCACAATACAGCACATTCG (SEQ ID NO: 1012) |
| rs2033003 | chrY | TTACTTAGAGGCAGGGTGTTGC (SEQ ID NO: 1013) |
| | | TAAGACATTGCACTCAGCCTCAAG (SEQ ID NO: 1014) |
| rs16980610 | chrY | TAGTTTCGGAGCTAGCCCGTTTT (SEQ ID NO: 1015) |
| | | TTGGCCACTCCAAAGAGTGAAC (SEQ ID NO: 1016) |
| rs3848982 | chrY | ACGTTGGATGTATAGCGGCATACTTGCCTC (SEQ ID NO: 1017) |
| | | ACGTTGGATGGGTTCTTGATTAGGCTAAGG (SEQ ID NO: 1018) |
| rs2075640 | chrY | AGATCATCAGCTCTGTGACTGG (SEQ ID NO: 1019) |
| | | CCATCGATTTCCAGCATTTC (SEQ ID NO: 1020) |
| rs16980711 | chrY | AATTGCCAGGTGGTGTTACAG (SEQ ID NO: 1021) |
| | | TCATGGCTGTTTTGTCTCCA (SEQ ID NO: 1022) |
| rs9786707 | chrY | TTTTCTATGTAGGGGAGTTGTGAG (SEQ ID NO: 1023) |
| | | AAGCTGCAAAATTGAGTGAGC (SEQ ID NO: 1024) |
| rs17276345 | chrY | GAGAGGAAGGAACCCTCCAG (SEQ ID NO: 1025) |
| | | ATTGCAGGAAAAGGGTGGAA (SEQ ID NO: 1026) |
| rs9786479 | chrY | TCTCACTATGATCCAGGCAATG (SEQ ID NO: 1027) |
| | | CATAAATTACCCAGATTCGGGAAG (SEQ ID NO: 1028) |
| rs9306845 | chrY | ACAATGCTCAGGCTTCCACGAGA (SEQ ID NO: 1029) |
| | | TGAGGGGCCAGACTTGACTTGTGT (SEQ ID NO: 1030) |
| rs11096433 | chrY | ATCAACTGGCCATGTGAGTG (SEQ ID NO: 1031) |
| | | TGCTGACTAACATATTACCCCAAAC (SEQ ID NO: 1032) |
| rs52812045 | chrY | ACGTTGGATGGGCCCCAAATCCAACTAATG (SEQ ID NO: 1033) |
| | | ACGTTGGATGGTGAGACCACGAAGAGACTG (SEQ ID NO: 1034) |
| rs4589047 | chrY | ACGTTGGATGACACTGACCAACTTGAGAGG (SEQ ID NO: 1035) |
| | | ACGTTGGATGAGACTTTTCTGGGACCTGAG (SEQ ID NO: 1036) |
| rs17250121 | chrY | ACGTTGGATGGAATTCAAAGAGGGAGAAAC (SEQ ID NO: 1037) |
| | | ACGTTGGATGGAGTTTCTTCCTCCACTGAC (SEQ ID NO: 1038) |
| rs2032650 | chrY | ACGTTGGATGGGGATCAAATGGAGTTCCTG (SEQ ID NO: 1039) |
| | | ACGTTGGATGTCTCCTAAGCCTACAGGTTG (SEQ ID NO: 1040) |

It should be noted that based on the above SNP locus combination for paternity testing, the primer pair for detecting the SNP locus combination can be designed to detect the SNP locus combination, so as to achieve the purpose of prenatal paternity testing.

Still another embodiment of the disclosure provides a detection reagent, which may include the above detection primer pair. It should be noted that the above detection primer pair can be used in combination with detection auxiliary reagents for detection. The detection auxiliary reagents are known in the art, such as other reagents required for DNA library construction reactions, reagents required for linker sequence PCR reactions, and reagents required for magnetic bead purification.

Even still another embodiment of the disclosure provides a detection kit, which may include the above detection primer pair or the above detection reagent.

It should be noted that the above detection primer pair or the above detection reagent can be prepared in the form of a kit. The kit is a kit known in the art, including conventional components such as reagent bottles or instructions.

Further still another embodiment of the disclosure provides an application of the above detection primer pair or the above detection reagent in the preparation of products for prenatal paternity testing.

It should be noted that the above prenatal paternity testing products include kits or chips, etc.

Even further still another embodiment of the disclosure provides a method for prenatal paternity testing, which includes: step (1) DNA extraction: cell-free DNA of a fetus in maternal plasma, genomic DNA of a pregnant woman, and genomic DNA of a suspected father; step (2) multiplex polymerase chain reaction (PCR) amplification: performing the PCR amplification on a to-be-tested sample using the detection primer pair, the detection reagent or the detection kit to obtain a multiplex PCR amplification product; step (3) library construction: performing library construction on the multiplex PCR amplification product sequentially through two rounds of multiplex PCR reactions and two rounds of magnetic bead purification processes; step (4) library quality control: measuring and recording a concentration and a fragment length of the library after the library is constructed to prepare for sequencing on a machine; step (5) sequencing on the machine: performing sequencing on the machine by using a sequencing platform to obtain sequencing results; and step (6) data analysis: comparing the sequencing results with a human reference genome for bioinformatics analysis and determination of kinship by using a software, analyzing genotyping results of each SNP locus of the fetus, the pregnant woman and the suspected father, and performing comparison to identify whether there is a kinship between the suspected father and the fetus.

It should be noted that the method contains a large number of genetic markers, a large amount of genetic information content, strong system efficiency and good accuracy. It is suitable for forensic trace degraded samples, especially the detection of fetal cell-free DNA. It can improve the analysis ability of mixed samples in forensic genetic identification and provide an effective detection method for non-invasive prenatal paternity testing. It has high application value in forensic judicial identification.

In some specific embodiments, the genomic DNA of the pregnant woman is from blood, saliva, hair, or tissue, and the genomic DNA of the suspected father is from blood, semen, saliva, hair, or tissue.

In some specific embodiments, in the step (3), a first round reaction system of the two rounds of multiplex PCR reactions is 30 microliters (μL), including: 10 μL IGT-EM808 polymerase mixture, 5 μL primer pool, 3.5 μL enhancer buffer NB at 1 normality (IN), 2.5 μL enhancer buffer M. It should be noted that the reagents in the system are from iGeneTech Bioscience, Beijing China.

In some specific embodiments, in the step (3), first round reaction conditions of the two rounds of multiplex PCR reactions include: i) maintaining at 95° C. for 3 minutes and 30 seconds; ii) maintaining at 98° C. for 20 seconds, then maintaining at 60° C. for 8 minutes; repeating the step ii) 24 times; iii) maintaining at 72° C. for 5 minutes.

In some specific embodiments, in the step (3), a second round reaction system of the two rounds of multiplex PCR reactions is 30 μL, including: 10 μL IGT-EM808 polymerase mixture, 1 μL of 10 micromoles per liter (μM) IGT-15 Index, 1 μL of 10 μM of IGT-17 Index, 2.5 μL enhancer buffer M, and 13.5 μL PCR product mixture after first round of the two rounds of multiplex PCR reactions. In the step (3), second round reaction conditions of the two rounds of multiplex PCR reactions include: i) maintaining at 95° C. for 3 minutes and 30 seconds; ii) maintaining at 98° C. for 2 minutes, then maintaining at 58° C. for 1 minute, then maintaining at 72° C. for 30 seconds; repeating the step ii) 9 times; iii) maintaining at 72° C. for 5 minutes. It should be noted that the reagents in the system are from iGeneTech Bioscience, Beijing China.

To better understand the disclosure, the content of the disclosure is further illustrated below in combination with specific embodiments, but the content of the disclosure is not limited only to the following embodiments.

### Embodiment 1

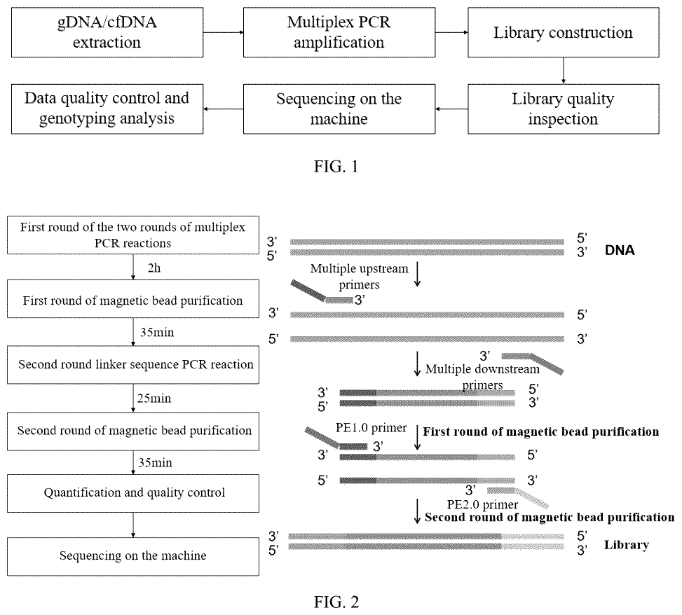

A method for non-invasive prenatal paternity testing by detecting SNP loci based on second-generation sequencing technology (referring to FIG. 1), including the following steps.

### Step 1: DNA Extraction

1. QIAamp DNA Blood Mini Kit (Cat. No./ID: 51104, from QIAGEN) is used to extract gDNA from peripheral blood as follows. The specific operation steps are as follows: (1) absolute ethanol is added to AW1 and AW2 according to the instructions and then fully mixed for later use; (2) 20 μL protease K and 200 μL sample are added into a 1.5 milliliters (mL) centrifuge tube; If the sample volume is insufficient, an appropriate amount of phosphate-buffered saline (PBS) solution can be added; (3) 200 μL Buffer AL solution is added and vortexed for 15 seconds; (4) the centrifuge tube is placed in a water bath at 56° C. for constant temperature incubation for 10 minutes; (5) the centrifuge tube is taken out and centrifuged briefly to remove the residual liquid on the tube cap; (6) 200 μL absolute ethanol is added, vortexed for 15 seconds, and then centrifuged briefly to remove the residual liquid on the tube cap; (7) the mixture in the centrifuge tube is transferred to a centrifugal column, taking care not to wet the edges, and the centrifugal column is placed in a collection tube; (8) the collection tube with the centrifugal column is centrifuged at 8,000 revolutions per minute (rpm) for 1 minute, then the centrifugal column is placed in a clean collection tube, and the collection tube containing the filtrate is discarded; (9) 500 μL Buffer AW1 is added to the centrifugal column, centrifuged at 8,000 rpm for 1 minute, then the centrifugal column is placed in another clean collection tube, and the collection tube containing the filtrate is discarded; (10) 500 μL Buffer AW2 is added to the centrifugal column, and centrifuged at 14,000 rpm for 3 minutes; (11) the centrifugal column is placed in still another clean collection tube, the collection tube containing the filtrate is discarded, and centrifuged at 14,000 rpm for 1 minute; (12) the centrifugal column is placed on a new 1.5 mL centrifuge tube, 200 μL Buffer TE is added dropwise in suspension, incubated at room temperature for 5 minutes, and then centrifuged at 8,000 rpm for 1 minute; (13) the gDNA in the centrifuge tube is collected and stored at 4° C. for standby.

2. QIAamp DNA Investigator Kit (Cat. No./ID: 56504, from QIAGEN) is used to extract gDNA from other human biological samples as follows. The specific operation steps are as follows: (1) absolute ethanol is added to AW1 and AW2 according to the instructions and fully mixed for later use; (2) saliva swabs, hair with hair follicles or nails are placed into a 2 mL centrifuge tube; (3) different reagents are added according to different samples: a. for saliva swabs: 20 μL protease K and 600 μL Buffer ATL are added, and shaken vigorously for 20 seconds; b. for hair with hair follicles or nails: 20 μL protease K, 300 μL Buffer ATL and 20 μL dichlorodiphenyltrichloroethane (DDT) are added, and shaken vigorously for 20 seconds; (4) the centrifuge tube is heated at a constant temperature of 56° C. and shaken at 900 rpm for 90 minutes; (5) the centrifuge tube is taken out and centrifuged briefly to remove the residual liquid on the tube cap; (6) different reagents are according to different samples: a. for saliva swabs: 600 μL Buffer AL and 1 μL carrier RNA are added, and shaken vigorously for 30 seconds; b. for hair with hair follicles or nails: 300 μL Buffer AL and 1 μL carrier RNA are added, and shaken vigorously for 30 seconds; (7) the centrifuge tube is heated at a constant temperature of 70° C. and shaken at 900 rpm for 10 minutes; (8) the centrifuge tube is taken out and centrifuged briefly to remove the residual liquid on the tube cap; (9) different reagents are added according to different samples: a. for saliva swabs: 300 μL absolute ethanol is added and shaken vigorously for 30 seconds; b. for hair with hair follicles or nails: 150 μL absolute ethanol is added and shaken vigorously for 30 seconds; (10) the centrifuge tube is taken out and centrifuged briefly to remove the residual liquid on the tube cap; (11) 700 μL of liquid in the centrifuge tube is transferred to a centrifugal column, taking care not to wet the edges, and centrifuged at 8,000 rpm for 1 minute, the waste liquid is poured out after centrifugation, the centrifugal column is placed into a collection tube, the remaining liquid is transferred to the centrifugal column for centrifugation, and then placed the centrifugal column on a clean collection tube, if the liquid is not completely centrifuged through the centrifugal column, the operation is repeated until all the liquid passes through the column; (12) 500 μL Buffer AW1 is added and centrifuged at 8,000 rpm for 1 minute; (13) 700 μL Buffer AW2 is added and centrifuged at 8,000 rpm for 1 minute; (14) 700 μL absolute ethanol is added and centrifuged at 8,000 rpm for 1 minute; (15) Centrifugation is performed at 14,000 rpm for 3 minutes without loading; (16) the centrifugal column is placed into a new 1.5 mL centrifuge tube with the cap open for 10 minutes or heated at room temperature for 10 minutes or heated at 56° C. for 3 minutes; (17) 40 μL Buffer ATE is added dropwise in suspension in the center of the filter membrane, and incubated at room temperature with the cap closed for 5 min followed by centrifugation at 14,000 rpm for 1 minute; (18) the gDNA is collected in the centrifuge tube and stored at 4° C. for standby.

3. Extraction of Fetal Cell-Free DNA from Plasma in Pregnant Women

Maternal peripheral blood samples are collected in a blood collection and preservation tube containing special anticoagulant components, and then separated maternal plasma from the peripheral blood by a two-step centrifugation method. The specific operation steps are as follows: (1) the preservation tube is centrifuged at 4,000 g at room temperature for 10 minutes, and then the supernatant is taken into a centrifuge tube; and (2) the supernatant is centrifuged again at 14,000 g at 4° C. for 10 minutes, the supernatant (i.e., maternal plasma) is taken into a plasma preservation tube and stored at −20° C. for cfDNA extraction.

The cfDNA is extracted by using MagicPure® Cell-Free DNA Kit II (Cat. No./ID: EC211-01, from TransGen Biotech Co., Ltd.). The specific operation steps are as follows: (1) protease K, plasma, and LB36 are sequentially added to a centrifuge tube according to the volume of the sample used; (2) the centrifuge tube is vortexed and mixed for 15 seconds, and incubated at 60° C. for 20 minutes (inverted and mixed 2-3 times during this period); (3) the centrifuge tube is placed on ice for 3 minutes, and sequentially added with an appropriate amount of binding buffer 36 (BB36) and magnetic bead; (4) the centrifuge tube is vortexed and mixed for 15 seconds and allowed to stand at room temperature for 10 minutes (inverted and mixed 2-3 times during this period); (5) the centrifuge tube is placed on an appropriate magnetic rack for magnetic separation; (6) the supernatant is aspirated and discarded, the supernatant close to the tube wall on the opposite side of the centrifuge rack to avoid accidentally aspirating magnetic beads, the centrifuge tube is then removed, 1 mL CB36 is added into the centrifuge tube, the centrifuge tube is vortexed and mixed for 15 seconds, and then placed on the magnetic rack for magnetic bead separation; (7) the supernatant is aspirated and discarded, the centrifuge tube is then removed, 1 mL CB36 is added into the centrifuge tube, the centrifuge tube is vortexed and mixed for 15 seconds for magnetic separation; (8) the supernatant is aspirated and discarded, the centrifuge tube is then removed, 1 mL WB36 is added into the centrifuge tube, the centrifuge tube is vortexed and mixed for 15 seconds for magnetic separation; (9) the step (8) is repeated once; (10) the supernatant is aspirated and discarded, taking care to aspirating the residual liquid on the tube cap; (11) the centrifuge tube is allowed to stand at room temperature for 5-10 minutes and the magnetic beads are air dried; (12) the centrifuge tube is removed, added with an appropriate amount of EB36, and vortexed vigorously for 5 minutes; (13) the centrifuge tube is placed on the magnetic rack for magnetic separation, and the liquid other than the magnetic beads is transferred to a low nucleic acid adsorption centrifuge tube to avoid aspirating magnetic beads; and (14) the obtained cfDNA is stored at −20° C.

### Step 2: Selection of SNP Loci in a Composite Detection System Used for Multiplex PCR Amplification

The genomic data of projects such as 1000 Genomes, Avon Longitudinal Study of Parents and Children (ALSPAC), Genome Aggregation Database (GnomAD), Trans-Omics for Precision Medicine (TOPMED), etc. included in the National Center for Biotechnology Information (NCBI) public database and the SNP locus information in published literature are integrated, and eligible SNP loci are screened according to the following requirements: (1) located in the intron region of the genome; (2) minor allele frequency (MAF)≥0.3; (3) biallelic polymorphism; (4) the physical distance between loci is greater than 5 megabases (Mb), and all loci are in a state of linkage equilibrium in the population; (5) the genotype frequency distribution of each locus conforms to Hardy-Weinberg equilibrium in the population (after Bonferroni correction); (6) PCR primers suitable for SGS sequencing can be designed; (7) have human-specific species specificity; (8) not closely related to certain diseases; and (9) irrelevant to genomic functional elements.

Based on the chromosomal positions and other information of the 520 selected SNP loci in the human reference genome 37 (Genome Reference Consortium Human Build 37/human genome build 19, abbreviated as GRCh37/hg19), multiplex primers are designed for each locus, requiring that the amplicon contains 50 base pairs (bp) sequences upstream and downstream of the target site. The relevant information of the 520 SNP loci selected by the disclosure and the positions and primer sequences of their amplification primers on the chromosome are shown in Table 1 above.

### Step 3: Library Construction

The library construction process is shown in FIG. 2, which involves two rounds of multiplex PCR reactions and two rounds of magnetic bead purification processes (reaction conditions are shown in Tables 2 to 5 below), and after quantification and quality control processes, it proceeds to sequencing on the machine.

TABLE 2
Composition of the first round of multiplex PCR system

| Reaction components | Volume of each component (μL) |
|---|---|
| deionized distilled water (ddH₂O) | 9-x |
| Enhancer buffer NB (1N) | 3.5 |
| Enhancer buffer M | 2.5 |
| Primer pool | 5 |
| DNA | x |
| IGT-EM808 polymerase mixture | 10 |

TABLE 3
Reaction conditions of the first round of multiplex PCR

| Procedures | Temperature and time |
|---|---|
| Denaturation | 95° C., 3 min 30 s |
| 24 cycles | 98° C., 20 s |
| | 60° C., 8 min |
| Extension | 72° C., 5 min |

TABLE 4
Composition of the second round linker
sequence PCR reaction system

| Reaction components | Volume of each component (μL) |
|---|---|
| PCR product mixture[1] | 13.5 |
| Enhancer buffer M | 2.5 |
| ddH₂O | 2 |
| IGT-15 Index(10 μM) | 1 |
| IGT-17 Index(10 μM) | 1 |
| IGT-EM808 polymerase mixture | 10 |

TABLE 5
Reaction conditions of the second round linker sequence PCR

| Procedures | Temperature and time |
|---|---|
| Denaturation | 95° C., 3 min 30 s |
| 9 cycles | 98° C., 20 s |
| | 58° C., 1 min |
| | 72° C., 30 s |
| Extension | 72° C., 5 min |

In addition, the specific process of magnetic bead purification is as follows: (1) AMPure XP magnetic beads balanced at room temperature are added to the PCR product, and mixed by gentle pipetting using a pipette; (2) after the mixed solution is incubated at room temperature for 5 minutes, the PCR tube is placed on a magnetic rack for standing for 3 minutes to wait for stratification; (3) after complete removal of the supernatant, the PCR tube is removed from the magnetic rack, a YF buffer B solution is added, and then mixed by gentle pipetting using a pipette; (4) after incubation at room temperature for 5 minutes, the PCR tube is placed on the magnetic rack again for standing for 3 minutes; (5) after removal of the supernatant, a 80% ethanol solution is added to the PCR tube placed on the magnetic rack, and then the PCR tube is left stand for 30 seconds; (6) the operation (5) above is repeated once, and after completely removal of the supernatant, the PCR tube is left stand at room temperature for 3 minutes to completely evaporate the residual ethanol; (7) the PCR tube is removed from the magnetic rack, nuclease-free water is added, fully mixed by using a pipette to gently pipetting resuspended magnetic beads, avoiding air bubbles during the process, and then the PCR tube is left stand at room temperature for 2 minutes; (8) the PCR tube is placed on the magnetic rack and is left stand for 3 minutes; (9) the supernatant is aspirated and transferred to a new PCR tube.

### Step 4: Library Quality Control

After library construction is completed, the library concentration is measured and recorded on the Qubit™ 3.0 Fluorometer using Qubit™ dsDNA HS Assay Kit, with a concentration greater than 1.0 nanogram per microliter (ng/μL) being considered acceptable; and the library fragment length is measured and recorded using the Qsep400 fully automatic nucleic acid protein analysis system.

### Step 5: Sequencing on the Machine

The sequencing on the machine is performed using the NovaSeq 6000 sequencing platform (Illumina, USA).

### Step 6: Data Analysis

The sequencing results are compared with the human reference genome (GRCh37/hg19) for bioinformatics analysis by using a software. The data analysis process is as follows. (1) Sequencing data quality assessment: the downloaded raw data is subjected to quality control, the Trimmomatic software is used to preliminarily filter the fastq file, the adapter information, low-quality bases and undetected bases are filter out to obtain clean reads (i.e., clean data). (2) Alignment: then, the clean data is aligned with the reference genome using the bwa mem mode of the BWA software, while also performing sorting to obtain a sam file. The Samtools are used to convert the sam file into the bam format (i.e., bam result file). Finally, data statistics is performed on the bam result file. The statistical content includes indicators such as average library length, alignment rate, coverage rate, capture rate, sequencing depth, and uniformity to evaluate whether the constructed library meets the standards. Only after meeting the standards can subsequent analysis be conducted. (3) Mutation detection: based on the bam result compared with the genome reference sequence, softwares such as samtools and Genome Analysis Toolkit (GATK) are used to detect the mutation information in the sample, and then the ANNOVAR software is used to statistically analyze and annotate the detected SNPs, insertions and deletions (InDels) and other mutations to determine the gene information, functional information, harmfulness information, etc. corresponding to the mutation site.

### Embodiment 2

The embodiment involves the forensic validation of the SNP detection method based on SGS technology as provided in provided in the embodiment 1. Specifically, it includes: evaluation of the sequencing quality, repeatability, accuracy, and sample applicability of the method constructed in the embodiment 1 according to the requirements of the Scientific Working Group for DNA Analysis Methods (SWGDAM), and calculation of forensic parameters.

The same sample is sequenced three times according to the complete operation process. The results show that the allele genotyping results of the three sequencings are consistent, and the repeatability is good. The three repeated sequencing situations are shown in Table 6.

TABLE 6
Sequencing situation of repeatability verification

| | | | | | Target |
|---|---|---|---|---|---|
| | Raw | Clean | | Coverage | mean |
| | bases | bases | QCrate | rate | depth |
| Sample | (Mb) | (Mb) | (%) | (%) | (×) |
| Replicate 1 | 610.19 | 431.06 | 74.09 | 96.19 | 3202.27 |
| Replicate 2 | 613.52 | 446.62 | 72.80 | 96.39 | 3458.60 |
| Replicate 3 | 615.66 | 461.55 | 74.97 | 96.08 | 3689.01 |

Verification is performed using different samples such as peripheral blood, hair, nails, and saliva swabs from the same individual. The results show that all four different types of samples are sequenced successfully. The sequencing depths of each sample are between 2174.73-3324.19×. The sequencing quality could meet the requirements of data analysis. The sequencing situations of the four different samples are shown in Table 7.

TABLE 7
Sequencing situation of repeatability verification

| | | | | | Target |
|---|---|---|---|---|---|
| | Raw | Clean | | Coverage | mean |
| | bases | bases | Qcrate | rate | depth |
| Sample | (Mb) | (Mb) | (%) | (%) | (×) |
| Peripheral blood | 642.02 | 446.49 | 79.54 | 96.08 | 3324.19 |
| Hair | 354.33 | 274.17 | 77.38 | 95.86 | 2174.93 |
| Nails | 437.75 | 333.52 | 76.19 | 95.65 | 2652.53 |
| Saliva swabs | 450.86 | 340.98 | 75.63 | 95.97 | 2706.50 |

Among the 450 A-chromosome SNPs (A-SNPs), the loci with the lowest diplotype frequency (DP) values are rs3759236 and rs2288201 (0.5014), and the locus with the highest DP value is rs647347 (0.6592). Each SNP locus is independent of each other. The product principle is used to calculate that cumulative probability of discrimination (CPD) is 1-7.59×10⁻¹⁷⁶, CPE_{duo} is 1-3.31×10⁻²², and CPEₜᵣᵢₒ is 1-1.48×10⁻⁴⁴. The average DP, observed heterozygosity (Ho), observed heterozygosity (He), polymorphism information content (PIC), Pe_{duo}, and Peₜᵣᵢₒ are 0.5902, 0.4567, 0.4555, 0.3495, 0.1039, and 0.2007 respectively.

The results show that the library construction of the composite PCR amplification system constructed in the embodiment 1 is successfully constructed and the sequencing quality is good. The system has good repeatability, high accuracy, and is suitable for DNA typing of multiple types of samples including blood, saliva, semen, hair, bones, etc.

### Embodiment 3

In the embodiment of the disclosure, the multiplex PCR amplification system and detection method in the embodiment 1 are used to detect 15 groups of pregnant women and their corresponding suspected fathers, and determine the kinship between the fetal components in the peripheral blood of pregnant women and the suspected fathers. Then the judgment results are compared with the kinship judgment results based on STR typing. The results are shown in Table 8.

TABLE 8
Summary of sequencing results of 15 groups of triplex samples

| | | | | | Combined | | |
|---|---|---|---|---|---|---|---|
| | | | | | paternity index, | | |
| | | | | | CPI | | |
| | | | | | (Non-invasive | Paternity | Paternity |
| | Sequencing | | Effective | Number of | prenatal | relationship | relationship |
| | failed bit | Classification | SNP | mismatch | paternity testing, | (based on this | (based on |
| Sample | number | success rate | number | points | NIPPT) | method) | STR) |
| case 1 | 0 | 100.00% | 131 | 0 | 32.19 | Identification | Identification |
| case2 | 5 | 99.15% | 95 | 40 | −89.97 | Exclusion | Exclusion |
| case3 | 2 | 99.66% | 121 | 0 | 30.15 | Identification | Identification |
| case4 | 4 | 99.32% | 80 | 0 | 18.90 | Identification | Identification |
| case5 | 0 | 100.00% | 112 | 0 | 21.80 | Identification | Identification |
| case6 | 0 | 100.00% | 72 | 15 | −27.39 | Exclusion | Exclusion |
| case7 | 3 | 99.49% | 125 | 0 | 28.75 | Identification | Identification |
| case8 | 2 | 99.66% | 130 | 0 | 29.40 | Identification | Identification |
| case9 | 0 | 100.00% | 152 | 0 | 34.31 | Identification | Identification |
| case10 | 0 | 100.00% | 83 | 0 | 14.02 | Identification | Identification |
| case11 | 0 | 100.00% | 116 | 0 | 26.32 | Identification | Identification |
| case12 | 0 | 100.00% | 58 | 0 | 15.32 | Identification | Identification |
| case13 | 0 | 100.00% | 140 | 0 | 31.52 | Identification | Identification |
| case14 | 4 | 99.32% | 131 | 0 | 29.57 | Identification | Identification |
| case15 | 2 | 99.66% | 100 | 0 | 22.87 | Identification | Identification |

The results show that all SNP loci are sequenced successfully in 45 samples, and the analysis results of sequencing quality indicate that the library constructed this time is of high quality and the sample data is true and reliable; correct kinship can be obtained for 15 groups of triplex samples, and the biological fathers in respective groups of samples can be distinguished from unrelated male individuals.

In summary, the method for detecting SNPs based on SGS technology described in the disclosure can be applied in forensic practices such as individual identification and non-invasive prenatal paternity testing, providing a new method with a large amount of genetic information, high accuracy and strong system efficiency for forensic judicial identification.

Finally, it should be noted that the above embodiments are only used to illustrate the technical solutions of the disclosure and are not limited. Although the disclosure has been described in detail with reference to illustrated embodiments, those skilled in the art should understand that the technical solutions of the disclosure can be modified or equivalently replaced without departing from the purpose and scope of the technical solutions of the disclosure. All of them should be included by the scope of the claims of the disclosure.

What is claimed is:

1. A combination of single nucleotide polymorphism (SNP) loci for paternity testing, comprising 520 SNP loci:
rs868163, rs3007429, rs3007430, rs2076605, rs7531163, rs607254, rs2361660, rs7516939, rs494198, rs6588052, rs11208265, rs472908, rs3736296, rs2274570, rs3811003, rs9428083, rs9427094, rs703116, rs1920149, rs7536561, rs647347, rs2274064, rs12042179, rs4844573, rs2076355, rs12031285, rs630120, rs12042900, rs7527925, rs11578981, rs7552783, rs2045955, rs2618702, rs2805396, rs1332777, rs10802626, rs2256242, rs10927011, rs2280132, rs2271621, rs7561070, rs4072407, rs455060, rs3738984, rs3771274, rs2020911, rs2276625, rs2244182, rs2271244, rs72481904, rs1061305, rs16830136, rs2288201, rs12620077, rs1867864, rs1542484, rs830973, rs12613075, rs2366751, rs2303838, rs2042995, rs2562839, rs12997453, rs3024866, rs1208081, rs2242074, rs17552879, rs3213784, rs6704780, rs12991025, rs34490902, rs2304774, rs17868324, rs1105879, rs4234097, rs6756901, rs456168, rs2733532, rs3729587, rs3816526, rs2507940, rs13097628, rs7641844, rs1799987, rs936175, rs13066214, rs749512, rs6445873, rs4681982, rs2290174, rs533852, rs1463138, rs2713604, rs1683787, rs2681092, rs16861582, rs2303909, rs9290259, rs1515442, rs2593813, rs2028572, rs9851685, rs2286342, rs899691, rs2306400, rs13121363, rs13144326, rs7655165, rs12650452, rs2125314, rs225170, rs6856362, rs7660664, rs34840208, rs7677541, rs227365, rs17009761, rs62333013, rs2166337, rs4429853, rs13170062, rs162850, rs7710284, rs6451206, rs11957963, rs476569, rs1801033, rs706714, rs182190, rs309557, rs4916683, rs274557, rs2303068, rs4072924, rs438464, rs2236277, rs2842949, rs2071303, rs3737095, rs3818554, rs167772, rs3920621, rs10948667, rs9474140, rs9367689, rs4712138, rs2230862, rs4710457, rs1936439, rs2305837, rs519068, rs1875845, rs9448893, rs3828753, rs3734353, rs6922753, rs2273752, rs7754205, rs3813328, rs902373, rs3798663, rs6914831, rs3762000, rs22908, rs6904364, rs3818110, rs9371581, rs9383985, rs9397102, rs1873176, rs214968, rs3734440, rs9356399, rs4716346, rs2527509, rs852423, rs6461252, rs7782939, rs1023542, rs2240401, rs10261071, rs12669315, rs6950777, rs2072454, rs2286822, rs733957, rs6957975, rs7779997, rs362726, rs607755, rs2520272, rs213950, rs3815221, rs3779032, rs7830, rs3802100, rs1550697, rs3020221, rs2073574, rs12675283, rs2272730, rs2737335, rs7842714, rs3098659, rs6511, rs10093032, rs2261147, rs2069568, rs2272653, rs11780911, rs3817681, rs17855765, rs4244610, rs4251691, rs12683181, rs11243704, rs12358961, rs10828545, rs10491056, rs3817419, rs3740232, rs2435352, rs7089209, rs3812658, rs2105702, rs3750863, rs3758626, rs10999338, rs780668, rs7917781, rs4747203, rs2131956, rs7074064, rs1903858, rs1409332, rs10786229, rs3740486, rs813944, rs196294, rs2278202, rs2272599, rs7942159, rs3758680, rs1799937, rs11037882, rs3740878, rs3729802, rs589691, rs630394, rs736894, rs11604536, rs1109977, rs883223, rs2196168, rs641936, rs1784424, rs591058, rs10895417, rs693147, rs2276424, rs1954787, rs2241501, rs880054, rs10848683, rs2267549, rs216868, rs12426675, rs2518144, rs3759236, rs10772008, rs7955902, rs2276456, rs1034762, rs3803184, rs4075913, rs2071218, rs706815, rs2270267, rs7397862, rs2302629, rs6606734, rs2464195, rs3741591, rs2075784, rs9564692, rs3752451, rs9534342, rs2296533, rs6313, rs9518061, rs661585, rs589985, rs482757, rs3803236, rs2281974, rs178642, rs2073349, rs1152590, rs229581, rs3759753, rs2075179, rs12432149, rs3742656, rs3751582, rs1800419, rs1800404, rs173224, rs422388, rs2279580, rs1801376, rs140605, rs2306187, rs58156069, rs1147129, rs762551, rs1370275, rs9806604, rs2307438, rs9672296, rs3815003, rs2272037, rs7496614, rs224204, rs1641010, rs2280764, rs3115446, rs215901, rs2072061, rs249954, rs7203525, rs243834, rs2304483, rs1532625, rs11076207, rs483053, rs3991715, rs2548861, rs12918876, rs3803679, rs3752827, rs238238, rs12453262, rs17671352, rs1565817, rs2304908, rs876660, rs3744586, rs1736219, rs548973, rs2952976, rs9894648, rs3110641, rs8067890, rs8176161, rs850730, rs2289677, rs2258689, rs3809863, rs2256835, rs3744093, rs758596, rs4342, rs4362, rs3744024, rs11077379, rs3816256, rs3213926, rs7232329, rs3748422, rs8097342, rs2510344, rs12960081, rs1058396, rs1450425, rs1788027, rs2298712, rs1893788, rs6567271, rs1629038, rs3764640, rs2277984, rs2860178, rs620744, rs539887, rs8104339, rs2114724, rs280520, rs3826803, rs2075021, rs2738447, rs6413504, rs2304154, rs8113582, rs1043996, rs2302603, rs10424582, rs55742440, rs1469699, rs3745859, rs10880, rs2246095, rs17 997 83, rs392891, rs20580, rs3730849, rs2387588, rs4002462, rs1062798, rs12981413, rs3745405, rs269950, rs3729836, rs2076653, rs2073193, rs2281575, rs3803953, rs2295435, rs2295179, rs363050, rs2273060, rs2273061, rs143383, rs2072792, rs4608591, rs2425906, rs6021437, rs2234487, rs3746657, rs6062700, rs41309367, rs2073421, rs2073425, rs8130564, rs872331, rs754507, rs62215498, rs2839077, rs7283989, rs2236486, rs7279347, rs7279622, rs9982233, rs2839226, rs2839257, rs112544, rs2072708, rs2235324, rs5756516, rs1135360, rs743616, rs6639398, rs5980274, rs1458178, rs5986751, rs9781645, rs2209420, rs5984589, rs2808742, rs5917032, rs6529455, rs5931302, rs6643690, rs1781104, rs1781116, rs318173, rs4825889, rs933315, rs1991961, rs1937193, rs652, rs5964206, rs149910, rs1299087, rs5920670, rs6631828, rs973212, rs5933388, rs6649211, rs5916197, rs4898214, rs2071182, rs4826645, rs35284970, rs13447361, rs2267801, rs9786139, rs2075181, rs17316007, rs17269396, rs8179021, rs16980601, rs9341278, rs917759, rs17269816, rs17316543, rs17316592, rs17269928, rs17276358, rs16980641, rs11096432, rs9786502, rs2032652, rs16980426, rs13447443, rs17323322, rs13447354, rs2033003, rs16980610, rs3848982, rs2075640, rs16980711, rs9786707, rs17276345, rs9786479, rs9306845, rs11096433, rs52812045, rs4589047, rs17250121, and rs2032650.

2. A detection primer pair, wherein the detection primer pair is configured to detect the combination of SNP loci for paternity testing as claimed in claim 1, and the nucleotide sequences of the detection primer pair are SEQ ID NO: 1 to SEQ ID NO: 1040 shown as follows:

| SNP locus | chromosome | the nucleotide sequences of the detection primer pair |
|---|---|---|
| rs868163 | chr1 | GTGGAGACTGGAAGCATTCTCAA (SEQ ID NO: 1) |
| | | TGCAGAGAAGCTCTGGAACTTG (SEQ ID NO: 2) |
| rs3007429 | chr1 | AAGCATTGCCCAGGAGAATAGG (SEQ ID NO: 3) |
| | | CTCCCTGGTCTTCATCTCACCA (SEQ ID NO: 4) |
| rs3007430 | chr1 | AGCGCTCAGTGACCTTTTGG (SEQ ID NO: 5) |
| | | TGCATCGCTCCCTTTAGAGACAG (SEQ ID NO: 6) |
| rs2076605 | chr1 | CACTGGGAGCAGGAGAGTCT (SEQ ID NO: 7) |
| | | CCGGCACCTCTCTCCCAT (SEQ ID NO: 8) |
| rs7531163 | chr1 | TTCTCGTAGTTGGGCAGGTTGTCT (SEQ ID NO: 9) |
| | | TCAGTCAGCTCCCTACTCACCAT (SEQ ID NO: 10) |
| rs607254 | chr1 | AAGATGACGCGGGCATTGTTCCT (SEQ ID NO: 11) |
| | | GACCCAAGCAGTTCCAAGTAAGG (SEQ ID NO: 12) |
| rs2361660 | chr1 | TGGTCTACGCGCATAGCAAGGTG (SEQ ID NO: 13) |
| | | GGCGAGGTCAGAGGGGCTGTCT (SEQ ID NO: 14) |
| rs7516939 | chr1 | GCTCCAAGAAATTCTTGGCTATATGGA (SEQ ID NO: 15) |
| | | TCTTCTATAGGACAAGGTGCAACG (SEQ ID NO: 16) |
| rs494198 | chr1 | TTGTGGGAAGGGCGTTCATCCAT (SEQ ID NO: 17) |
| | | AGGCCAGCAAGTGTGACAGTCA (SEQ ID NO: 18) |
| rs6588052 | chr1 | CCATTCTTCTCCCCAGAATGCAT (SEQ ID NO: 19) |
| | | TTGTGGGACCGTATGTAAAGAAGATCC (SEQ ID NO: 20) |
| rs11208265 | chr1 | TGGAAGTAGGCAGTCAGACGTA (SEQ ID NO: 21) |
| | | TGTCTTCCTCAGGTCATGTTGG (SEQ ID NO: 22) |
| rs472908 | chr1 | TCCTGTTATCACTCATGAGAGTTTCTC (SEQ ID NO: 23) |
| | | CCAAGGTCGCTTAAAAACCCAACAAG (SEQ ID NO: 24) |
| rs3736296 | chr1 | AGATCAGCGTATGATGCTCTTCCAA (SEQ ID NO: 25) |
| | | AAACCTACATGGCCAACAACTTTGG (SEQ ID NO: 26) |
| rs2274570 | chr1 | AATCCCAGCCACACCAAGGTAGT (SEQ ID NO: 27) |
| | | AAAATTTCCCTGCCTTCTTCCCTTC (SEQ ID NO: 28) |
| rs3811003 | chr1 | TGAGGTTGTGACAGCAACTGAGGG (SEQ ID NO: 29) |
| | | CAACCTGTTTCAGGATCTCCAGGA (SEQ ID NO: 30) |
| rs9428083 | chr1 | TATTTGAGGAGAGGCAGCTCCA (SEQ ID NO: 31) |
| | | CTGCAAACTCGCCATCAAACTC (SEQ ID NO: 32) |
| rs9427094 | chr1 | TGAGACTGCAGAGGTATGATGC (SEQ ID NO: 33) |
| | | TCCAGGATCTCCAGGTCATAGCCAT (SEQ ID NO: 34) |
| rs703116 | chr1 | ACGTTTAAATGACTTCTATTCTTTTCCCAT (SEQ ID NO: 35) |
| | | AGATTACCTTTTCCCGAACTTCATCAGA (SEQ ID NO: 36) |
| rs1920149 | chr1 | GCATCTATTCACAAGGTCGCTTC (SEQ ID NO: 37) |
| | | ACAGCAGGGACTATAAAGAACCAGGT (SEQ ID NO: 38) |
| rs7536561 | chr1 | TATGGAATCCCCCAGTCTCCATC (SEQ ID NO: 39) |
| | | AATTTGGGCATCATTGCGCATG (SEQ ID NO: 40) |
| rs647347 | chr1 | AGAATGGCTTTTACCGGCACAGAG (SEQ ID NO: 41) |
| | | ACAAGAGGGAAGGAAGGAAGGA (SEQ ID NO: 42) |
| rs2274064 | chr1 | CCACTTGTCTCTCATTTGGTCGAA (SEQ ID NO: 43) |
| | | CCCTTATGTGAAAATGGGGGTGA (SEQ ID NO: 44) |
| rs12042179 | chr1 | AGTTTGGTTGAGGCAGCACAA (SEQ ID NO: 45) |
| | | ACCAGGTGCCTCTCAAATTCTTGC (SEQ ID NO: 46) |
| rs4844573 | chr1 | GGACAGGCTTATTGACTGCTGTGGC (SEQ ID NO: 47) |
| | | TTACCAGACATTCCACATGCTTCCT (SEQ ID NO: 48) |
| rs2076355 | chr1 | AAGGGTTATAGGGCACCTTCCA (SEQ ID NO: 49) |
| | | TGAAGCCACATTCTCTACTCGGTGA (SEQ ID NO: 50) |
| rs12031285 | chr1 | TCCTGTACACAGCTCTGTGTCTCT (SEQ ID NO: 51) |
| | | TTTTCTTCAGGTCCTTGCACTTGG (SEQ ID NO: 52) |
| rs630120 | chr1 | CCATAACTGAACCCCTTTCTTAGTTGAA (SEQ ID NO: 53) |
| | | GAGCAATGTGAATTCATACCTTGGTCAG (SEQ ID NO: 54) |
| rs12042900 | chr1 | GTTTATTCACGCAGGATTAAAAGGGAA (SEQ ID NO: 55) |
| | | CTTTGCTATTTGCTGAAGTTTGATATCTATGTG (SEQ ID NO: 56) |
| rs7527925 | chr1 | CCTATAGGTGGTTTCTTCAGCTCTAT (SEQ ID NO: 57) |
| | | AATACAGGATGACATTGGATTCCTACC (SEQ ID NO: 58) |
| rs11578981 | chr1 | AGAAAGACAAGTGCCCTGAAGAC (SEQ ID NO: 59) |
| | | TTTGGATGTACTGGGAGTGGAGA (SEQ ID NO: 60) |
| rs7552783 | chr1 | TCTCGCCGCCATTTATCCTTC (SEQ ID NO: 61) |
| | | GATTTTGGCACTCAGAGCACCA (SEQ ID NO: 62) |
| rs2045955 | chr1 | TCATGATGGCTCTGTGGAGGTA (SEQ ID NO: 63) |
| | | AGGATACTGAGGAAGCCAGGTT (SEQ ID NO: 64) |
| rs2618702 | chr1 | AATTGAGCTTGGCTGGCAGT (SEQ ID NO: 65) |
| | | TTCTGCCTTTTCCCGCTATGG (SEQ ID NO: 66) |
| rs2805396 | chr1 | TGTTAGGTCGTGACAGAGTGGTGG (SEQ ID NO: 67) |
| | | AATGAACACTATGGGCGCTCTTG (SEQ ID NO: 68) |
| rs1332777 | chr1 | CCCTCTCGTCTGAAACAAAGGTTAC (SEQ ID NO: 69) |
| | | TTTTCCTCCTTCTCCTCCTCCT (SEQ ID NO: 70) |
| rs10802626 | chr1 | GGGGATTGACGATGTGATTGAGAC (SEQ ID NO: 71) |
| | | GGCCAACTGTTGCCGTTTTCTCTG (SEQ ID NO: 72) |
| rs2256242 | chr1 | AAGTGCCAGAGTCAGCATTCTGG (SEQ ID NO: 73) |
| | | AAATGTTTGCTGACCTCTCCCTGA (SEQ ID NO: 74) |
| rs10927011 | chr1 | CAGCAGAGCTTTAGCAAGGAAGCAA (SEQ ID NO: 75) |
| | | GGGAAAGATTAGAGCCTGGGCACT (SEQ ID NO: 76) |
| rs2280132 | chr2 | TAGAGAGGCAGCTGCGGAAC (SEQ ID NO: 77) |
| | | GCCCGGCATCCCCGGAGAGA (SEQ ID NO: 78) |
| rs2271621 | chr2 | CTGGTCGGACATGAAATAACTTGCTAT (SEQ ID NO: 79) |
| | | CTACTTTGACCCATGTTAGGAACTCAGA (SEQ ID NO: 80) |
| rs7561070 | chr2 | AGGTAAGCCCAGAAGACAAAGCA (SEQ ID NO: 81) |
| | | TGAAGATAGGCCCTAGTGCTGAGT (SEQ ID NO: 82) |
| rs4072407 | chr2 | GCCATCTGACTGTTTCTCAGAGTCAA (SEQ ID NO: 83) |
| | | CTACCTGGATTTGCCGAAGCAA (SEQ ID NO: 84) |
| rs455060 | chr2 | TTCCATGTGGATTCCACCTGTGT (SEQ ID NO: 85) |
| | | TAAGTAATCGGGGATGTTCCCTGA (SEQ ID NO: 86) |
| rs3738984 | chr2 | CACATCCTGGATGGAAAACATGCC (SEQ ID NO: 87) |
| | | GCCTTTGCTGTCCAGTTATCTCT (SEQ ID NO: 88) |
| rs3771274 | chr2 | TCTCCCCCAGAAGGTAAGAAGTACTGC (SEQ ID NO: 89) |
| | | CAACCTCCCTGTATAGGCACAGT (SEQ ID NO: 90) |
| rs2020911 | chr2 | GGGCAAGTCTACGCTTATGAGAC (SEQ ID NO: 91) |
| | | AAAGTCAGCCAGTGACTTAATAGGAAG (SEQ ID NO: 92) |
| rs2276625 | chr2 | TGAAAGACATGGCAGCATATATTACCT (SEQ ID NO: 93) |
| | | GAGCCTTTTGTGTAGGGAGACAG (SEQ ID NO: 94) |
| rs2244182 | chr2 | GTGAGAGGGAGCACTTCTTACAGTTA (SEQ ID NO: 95) |
| | | AATCAGGCAACTGGGACTGAAG (SEQ ID NO: 96) |
| rs2271244 | chr2 | GCATGAGCTCTGGTTGTAAGAAGTA (SEQ ID NO: 97) |
| | | GAGTACAGGCACTTCCAAAATGC (SEQ ID NO: 98) |
| rs72481904 | chr2 | CAGGCTGGGCACCACAGCA (SEQ ID NO: 99) |
| | | GGGACGGCAGCTCCTCCT (SEQ ID NO: 100) |
| rs1061305 | chr2 | TGGTGCTTCTGAATGCTCAGACTTC (SEQ ID NO: 101) |
| | | TTGGGGGAGATGTAGCAAACATG (SEQ ID NO: 102) |
| rs16830136 | chr2 | CCCCTTATATCTTTGTGCATTTTCTCAAAC (SEQ ID NO: 103) |
| | | CTGTAATTCTGTGGCCTCTTTTAGGTGA (SEQ ID NO: 104) |
| rs2288201 | chr2 | TCCCGATACAGTCTCTACGTTGGAG (SEQ ID NO: 105) |
| | | GAAGAGGGGAGGAAATATCGAGGA (SEQ ID NO: 106) |
| rs12620077 | chr2 | ACGTCATTGCTTATGGTCACTCA (SEQ ID NO: 107) |
| | | TTGGCCAATTCAAAGCTCATGGCAT (SEQ ID NO: 108) |
| rs1867864 | chr2 | CCAGCTCTTAACTCTCTTCATCTCATTT (SEQ ID NO: 109) |
| | | CTTTTCTCTCCAAGACGCAACAGT (SEQ ID NO: 110) |
| rs1542484 | chr2 | GGCTGTTATCTTCAGTTTCTATAAAAAGAGAGA (SEQ ID NO: 111) |
| | | CAGAGCTATTTCCACATAGTAGTGCATC (SEQ ID NO: 112) |
| rs830973 | chr2 | GCCATTTGATTTTAAGCTCTGGGATGTG (SEQ ID NO: 113) |
| | | CAAGTCCTCCCATCACTTTCTAACATG (SEQ ID NO: 114) |
| rs12613075 | chr2 | TACTGGTAGTGAACGGAGACCAC (SEQ ID NO: 115) |
| | | TCGTAAGGTCACAGCTGTACAC (SEQ ID NO: 116) |
| rs2366751 | chr2 | GTGCTCCACTTTTCACTCCCTTT (SEQ ID NO: 117) |
| | | TGTAGCTGTATTACGGGTCACATCAAC (SEQ ID NO: 118) |
| rs2303838 | chr2 | CTCTGCATCACGTTTTTGTACCA (SEQ ID NO: 119) |
| | | TGGCCCAGGAGTATCTGGATAAATAGTAGG (SEQ ID NO: 120) |
| rs2042995 | chr2 | ACCTTTAGGTGGTGGTTCAACCCTT (SEQ ID NO: 121) |
| | | GATATGCCATGTTGTGGGTGGT (SEQ ID NO: 122) |
| rs2562839 | chr2 | TCACTCGGTGCTACATTGATGATC (SEQ ID NO: 123) |
| | | CCAGCTACTGTACACTCCAAGGTACAG (SEQ ID NO: 124) |
| rs12997453 | chr2 | GATCCTAAGTCTGATCAATTGTTTGTCAG (SEQ ID NO: 125) |
| | | TTCTTGCACATCATCAGAGCTGT (SEQ ID NO: 126) |
| rs3024866 | chr2 | CTCCCATTGGAAGATTCTTCAATAGACATG (SEQ ID NO: 127) |
| | | TACAAGAGCAGCAACAAAGTTTCCA (SEQ ID NO: 128) |
| rs1208081 | chr2 | TGTGGGATATGGATAGGGGGAAAG (SEQ ID NO: 129) |
| | | TGTCTTCTTTGAACAGGAGCCTCT (SEQ ID NO: 130) |
| rs2242074 | chr2 | ATCCAGCAGCCGCTGTCCA (SEQ ID NO: 131) |
| | | AAGGCAAGGCGGGACAAG (SEQ ID NO: 132) |
| rs17552879 | chr2 | AGTTGCTTTCTTAGGAAATGTAGTTGCTTT (SEQ ID NO: 133) |
| | | TAAAAGATGGGTGTTTGAAGGAGCA (SEQ ID NO: 134) |
| rs3213784 | chr2 | ACCTTCTTATTCAGCCCCGACTGGA (SEQ ID NO: 135) |
| | | TTCCTTTTCCAGTGGGTCTGCTA (SEQ ID NO: 136) |
| rs6704780 | chr2 | GGTATGTCGCCCTAGAAAAATGAACAA (SEQ ID NO: 137) |
| | | ATCGTTTTCTCTTAAGGCTACTGTTCATG (SEQ ID NO: 138) |
| rs12991025 | chr2 | GTCGTTTCCACTGCCAGCT (SEQ ID NO: 139) |
| | | AGTGCCCTTCTTTTCCCCTTG (SEQ ID NO: 140) |
| rs34490902 | chr2 | CGCCTGGAAGCGGACTTCAC (SEQ ID NO: 141) |
| | | TGAGGAGCGTTGGGGACGAAG (SEQ ID NO: 142) |
| rs2304774 | chr2 | CGAATAGGACATGGCCAAGACAAAA (SEQ ID NO: 143) |
| | | CCAACAGTCCTGTGGGGTTGACT (SEQ ID NO: 144) |
| rs17868324 | chr2 | CGAAGTGCATTTTCTCTATTAACAAGTTCA (SEQ ID NO: 145) |
| | | GTTTTGATGCAGTGTTTCTCGATCC (SEQ ID NO: 146) |
| rs1105879 | chr2 | ACAATCACTTTGCTGAGCGATCAT (SEQ ID NO: 147) |
| | | CACCCTGAACTTCTTTAAGGAGAGCA (SEQ ID NO: 148) |
| rs4234097 | chr2 | GGCTGCTGCAGAAGTGACACC (SEQ ID NO: 149) |
| | | ACCTCCCTGAGGAAGGACAAC (SEQ ID NO: 150) |
| rs6756901 | chr2 | GATCTTGGGAGGGGCTGTTG (SEQ ID NO: 151) |
| | | TGGTAACCTGCACCTCAATGTGA (SEQ ID NO: 152) |
| rs456168 | chr3 | ACTGTAAGCCATGATGCCCTTCC (SEQ ID NO: 153) |
| | | CCACAGAGGTACGCACTGACACA (SEQ ID NO: 154) |
| rs2733532 | chr3 | AAGAGAGTCCACCTCCTGCAT (SEQ ID NO: 155) |
| | | TCAGATACCAGGAAGCCCCAT (SEQ ID NO: 156) |
| rs3729587 | chr3 | TGAGGATGCAAAGGCTCAGAGAGA (SEQ ID NO: 157) |
| | | AAGGAAAAGTCCTTCCCCATCATTCC (SEQ ID NO: 158) |
| rs3816526 | chr3 | TGTCTCTGATTCCAGAGCCAGTA (SEQ ID NO: 159) |
| | | ATCAGCCCTGTAGGACTTCTGA (SEQ ID NO: 160) |
| rs2507940 | chr3 | CTGTGGTTTGCTTCTCTCTCCTTCC (SEQ ID NO: 161) |
| | | TGCTGTCTGGGCATTTCTGTTCTCT (SEQ ID NO: 162) |
| rs13097628 | chr3 | TGTACCCAGAATCTGTGCTCAAGT (SEQ ID NO: 163) |
| | | CATCACATGGAACGAACTGTGTCATG (SEQ ID NO: 164) |
| rs7641844 | chr3 | ACCCTGAGTCAGATCCATTGCCAC (SEQ ID NO: 165) |
| | | TGGTTCTGAACCCTCCAACCCAAG (SEQ ID NO: 166) |
| rs1799987 | chr3 | CAGGTTGTTTCCGTTTACAGAGAACA (SEQ ID NO: 167) |
| | | CCAGGATCCCCCTCTACATTTAAAGTTG (SEQ ID NO: 168) |
| rs936175 | chr3 | TTGCTAGGTCCACTCTGGGATC (SEQ ID NO: 169) |
| | | GGAAGCATCAAACTCGACCTCCT (SEQ ID NO: 170) |
| rs13066214 | chr3 | CTCTCCCATCTTCCTCCTCTCTTCT (SEQ ID NO: 171) |
| | | GAGCCCGATGTCCAAAAGGTA (SEQ ID NO: 172) |
| rs749512 | chr3 | CCAAAACATGATGCCCCTCCA (SEQ ID NO: 173) |
| | | GTTTAAGGAGCGCATCGGCTAC (SEQ ID NO: 174) |
| rs6445873 | chr3 | TGTATCATCTCTCTGTTGGGGCTAC (SEQ ID NO: 175) |
| | | GTCACCGGGACAGCAATAAAGCT (SEQ ID NO: 176) |
| rs4681982 | chr3 | GGGAAGCTAATACAATATGTAAAGTTTTCTCATAA (SEQ ID NO: 177) |
| | | TAGAGGCACATATTTAAAATAATTTGAAATACGGAA (SEQ ID NO: 178) |
| rs2290174 | chr3 | GCTCTGGTATTCCTGCAGGTTCAG (SEQ ID NO: 179) |
| | | ACATGGCCAGTCTTAGAGGTACTG (SEQ ID NO: 180) |
| rs533852 | chr3 | GGACACTACATGAAGTCCTGTGTA (SEQ ID NO: 181) |
| | | CTCACACTCTCCTCTGGCCTT (SEQ ID NO: 182) |
| rs1463138 | chr3 | GAAGCTGTGCTTCTCCATGAGA (SEQ ID NO: 183) |
| | | AATCAGTTTCTGCCAAAGGGACT (SEQ ID NO: 184) |
| rs2713604 | chr3 | CCACCCTGACCCTTTTTAAGCAGA (SEQ ID NO: 185) |
| | | CCCTCTTACGGGAAGCCCTTCTG (SEQ ID NO: 186) |
| rs1683787 | chr3 | GCACACGTGGCACTACCATGG (SEQ ID NO: 187) |
| | | CCGGATGTACATCGCCCTGAC (SEQ ID NO: 188) |
| rs2681092 | chr3 | TGAAAGAAGAGAACCGGGTATGCTT (SEQ ID NO: 189) |
| | | AGGCTGTGTGGGTCCATTGAGCAT (SEQ ID NO: 190) |
| rs16861582 | chr3 | ACAAACAATCAGGGGGCCAATTA (SEQ ID NO: 191) |
| | | AGGGCCTCAGGAATTTTGGAGA (SEQ ID NO: 192) |
| rs2303909 | chr3 | ATGCAGCGTTTATGCAGATACCCAT (SEQ ID NO: 193) |
| | | GATGGAGACTTTGGGTAAGTGCT (SEQ ID NO: 194) |
| rs9290259 | chr3 | GTAGATACTCCTGACAATTTCCTACAGTTCT (SEQ ID NO: 195) |
| | | TACCACTTCTTTCACTACATCTAGTGAC (SEQ ID NO: 196) |
| rs1515442 | chr3 | AGGGTCTACTATTTCAGCAGCTTGG (SEQ ID NO: 197) |
| | | TAGACCAACTTCATTTCCACATCCA (SEQ ID NO: 198) |
| rs2593813 | chr3 | TGAACGCTTTCAGGCAGTCAC (SEQ ID NO: 199) |
| | | TCAGGTACTAGTGAATCAGGCAGAACC (SEQ ID NO: 200) |
| rs2028572 | chr3 | TGTTCATCGAAGAGATCCCTGTCAT (SEQ ID NO: 201) |
| | | CACGATCAGTGGTGAAGTGTTTCTG (SEQ ID NO: 202) |
| rs9851685 | chr3 | ATAGGTTGCTTGGGAGACCCTACAA (SEQ ID NO: 203) |
| | | AGTGGAATGACTTTGCGGAGGA (SEQ ID NO: 204) |
| rs2286342 | chr4 | AGGAGAGAGAATACTCTGAACAGCTAAT (SEQ ID NO: 205) |
| | | GCCTAATACTTGAATTCCCCTATCG (SEQ ID NO: 206) |
| rs899691 | chr4 | GCTGTTAATTAACATGCCCTGGTGGT (SEQ ID NO: 207) |
| | | TGATTGAGTTTCTGAAGCTGCAAGTC (SEQ ID NO: 208) |
| rs2306400 | chr4 | ACCAATCCAGGTTGGTGAGTAGG (SEQ ID NO: 209) |
| | | CTGTCCCTGTCCTTCCTTTCTTC (SEQ ID NO: 210) |
| rs13121363 | chr4 | GTGAGACCCTATCTCAATAAATAAATAATTGTCTTC (SEQ ID NO: 211) |
| | | TCTTCAAGGATGTTCAGTGAAAAGCATG (SEQ ID NO: 212) |
| rs13144326 | chr4 | GGCCTGGTCTATAAGGATTGCTTAA (SEQ ID NO: 213) |
| | | ACCTGATTTTCACCCCAGGCCAT (SEQ ID NO: 214) |
| rs7655165 | chr4 | AGAGTATCTTAAACTTTCTGGCTTTTGTACAC (SEQ ID NO: 215) |
| | | TTGTTTTGTGGAAAAAGATTCAGATTTTTGC (SEQ ID NO: 216) |
| rs12650452 | chr4 | GTTTGCTCTCAAAGCCTCAGTGGAA (SEQ ID NO: 217) |
| | | TAATAGCTCGAGGCTACTGACACT (SEQ ID NO: 218) |
| rs2125314 | chr4 | TCCCCAACATGAAGATTATGGACTGT (SEQ ID NO: 219) |
| | | GCACTTCGTGAGGCAGATGTAGACTG (SEQ ID NO: 220) |
| rs225170 | chr4 | GCAGGCCACTTTCATGAAACTCCAT (SEQ ID NO: 221) |
| | | AGGCCGCATACATTTAATGTAATTGGA (SEQ ID NO: 222) |
| rs6856362 | chr4 | AGGACTTGAGGCATGTGCAAAC (SEQ ID NO: 223) |
| | | GCTGTTATAGATGCTTGGGGGCATG (SEQ ID NO: 224) |
| rs7660664 | chr4 | TGTCACATTCACCATCGTGCAGCC (SEQ ID NO: 225) |
| | | CATGACGGCAGTAACTCCCTCA (SEQ ID NO: 226) |
| rs34840208 | chr4 | CGGTTCACCTTCACTGTTTCTGAT (SEQ ID NO: 227) |
| | | GACACTGGAGGAGGTAGCCTAG (SEQ ID NO: 228) |
| rs7677541 | chr4 | AGGTGTTTTCCTGGGAGTCTGAAT (SEQ ID NO: 229) |
| | | GGCCCAGATGCCATTGCGATTCTGA (SEQ ID NO: 230) |
| rs227365 | chr4 | AGTCTGGTTTCCATGTCCATGAG (SEQ ID NO: 231) |
| | | AAGCATTACATCAATTTCTCAAATAGTCAAGTAC (SEQ ID NO: 232) |
| rs17009761 | chr4 | GTAGTGTTGGCTGGAAAGGCTA (SEQ ID NO: 233) |
| | | TGTGGACTCCTGTTCTGAGAACCA (SEQ ID NO: 234) |
| rs62333013 | chr4 | TGCTCGTCGCCTGCCA (SEQ ID NO: 235) |
| | | GCGTGAGTAACCGCCGCGGT (SEQ ID NO: 236) |
| rs2166337 | chr5 | GGACAGGAGTAAAACCGAGGATC (SEQ ID NO: 237) |
| | | CCCAAGGAGAATGCAGAGCCAT (SEQ ID NO: 238) |
| rs4429853 | chr5 | CTGAGACGAAGAAAAACCGAGGAAA (SEQ ID NO: 239) |
| | | ATGGGAAAAGGGGAGAAGATTGTGT (SEQ ID NO: 240) |
| rs13170062 | chr5 | CTGTCTTCATTACTCTGCAAAGCAG (SEQ ID NO: 241) |
| | | ATATGAAACCATTTCTGCACCCCAT (SEQ ID NO: 242) |
| rs162850 | chr5 | GTGGTAACATGTTATTTACCCATTCAGTATT (SEQ ID NO: 243) |
| | | AGTGTCTGTCTGTGGAGTGTATCCTTC (SEQ ID NO: 244) |
| rs7710284 | chr5 | ACTCTTGTCCAAGAAGCTATCCAAG (SEQ ID NO: 245) |
| | | TGCTCTACCAGTTCTGCAAGAGGAGA (SEQ ID NO: 246) |
| rs6451206 | chr5 | GTCTAACAAAATATTCATGAGTTGTCCTGTT (SEQ ID NO: 247) |
| | | GAAGAAGCTCTTACAGGCTGCA (SEQ ID NO: 248) |
| rs11957963 | chr5 | AGCAATCTAAAGCATAGCCACCAT (SEQ ID NO: 249) |
| | | TTAATGAGAGCTGGTTACAGGACAC (SEQ ID NO: 250) |
| rs476569 | chr5 | GATCGCATTGTGACCATTCACTC (SEQ ID NO: 251) |
| | | ATAATGACCATTGTGTATATCTGTTTCCAAG (SEQ ID NO: 252) |
| rs1801033 | chr5 | GAATGCATGGTTGAAAGGCTACCAG (SEQ ID NO: 253) |
| | | AGATATAACTCTGAGGCAGGGTCAAG (SEQ ID NO: 254) |
| rs706714 | chr5 | TTCCTGTTGCACCAGGTTCTTCGAA (SEQ ID NO: 255) |
| | | GTCGTTATGTTCTGGGCAGAAG (SEQ ID NO: 256) |
| rs182190 | chr5 | GAAACAGAGAAAAATGCTTCCAAAGCAA (SEQ ID NO: 257) |
| | | GCAAATTGGCATGTGTACATGGT (SEQ ID NO: 258) |
| rs309557 | chr5 | CACAGGTTTTCCTTTGGAGGAAGATT (SEQ ID NO: 259) |
| | | ATTTAGGGACACCCAGACTTCAC (SEQ ID NO: 260) |
| rs4916683 | chr5 | GCCTCTAAGTAACTGGCTTTAATCTCT (SEQ ID NO: 261) |
| | | AACATAGACTCTGATCCTGATGGTGA (SEQ ID NO: 262) |
| rs274557 | chr5 | CTGCCACTGTTTGCTTACTTCATC (SEQ ID NO: 263) |
| | | CCCACTGGCAGGATGATTTCTGTCT (SEQ ID NO: 264) |
| rs2303068 | chr5 | CTAAGAGTACAGCTTCCTTTGAGGT (SEQ ID NO: 265) |
| | | CATGTTCAGGGAACACGTGCAT (SEQ ID NO: 266) |
| rs4072924 | chr5 | TCCTAACCTTCTTCATGGAGCCTAT (SEQ ID NO: 267) |
| | | CAACAGGTTGCTGCCGACT (SEQ ID NO: 268) |
| rs438464 | chr5 | CCCGTCTTCCCCTATGGTCT (SEQ ID NO: 269) |
| | | GCCAGCTTCACGGGCAGCTTC (SEQ ID NO: 270) |
| rs2236277 | chr6 | TTTCCCTTTGCCCCCATGTAG (SEQ ID NO: 271) |
| | | TTCTGCGAGAACATCCATGATGG (SEQ ID NO: 272) |
| rs2842949 | chr6 | CTCCATGTATATATGCAGTATGCTTCCTA (SEQ ID NO: 273) |
| | | ACAACTTTACCTGGATGTTTAGTTGGATC (SEQ ID NO: 274) |
| rs2071303 | chr6 | GTGGGATCACATGTTCACTGTTGACT (SEQ ID NO: 275) |
| | | TTGAAGGAAACAGCTGGAAGTCTGAG (SEQ ID NO: 276) |
| rs3737095 | chr6 | GTGGCCCCTCTCCTTTCTTG (SEQ ID NO: 277) |
| | | TACAGGTCTGGTTCAGTGAGAAGATG (SEQ ID NO: 278) |
| rs3818554 | chr6 | CAAAGCCCTAGGGAACCACAGGAA (SEQ ID NO: 279) |
| | | AGCAGAGGCCCTCTGTTGATG (SEQ ID NO: 280) |
| rs167772 | chr6 | CCTCTTTCCCCAAAAGCTACGAACT (SEQ ID NO: 281) |
| | | GCCCGCATTGACAAAAAGCA (SEQ ID NO: 282) |
| rs3920621 | chr6 | GAAGCTTTTGTTCCTTTTCTAAGGGATTT (SEQ ID NO: 283) |
| | | ATGCAATGTAGATACTGTGAAGTAGACT (SEQ ID NO: 284) |
| rs10948667 | chr6 | TGACAACCTCCCAGGATGTTGTTC (SEQ ID NO: 285) |
| | | GAAAGTGTCCTCCTAGAGGTGGA (SEQ ID NO: 286) |
| rs9474140 | chr6 | GTGCCAACTGCTTACAATTTATTGCCA (SEQ ID NO: 287) |
| | | ACACACTTACCTATCAATGTACTCAGCA (SEQ ID NO: 288) |
| rs9367689 | chr6 | AACTACTGTTTGCCTCTAAGATTTTCTC (SEQ ID NO: 289) |
| | | TTCACCATTTAATACTGCTCCACCT (SEQ ID NO: 290) |
| rs4712138 | chr6 | TCCAGCTCCTTTTTAGACTGTTCTG (SEQ ID NO: 291) |
| | | TCACCATTCTCTTTTAAGAAGTTCTCTGTG (SEQ ID NO: 292) |
| rs2230862 | chr6 | TGGCAGCTGTTAGTTCATCTACCTG (SEQ ID NO: 293) |
| | | CTGCATGATGTAGCCTGTGTGATC (SEQ ID NO: 294) |
| rs4710457 | chr6 | CTGATGCAGGTGCCATTGTTG (SEQ ID NO: 295) |
| | | AGGTCTAGAATGCAGCCCCTGAAC (SEQ ID NO: 296) |
| rs1936439 | chr6 | ACATTCTTCAAAAACCAACATGGATTTTTTG (SEQ ID NO: 297) |
| | | ATAACATAAACAGCTAATTTTTAAAACCACAGTG (SEQ ID NO: 298) |
| rs2305837 | chr6 | TTTAAGGCGCGTTTATCCCTTG (SEQ ID NO: 299) |
| | | GATTTAGCAGATCAGGCAACAAAACC (SEQ ID NO: 300) |
| rs519068 | chr6 | CTTACCGGTTTGCCTGAATTTCCC (SEQ ID NO: 301) |
| | | ATCAATCTGCCTTACCCAGACTG (SEQ ID NO: 302) |
| rs1875845 | chr6 | TCAGCTGCTTTTACTGCTGGTTT (SEQ ID NO: 303) |
| | | CATGTTCTTGATCTCTGCTGCCTT (SEQ ID NO: 304) |
| rs9448893 | chr6 | CTTTCTTTGTACCTTGATTTTAGTCAAGTTGTA (SEQ ID NO: 305) |
| | | AATGAATCTTTTCCAGTCTGTAACAAGTG (SEQ ID NO: 306) |
| rs3828753 | chr6 | CATGCAGATCAGTTCCTGAGACTT (SEQ ID NO: 307) |
| | | ATTTCAGGTTCATGTGATCCGAGAG (SEQ ID NO: 308) |
| rs3734353 | chr6 | TTTTGGTAGCAGCCGTCGAAGG (SEQ ID NO: 309) |
| | | GGTAGGAAGAGCCCCCAAAAGGT (SEQ ID NO: 310) |
| rs6922753 | chr6 | CCTTGCAGTGTAATCGACATAAACCC (SEQ ID NO: 311) |
| | | GAACATCTGCCTTGTCTCTTTTGTTG (SEQ ID NO: 312) |
| rs2273752 | chr6 | GAGTTGAATGTCAGCCAATTTCCATA (SEQ ID NO: 313) |
| | | GGGGTATTTGGGATCTGTAGTGAGCCTT (SEQ ID NO: 314) |
| rs7754205 | chr6 | CCATAGCCATTGTACCCATCCAAG (SEQ ID NO: 315) |
| | | GCAGTGCAGCAGATAAGGTCACAG (SEQ ID NO: 316) |
| rs3813328 | chr6 | AGGAAAAGCGGATTCAAAATTGCAG (SEQ ID NO: 317) |
| | | CCTTACTTTTTCCCTTGGGTTGTTC (SEQ ID NO: 318) |
| rs902373 | chr6 | CCCAATACCATTGGAGAGAAATGTTCTA (SEQ ID NO: 319) |
| | | TTGTAGAGCTCTGTAATTGGGCAT (SEQ ID NO: 320) |
| rs3798663 | chr6 | GCCACAACCTGTGATTCAGAGACTA (SEQ ID NO: 321) |
| | | TAGGGAAAACCTCCCTCAATTCTGT (SEQ ID NO: 322) |
| rs6914831 | chr6 | AGAGAGTGAAAATCAAAGTACATATGTAATACG (SEQ ID NO: 323) |
| | | AGGAGGGGATCGCTCCTTTATCTCA (SEQ ID NO: 324) |
| rs3762000 | chr6 | CATGATTTTACACATTTGTATTAATCACAGTACCAA (SEQ ID NO: 325) |
| | | CTGTATACAGTACCTGTATGCTGGTTGTTC (SEQ ID NO: 326) |
| rs22908 | chr6 | TTCAGCACCTACTTGGAGATGGT (SEQ ID NO: 327) |
| | | TAGGCCTGACAGAATTGATCACTG (SEQ ID NO: 328) |
| rs6904364 | chr6 | GACCCAAACCCATCATCACCTAATG (SEQ ID NO: 329) |
| | | TTTTGCAGAATTTTCCACACCCATC (SEQ ID NO: 330) |
| rs3818110 | chr6 | AGATCCCTTTTCTGCTGCTGTGTAA (SEQ ID NO: 331) |
| | | TTGATCTGGAGCTCGATGGTCT (SEQ ID NO: 332) |
| rs9371581 | chr6 | CAGGGGAAACTGTTCTCTTCCATT (SEQ ID NO: 333) |
| | | CTCCTGTATGTTACTGGTGGCAA (SEQ ID NO: 334) |
| rs9383985 | chr6 | AGGTGGACAGCTGTACTCTCT (SEQ ID NO: 335) |
| | | TTTCTTTTTGGAGACTGAGTTTTGCTCT (SEQ ID NO: 336) |
| rs9397102 | chr6 | CTTGGTTGCCCTGTTAGACTGACG (SEQ ID NO: 337) |
| | | TTAGCCTCAATTTGTTCACCACGTT (SEQ ID NO: 338) |
| rs1873176 | chr6 | TAACTCAAGGGTGCTGAGCATG (SEQ ID NO: 339) |
| | | GCAGATCATAGAGGCGATTGCTGCT (SEQ ID NO: 340) |
| rs214968 | chr6 | CATCAAGCGACTGTTGGTTTCCA (SEQ ID NO: 341) |
| | | CATTACTGAAAGGATCATTTTGTTAGAGGGTA (SEQ ID NO: 342) |
| rs3734440 | chr6 | TGGGTGTTACCCAGAAAACCTTC (SEQ ID NO: 343) |
| | | TTTCTTAGGATCTGTCTGGCTCCA (SEQ ID NO: 344) |
| rs9356399 | chr6 | CCTGGCCTGATTAATTTTCCATAGCTA (SEQ ID NO: 345) |
| | | CAGCAAATGGATTCCGCAGT (SEQ ID NO: 346) |
| rs4716346 | chr6 | GTGCTGGTTGTGCTCCGAAGC (SEQ ID NO: 347) |
| | | ACCTGCGTATGTGGAGTAGGT (SEQ ID NO: 348) |
| rs2527509 | chr7 | GGCTTAGGGGCTCCTAAATGTCACC (SEQ ID NO: 349) |
| | | TTGACCGAGTGCACGAAGC (SEQ ID NO: 350) |
| rs852423 | chr7 | GGGTGTTGAAGGTCTCAAACATGATC (SEQ ID NO: 351) |
| | | AAGAACACGGCTAAGTGTGCT (SEQ ID NO: 352) |
| rs6461252 | chr7 | CAAATGAACCACACTACAGTGCTTGTCC (SEQ ID NO: 353) |
| | | GACACCTTCCTCAACAAATGGTCTCAC (SEQ ID NO: 354) |
| rs7782939 | chr7 | TCTGCAGGGAGGCGGAAGCC (SEQ ID NO: 355) |
| | | CGCTAGCCTCGGGCCGATGC (SEQ ID NO: 356) |
| rs1023542 | chr7 | CTTGGGTGTAAGGAAAGAAGAATTGGGC (SEQ ID NO: 357) |
| | | GGTTGTCCAGCTTGAGGAACTGT (SEQ ID NO: 358) |
| rs2240401 | chr7 | GGCTCTGTGTAAGGTTTCCTGA (SEQ ID NO: 359) |
| | | CAGCATAGTCCAAGACCTAGCCAA (SEQ ID NO: 360) |
| rs10261071 | chr7 | GAAAATTTATCCAAAAGTAACAGGAAAAGACTTT (SEQ ID NO: 361) |
| | | GGTAAATGTTCATTATAGAAACTTTGAAAAATGCA (SEQ ID NO: 362) |
| rs12669315 | chr7 | CCAGGAGTAGCTATGACTGATCTTGC (SEQ ID NO: 363) |
| | | TGATTGTAACCTACTGTCATCCCAGGT (SEQ ID NO: 364) |
| rs6950777 | chr7 | CAGCCGATGGATCACTTTGGTCCG (SEQ ID NO: 365) |
| | | AACATAGCGAAGGCATTGGTACCT (SEQ ID NO: 366) |
| rs2072454 | chr7 | CCATTCTCCCGCAGAAATCCT (SEQ ID NO: 367) |
| | | ATGTCGATGGACTTCCAGAACC (SEQ ID NO: 368) |
| rs2286822 | chr7 | AGGAGCTGCTGCGCAAG (SEQ ID NO: 369) |
| | | GCCCCAATCAGCCCCATCTCAC (SEQ ID NO: 370) |
| rs733957 | chr7 | CCAGCAGCAACTTAAAATTACAACAGA (SEQ ID NO: 371) |
| | | TTAAAGGGCAAAATGGTTATAATGTTGCT (SEQ ID NO: 372) |
| rs6957975 | chr7 | CACTTACACAGACGTGCACAGAAG (SEQ ID NO: 373) |
| | | TAAACAAAGCATCAGGGGCACA (SEQ ID NO: 374) |
| rs7779997 | chr7 | GACCATGCTATTTCTTTGGCACA (SEQ ID NO: 375) |
| | | CTATGGATATGTGTCTGGAGGTTCC (SEQ ID NO: 376) |
| rs362726 | chr7 | CCAGGAAGGACATGAAGTCCAAC (SEQ ID NO: 377) |
| | | TTTCCTAGAGGGGAAGGAACAGT (SEQ ID NO: 378) |
| rs607755 | chr7 | TTGCAGTTGGTGGTAGGAGTCAAA (SEQ ID NO: 379) |
| | | AAACACAAGTGCCAACATGCTGT (SEQ ID NO: 380) |
| rs2520272 | chr7 | AGTCACACTGGTAAGTATGTACTTCTTCATG (SEQ ID NO: 381) |
| | | CTGTAAGTGTAGTTCTCTGTCCAACTG (SEQ ID NO: 382) |
| rs213950 | chr7 | GCAAGTGAATCCTGAGCGTGAT (SEQ ID NO: 383) |
| | | AGAACTGGAGCCTTCAGAGGGTA (SEQ ID NO: 384) |
| rs3815221 | chr7 | GCTTGACATCTGGGGTTTTAGTTGC (SEQ ID NO: 385) |
| | | AAATCGATTTTGGTGTAAGCGTGAAG (SEQ ID NO: 386) |
| rs3779032 | chr7 | TGACGTAGACCACAGGAACATGGC (SEQ ID NO: 387) |
| | | TCGCATTATAGTCCCTGTCCCTA (SEQ ID NO: 388) |
| rs7830 | chr7 | GCGGCTGCATGACATTGAGAG (SEQ ID NO: 389) |
| | | AGAGAGGGCAGGAAACAAAGTCCA (SEQ ID NO: 390) |
| rs3802100 | chr7 | ACCACCGTGATTTGCCCATCCCC (SEQ ID NO: 391) |
| | | TGAAGTTCTAGGCGTGCAGAGACA (SEQ ID NO: 392) |
| rs1550697 | chr8 | GCCTCGGTTTACTCTTAAATGTAAATAGAAC (SEQ ID NO: 393) |
| | | GGCCTATGTTGAAGTGTGGTCATCCA (SEQ ID NO: 394) |
| rs3020221 | chr8 | CTTACAGTTTGATGTGGACATCATAGTC (SEQ ID NO: 395) |
| | | TGAATTATTCACCGTGGCAGTCAC (SEQ ID NO: 396) |
| rs2073574 | chr8 | AGCAAGCCCAAATTTCAGAAGTTC (SEQ ID NO: 397) |
| | | TGTCTTCTGCTACTATTGAAGTACAAATGG (SEQ ID NO: 398) |
| rs12675283 | chr8 | GCCAGGAAAGTTGCCAAGTAGGC (SEQ ID NO: 399) |
| | | AGCATGCTTTGAAATCTAGTTCTTGGACA (SEQ ID NO: 400) |
| rs2272730 | chr8 | GAGGCGCAGGTAACCTCTGGAG (SEQ ID NO: 401) |
| | | AATGTCACGGGGAAAGAGCTT (SEQ ID NO: 402) |
| rs2737335 | chr8 | TGCGCCCTTACGAAAAAGGTA (SEQ ID NO: 403) |
| | | ACAGAATCTCAGAGCCTCATCCTTC (SEQ ID NO: 404) |
| rs7842714 | chr8 | AAGTCCCAAAGGAAGCCAGTACCCC (SEQ ID NO: 405) |
| | | GGCCGAGGCGCTTACCTGGT (SEQ ID NO: 406) |
| rs3098659 | chr8 | CTGCTCCCGGTGATGCAAACCC (SEQ ID NO: 407) |
| | | GTGCGTCATGGGCAACATCC (SEQ ID NO: 408) |
| rs6511 | chr8 | GTGGTTCAGTGTCACAATGCATT (SEQ ID NO: 409) |
| | | AGGCAAAAGCAGCAGGTCACCAGA (SEQ ID NO: 410) |
| rs10093032 | chr8 | TTGCCCTGGGCACATTGTTC (SEQ ID NO: 411) |
| | | CATCCTCAACATGGAGAGCATGGT (SEQ ID NO: 412) |
| rs2261147 | chr8 | GAAAATAACTCTACAGGCCCATTGCCTC (SEQ ID NO: 413) |
| | | GGCAAGATGTGCAGTGCTGACTACG (SEQ ID NO: 414) |
| rs2069568 | chr8 | CCAGGACAACTGATTATTGTTGCATCCAA (SEQ ID NO: 415) |
| | | GAGCACTGAAGAGGTCTTTATGGGTAG (SEQ ID NO: 416) |
| rs2272653 | chr8 | CTTGCCTGGACATCAAACTCTTGC (SEQ ID NO: 417) |
| | | GGGACATATGGGCCTTTCTATGACCT (SEQ ID NO: 418) |
| rs11780911 | chr8 | GGTGAGGACATGGCCACACCCT (SEQ ID NO: 419) |
| | | GGGTGGCGGGGCTTCAGCT (SEQ ID NO: 420) |
| rs3817681 | chr8 | TTCTGACTCCGGCTCTGCATCC (SEQ ID NO: 421) |
| | | AAAAGAACTGGCTGTGACCTTTGC (SEQ ID NO: 422) |
| rs17855765 | chr8 | CTTGGGGCTCTCGAGCAGGG (SEQ ID NO: 423) |
| | | CGGGCGACGTACCTGGC (SEQ ID NO: 424) |
| rs4244610 | chr8 | GCCTGTCAGAGCTGATCACTGC (SEQ ID NO: 425) |
| | | CACACGGCCATAGAGGAAGTC (SEQ ID NO: 426) |
| rs4251691 | chr8 | CCACTGCAGCTGGCAGA (SEQ ID NO: 427) |
| | | CCAAGCACACAGCCAAAGG (SEQ ID NO: 428) |
| rs12683181 | chr9 | CCAGCCAACTTCTAGCAAATTCG (SEQ ID NO: 429) |
| | | GTTATGATTTGGGCTGTGCGTTG (SEQ ID NO: 430) |
| rs11243704 | chr9 | GAGCCATCATTGACTGGTCGTA (SEQ ID NO: 431) |
| | | TACGTATATGGCAACGTTCAGGT (SEQ ID NO: 432) |
| rs12358961 | chr10 | TTATAGAAGGCAGGGCAGACCT (SEQ ID NO: 433) |
| | | ACTGGCTGCATTGGACTTTGCA (SEQ ID NO: 434) |
| rs10828545 | chr10 | AGCCAGCTGACGTAATTCAGAG (SEQ ID NO: 435) |
| | | CTGCCTAAGTGGTTCTGGAACAG (SEQ ID NO: 436) |
| rs10491056 | chr10 | CTCTGAATGCAATGCCCTCAGAAAG (SEQ ID NO: 437) |
| | | TTCAGATGTCCTCTTGGCTCGA (SEQ ID NO: 438) |
| rs3817419 | chr10 | CTATGTGGGAGACATACTCATTGCTCTT (SEQ ID NO: 439) |
| | | GGGTGCATTCTGTTACTTCATACAC (SEQ ID NO: 440) |
| rs3740232 | chr10 | GAAACCACCAATGCTGTGGAGAG (SEQ ID NO: 441) |
| | | AGTAAGCCCCAAACAGAAGTCTGTC (SEQ ID NO: 442) |
| rs2435352 | chr10 | TGTGGTCTACCCAGTGTCTGTCT (SEQ ID NO: 443) |
| | | TCGCGCTTTCATTTGTCCTTCG (SEQ ID NO: 444) |
| rs7089209 | chr10 | ATCTCCAGAATGCGTCCTCCTTC (SEQ ID NO: 445) |
| | | ATTAATGATGCTAGCAGAGAAAGAGCA (SEQ ID NO: 446) |
| rs3812658 | chr10 | GTCTGGTACCACTGTGTCATTAGGAA (SEQ ID NO: 447) |
| | | CAACCAGAAGTCATCTCTAGCCCTT (SEQ ID NO: 448) |
| rs2105702 | chr10 | GGCCAACAAGTCCTGTTTACAAGATG (SEQ ID NO: 449) |
| | | ATACAGTATTCACATGGCAGGTACCT (SEQ ID NO: 450) |
| rs3750863 | chr10 | CCTCACAGCTCTGCAAAAGACTA (SEQ ID NO: 451) |
| | | CTTTCTTTGCGAACTTCCTCAAGTGA (SEQ ID NO: 452) |
| rs3758626 | chr10 | TCTTGTAGCTTTTCTGGGGCAAAG (SEQ ID NO: 453) |
| | | GGCTGGATCAGAGCTCTACAAAAGCA (SEQ ID NO: 454) |
| rs10999338 | chr10 | CCCAACCCACAGCACTTCC (SEQ ID NO: 455) |
| | | GTAGGCTGCGGATGATGTCTG (SEQ ID NO: 456) |
| rs780668 | chr10 | TGGTGATAACTGCACTGGTGAAG (SEQ ID NO: 457) |
| | | ACTGTCTTCAGCAGCAGCATCTAC (SEQ ID NO: 458) |
| rs7917781 | chr10 | CTGCTTTCTTCTCCTTGCCCTTTC (SEQ ID NO: 459) |
| | | ACGTGACCCACTCTTTTCCACA (SEQ ID NO: 460) |
| rs4747203 | chr10 | AAGGAAGGATCCATCACCTCCACCA (SEQ ID NO: 461) |
| | | CGAGCATAGTGGGGCACAAAAGG (SEQ ID NO: 462) |
| rs2131956 | chr10 | ATTTCTCATCCTTCCCGCCATCG (SEQ ID NO: 463) |
| | | ACGATTACGAACCTGAGCTGCTGT (SEQ ID NO: 464) |
| rs7074064 | chr10 | AGCAACCATTTTTGTGCCCATGTT (SEQ ID NO: 465) |
| | | TGGTACCGAGTGATCCGTCATACGA (SEQ ID NO: 466) |
| rs1903858 | chr10 | ACCACCTTTTATTACTCCAGCTATAGTG (SEQ ID NO: 467) |
| | | GATTGCTGCATATTTCAGATATTTCTTTCC (SEQ ID NO: 468) |
| rs1409332 | chr10 | CTATTTTGGAGAGGCACCACCTG (SEQ ID NO: 469) |
| | | AAAGGGTGGCCTTGAGTTACAG (SEQ ID NO: 470) |
| rs10786229 | chr10 | ACCAAACAATCCCATCCCCTTCCCC (SEQ ID NO: 471) |
| | | GCCTGGAAGTTGGTTGCATTGAC (SEQ ID NO: 472) |
| rs3740486 | chr10 | GTTGTGGTAGTTTGTGGGGAGAT (SEQ ID NO: 473) |
| | | TCGGGGTCTTTCGGAAGTTTGCAAC (SEQ ID NO: 474) |
| rs813944 | chr10 | CCTTCCACAAAAATGGCCAGTG (SEQ ID NO: 475) |
| | | TACCGGGAAGTCCTGATGTGA (SEQ ID NO: 476) |
| rs196294 | chr10 | GCTACAAACAATTTCTGTGACTTTCAGTC (SEQ ID NO: 477) |
| | | CTGTTTCCCAGCCTGAAAACAAACC (SEQ ID NO: 478) |
| rs2278202 | chr10 | GCATGTCTCACAAGACAACCAAGGA (SEQ ID NO: 479) |
| | | CCAACTGCTTGAACGTTGGTCTC (SEQ ID NO: 480) |
| rs2272599 | chr10 | TTCCCAGACGTGATCTCAGGAG (SEQ ID NO: 481) |
| | | TTCTTGTTGTTCCTGTGGGGGTAG (SEQ ID NO: 482) |
| rs7942159 | chr11 | CAACACCAGCATCCAGTTCAAC (SEQ ID NO: 483) |
| | | ACCAAGGGAGAACACTGATCCT (SEQ ID NO: 484) |
| rs3758680 | chr11 | CATGGAGATTCCAGTGGTAAGCAT (SEQ ID NO: 485) |
| | | AGTAGGATGGAATGGTTTCCCGAA (SEQ ID NO: 486) |
| rs1799937 | chr11 | GACCGGGCAAACTTTTTCTGACA (SEQ ID NO: 487) |
| | | AACTATCAAGGCCCGAGTGAAG (SEQ ID NO: 488) |
| rs11037882 | chr11 | GGAGGTTTGGGATGTTGTTTCTGCT (SEQ ID NO: 489) |
| | | GAGCATCTGTGGTTGTACCAGA (SEQ ID NO: 490) |
| rs3740878 | chr11 | GCAGTGTGGTGTCACAAGCATGA (SEQ ID NO: 491) |
| | | ATTCAAGTGTCCTGAGTGCACAG (SEQ ID NO: 492) |
| rs3729802 | chr11 | ATACACCCCAAGGTGGAGAGAAA (SEQ ID NO: 493) |
| | | GCGCCTAAAGTTCCCTACCTT (SEQ ID NO: 494) |
| rs589691 | chr11 | TTAACCTCTCTGAGCCTCAGCAT (SEQ ID NO: 495) |
| | | TTCATCCTCACCTGTGTGTCCA (SEQ ID NO: 496) |
| rs630394 | chr11 | TGTCTGATGACGGAGGGTGAGG (SEQ ID NO: 497) |
| | | CTTCCTGGACTCACCCACACA (SEQ ID NO: 498) |
| rs736894 | chr11 | TCAGTGCTCAGGGCTTTACAACC (SEQ ID NO: 499) |
| | | AAGAAGTAGCCCTTGACCATGG (SEQ ID NO: 500) |
| rs11604536 | chr11 | ACGACGGTGAGTTCTGGTGA (SEQ ID NO: 501) |
| | | ATGCCCCATCATCAATTTGCTGTG (SEQ ID NO: 502) |
| rs1109977 | chr11 | GAAGGTAGAAGGGCTGAGAGGAGT (SEQ ID NO: 503) |
| | | GCCCTTCCTTCATTGGACATCAGCT (SEQ ID NO: 504) |
| rs883223 | chr11 | ATGGCAGAGCTGGCTTTTCCCTC (SEQ ID NO: 505) |
| | | CCCCCAGGATATCCTCACCACTCA (SEQ ID NO: 506) |
| rs2196168 | chr11 | GGCTATACCTGTAAATGGTGGTCTA (SEQ ID NO: 507) |
| | | AGACCAAAGGCAATGTTACCCTCTTG (SEQ ID NO: 508) |
| rs641936 | chr11 | CAGTTTTTAATAAAGATTCCTTCACAAATCCTAT (SEQ ID NO: 509) |
| | | ATAATGTCTTTTGGATTAGCTACCCGAT (SEQ ID NO: 510) |
| rs1784424 | chr11 | AAAGGATGAGCTGGAGTCACAGAGG (SEQ ID NO: 511) |
| | | AACGGGAATTTGGAGTTCACACAC (SEQ ID NO: 512) |
| rs591058 | chr11 | AGGGAGTGGCCAATTTCATGAGCAG (SEQ ID NO: 513) |
| | | CAGGTCTGCTGTGCATTGTATTGG (SEQ ID NO: 514) |
| rs10895417 | chr11 | CTCTCAGCAAAGTCATCAGAGGAA (SEQ ID NO: 515) |
| | | TGTTATAAGTGACTCCTCCATCACAC (SEQ ID NO: 516) |
| rs693147 | chr11 | AGATGGTTCTGACCCACTTCACCCC (SEQ ID NO: 517) |
| | | AGCAGCACCTGTTGAGAGAAGTGA (SEQ ID NO: 518) |
| rs2276424 | chr11 | TAGAAGCCACCAGTCTCAGGTTCAC (SEQ ID NO: 519) |
| | | CAGGAGGCAGGGTTAGAACTCTTCAAG (SEQ ID NO: 520) |
| rs1954787 | chr11 | AAAGGACCGAAGTAGGGTGGTG (SEQ ID NO: 521) |
| | | GCGGAATTGGGTGAAGGCACGAT (SEQ ID NO: 522) |
| rs2241501 | chr11 | GCTCCTCGTCTTCATGTTTCCA (SEQ ID NO: 523) |
| | | GCAAGCTACCCTCATTCGTGT (SEQ ID NO: 524) |
| rs880054 | chr12 | TTCTCTCCTGCTCTCCTTTCCAT (SEQ ID NO: 525) |
| | | AATCTTGCTTTTGGCAGCCTTG (SEQ ID NO: 526) |
| rs10848683 | chr12 | GTCCAGAGCTAAAGATGACCTGACC (SEQ ID NO: 527) |
| | | TCAGGAAAGCAAACCCCTCTAGGT (SEQ ID NO: 528) |
| rs2267549 | chr12 | TCGCCTTTGAAATACAAGTCATGTCTT (SEQ ID NO: 529) |
| | | AGTAGTGAGAGATGCATTTCCGGAAG (SEQ ID NO: 530) |
| rs216868 | chr12 | GACACTCTTTTGCACTCCTCCT (SEQ ID NO: 531) |
| | | TGCTATAGGGTGCCAGGGAGCTT (SEQ ID NO: 532) |
| rs12426675 | chr12 | CTCACCTCTTTTTCAGGTGTCGA (SEQ ID NO: 533) |
| | | GGGACTTGTGGTGACATGCCTCAG (SEQ ID NO: 534) |
| rs2518144 | chr12 | CAGGTGGCTCTAAAAGCACTCAC (SEQ ID NO: 535) |
| | | TCTTCACTACGTAGCACAGGTAGGT (SEQ ID NO: 536) |
| rs3759236 | chr12 | CAGGAGGCTTTACTTTCTGAGGA (SEQ ID NO: 537) |
| | | CCTTTTCTACATTTTCATTTCAGGTGAC (SEQ ID NO: 538) |
| rs10772008 | chr12 | CCGAAGCCCTCTGTAATTTCTGA (SEQ ID NO: 539) |
| | | TAGGAAATCAGGCCCAATACTCAC (SEQ ID NO: 540) |
| rs7955902 | chr12 | AAGAACAAGCACGTGCAGGTAGGAC (SEQ ID NO: 541) |
| | | GCATGCTGGGCACTAAATAATCTCCT (SEQ ID NO: 542) |
| rs2276456 | chr12 | ACCCTGAGGGAAGAGAAAACCAGCC (SEQ ID NO: 543) |
| | | CTGCCCAAGAGGAATTTGCTGTGGT (SEQ ID NO: 544) |
| rs1034762 | chr12 | CTTGAAATCCTTGAGGCCCCTAA (SEQ ID NO: 545) |
| | | CAGATACTTACAGGAGCACCTGCA (SEQ ID NO: 546) |
| rs3803184 | chr12 | TGAGGGACGCATGGAAAGCAG (SEQ ID NO: 547) |
| | | CCCGAGGCGAATCATGGCTCA (SEQ ID NO: 548) |
| rs4075913 | chr12 | AAGGGGCTTGGTGTTAAGGTAC (SEQ ID NO: 549) |
| | | AGAATGGCCATTGTTGGGACA (SEQ ID NO: 550) |
| rs2071218 | chr12 | CCGAGTTCGTCAACCACTACTGCT (SEQ ID NO: 551) |
| | | CTTCCCTCCTTTCCTCTCATGC (SEQ ID NO: 552) |
| rs706815 | chr12 | GGACATGAAGAAGGTGGTGTGTG (SEQ ID NO: 553) |
| | | GGGATTTCTGGGCCCAGGAACTTG (SEQ ID NO: 554) |
| rs2270267 | chr12 | CTGCACTGGGAAGACTTTTCCA (SEQ ID NO: 555) |
| | | CATTCTCCACCTCGGCTGTCA (SEQ ID NO: 556) |
| rs7397862 | chr12 | CTTTGGGTGTGGAAGGTGTCAT (SEQ ID NO: 557) |
| | | ATATCTGAAATGACGAGGGGGTCT (SEQ ID NO: 558) |
| rs2302629 | chr12 | AGGTGGAACGTGAACATATGTGT (SEQ ID NO: 559) |
| | | GAGACATTATGCAATTAAATGCAGGGAT (SEQ ID NO: 560) |
| rs6606734 | chr12 | TGGTGATTATGGGGAAGTGATTCC (SEQ ID NO: 561) |
| | | GAGCATGCCAAGGTTGATTAGCAGTG (SEQ ID NO: 562) |
| rs2464195 | chr12 | ACCTGTGCAGAGCCATGTGAC (SEQ ID NO: 563) |
| | | TGGCTGTCAATGGATGCAGGGGAA (SEQ ID NO: 564) |
| rs3741591 | chr12 | GATGGCCATGGCACTTACCA (SEQ ID NO: 565) |
| | | TGGCCTACCTGGGGGATG (SEQ ID NO: 566) |
| rs2075784 | chr12 | TCAGGCGCGGCGAGTGC (SEQ ID NO: 567) |
| | | CACCTGCTGGCCTCGCCATC (SEQ ID NO: 568) |
| rs9564692 | chr13 | AAGGTGCATGGACCATACTTCCGTT (SEQ ID NO: 569) |
| | | GAGGGTGTGGTTTTCCTCAGTCAGT (SEQ ID NO: 570) |
| rs3752451 | chr13 | GTCTTTGATAAGTGTGTTAGTAACTGACAAT (SEQ ID NO: 571) |
| | | TTTAGTACGATGTGATTAACGTAGAATAGCT (SEQ ID NO: 572) |
| rs9534342 | chr13 | TGACTGTTATGTGCTAGACAGTGTT (SEQ ID NO: 573) |
| | | CCTCATAGAGCTTACATTATGAGGTTGG (SEQ ID NO: 574) |
| rs2296533 | chr13 | GCCAGCAGAGACTACACCAA (SEQ ID NO: 575) |
| | | CCTCCCGCTCCATGTTCGT (SEQ ID NO: 576) |
| rs6313 | chr13 | CGATTTTCAGAGTCGACTGTCCAG (SEQ ID NO: 577) |
| | | TAGGGAGTTCGTAGTTGAGCTCA (SEQ ID NO: 578) |
| rs9518061 | chr13 | GCCTGTGCATTTTATATCAAAGGGAAATT (SEQ ID NO: 579) |
| | | TGGGAGCTCTCAGTAAAATTGCA (SEQ ID NO: 580) |
| rs661585 | chr13 | GGATTCCAGGACCCTAAAGAACTCTGC (SEQ ID NO: 581) |
| | | GTCCTCAATTGCTGAGCGTTTGG (SEQ ID NO: 582) |
| rs589985 | chr13 | ACCCTTAGAGCCTGTGATTCCTG (SEQ ID NO: 583) |
| | | CCCCTCAATGTTCTGGAAAGCA (SEQ ID NO: 584) |
| rs482757 | chr13 | CCTGAAACCGAAGAGAGAAGCAGT (SEQ ID NO: 585) |
| | | ACACTTACGGGACTCCCTTTTTCC (SEQ ID NO: 586) |
| rs3803236 | chr13 | TGGGAAAGGAAACAGGGAAGTCGA (SEQ ID NO: 587) |
| | | GAATACCTGGAACGAAGGGTACTC (SEQ ID NO: 588) |
| rs2281974 | chr13 | CTCCAGGACTGCATGGATTCCCAG (SEQ ID NO: 589) |
| | | TAAACGAGTGGGGTCCTCACT (SEQ ID NO: 590) |
| rs178642 | chr14 | CCAGCTCATGCACATTCTTTCCTCC (SEQ ID NO: 591) |
| | | CACCAACTCATCTCTGGCCTCT (SEQ ID NO: 592) |
| rs2073349 | chr14 | TTCTCCAGCATCTCTCTCTCCT (SEQ ID NO: 593) |
| | | ATTCCCACTGGGATTTCTCCTCA (SEQ ID NO: 594) |
| rs1152590 | chr14 | CTCTCACGAAGAGGACGAGGA (SEQ ID NO: 595) |
| | | CTTAGCAACACGGCCAGCT (SEQ ID NO: 596) |
| rs229581 | chr14 | CCAAACCATGCCTTTGCTGAAG (SEQ ID NO: 597) |
| | | CGCAGGTCCTTGTAGAGATCGGTGA (SEQ ID NO: 598) |
| rs3759753 | chr14 | CAAAATGTCTTTTGGTCAGCAATAGCTTA (SEQ ID NO: 599) |
| | | GAAGACCGCAGTGGGATAAATCTC (SEQ ID NO: 600) |
| rs2075179 | chr14 | CTCTCTGTTGGTTGTAGGAAGCTGTA (SEQ ID NO: 601) |
| | | TGTGTAAGTCAAGGGTAGCCATG (SEQ ID NO: 602) |
| rs12432149 | chr14 | GTCAGGGGTCTCTATGTATACATCACAC (SEQ ID NO: 603) |
| | | TGTCATTTCACAGCACATGCTTCCA (SEQ ID NO: 604) |
| rs3742656 | chr14 | CCACCTCCACACATTTCTGTCTTT (SEQ ID NO: 605) |
| | | CTCACAGTGGTTGTCCAGCTT (SEQ ID NO: 606) |
| rs3751582 | chr15 | GTACAAGAAGGGTGTGTGGCTT (SEQ ID NO: 607) |
| | | CCGGTTGCTTTCGCTCTTTGAACG (SEQ ID NO: 608) |
| rs1800419 | chr15 | AGTCAGCAGCCCCTTACCTCC (SEQ ID NO: 609) |
| | | TTGATGCCACGTCCCATGG (SEQ ID NO: 610) |
| rs1800404 | chr15 | TCTTTGAGCTGACATCCCACTGAG (SEQ ID NO: 611) |
| | | GCACGATCTGGAAAGAAGCACAGGA (SEQ ID NO: 612) |
| rs173224 | chr15 | GCTTGCAGTTCGTTCATCCATC (SEQ ID NO: 613) |
| | | AACAACGTAGTCCACCAGATTAATCC (SEQ ID NO: 614) |
| rs422388 | chr15 | TCGGTGTCGAGAGTATTTGTCATCTGG (SEQ ID NO: 615) |
| | | GTATGTCAGTACAGGAGTGATGAAATGG (SEQ ID NO: 616) |
| rs2279580 | chr15 | GTCTTTCAGTCCTCTTGATCATACCACC (SEQ ID NO: 617) |
| | | CTCTCTCCAGATACTGCATAGAACGT (SEQ ID NO: 618) |
| rs1801376 | chr15 | GTGCTTCCCAGTTTCACTCCAT (SEQ ID NO: 619) |
| | | GGCACCTATTCTACTTCCCAAAGG (SEQ ID NO: 620) |
| rs140605 | chr15 | GGCAAATATCAGGATCTAGTGCACATT (SEQ ID NO: 621) |
| | | AAATGTAATGACCTTAGGAGCTACAGGA (SEQ ID NO: 622) |
| rs2306187 | chr15 | AGAGGGGACATTAAGTCAGACGAT (SEQ ID NO: 623) |
| | | ACCAGCTTCTGCTTCTGTTGC (SEQ ID NO: 624) |
| rs58156069 | chr15 | GGCTGAGAGTCAAACCATCCTTTTC (SEQ ID NO: 625) |
| | | ATCCCAATCAGATGTCACGTCTGT (SEQ ID NO: 626) |
| rs1147129 | chr15 | TCCAGCAGGAACTGAGTAGTCT (SEQ ID NO: 627) |
| | | GGCATCTAACTGATGATCGGTTGC (SEQ ID NO: 628) |
| rs762551 | chr15 | GCGTTCATGTTGGGAATCTTGAGG (SEQ ID NO: 629) |
| | | GGAGCTTAGTCTTTCTGGTATCCAGCT (SEQ ID NO: 630) |
| rs1370275 | chr15 | TTCAGTCCGCTCTGCATACAGGGC (SEQ ID NO: 631) |
| | | CTAGCCAAGACCGAGGATAGGT (SEQ ID NO: 632) |
| rs9806604 | chr15 | TTTAGGCAGCCTGTCATCCTCTC (SEQ ID NO: 633) |
| | | TGCGTATCATTCATGTGCATCGATG (SEQ ID NO: 634) |
| rs2307438 | chr15 | GGCAAACATGCACCTGAAAGAG (SEQ ID NO: 635) |
| | | AACACCCCATCTGTTCACTTAGC (SEQ ID NO: 636) |
| rs9672296 | chr15 | TCTTCTCGGACAGCTCCTTCTCC (SEQ ID NO: 637) |
| | | GGACAGAAAGGGCCGGATGTTAAGG (SEQ ID NO: 638) |
| rs3815003 | chr15 | TTTCCTTCTGTTCCGGTGATGG (SEQ ID NO: 639) |
| | | TATTTGAGAACCCTGGGGCAGT (SEQ ID NO: 640) |
| rs2272037 | chr15 | GCCTCTGGGGAAGATTTTTGAGG (SEQ ID NO: 641) |
| | | CACAGAGTATGATGGGCAGGATG (SEQ ID NO: 642) |
| rs7496614 | chr15 | GTCTTCCTCACTTGAGGAGATGAGG (SEQ ID NO: 643) |
| | | TTCATGACGGTCAGGATGAACC (SEQ ID NO: 644) |
| rs224204 | chr16 | AGGAGGAGTCTGGAATCACAGA (SEQ ID NO: 645) |
| | | CTGTACCTGTGCAAGATGTCTCCA (SEQ ID NO: 646) |
| rs1641010 | chr16 | AGCTATTTTGGCAAGAGTGTGGACTT (SEQ ID NO: 647) |
| | | AATTCAGGTAAGTGAGGAGGAGGT (SEQ ID NO: 648) |
| rs2280764 | chr16 | TCTTGTCCAGGGCTTTGTTCAC (SEQ ID NO: 649) |
| | | CCGGTTATTCTCAATGGGCTCCA (SEQ ID NO: 650) |
| rs3115446 | chr16 | GCTCTTCCCTTCACATACCAGT (SEQ ID NO: 651) |
| | | CCATGGTCAATCCACTGCCTCAG (SEQ ID NO: 652) |
| rs215901 | chr16 | CTGATAGGGCCTCACAGTGTTGGG (SEQ ID NO: 653) |
| | | AAGGCCTGAGGAGCTTTTGAGGTAG (SEQ ID NO: 654) |
| rs2072061 | chr16 | AAAGGCCTCCTCCACAAGGACC (SEQ ID NO: 655) |
| | | CCAACCCACCCAAACCGTCTCT (SEQ ID NO: 656) |
| rs249954 | chr16 | CCAATCCAAATCTGTTTTTCTGAATCTGTTT (SEQ ID NO: 657) |
| | | ACACTGGAAGAGAATATTCTTCTGACCT (SEQ ID NO: 658) |
| rs7203525 | chr16 | CTATCTTCAGGAGGAGGAGAAGTCTCCT (SEQ ID NO: 659) |
| | | AAATTGGCAGCAAATGTAACAGTTACTC (SEQ ID NO: 660) |
| rs243834 | chr16 | TCCTTTAGAGAGGCCCTGCTG (SEQ ID NO: 661) |
| | | AAGCTCATCGCAGATGCCTGGAA (SEQ ID NO: 662) |
| rs2304483 | chr16 | TGGCCTAGAAAGAGGCTCGAC (SEQ ID NO: 663) |
| | | TGGGGCTATCATCTCCGTGGTCATC (SEQ ID NO: 664) |
| rs1532625 | chr16 | TGGCCGATTTTGTCCAGACAAG (SEQ ID NO: 665) |
| | | GGGACTGTCACTCTTCCTGTCTAGGT (SEQ ID NO: 666) |
| rs11076207 | chr16 | TAAATGTGTGGCCCACTCTTGG (SEQ ID NO: 667) |
| | | TACCTGTTCCACCAACTCTTGCT (SEQ ID NO: 668) |
| rs483053 | chr16 | CCCAGCAGTCCTACCTTTAAACAGC (SEQ ID NO: 669) |
| | | CCATGGGGGTAAAACGGATGACT (SEQ ID NO: 670) |
| rs3991715 | chr16 | CACGAAGAACAGCCATAGGACA (SEQ ID NO: 671) |
| | | GCTCTTCACCAGTTGGATCAGGTAG (SEQ ID NO: 672) |
| rs2548861 | chr16 | CGGTTTGCTTTAGAGTGCATTAACTTCTTT (SEQ ID NO: 673) |
| | | AATAGCTTCTGTGTGTTTCCTATTTTTGG (SEQ ID NO: 674) |
| rs12918876 | chr16 | TTCCAGTGCCTGGAGGAGACCCC (SEQ ID NO: 675) |
| | | ACACACCACTTCTCCCTCAGCA (SEQ ID NO: 676) |
| rs3803679 | chr16 | GCGACGTGGTGGAAGTCTACCTG (SEQ ID NO: 677) |
| | | CTGTGGGCTCCTGTGAATGAG (SEQ ID NO: 678) |
| rs3752827 | chr17 | CAGTACATCCAGAATGTCACAACAGATTA (SEQ ID NO: 679) |
| | | TATCTCTGGTTTTGAGGTAGAGAGCT (SEQ ID NO: 680) |
| rs238238 | chr17 | GCCCAGTTGATTGAGCTCCAAA (SEQ ID NO: 681) |
| | | CCTGCTCTGCTGCAAAAGGCAAG (SEQ ID NO: 682) |
| rs12453262 | chr17 | CTCCGGGATAGGATGGGGTAGACC (SEQ ID NO: 683) |
| | | CCTGCTCCCTCACTATGCCACT (SEQ ID NO: 684) |
| rs17671352 | chr17 | AGTCTCAGCGGACTTGTCCA (SEQ ID NO: 685) |
| | | TCTTAAGGCAGTACGGGCTCT (SEQ ID NO: 686) |
| rs1565817 | chr17 | CTTTGTTCCCAGCCTGACACTGC (SEQ ID NO: 687) |
| | | ACACAGAAGCCTTCATGGGCATC (SEQ ID NO: 688) |
| rs2304908 | chr17 | GTTGGGGGAGAGGGAAAGTTCTT (SEQ ID NO: 689) |
| | | ACGAAGAAGGAGGCCGTCTTGAG (SEQ ID NO: 690) |
| rs876660 | chr17 | GAATTGTCCCTCAGGGATTGCACT (SEQ ID NO: 691) |
| | | ACATGTAGTTCAGGCCTCTCTTCCT (SEQ ID NO: 692) |
| rs3744586 | chr17 | CTCCAGTCACAACTCCTGATAAGTCC (SEQ ID NO: 693) |
| | | TGCAGACAAACTGATTCAGGTCGT (SEQ ID NO: 694) |
| rs1736219 | chr17 | GCTGCTCATCTCCGAAGAAGATGGG (SEQ ID NO: 695) |
| | | AGACTTTTCAGAGTCAGCTGGCACA (SEQ ID NO: 696) |
| rs548973 | chr17 | GCCTCTCTTCCCAAAACCCATCT (SEQ ID NO: 697) |
| | | GGGAACACTGAGGCATGGAATCCT (SEQ ID NO: 698) |
| rs2952976 | chr17 | TGGAAAAATGTCTTGCTGGGGTA (SEQ ID NO: 699) |
| | | AAGTACAGATGTGGACCAAGAGGA (SEQ ID NO: 700) |
| rs9894648 | chr17 | AGAGGACCTGAAGGTATTCCACAATG (SEQ ID NO: 701) |
| | | TAACTAGACTATATCCTGGCCTCCCTAG (SEQ ID NO: 702) |
| rs3110641 | chr17 | GTGGAAAACAGGGTCCTTGTTG (SEQ ID NO: 703) |
| | | GTCATGGGCCATTAGTTTGGCTTC (SEQ ID NO: 704) |
| rs8067890 | chr17 | GATGTTCTCCACCGACGAGTACAG (SEQ ID NO: 705) |
| | | ACCTCCACCCTGTAGCAATTCCAT (SEQ ID NO: 706) |
| rs8176161 | chr17 | AGAGCCTGGTTCTATGATGAAGATG (SEQ ID NO: 707) |
| | | TTACATTATCAAATGGTCATATATATGGTAAAGCTT (SEQ ID NO: 708) |
| rs850730 | chr17 | CTCACCAACATCGCGATTCCTATC (SEQ ID NO: 709) |
| | | AGTCTTCACCCTCCAAGATCACCA (SEQ ID NO: 710) |
| rs2289677 | chr17 | AGGGCTAAAACATGAGCAGGTACA (SEQ ID NO: 711) |
| | | AAAGGGTCACATTTCTGATGAGCTCT (SEQ ID NO: 712) |
| rs2258689 | chr17 | TTAGCCCCAAACACCCCACTC (SEQ ID NO: 713) |
| | | CTGTCACTTCCCGAACTGGCAGT (SEQ ID NO: 714) |
| rs3809863 | chr17 | CCAGAGCAAAATGGGACACAGTA (SEQ ID NO: 715) |
| | | GTCATGTTCAGCCAGTACCATCGT (SEQ ID NO: 716) |
| rs2256835 | chr17 | AATTCGAGGGCAGGAGATTACC (SEQ ID NO: 717) |
| | | GCCAAGGTTTGCTAATGCTGCT (SEQ ID NO: 718) |
| rs3744093 | chr17 | CAACACCAGCAAACACACCTTCCAA (SEQ ID NO: 719) |
| | | CAGTCCTGTGCGTCCAAAGC (SEQ ID NO: 720) |
| rs758596 | chr17 | CCCTGGAGTAATCACCTGCTCT (SEQ ID NO: 721) |
| | | GCTGTATGTCCACCCGGATTCTCCT (SEQ ID NO: 722) |
| rs4342 | chr17 | AGACCTGCTGCCTATACAGTCAC (SEQ ID NO: 723) |
| | | CAAACCCCTACCAGATCTGACGA (SEQ ID NO: 724) |
| rs4362 | chr17 | TGGCACAAGGCCCTCAACC (SEQ ID NO: 725) |
| | | AGTTCCACGAGGCACTGTG (SEQ ID NO: 726) |
| rs3744024 | chr17 | AGGACTCCATCCCTGGGGATTG (SEQ ID NO: 727) |
| | | CCCACCTCTCTGAGGAGCTCATGC (SEQ ID NO: 728) |
| rs11077379 | chr17 | CTCCCTCATCTCTATGAGGTCTGTGTT (SEQ ID NO: 729) |
| | | GGCCTTGAGAAGTGGTTCTCTGT (SEQ ID NO: 730) |
| rs3816256 | chr17 | ATGGTCCCACATCCATGTGTGG (SEQ ID NO: 731) |
| | | AGATCCAGCTAACAGGCGCTA (SEQ ID NO: 732) |
| rs3213926 | chr18 | TCCAGTCTTTGCTCGAGACTTG (SEQ ID NO: 733) |
| | | TTTACTTTTGTACAGATGTTAAATTTGTGACAC (SEQ ID NO: 734) |
| rs7232329 | chr18 | CCATCCCAAGGTCTTTGAAGGT (SEQ ID NO: 735) |
| | | TTCATCTGGACACCTCCTCTCA (SEQ ID NO: 736) |
| rs3748422 | chr18 | CCATAAGTGACTGTGACGTTGCA (SEQ ID NO: 737) |
| | | AAGCAGACAACCCGAAGTAAACAC (SEQ ID NO: 738) |
| rs8097342 | chr18 | AGGGACAGAACACAGTGAACC (SEQ ID NO: 739) |
| | | CAACAGGCACCCGATTTTCTC (SEQ ID NO: 740) |
| rs2510344 | chr18 | GCCTCAGGATAGAATTCCCTTTCAGT (SEQ ID NO: 741) |
| | | GACCATGGCCAAATACATCCTGA (SEQ ID NO: 742) |
| rs12960081 | chr18 | GAAGAAATGGCAGCATCTGAACCCGT (SEQ ID NO: 743) |
| | | CCCAATGTTGTAGTAACCGAAGC (SEQ ID NO: 744) |
| rs1058396 | chr18 | CATCCTGCCTTTAGTCCTGAGTTCT (SEQ ID NO: 745) |
| | | GCAATGGGAGGAATGTTCATGG (SEQ ID NO: 746) |
| rs1450425 | chr18 | TCAGCTAGGAGAGGGGAATAAGGCAC (SEQ ID NO: 747) |
| | | CAAGAAGAGCTGGCACCTAGATC (SEQ ID NO: 748) |
| rs1788027 | chr18 | GCATGGAAAGACAGAGGACGAG (SEQ ID NO: 749) |
| | | TTCATGTGGATGCAGGCTACAG (SEQ ID NO: 750) |
| rs2298712 | chr18 | GCAGTAGAGTATGAGTTCTTCCTTTCCTA (SEQ ID NO: 751) |
| | | TCCCTCTTTACTATGTGCAGGTGCTCT (SEQ ID NO: 752) |
| rs1893788 | chr18 | TTCTGTTGGTGATCACCCTCCT (SEQ ID NO: 753) |
| | | CACCAGTGATATACTTGCCCAGGTCAG (SEQ ID NO: 754) |
| rs6567271 | chr18 | GGCAATCTGGGAGAGTTTTGCA (SEQ ID NO: 755) |
| | | GGACAGAGAAATCCGATGCGGTT (SEQ ID NO: 756) |
| rs1629038 | chr19 | CCTGGAGGACGCGTGAGTGC (SEQ ID NO: 757) |
| | | CGACGGGAAGGTGCAGGTTC (SEQ ID NO: 758) |
| rs3764640 | chr19 | AGGGCCGTCAAGATCCTCA (SEQ ID NO: 759) |
| | | CACGGTGCTGATGGTTCTGTC (SEQ ID NO: 760) |
| rs2277984 | chr19 | ATGGTAGAACCGGGTACAGCTTTCC (SEQ ID NO: 761) |
| | | AGATGCTAGGTCTTCAACCCCTCA (SEQ ID NO: 762) |
| rs2860178 | chr19 | CACCAGTCCATGGAAAAACCATCTTC (SEQ ID NO: 763) |
| | | CATTGGTCTTCAGGGCAATGTC (SEQ ID NO: 764) |
| rs620744 | chr19 | TATGAGCGTGGCCGGATCTCC (SEQ ID NO: 765) |
| | | GAGCATCAGGAGGTCACAAGC (SEQ ID NO: 766) |
| rs539887 | chr19 | TGTGGCCAGGTGGTGGTGG (SEQ ID NO: 767) |
| | | CCTTAAGCAGCTAGTCCTGCTCCA (SEQ ID NO: 768) |
| rs8104339 | chr19 | TGGTGGGACCAGAGAACCAG (SEQ ID NO: 769) |
| | | TCAAGAAGGATGGGGAGTGGAAG (SEQ ID NO: 770) |
| rs2114724 | chr19 | GACAGAGGATGTGGGCCATGCT (SEQ ID NO: 771) |
| | | GAAGATGGGCTGCTCATCACTG (SEQ ID NO: 772) |
| rs280520 | chr19 | GGAACTTTCGGAGCCGCAAG (SEQ ID NO: 773) |
| | | ACCAGGCAGGGAACGTGTCCA (SEQ ID NO: 774) |
| rs3826803 | chr19 | AAAGCATTTCTCCCCGCAGTC (SEQ ID NO: 775) |
| | | TGCCGAGTTTTTGCACTGCAAGTC (SEQ ID NO: 776) |
| rs2075021 | chr19 | CATGTTCCAGCACATCGAGGTGAG (SEQ ID NO: 777) |
| | | CTCGACAGCTCTTTAAAAACAGACTC (SEQ ID NO: 778) |
| rs2738447 | chr19 | CTCTCCAGGTGCTTTTCTGCTAGG (SEQ ID NO: 779) |
| | | ACCTCTCCTTATCCACTTGTGTGTC (SEQ ID NO: 780) |
| rs6413504 | chr19 | TGAGAAGTAGGTGGCCTCCA (SEQ ID NO: 781) |
| | | ACAGATGGTCAGTCTGGAGGATGAC (SEQ ID NO: 782) |
| rs2304154 | chr19 | TCTGAGGTTTGCTTCCAGTGTGT (SEQ ID NO: 783) |
| | | AAAACACAGGGCACACGTGA (SEQ ID NO: 784) |
| rs8113582 | chr19 | GGACAATGGGCAGATACACAGTAG (SEQ ID NO: 785) |
| | | CTTCTGTGGACAATGACCCAGTC (SEQ ID NO: 786) |
| rs1043996 | chr19 | ACTCACTGGGGTCACAGTCAT (SEQ ID NO: 787) |
| | | CTGCCAGGTTGGTGCAGATACCAT (SEQ ID NO: 788) |
| rs2302603 | chr19 | TATGATGCTGGGACCAGGTGA (SEQ ID NO: 789) |
| | | CGAGGGGCTGCAGCTTTTGTC (SEQ ID NO: 790) |
| rs10424582 | chr19 | TGACGTAGTTCACCAGCATGG (SEQ ID NO: 791) |
| | | CGGCTTCGTTAAAGAGCCTCT (SEQ ID NO: 792) |
| rs55742440 | chr19 | ACAGGACAGGCTGGCTCT (SEQ ID NO: 793) |
| | | AGCATGCCTGTCCCCCACAGA (SEQ ID NO: 794) |
| rs1469699 | chr19 | ATCCTCAACGAGTTCGCCAAGA (SEQ ID NO: 795) |
| | | GCCCAGCCCAATAAACCTGCACT (SEQ ID NO: 796) |
| rs3745859 | chr19 | CAGTGCTCACTGTCTTTGTGTG (SEQ ID NO: 797) |
| | | AGTCCTGGTCATCCTCTCCTT (SEQ ID NO: 798) |
| rs10880 | chr19 | TCCCTATCTATGCCAGCCTCAGT (SEQ ID NO: 799) |
| | | GGCACCTGCTAGCCCCGTTCTG (SEQ ID NO: 800) |
| rs2246095 | chr19 | TCAGTGTGATCTCCCTTGTGGTGCC (SEQ ID NO: 801) |
| | | TTCCGCAGGTCCTGGCTT (SEQ ID NO: 802) |
| rs1799783 | chr19 | CCCTCCTGCTTCTCATAGAAGTTG (SEQ ID NO: 803) |
| | | CAGTCTCTTCCCCAGCTTCCCAA (SEQ ID NO: 804) |
| rs392891 | chr19 | CCCAATAAACCCCGCACTGTGCC (SEQ ID NO: 805) |
| | | CGGCTGATGATGTCCGGGTACTTC (SEQ ID NO: 806) |
| rs20580 | chr19 | ACCAGAAACTCACTGGAGGTCTTTA (SEQ ID NO: 807) |
| | | CTGACGATAGACAGAACGGTCAGATG (SEQ ID NO: 808) |
| rs3730849 | chr19 | GAGGACTTGGAGAAGGAAAGACTCAG (SEQ ID NO: 809) |
| | | CAGCTGCTCCTGGAACAGAAATCCA (SEQ ID NO: 810) |
| rs2387588 | chr19 | CCACACCCCATTCCCCAAC (SEQ ID NO: 811) |
| | | CAGTAGCCGGCACAGATGGT (SEQ ID NO: 812) |
| rs4002462 | chr19 | CAGGATGGCATTGATGGGGT (SEQ ID NO: 813) |
| | | ACCGCAGCCCCGAGTCCTG (SEQ ID NO: 814) |
| rs1062798 | chr19 | GCTGCTGTATTGCTGGGGATC (SEQ ID NO: 815) |
| | | GGTGGATGTTGTTGTGGAGGT (SEQ ID NO: 816) |
| rs12981413 | chr19 | GTTCCTGCTGTGTGTCACTCTGA (SEQ ID NO: 817) |
| | | GATCCTGACACCCAATGCCATC (SEQ ID NO: 818) |
| rs3745405 | chr19 | TGACGAAACAGAAGACCCGAAC (SEQ ID NO: 819) |
| | | GGAAGGCAATGACAGCTGACAGAG (SEQ ID NO: 820) |
| rs269950 | chr19 | GCCTACCGTAGGTGTTTTAGGTTA (SEQ ID NO: 821) |
| | | CGCCTCTGAGAGATATCTACAGCCAAGC (SEQ ID NO: 822) |
| rs3729836 | chr19 | GCATAAGCGCGGTAGTTGGAGGA (SEQ ID NO: 823) |
| | | CCTTACCGTACCGCACCCTCT (SEQ ID NO: 824) |
| rs2076653 | chr20 | CAGAATGGCAAGAGGACTGACAGTC (SEQ ID NO: 825) |
| | | GTTTGACATCACCCCCTCCAA (SEQ ID NO: 826) |
| rs2073193 | chr20 | TCACCTTGAACACCTCCTTGACG (SEQ ID NO: 827) |
| | | GGGGGAAGAGGGGAGAAGTAAAGATAGAG (SEQ ID NO: 828) |
| rs2281575 | chr20 | GCGTCTCCTTCACGTTCACA (SEQ ID NO: 829) |
| | | CCCTATGTCCCATGTGGCCAGAG (SEQ ID NO: 830) |
| rs3803953 | chr20 | GTGGTGATGTATTTGCCCACAG (SEQ ID NO: 831) |
| | | ACGTACCATCAGTGAAGTCCAAG (SEQ ID NO: 832) |
| rs2295435 | chr20 | GCAATGCTTCTGTTCCCACCA (SEQ ID NO: 833) |
| | | AAATGAGAGAGGGCCCAGCTT (SEQ ID NO: 834) |
| rs2295179 | chr20 | TGCAGAAGAGGAACCTGTCATCAC (SEQ ID NO: 835) |
| | | CCTCCTAAGGTAGAAAGTCCCAGTG (SEQ ID NO: 836) |
| rs363050 | chr20 | TCTATACAGTGCTGGGAGGACTCT (SEQ ID NO: 837) |
| | | ATTCAGCAAGCCTGAGGAGTG (SEQ ID NO: 838) |
| rs2273060 | chr20 | ATCATGCCCGAGTGAGAAGCCTT (SEQ ID NO: 839) |
| | | CTTCTTCCCTGACCATTTTGGCTT (SEQ ID NO: 840) |
| rs2273061 | chr20 | CTACCCAGAGACAACCTGTTACC (SEQ ID NO: 841) |
| | | ATGTGTGCATGTGAGTCGTTTGG (SEQ ID NO: 842) |
| rs143383 | chr20 | CGGGTGTGTGTTTGTATCCAGTC (SEQ ID NO: 843) |
| | | CCTTTCTCCTGCACAACTGACT (SEQ ID NO: 844) |
| rs2072792 | chr20 | ACCCAGAGCTACCAATGACTTC (SEQ ID NO: 845) |
| | | GCACTCTAGGAAAAGTCCCACA (SEQ ID NO: 846) |
| rs4608591 | chr20 | AGCTGCCACAATGGGACATGTGC (SEQ ID NO: 847) |
| | | ATCGGCAGGTTTCTCAGGGAT (SEQ ID NO: 848) |
| rs2425906 | chr20 | GAAACAAAAGGCCAGTCGTTGGCA (SEQ ID NO: 849) |
| | | CCCAAGCACACAGCTTCAGGAT (SEQ ID NO: 850) |
| rs6021437 | chr20 | CCGAATATGTTGCTGCAGCATCA (SEQ ID NO: 851) |
| | | CCCCAAGGTGTGTCTTCAGGTT (SEQ ID NO: 852) |
| rs2234487 | chr20 | GTTTGCTCCAACTGACACTTCAG (SEQ ID NO: 853) |
| | | AGATATGGAATTGTGGGTGCATGC (SEQ ID NO: 854) |
| rs3746657 | chr20 | CCTGACATCCGCGGCATGG (SEQ ID NO: 855) |
| | | TAGGGTGGGAGAGCACAAAGC (SEQ ID NO: 856) |
| rs6062700 | chr20 | CTCACAGGGAGAGGCTGGT (SEQ ID NO: 857) |
| | | CTGGGTGGGTTGGTCACTG (SEQ ID NO: 858) |
| rs41309367 | chr20 | TTGCCCAGGGCTGAACATGG (SEQ ID NO: 859) |
| | | GAGGAGGTGGGTGACACCT (SEQ ID NO: 860) |
| rs2073421 | chr21 | TTCCTGCGGTGTCTGAAACATCA (SEQ ID NO: 861) |
| | | AGGAGGGACAGGGTTTGCAT (SEQ ID NO: 862) |
| rs2073425 | chr21 | GAGCTGGGAGGTAGTTCTAAGTGCAC (SEQ ID NO: 863) |
| | | AAATTTAGAGACAGGATCTCATTATGTTGC (SEQ ID NO: 864) |
| rs8130564 | chr21 | AGTCACCTTGTCATCTGGCAAGAGG (SEQ ID NO: 865) |
| | | CAGACCCGACGTGGTAACTTTACAC (SEQ ID NO: 866) |
| rs872331 | chr21 | TCCCCGTGGTACCAAAGCTGAA (SEQ ID NO: 867) |
| | | GTATGACCTGCTGCCCTTCCT (SEQ ID NO: 868) |
| rs754507 | chr21 | CAAAGTCTTCTCGGTGGCCATCA (SEQ ID NO: 869) |
| | | TGGGACCGTCTTTTGGTCCT (SEQ ID NO: 870) |
| rs62215498 | chr21 | CATGTGCCTGGGACATGTGTCC (SEQ ID NO: 871) |
| | | TTCCCACTGACTCGTCTCCAT (SEQ ID NO: 872) |
| rs2839077 | chr21 | TTGGTCTGAGACCCTCAGCTTA (SEQ ID NO: 873) |
| | | TGGCCCTATCGGACCTAAAGG (SEQ ID NO: 874) |
| rs7283989 | chr21 | TAGCTCCTGAGAGAATGGATCCCGGG (SEQ ID NO: 875) |
| | | CTCCAAGCCAGCAGGGTTC (SEQ ID NO: 876) |
| rs2236486 | chr21 | GCTGGAGGATGCCTTCCTGAAGA (SEQ ID NO: 877) |
| | | GCGTAGGTGCACGCGGGGC (SEQ ID NO: 878) |
| rs7279347 | chr21 | GTAAGTGGCCTTGTCAGGGTACGG (SEQ ID NO: 879) |
| | | AGAGGGCCTTTCTGGAAACACTG (SEQ ID NO: 880) |
| rs7279622 | chr21 | AGGCAGGGTGCGAATGGA (SEQ ID NO: 881) |
| | | CAGAGCTGTCCGTGGCACAGT (SEQ ID NO: 882) |
| rs9982233 | chr21 | TTGCCATTACTGACCTGGAGAG (SEQ ID NO: 883) |
| | | CTGTTATCCCCACAGGGCACAGT (SEQ ID NO: 884) |
| rs2839226 | chr21 | GGCATTTCCTGTGGGAAACTGACT (SEQ ID NO: 885) |
| | | ATCACCGAGAAATTCAGTGCGGAA (SEQ ID NO: 886) |
| rs2839257 | chr21 | TCTGGCCCACGTGGTCAGA (SEQ ID NO: 887) |
| | | GCAGAGCCAGCTGGAGGAGGA (SEQ ID NO: 888) |
| rs112544 | chr22 | AGGATGGTGGGGGTCTCTGG (SEQ ID NO: 889) |
| | | CTGACCTATGAGGAGCGGGTT (SEQ ID NO: 890) |
| rs2072708 | chr22 | GCTAACAAGCCCCTCTTCTCTC (SEQ ID NO: 891) |
| | | CTCTCACACTGTGGGCATCTG (SEQ ID NO: 892) |
| rs2235324 | chr22 | CACTCCAGCCGGAGTTTGAGCA (SEQ ID NO: 893) |
| | | CGTAGCTGTAGCGGTAACAACC (SEQ ID NO: 894) |
| rs5756516 | chr22 | AGAGGAGGGAAGTGCATCTCA (SEQ ID NO: 895) |
| | | CCCACTGGCTTCCCTATGGTCAGA (SEQ ID NO: 896) |
| rs1135360 | chr22 | CCAGGTTCAAGTATGTCCTGGTCA (SEQ ID NO: 897) |
| | | GCAGTGGTGGTGGTGATGCT (SEQ ID NO: 898) |
| rs743616 | chr22 | GGTTACCCTGGGTGAAGAAGTGAG (SEQ ID NO: 899) |
| | | GGACGGGTAGAAGAAGAGAGACTG (SEQ ID NO: 900) |
| rs6639398 | chrX | CTACAAACCTGTCCAGAAAGAGGT (SEQ ID NO: 901) |
| | | AGACTGGTATAAGCTGAAAAAGGACAG (SEQ ID NO: 902) |
| rs5980274 | chrX | CAGTAGCAAATAGAAGCCATCAAGG (SEQ ID NO: 903) |
| | | TCAGTGATAGCAGTCTCCAAAACAAG (SEQ ID NO: 904) |
| rs1458178 | chrX | CTTCCCTGTGGAAGTGATGCTG (SEQ ID NO: 905) |
| | | CACTGTGGCAAGAGGGAATGTC (SEQ ID NO: 906) |
| rs5986751 | chrX | AGTTCTCATGAGCCTTGCTCAC (SEQ ID NO: 907) |
| | | CCTGGATCACAACAGGCTTTCTGGT (SEQ ID NO: 908) |
| rs9781645 | chrX | GGGCATTCATTTTCAAAGGAGGTGAT (SEQ ID NO: 909) |
| | | CCTCTAAAGCTCAGCCTTACTTGACA (SEQ ID NO: 910) |
| rs2209420 | chrX | CCGGTAACCAATCTTTCTTGATTCTCCCT (SEQ ID NO: 911) |
| | | GCAATATTTGTCTTCCCATGCCTGAC (SEQ ID NO: 912) |
| rs5984589 | chrX | AGTTCCTGGTTTCATTTGTCACAACATT (SEQ ID NO: 913) |
| | | GGGGATTTCTGGCCAGTCATGAGT (SEQ ID NO: 914) |
| rs2808742 | chrX | GACTCTAAAACAGGGCTGAATCAAGCA (SEQ ID NO: 915) |
| | | CTTATGACCCATTACTTCCCAGCA (SEQ ID NO: 916) |
| rs5917032 | chrX | CTGGCCAGTTTTTGTAGAACATCCCT (SEQ ID NO: 917) |
| | | GGATAGCACAGTGGTGATATGCT (SEQ ID NO: 918) |
| rs6529455 | chrX | GTAAGACAGACGGGCAGATGTC (SEQ ID NO: 919) |
| | | CACTCTGGAGAGAACTGGGAGA (SEQ ID NO: 920) |
| rs5931302 | chrX | GGGACGGAAAAGTTGCATTCAGTG (SEQ ID NO: 921) |
| | | GCTGGTGGATGGATAGTTCCCTGA (SEQ ID NO: 922) |
| rs6643690 | chrX | CCTCAGTCACATTAGCCAAATTTCAAGGGT (SEQ ID NO: 923) |
| | | TGAACCTCATAAGACAGGGTCTGTTC (SEQ ID NO: 924) |
| rs1781104 | chrX | TGTCTTGTCCAATGCCGAGA (SEQ ID NO: 925) |
| | | GGCAAATTGACTGGAAGCCA (SEQ ID NO: 926) |
| rs1781116 | chrX | GCAAATATGTTGGCAAGACGTAT (SEQ ID NO: 927) |
| | | CCCTTCCCCACCTTGGATAG (SEQ ID NO: 928) |
| rs318173 | chrX | CATCGCCCAAGGAGTGATATA (SEQ ID NO: 929) |
| | | CTCTGGGTATATTCAGAAGCC (SEQ ID NO: 930) |
| rs4825889 | chrX | TCTTTGGCTCCCCTAAATTGCT (SEQ ID NO: 931) |
| | | ATGAACCGAGTTTTGATTTGGAAT (SEQ ID NO: 932) |
| rs933315 | chrX | TGGGAAGACCAAAAACAAGCAA (SEQ ID NO: 933) |
| | | TGCCCCACAGAGATTTTAGTG (SEQ ID NO: 934) |
| rs1991961 | chrX | GTACGAGATGACTGACCACCA (SEQ ID NO: 935) |
| | | CTGACTAAGTGGAGACAACCG (SEQ ID NO: 936) |
| rs1937193 | chrX | GTCTCTCATATCCAGTGTAGGT (SEQ ID NO: 937) |
| | | TGCCTCAAACTCTTTCTTCTCAT (SEQ ID NO: 938) |
| rs652 | chrX | TGTCCCACACCTACAGTGCG (SEQ ID NO: 939) |
| | | ACCTGTGCATGCATTTGTGTAC (SEQ ID NO: 940) |
| rs5964206 | chrX | TGTCCTTCATCAGAAAAAATTCTC (SEQ ID NO: 941) |
| | | CAACACGGCTCTATATCACTGA (SEQ ID NO: 942) |
| rs149910 | chrX | GAAGATTTACTCAAATAGAGTG (SEQ ID NO: 943) |
| | | TCCATTGTTCATTTCAAGGAAT (SEQ ID NO: 944) |
| rs1299087 | chrX | TTTTAGAGGGATTCAGACACAAA (SEQ ID NO: 945) |
| | | CGCTTTGCAATTGGTTGTAT (SEQ ID NO: 946) |
| rs5920670 | chrX | ACGTTGGATGTGAAGAATTCTTGGGTCTGC (SEQ ID NO: 947) |
| | | ACGTTGGATGAGGAAATAAGAGTACGCTCC (SEQ ID NO: 948) |
| rs6631828 | chrX | ACGTTGGATGGTTGAAATGGTAGTGTTCAG (SEQ ID NO: 949) |
| | | ACGTTGGATGTTTCAGACCATGCATCTAGG (SEQ ID NO: 950) |
| rs973212 | chrX | ACGTTGGATGTATTCTCTCTTAGGCACTGG (SEQ ID NO: 951) |
| | | ACGTTGGATGCTTAGTCACATCAAAGGAGC (SEQ ID NO: 952) |
| rs5933388 | chrX | ACGTTGGATGATCACCCCAAGAAGAAACCC (SEQ ID NO: 953) |
| | | ACGTTGGATGGGAGGGGTTGGAAAAATATG (SEQ ID NO: 954) |
| rs6649211 | chrX | ACGTTGGATGGAGTTAAAAATTCTCCCATCC (SEQ ID NO: 955) |
| | | ACGTTGGATGCTGTGACAGTTGGTAAGGTG (SEQ ID NO: 956) |
| rs5916197 | chrX | CAGCAGGAATTTATCTGAGCAC (SEQ ID NO: 957) |
| | | GGCACTAAGGCATACACAACA (SEQ ID NO: 958) |
| rs4898214 | chrX | GAATTTACCTTTACTGGAGAACGCT (SEQ ID NO: 959) |
| | | AGGATTCTGGGTAGTACCTCAA (SEQ ID NO: 960) |
| rs2071182 | chrX | TACAGGTAGAAGAAGGCTCCAAG (SEQ ID NO: 961) |
| | | ATCAAATCCTCAGGCTAATGCT (SEQ ID NO: 962) |
| rs4826645 | chrX | TTGATTTCAGAGTGCCCAGAGT (SEQ ID NO: 963) |
| | | CAGTGTGGCTTTGTGTGTAGGA (SEQ ID NO: 964) |
| rs35284970 | chrY | GGTGGGATGTTGTTTTTCTCTCCT (SEQ ID NO: 965) |
| | | GCATTCGACTTACTGTTGCTGAAG (SEQ ID NO: 966) |
| rs13447361 | chrY | AGCTAGTGTGAGGAACATAGCAAGA (SEQ ID NO: 967) |
| | | TATCCCTGGAAATCAATTTGTTCCCTT (SEQ ID NO: 968) |
| rs2267801 | chrY | CTGTGCAAGTTGTGTGATCTTGGT (SEQ ID NO: 969) |
| | | GAGGAACTACCTTGGGATTTGGGGTT (SEQ ID NO: 970) |
| rs9786139 | chrY | GCTTGCAGGGAGCCAAAATTGT (SEQ ID NO: 971) |
| | | ACAGAAATATCCTTTGTCCCCACAATCT (SEQ ID NO: 972) |
| rs2075181 | chrY | AGCCAACATACTCGCCAATCC (SEQ ID NO: 973) |
| | | GCCTACTAGCAGGGCTCTGGAA (SEQ ID NO: 974) |
| rs17316007 | chrY | TCAGCTTCCAGATATTACCAGCATGCA (SEQ ID NO: 975) |
| | | TAAAGAGGATGAATGGGAGGAAAAAGAG (SEQ ID NO: 976) |
| rs17269396 | chrY | CAAGGGGTCGATTGATTAAAATAACGAA (SEQ ID NO: 977) |
| | | TACTTTAGGTTCAAGGGCACATGTG (SEQ ID NO: 978) |
| rs8179021 | chrY | GCTTTCTACGGCATAGAAAGTTTGTG (SEQ ID NO: 979) |
| | | GCCACCGCTTCAGAGTTGTTCTAGG (SEQ ID NO: 980) |
| rs16980601 | chrY | TCTCGCAAATAGTAGCAGAAACCTTATT (SEQ ID NO: 981) |
| | | GCTCCTACCACATGTCAAATGTTAGCA (SEQ ID NO: 982) |
| rs9341278 | chrY | GGAAAATGTGGGCTCGTTTTAATTATATTCA (SEQ ID NO: 983) |
| | | GATCGTCAAAGAATTCATACCTGTAATTTCTG (SEQ ID NO: 984) |
| rs917759 | chrY | TCCTGCAAATCTTAGGGGAACCCA (SEQ ID NO: 985) |
| | | GCTTACAATCCCAGCACTTTGG (SEQ ID NO: 986) |
| rs17269816 | chrY | CATGGTCTCTTTGGGTGAAAGACTAC (SEQ ID NO: 987) |
| | | CCTGGAAAATGTAGACTGGTGTTACATGC (SEQ ID NO: 988) |
| rs17316543 | chrY | ACCCCATTCTTTCTCAAGTTAATTGCA (SEQ ID NO: 989) |
| | | GCACAGCATTCTCACGGACATG (SEQ ID NO: 990) |
| rs17316592 | chrY | TGGTTCACACTCCCTTCTGTGT (SEQ ID NO: 991) |
| | | ATTCAACAGTCAAGGCCAGAAAGAC (SEQ ID NO: 992) |
| rs17269928 | chrY | TGAGAAAAGATTGCTCTAATCAGCCTATT (SEQ ID NO: 993) |
| | | GCTCTTCGGAAGTGTAAAGTCATTTAGC (SEQ ID NO: 994) |
| rs17276358 | chrY | GGGAAAACATTGCTTTCACCTCTGAG (SEQ ID NO: 995) |
| | | GAAAGGTTATTACCCTTCTGCTGTCCT (SEQ ID NO: 996) |
| rs16980641 | chrY | GACAAACCCAACCAAGAAGACATCCCT (SEQ ID NO: 997) |
| | | GCTGAAATTCTAACCCACAGTACTG (SEQ ID NO: 998) |
| rs11096432 | chrY | AGTGTGGGATACAGTGAAATCAGTG (SEQ ID NO: 999) |
| | | GGGTCAAGGGCAGAAACTGACAAC (SEQ ID NO: 1000) |
| rs9786502 | chrY | GGCAAAAATAAAATTTCCCCCAACCAT (SEQ ID NO: 1001) |
| | | CATTTCTCACCATGCCCACTCA (SEQ ID NO: 1002) |
| rs2032652 | chrY | TCCTGGATTCAGCTCTCTTCCTAA (SEQ ID NO: 1003) |
| | | AGCCTCTCCTGATACTCTGTGT (SEQ ID NO: 1004) |
| rs16980426 | chrY | TGCCAGAAGAGCAAATACGGTAAT (SEQ ID NO: 1005) |
| | | TGAAAACACAAAGCTACTGGCAAGC (SEQ ID NO: 1006) |
| rs13447443 | chrY | AGAAAGGAAACGAGATAAAATATTAAACGACTT (SEQ ID NO: 1007) |
| | | TGGTATCACAATAGAAGGGGAATGATC (SEQ ID NO: 1008) |
| rs17323322 | chrY | CTTGGGCAAGTACTGTAGACCATC (SEQ ID NO: 1009) |
| | | TTGGTGCAGGCTGAATTTTCAGT (SEQ ID NO: 1010) |
| rs13447354 | chrY | CTACATGGAAATGGTTGGCTATTGAGT (SEQ ID NO: 1011) |
| | | AAAAATACAGCACAATACAGCACATTCG (SEQ ID NO: 1012) |
| rs2033003 | chrY | TTACTTAGAGGCAGGGTGTTGC (SEQ ID NO: 1013) |
| | | TAAGACATTGCACTCAGCCTCAAG (SEQ ID NO: 1014) |
| rs16980610 | chrY | TAGTTTCGGAGCTAGCCCGTTTT (SEQ ID NO: 1015) |
| | | TTGGCCACTCCAAAGAGTGAAC (SEQ ID NO: 1016) |
| rs3848982 | chrY | ACGTTGGATGTATAGCGGCATACTTGCCTC (SEQ ID NO: 1017) |
| | | ACGTTGGATGGGTTCTTGATTAGGCTAAGG (SEQ ID NO: 1018) |
| rs2075640 | chrY | AGATCATCAGCTCTGTGACTGG (SEQ ID NO: 1019) |
| | | CCATCGATTTCCAGCATTTC (SEQ ID NO: 1020) |
| rs16980711 | chrY | AATTGCCAGGTGGTGTTACAG (SEQ ID NO: 1021) |
| | | TCATGGCTGTTTTGTCTCCA (SEQ ID NO: 1022) |
| rs9786707 | chrY | TTTTCTATGTAGGGGAGTTGTGAG (SEQ ID NO: 1023) |
| | | AAGCTGCAAAATTGAGTGAGC (SEQ ID NO: 1024) |
| rs17276345 | chrY | GAGAGGAAGGAACCCTCCAG (SEQ ID NO: 1025) |
| | | ATTGCAGGAAAAGGGTGGAA (SEQ ID NO: 1026) |
| rs9786479 | chrY | TCTCACTATGATCCAGGCAATG (SEQ ID NO: 1027) |
| | | CATAAATTACCCAGATTCGGGAAG (SEQ ID NO: 1028) |
| rs9306845 | chrY | ACAATGCTCAGGCTTCCACGAGA (SEQ ID NO: 1029) |
| | | TGAGGGGCCAGACTTGACTTGTGT (SEQ ID NO: 1030) |
| rs11096433 | chrY | ATCAACTGGCCATGTGAGTG (SEQ ID NO: 1031) |
| | | TGCTGACTAACATATTACCCCAAAC (SEQ ID NO: 1032) |
| rs52812045 | chrY | ACGTTGGATGGGCCCCAAATCCAACTAATG (SEQ ID NO: 1033) |
| | | ACGTTGGATGGTGAGACCACGAAGAGACTG (SEQ ID NO: 1034) |
| rs4589047 | chrY | ACGTTGGATGACACTGACCAACTTGAGAGG (SEQ ID NO: 1035) |
| | | ACGTTGGATGAGACTTTTCTGGGACCTGAG (SEQ ID NO: 1036) |
| rs17250121 | chrY | ACGTTGGATGGAATTCAAAGAGGGAGAAAC (SEQ ID NO: 1037) |
| | | ACGTTGGATGGAGTTTCTTCCTCCACTGAC (SEQ ID NO: 1038) |
| rs2032650 | chrY | ACGTTGGATGGGGATCAAATGGAGTTCCTG (SEQ ID NO: 1039) |
| | | ACGTTGGATGTCTCCTAAGCCTACAGGTTG (SEQ ID NO: 1040) |

3. A detection reagent, comprising the detection primer pair as claimed in claim 2.

4. A detection kit, comprising the detection primer pair as claimed in claim 2.

5. An application of the detection primer pair as claimed in claim 2 in preparation of products for prenatal paternity testing.

6. A method for prenatal paternity testing, comprising:
step (1) extracting DNA, comprising: cell-free DNA of a fetus in maternal plasma, genomic DNA of a pregnant woman, and genomic DNA of a suspected father;
step (2) performing multiplex polymerase chain reaction (PCR) amplification, comprising:
performing the PCR amplification on a to-be-tested sample using the detection primer pair as claimed in claim 2 to obtain a multiplex PCR amplification product;
step (3) constructing a library, comprising: performing library construction on the multiplex PCR amplification product sequentially through two rounds of multiplex PCR reactions and two rounds of magnetic bead purification processes;
step (4) performing quality control on the library, comprising: measuring and recording a concentration and a fragment length of the library after the library is constructed to prepare for sequencing on a machine;
step (5) performing sequencing on the machine, comprising: performing sequencing on the machine by using a sequencing platform to obtain sequencing results; and
step (6) performing data analysis, comprising: comparing the sequencing results with a human reference genome for bioinformatics analysis and determination of kinship by using a software, analyzing genotyping results of each SNP locus of the fetus, the pregnant woman and the suspected father, and performing comparison to identify whether there is a kinship between the suspected father and the fetus.

7. The method for prenatal paternity testing as claimed in claim 6, wherein the genomic DNA of the pregnant woman is from blood, saliva, hair, or tissue, and the genomic DNA of the suspected father is from blood, semen, saliva, hair, or tissue.

8. The method for prenatal paternity testing as claimed in claim 7, wherein in the step (3), a first round reaction system of the two rounds of multiplex PCR reactions is 30 microliters (μL), comprising: 10 μL IGT-EM808 polymerase mixture, 5 μL primer pool, 3.5 μL enhancer buffer NB at 1 normality (IN), 2.5 μL enhancer buffer M; and
wherein in the step (3), first round reaction conditions of the two rounds of multiplex PCR reactions comprise: i) maintaining at 95° C. for 3 minutes and 30 seconds; ii) maintaining at 98° C. for 20 seconds, then maintaining at 60° C. for 8 minutes; repeating the step ii) 24 times; iii) maintaining at 72° C. for 5 minutes.

9. The method for prenatal paternity testing as claimed in claim 7, wherein in the step (3), a second round reaction system of the two rounds of multiplex PCR reactions is 30 μL, comprising:
10 μL IGT-EM808 polymerase mixture, 1 μL of 10 micromoles per liter (UM) IGT-15 Index, 1 μL of 10 UM of IGT-17 Index, 2.5 μL enhancer buffer M, and 13.5 μL PCR product mixture after first round of the two rounds of multiplex PCR reactions; and
wherein in the step (3), second round reaction conditions of the two rounds of multiplex PCR reactions comprise: i) maintaining at 95° C. for 3 minutes and 30 seconds; ii) maintaining at 98° C. for 2 minutes, then maintaining at 58° C. for 1 minute, then maintaining at 72° C. for 30 seconds; repeating the step ii) 9 times; iii) maintaining at 72° C. for 5 minutes.
